(19) United States
(12) Patent Application Publication
(10) Pub. No.: US 2022/0220466 A1
(43) Pub. Date: Jul. 14, 2022

(54) CATALYTICALLY INACTIVE BOTULINUM NEUROTOXIN-LIKE TOXINS AND USES THEREOF

(71) Applicant: Children's Medical Center Corporation, Boston, MA (US)

(72) Inventors: Min Dong, Weatogue, CT (US); Shin-Ichiro Miyashita, Brookline, MA (US)

(73) Assignee: Children's Medical Center Corporation, Boston, MA (US)

(21) Appl. No.: 17/603,978

(22) PCT Filed: Apr. 17, 2020
(86) PCT No.: PCT/US2020/028742

§ 371 Date: Oct. 15, 2021

Related U.S. Application Data
(60) Provisional application No. 62/835,151, filed on Apr. 17, 2019.

(51) Int. Cl. C12N 9/52; C07K 14/33

(52) U.S. Cl. CPC C12N 9/52; A61K 38/00; C12Y 304/24069; C07K 14/33

(57) ABSTRACT

Provided herein, in aspects, are catalytically inactive BoNT-like toxins from Clostridium botulinum, serotype X (BoNT/NX) from Enterococcus faecium (BoNT/En) or from Paraclostridium bifermentans (BoNT/PMP1) and their use as delivery vehicles to deliver an agent (e.g., a therapeutic agent or a diagnostic agent) to a cell. Methods of treating a disease (e.g., botulism) are also provided.

### RELATED APPLICATIONS

This application claims the benefit under 35 U.S.C. § 119(e) of U.S. Provisional Application Ser. No. 62/835,151, entitled “CATALYTICALLY INACTIVE BOTULINUM NEUROTOXIN-LIKE TOXINS AND USES THEREOF” filed on Apr. 17, 2019, the entire contents of which is incorporated herein by reference.

### BACKGROUND

Many key therapeutic targets are in the cytosol of cells. To reach them, therapeutic and diagnostic agents have to be able to cross cell membranes, which is a formidable challenge for protein/peptide-based therapeutics and membrane impermeable small molecule therapeutics. Furthermore, the ability to specifically target a cell type such as neurons is needed for treating many neuronal disorders. Thus, there is a high demand for a delivery system that can efficiently and safely deliver membrane impermeable therapeutics into cells such as neurons. Botulinum neurotoxins (BoNTs, including eight serotypes BoNT/A-H) are a family of bacterial toxins that target motor nerve terminals with extreme specificity. These toxins are composed of three functional domains: the receptor-binding domain that is responsible for recognizing neurons, the translocation domain that translocates the toxin enzymatic protease domain across cell membranes into the cytosol of cells. The protease domain of BoNTs then cleaves key cellular proteins, which is the basis for the toxicity of BoNTs. Therefore, BoNTs naturally possess the ability to target and deliver a protein cargo (its own protease domain) into the cytosol of neurons, and can be potentially utilized for delivery of therapeutics into neurons. However, it was found that even BoNTs containing a catalytically inactive protease domain still maintained a level of toxicity in vivo and caused paralysis and death in animal models. This is a major barrier for the use of BoNTs as a delivery tool and thus an effective and safe delivery tool targeting the cytosol of cells (e.g. neurons) is still lacking.

### SUMMARY

The present disclosure, in some aspects, are compositions comprising catalytically inactive botulinum neurotoxins-like toxins from Clostridium botulinum, serotype X (BoNT/X), from Enterococcus faecium (BoNT/En) or from Paraclostridium bifermentans (BoNT/PMP1). BoNT/X, BoNT/En and BoNT/PMP1 share the overall domain arrangement and functionality as the eight traditional BoNTs (BoNT/A-H), but contain high levels of sequence variations from other BoNTs. Therefore, BoNT/X, BoNT/En and BoNT/PMP1 are considered a distinct branch within the BoNT super family. Specifically, BoNT/X, BoNT/En and BoNT/PMP1 contain the protease domain and the translocation domain like other BoNTs, but their receptor-binding domain lacks the ability to target mammalian neurons. The receptor-binding domain of BoNT/X, BoNT/En and BoNT/PMP1 can be replaced with the receptor-binding domain of a traditional BoNTs, which results in chimeric toxins that can target mammalian neurons.

Accordingly, in some aspects, the present disclosure provides catalytically inactive neurotoxins (BoNTs) from Clostridium botulinum, serotype X (BoNT/X) comprising an inactive protease domain and a translocation domain. In some embodiments, the inactive protease domain comprises one or more substitution mutation(s) in a position corresponding to R360, Y363, H227, E228, or H231 in SEQ ID NO: 1. In some embodiments, the inactive protease domain comprises amino acid substitutions corresponding to E228Q, R360A, and Y363F in SEQ ID NO: 1. In some embodiments, the catalytically inactive BoNT comprises the amino acid sequence of any one of SEQ ID NO: 3 or SEQ ID NO: 21.

Other aspects of the present disclosure provide catalytically inactive neurotoxins from Enterococcus faecium (BoNT/En) comprising an inactive protease domain and a translocation domain.

In some embodiments, the inactive protease domain comprises one or more substitution mutation(s) in a position corresponding to H225, E226, H229, R364, or Y367 in SEQ ID NO: 2. In some embodiments, the inactive protease domain comprises amino acid substitutions corresponding to E226Q, R364A, and Y367F in SEQ ID NO: 2. In some embodiments, the catalytically inactive BoNT comprises the amino acid sequence of any one of SEQ ID NO: 4 or SEQ ID NO: 22.

Further provided herein are catalytically inactive neurotoxins from Paraclostridium bifermentans (BoNT/PMP1) comprising an inactive protease domain and a translocation domain. In some embodiments, the inactive protease domain comprises one or more substitution mutation(s) in a position corresponding to H208, E209, H212, R344, or Y347 in SEQ ID NO: 85. In some embodiments, the inactive protease domain comprises amino acid substitutions corresponding to E209Q, R344A, and Y347F in SEQ ID NO: 85. In some embodiments, the catalytically inactive BoNT comprises the amino acid sequence of any one of SEQ ID NO: 86 or SEQ ID NO: 95.

Other aspects of the present disclosure provide chimeric Clostridium botulinum neurotoxins (BoNTs) comprising:

(a) a light chain comprising an inactive protease domain,

(b) a heavy chain comprising:
(i) a translocation domain, and
(ii) a receptor binding domain,
   wherein (a) and (b)(i) are from a neurotoxin in Clostridium botulinum, serotype X, Enterococcus faecium or Paraclostridium bifermentans, and wherein (b)(ii) is from a BoNT in Clostridium botulinum, serotype A, B, C, D, E, F, G or H.

In some embodiments, the chimeric BoNT comprises a modified linker between the light chain and the heavy chain. In some embodiments, the modified linker comprises a protease cleavage site.

In some embodiments, (a) and (b)(i) are from a neurotoxin in Clostridium botulinum, serotype X. In some embodiments, the inactive protease domain comprises one or more substitution mutation(s) in a position corresponding to R360, Y363, H227, E228, or H231 in SEQ ID NO: 1. In some embodiments, the inactive protease domain comprises amino acid substitutions corresponding to E228Q, R360A, and Y363F in SEQ ID NO: 1. In some embodiments, b(ii) is from a BoNT in Clostridium botulinum, serotype A (BoNT/A), serotype B (BoNT/B), serotype C (BoNT/C), serotype D (BoNT/D), serotype E (BoNT/E), serotype F (BoNT/F), serotype G (BoNT/G), or serotype H (BoNT/H). In some embodiments, the chimeric BoNT comprises an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 5-12 and 23-30, and comprises amino acid substitutions corresponding to E228Q, R360A, and Y363F in SEQ ID NO: 1. In some embodiments, the chimeric BoNT comprises the amino acid sequence of any one of SEQ ID NOs: 5-12 and 23-30. In some embodiments, the chimeric BoNT consists of the amino acid sequence of any one of SEQ ID NOs: 5-12 and 23-30.

In some embodiments, (a) and (b)(i) are from a neurotoxin in Enterococcus faecium. In some embodiments, the inactive protease domain comprises one or more substitution mutation(s) in a position corresponding to H225, E226, H229, R364, or Y367 in SEQ ID NO: 2. In some embodiments, the inactive protease domain comprises amino acid substitutions corresponding to E226Q, R364A, or Y367F in SEQ ID NO: 2. In some embodiments, b(ii) is from BoNT in Clostridium botulinum, serotype A (BoNT/A), serotype B (BoNT/B), serotype C (BoNT/C), serotype D (BoNT/D), serotype E (BoNT/E), serotype F (BoNT/F), serotype G (BoNT/G), or serotype H (BoNT/H). In some embodiments, the chimeric BoNT comprises an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 13-20 and 31-38, and comprises amino acid substitutions corresponding to E226Q, R364A, and Y367F in SEQ ID NO: 2. In some embodiments, the chimeric BoNT comprises the amino acid sequence of any one of SEQ ID NOs: 13-20 and 31-38. In some embodiments, the chimeric BoNT consists of the amino acid sequence of any one of SEQ ID NOs: 13-20 and 31-38.

In some embodiments, (a) and the (b)(i) are from a neurotoxin in Paraclostridium bifermentans. In some embodiments, the inactive protease domain comprises one or more substitution mutation(s) in a position corresponding to H208, E209, H212, R344, or Y347 in SEQ ID NO: 85. In some embodiments, the inactive protease domain comprises amino acid substitutions corresponding to E209Q, R344A, and Y347F in SEQ ID NO: 85.

In some embodiments, b(ii) is from BoNT in Clostridium botulinum, serotype A (BoNT/A), serotype B (BoNT/B), serotype C (BoNT/C), serotype D (BoNT/D), serotype E (BoNT/E), serotype F (BoNT/F), serotype G (BoNT/G), or serotype H (BoNT/H). In some embodiments, the chimeric BoNT comprises an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 87-94 and 96-103, and comprises amino acid substitutions corresponding to E209Q, R344A, and Y347F in SEQ ID NO: 85.

In some embodiments, the chimeric BoNT comprises the amino acid sequence of any one of SEQ ID NOs: 87-94 and 96-103. In some embodiments, the chimeric BoNT consists of the amino acid sequence of any one of SEQ ID NOs: 87-94 and 96-103.

In some embodiments, the light chain and the heavy chain are linked by a di-sulfide bond. Other aspects of the present disclosure provide nucleic acids encoding any one of the catalytically inactive BoNT/X, any one of the catalytically inactive BoNT/EN, any one of the catalytically inactive BoNT/PMP1, or any one of the chimeric BoNT described herein. Vectors comprising such nucleic acids, and cells comprising any one of the catalytically inactive BoNT/X, any one of the catalytically inactive BoNT/EN, any one of the catalytically inactive BoNT/PMP1, or any one of the chimeric BoNT, the nucleic acid, or the vector described herein are also provided.

Other aspects of the present disclosure provide compositions comprises any one of the catalytically inactive BoNT/X, any one of the catalytically inactive BoNT/EN, any one of the catalytically inactive BoNT/PMP1, or any one of the chimeric BoNT. In some embodiments, the composition is a pharmaceutical composition. In some embodiments, the composition further comprises a pharmaceutically acceptable carrier.

Further provided herein are uses of any one of the catalytically inactive BoNT/X, any one of the catalytically inactive BoNT/EN, any one of the catalytically inactive BoNT/PMP1, or any one of the chimeric BoNT described herein as a delivery vehicle.

Accordingly, some aspects of the present disclosure provide a complex comprising any one of the catalytically inactive BoNT/X, any one of the catalytically inactive BoNT/EN, any one of the catalytically inactive BoNT/PMP1, or any one of the chimeric BoNTs associated with an agent.

In some embodiments, the agent is associate with the catalytically inactive BoNT/X, the catalytically inactive BoNT/En, the catalytically inactive BoNT/PMP1, or the chimeric BoNT non-covalently. In some embodiments, the agent is fused to the catalytically inactive BoNT/X, the catalytically inactive BoNT/EN, the catalytically inactive BoNT/PMP1, or the chimeric BoNT via a covalent bond. In some embodiments, the agent is associated with the light chain or the heavy chain of the catalytically inactive BoNT/X, the catalytically inactive BoNT/En, the catalytically inactive BoNT/PMP1, or the chimeric BoNT.

In some embodiments, the complex comprises a chimeric BoNT associated with an agent, wherein the BoNT comprises:

(a) a light chain comprising an inactive protease domain,

(b) a heavy chain comprising:
(i) a translocation domain, and
(ii) a receptor binding domain,

wherein (a) and (b)(i) are from a neurotoxin in Clostridium botulinum, serotype X, and wherein (b)(ii) is from a BoNT in Clostridium botulinum, serotype A, B, C, D, E, F, G, or H,

and wherein the light chain and the heavy chain are linked via a disulfide bond.

In some embodiments, the complex comprises a chimeric BoNT associated with an agent, wherein the BoNT comprises:

(a) a light chain comprising an inactive protease domain,

(b) a heavy chain comprising:
(i) a translocation domain, and
(ii) a receptor binding domain,

wherein (a) and (b)(i) are from a neurotoxin in Enterococcus faecium, and wherein (b)(ii) is from a BoNT in Clostridium botulinum, serotype A, B, C, D, E, F, G, or H,

and wherein the light chain and the heavy chain are linked via a disulfide bond.

In some embodiments, the complex comprises a chimeric BoNT associated with an agent, wherein the BoNT comprises:
(a) a light chain comprising an inactive protease domain,
(b) a heavy chain comprising:
(i) a translocation domain, and
(ii) a receptor binding domain,

wherein (a) and (b)(i) are from a neurotoxin in Enterococcus faecium, and wherein (b)(ii) is from a BoNT in Paraclostridium bifermentans, serotype A, B, C, D, E, F, G, or H, and wherein the light chain and the heavy chain are linked via a disulfide bond. In some embodiments, in any one of the complexes described herein, the receptor binding domain is from a BoNT in Clostridium botulinum, serotype A. In some embodiments, the agent is fused to the N-terminus of the light chain. In some embodiments, the agent is a nucleic acid, a peptide/protein, or a small molecule. In some embodiments, the agent is a diagnostic agent. In some embodiments, the agent is a therapeutic agent.

In some embodiments, the therapeutic agent is an antibody. In some embodiments, the antibody is a single-domain antibody (also known as nanobody or VHH). In some embodiments, the antibody is an antibody against a BoNT light chain. In some embodiments, the antibody comprises the amino acid sequence of any one of SEQ ID NO: 57, SEQ ID NO: 58, SEQ ID NO: 67, SEQ ID NO: 113, and SEQ ID NO: 114. In some embodiments, the therapeutic agent is a fusion protein comprising two VHHs. In some embodiments, the fusion protein comprises a VHH against BoNT/A light chain fused to a VHH against BoNT/B light chain. In some embodiments, the complex comprises an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 65, 66, 71, 75, 76, 119, 128, 129, 133, 134, 137, 138, and 142-150. In some embodiments, the complex comprises an amino acid sequence of any one of SEQ ID NOs: 65, 66, 71, 75, 76, 119, 128, 129, 133, 134, 137, 138, and 142-150.

Other aspects of the present disclosure provide compositions comprising any one of the complexes described herein. In some embodiments, the composition is a pharmaceutical composition. In some embodiments, the composition further comprising a pharmaceutically acceptable carrier.

Uses of any one of the complexes described herein or compositions comprising such are also provided. In some embodiments, the complexes used for delivering the agent to a cell. In some embodiments, the complex is used for treating or diagnosing a disease.

Accordingly, some aspects of the present disclosure provide a method of delivering an agent to a cell, comprising contacting the cell with any one of the complexes or compositions described herein. In some embodiments, the cell is in vitro, in vivo, or ex vivo. In some embodiments, the cell is a neuron.

Other aspects of the present disclosure provide methods of diagnosing a disease, the method comprising administering to a subject in need thereof an effective amount of any one of the complexes or any one of the compositions described herein, wherein the agent is a diagnostic agent. Further provided herein are methods of treating a disease, the method comprising administering to a subject in need thereof an effective amount of any one of the complexes or any one of the compositions described herein, wherein the agent is a therapeutic agent. In some embodiments, the disease is botulism. In some embodiments, the subject has previously been administered a BoNT or been in contact with a BoNT. In some embodiments, the therapeutic agent neutralizes the BoNT. In some embodiments, the complex is administered by injection. In some embodiments, the subject is a human. In some embodiments, the subject is a rodent. In some embodiments, the rodent is a mouse or a rat.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. The patent or application file contains at least one drawing executed in color. Copies of this patent or patent application publication with color drawing(s) will be provided by the Office upon request and payment of the necessary fee. In the drawings:

FIGS. 1A-1B show the overall design of VHH fused with inactive LC-H_{N} of a BoNT-like toxin, and a Hc of a BoNT. (FIG. 1A) Schematic model showing the design of fusion proteins. The Hc/A is used as an example to generate VHH-^{ci}BoNT/XA, VHH-^{ci}BoNT/EnA, and VHH-^{ci}BoNT/PmA. TrxA, Thioredoxin A; VHH, a VHH against the LC of BoNT/A (also known as A8); ^{ci}LC, catalytically inactive light chain; HC, heavy chain; Hn, translocation domain; Hc, binding domain. The Hc/A can be replaced with the Hc from other BoNTs (BoNT/B-H). The three amino acid in the active site of LC/X mutated to abolish the metalloprotease activity (E228Q, R360A, and Y363F) were indicated as an example for generating catelytically inactive form (^{ci}LC/X, ^{ci}LC/En, and ^{ci}LC/Pm). The thrombin cleavage sites (*) were introduced for TrxA and His tag removal. A designed long linker containing a thrombin site was introduced to replace the original linker between ^{ci}LC and Hn domain. (FIG. 1B) Thrombin treated-VHH-^{ci}BoNT/XA was subjected to SDS-PAGE in the presence or absence of DTT, showing that the LC and HC are separated after thrombin treatment in the presence of DTT.

FIGS. 2A-2B show neutralization activity of VHH-^{ci}BoNT/XA on cultured neuron. (FIG. 2A) Schematic experiment of BoNT/A neutralization by VHH-^{ci}BoNT in cultured neuron. Neurons were exposed to 20 pM of BoNT/A for 12 h (0.5 days). The residual BoNT/A in medium were washed with culture medium. The intoxicated-neuron were further incubated with VHH-^{ci}BoNT/XA for indicated days. (FIG. 2B) Neutralization of BoNT/A in a neuron at 1, 3 and 5 days. Immunoblot analysis was carried out to detect SNAP-25. Actin was used as a loading control.

FIGS. 3A-3E show neutralization activity of VHH-^{ci}BoNT/XA via IM injection in vivo in mouse. (FIG. 3A) Muscle paralysis patterns following BoNT/A IM injection. The corresponding DAS score is listed on each picture. (FIG. 3B) Schematic experiment of BoNT/A-neutralization by VHH-^{ci}BoNT/XA in vivo. 5.8 pg of BoNT/A were injected in mouse hind limb muscle. After 18 hours, the mice showed paralysis (DAS score 2-3) and VHH-^{ci}BoNT/XA were injected in the same muscle and DAS score were recorded. (FIG. 3C) VHH-^{ci}BoNT/XA treated paralysis on mouse gastrocnemius muscle. 0.6 μg of VHH-^{ci}BoNT/XA or ^{ci}BoNT/XA were injected to BoNT/A-injected gastrocnemius muscle. (FIG. 3D) Time course of muscle paralysis recovery by VHH-^{ci}BoNT/XA (N=8). (FIG. 3E) 5.8 pg of BoNT/A were injected in mouse hind limb muscle. After 3 or 6 days, 0.6 ug of VHH-^{ci}BoNT/XA were injected in the same muscle and DAS score were recorded. VHH-^{ci}BoNT/XA treatment stopped muscle paralysis within 24 h.

FIGS. 4A-4D show IP-injected VHH-^{ci}BoNT/XA neutralized the BoNT/A induced paralysis at the leg. (FIG. 4A) 5.8 pg of BoNT/A were injected in mouse hind limb muscle. After 18 h, the mice showed paralysis (DAS score 2-3) and 600, 60, 6 ug of VHH-^{ci}BoNT/XA were administrated by IP injection. (FIG. 4B) BoNT/A were injected as described (FIG. 4A). 6 ug of VHH-^{ci}BoNT/C were administrated by IP injection. VHH-^{ci}BoNT/C serves as a control, showing that VHH-^{ci}BoNT/XA is superior in inhibiting BoNT/A-induced paralysis. (FIG. 4C) 6 ug of VHH-^{ci}BoNT/XA or /C were administrated by IP injected at 18 and 96 h after BoNT/A injection. (FIG. 4D) 6 ug of VHH-^{ci}BoNT/XA were administrated by IP injection per day.

FIGS. 5A-5B show the neutralization of BoNT/A by VHH-^{ci}BoNT/XA in a systematic mouse lethality model. (FIG. 5A) 20 pg of BoNT/A were administrated to mouse by IP. After 10 hours, the mice showed botulism phenotype were randomly separated into four groups (group 1; vehicle, 0.2% gelatin-saline, group 2; VHH and ^{ci}BoNT/XA mixture, group 3; 6 ug of VHH-^{ci}BoNT/XA, group 4; 0.6 ug of VHH-^{ci}BoNT/XA). VHH-^{ci}BoNT/XA were administrated to the mouse by IP and monitored for survival to 5 days. (FIG. 5B) A 90% survival was observed in groups treated with the 6 ug of VHH-^{ci}BoNT/XA compared to 0% survival in vehicle and the mixture of VHH and ^{ci}BoNT/XA.

FIGS. 6A-6C show the neutralization of BoNT/A and BoNT/B by a double VHH B8-B10-^{ci}BoNT/XA in DAS assay. (FIG. 6A) Schematic drawing of the VHH-B8-B10-^{ci}BoNT/XA constructs. B8 targets LC/A, while B10 targets LC/B. BoNT/A (5.8 pg) or BoNT/B (3.5 pg) were injected in mouse hind limb muscle. After 18 hours, 6 μg of VHH B8-B10-^{ci}BoNT/XA were injected in the same muscle and DAS score were recorded. (FIG. 6B) Representative image showing that VHH-B8-B10-^{ci}BoNT/XA treatment shortened the duration of muscle paralysis induced by BoNT/A (left panel) and BoNT/B (right panel). (FIG. 6C) DAS scores over time is recorded. VHH B8-B10-^{ci}BoNT/XA was effective in shortening the duration of paralysis induced by BoNT/A (left panel) and BoNT/B (right panel). *VHH B8-B10 is also referred to as “VHH A8-J10” herein.

FIGS. 7A-7E show a chimeric inactive toxin ^{ci}BoNT/XA delivered the fused nanobody against LC/A into neurons. (FIG. 7A) Schematic drawing of the A8-^{ci}BoNT/XA fusion protein. The LCH_{N}/X is fused with a BoNT-Hc (Hc/A, Hc/C, or Hc/D). LC/X is deactivated by three point-mutations. The linker region between LC/X and H_{N}/X is modified to include a thrombin cleavage site. A8: VHH-ALc-B8, a nanobody that neutralizes LC/A. (FIG. 7B) A schematic illustration of delivering nanobodies via fusion with ^{ci}BoNT/XA to neutralize BoNT-LC in neurons. LC/A cleaves SNAP-25 in neurons, thus blocking fusion of synaptic vesicles to plasma membranes, which is essential for neurotransmitter release. Nanobodies such as A8 cannot enter neurons by themselves. When fused with ^{ci}BoNT/XA, A8-^{ci}BoNT/XA targets and enters neurons via receptor-mediated endocytosis, followed with translocation of A8-^{ci}LC/X into the cytosol. A8-^{ci}LC/X then binds to and inhibits LC/A in neurons. (FIG. 7C) Cultured neurons were exposed to A8-^{ci}BoNT/XA for 12 h with or without bafilomycin A1. Neurons were washed and neuron lysates were harvested for immunoblot analysis under non-reducing conditions to detect A8 using a goat anti-llama antibody. Successful translocation of A8-^{ci}LC/X into the cytosol reduced the disulfide bond connecting it to the H_{N}-Hc, thus generating the A8-^{ci}LC/X band. A8-^{ci}BoNT/XA that did not translocate into the cytosol remained as a full-length protein under non-reducing conditions. Bafilomycin A1 inhibits acidification of endosomes, thus blocking translocation of A8-^{ci}LC/X. Bafilomycin A1 treatment did not affect binding of A8-^{ci}BoNT/XA to neurons, but reduced A8-^{ci}LC/X. Cell lysates were also analyzed in the presence of DTT, which reduces the inter-chain disulfide bond between A8-^{ci}LC and H_{N}-H_{C}, serving as a loading control. One of three independent experiments is shown. (FIG. 7D) Active forms of BoNT/XA and A8-BoNT/XA were generated via sortase-mediated ligation as described in FIGS. 13A and 13B. Cultured neurons were exposed to these toxins and cleavage of VAMP2 was analyzed by immunoblot. SNAP-25 served as a loading control. Representative blots (one of three independent experiments) and quantification of dose-dependent VAMP2 cleavage are shown. The efficacy of VAMP2 cleavage by A8-BoNT/XA is ˜7.4-fold lower than BoNT/XA. Date were shown as mean±s.e.m. (FIG. 7E) Cultured rat cortical neurons were first exposed to BoNT/A (20 pM, 12 h), washed, further incubated in toxin-free medium for 24 h, and then exposed to the indicated concentrations of either A8-^{ci}BoNT/XA or the control mixture of A8 and ^{ci}BoNT/XA proteins for 48 h. Cell lysates were analyzed by immunoblot to detect SNAP-25, Syntaxin 1, and VAMP2. Actin served as a loading control. Representative blots (one of three independent experiments) and quantification of SNAP-25 cleavage are shown. Cleavage of SNAP-25 by BoNT/A generates a smaller band marked by an arrow. A8-^{ci}BoNT/XA reduced cleavage of SNAP-25 by BoNT/A in neurons, while the control A8 and ^{ci}BoNT/XA mixture did not affect cleavage of SNAP-25. Data were analyzed by one-way ANOVA with Dunnett post-hoc tests, *P=0.0234 and **P=0.0055.

FIGS. 8A-8F show post-exposure treatment of BoNT/A-induced local paralysis using A8-^{ci}BoNT/XA. (FIG. 8A) Schematic illustration of the DAS assay and representative images showing the degrees of toe spreading. Score “0” represents no paralysis and score “4” represents the most severe paralysis. (FIG. 8B) Intramuscular (IM) injection of BoNT/A (6 pg) in the mouse hind leg induced persistent local paralysis that lasted 30-40 days. The indicated amounts of A8-^{ci}BoNT/XA were injected into the same leg muscle 18 h after the initial injection of BoNT/A. DAS scores were recorded and plotted over time. The DAS score in the first five days were enlarged (right-upper panel). Injection of A8 or ^{ci}BoNT/XA alone served as controls (right-lower panel). Vehicle, n=22; A8-^{ci}BoNT/XA at 6,000, 200, 60, and 20 ng, n=8; A8-^{ci}BoNT/XA at 600 ng, n=16. (FIG. 8C) A8-^{ci}BoNT/XA (600 ng) were injected via IM into the leg muscle 3 days (circle) or 6 days (triangle) after the initial injection of BoNT/A (6 pg) to the same muscle, and the DAS scores were plotted over time (vehicle, n=16; A8-^{ci}BoNT/XA at day 3, n=14; A8-^{ci}BoNT/XA at day 6, n=8). (FIG. 8D) A8-^{ci}BoNT/XA (600 ng and 60 ng) were injected via IM into the leg muscle 3 days after the initial injection of BoNT/A (6 pg). DAS scores were recorded every 3 h for 24 h (n=8 per group). The differences in DAS scores at 6 h were determined to be significant by two-way ANOVA with Dunnett post-hoc tests, ***P<0.0001. (FIG. 8E) A8-^{ci}BoNT/XA at the indicated doses was administered via IP injection 18 h after the initial injection of BoNT/A (6 pg) to the leg muscle. DAS scores were plotted over time (n=8 per group). (FIG. 8F) A8-^{ci}BoNT/XA (6 μg per mouse) was injected either twice (solid triangle, 18 and 42 h) after the initial injection of BoNT/A (6 pg) to the leg muscle, or once per day for seven days (circle, with the first one 18 h after the initial injection of BoNT/A). Vehicle, n=19; A8-^{ci}BoNT/XA, n=8.

FIGS. 9A-9D show post-exposure treatment of systemic toxicity of BoNT/A using A8-^{ci}BoNT/XA. (FIG. 9A) A systemic toxicity model of botulism and post-exposure treatment using A8-^{ci}BoNT/XA. Lethal dose of BoNT/A (19.5 pg) was first injected into mice via IP to induce systemic botulism. A8-^{ci}BoNT/XA or the control mixture of A8 and ^{ci}BoNT/XA proteins were injected via IP 9 h later when botulism symptoms had developed. (FIG. 9B) Experiments were carried out as described in panel A with the indicated concentrations of A8-^{ci}BoNT/X and the control A8/^{ci}BoNT/X mixture (vehicle, n=14; A8-^{ci}BoNT/XA at 30 and 0.6 μg, n=9; A8-^{ci}BoNT/XA at 6 μg, n=10; A8 (3 μg)/^{ci}BoNT/XA (27 μg), n=9; A8 (0.6 μg)/^{ci}BoNT/XA (5.4 μg), n=10). Survival rates are plotted (statistical analysis was conducted by log-rank test. ****P<0.0001). (FIG. 9C) Violin plots of clinical scores of each mouse. The humane endpoint was set as clinical scores above 5. (FIG. 9D) The body weight changes of control mice ((−)BoNT/A) and the indicated experimental groups are plotted.

FIGS. 10A-10I show delivery of two nanobodies using ^{ci}BoNT/XA for post-exposure treatment of BoNT/A and BoNT/B intoxication. (FIG. 10A) Schematic drawing of ^{ci}BoNT/XA with two nanobodies (A8 against LC/A and J10 against LC/B) fused to its N-terminus. The fusion protein is termed A8-J10-^{ci}BoNT/XA. (FIG. 10B) DAS assays were carried out with BoNT/A (6 pg). The indicated concentrations of A8-J10-^{ci}BoNT/XA were injected into the same leg muscle 18 h later and DAS scores were plotted over time. Mixtures of A8-J10 and ^{ci}BoNT/XA proteins did not affect the duration of paralysis (B, vehicle, n=9; A8-J10-^{ci}BoNT/XA of each group and A8-J10 (5 μg)/^{ci}BoNT/XA (27 μg), n=8). (FIG. 10C) DAS assays were carried out with BoNT/B (3.5 pg). The indicated concentrations of A8-J10-^{ci}BoNT/XA were injected into the same leg muscle 18 h later and DAS scores were plotted over time. Mixtures of A8-J10 and ^{ci}BoNT/XA proteins did not affect the duration of paralysis (C, vehicle, n=27; A8-J10-^{ci}BoNT/XA at 30, 6, and 0.06 μg, n=10; at 0.6 μg, n=9). (FIG. 10D) Lethal doses of BoNT/A (19.5 pg) were injected via IP administration into mice to induce systemic botulism. The indicated concentrations of A8-J10-^{ci}BoNT/XA were injected via IP 9 h after injection of BoNT/A. Mixtures of A8-J10 and ^{ci}BoNT/XA served as controls. Survival rates are shown (vehicle, n=12; other groups, n=8). ****P<0.0001, **P=0.00022 (log-rank test). (FIG. 10E) Lethal doses of BoNT/A (19.5 pg) were injected via IP administration into mice to induce systemic botulism. The indicated concentrations of A8-J10-^{ci}BoNT/XA were injected via IP 9 h after injection of BoNT/A. Mixtures of A8-J10 and ^{ci}BoNT/XA served as controls. Clinical scores are shown (vehicle, n=12; other groups, n=8). ****P<0.0001, **P=0.00022 (log-rank test). (FIG. 10F) Lethal doses of BoNT/A (19.5 pg) were injected via IP administration into mice to induce systemic botulism. The indicated concentrations of A8-J10-^{ci}BoNT/XA were injected via IP 9 h after injection of BoNT/A. Mixtures of A8-J10 and ^{ci}BoNT/XA served as controls. Body weight changes are shown (vehicle, n=12; other groups, n=8). ****P<0.0001, **P=0.00022 (log-rank test). (FIG. 10G) Lethal doses of BoNT/B (10 pg) were injected via IP administration into mice to induce systemic botulism. The indicated concentrations of A8-J10-^{ci}BoNT/XA were injected via IP 9 h after injection of BoNT/B. Survival rates are shown (vehicle, n=10; A8-J10-^{ci}BoNT/XA at 65 μg, n=8; at 32.5 μg, n=11; A8-J10 (12 μg)/^{ci}BoNT/XA (53 μg), n=8; A8-J10 (6 μg)/^{ci}BoNT/XA (26.5 μg), n=9). ****P<0.0001 (log-rank test). (FIG. 10H) Lethal doses of BoNT/B (10 pg) were injected via IP administration into mice to induce systemic botulism. The indicated concentrations of A8-J10-^{ci}BoNT/XA were injected via IP 9 h after injection of BoNT/B. Clinical scores are shown (vehicle, n=10; A8-J10-^{ci}BoNT/XA at 65 μg, n=8; at 32.5 μg, n=11; A8-J10 (12 μg)/^{ci}BoNT/XA (53 μg), n=8; A8-J10 (6 μg)/^{ci}BoNT/XA (26.5 μg), n=9). ****P<0.0001 (log-rank test). (FIG. 10I) Lethal doses of BoNT/B (10 pg) were injected via IP administration into mice to induce systemic botulism. The indicated concentrations of A8-J10-^{ci}BoNT/XA were injected via IP 9 h after injection of BoNT/B. Body weight changes are shown (vehicle, n=10; A8-J10-^{ci}BoNT/XA at 65 μg, n=8; at 32.5 μg, n=11; A8-J10 (12 μg)/^{ci}BoNT/XA (53 μg), n=8; A8-J10 (6 μg)/^{ci}BoNT/XA (26.5 μg), n=9). ****P<0.0001 (log-rank test).

FIGS. 11A-11F show production and Characterization of A8-^{ci}BoNT/XA, XC, and XD. (FIG. 11A) Schematic drawing of A8-^{ci}BoNT/XC and XD fusion proteins. (FIG. 11B) ^{ci}BoNT/XA, A8-^{ci}BoNT/XA, XC, and XD were generated as described in FIG. 7A. They were expressed and purified as His6-tagged proteins in E. coli, then activated by thrombin, which cleaves the linker region between the LC and H_{N}. Activated proteins were analyzed on SDS-PAGE gels with or without DTT, which reduces the disulfide bond connecting the LC and H_{N}. The activated proteins ran as a single band without DTT and were converted to two bands with DTT. (FIG. 11C) LC/A was incubated with rat brain detergent extracts (BDE) in the presence of A8 alone or A8-^{ci}BoNT/XA. Cleavage of SNAP-25 by LC/A was analyzed by immunoblot. A8-^{ci}BoNT/XA and A8 alone showed similar capability of neutralizing LC/A in vitro. One of two independent experiments is shown. (FIG. 11D) Cultured rat cortical neurons were exposed to BoNT/A (20 pM, 12 h), washed, and further incubated in toxin-free medium for 24 h. Neurons were then exposed to the indicated concentrations of A8-^{ci}BoNT/XA, XC, and XD for 48 h. Cell lysates were collected and analyzed by immunoblot to detect SNAP-25, Syntaxin 1, and VAMP2. Actin served as a loading control. A8-^{ci}BoNT/XA, XC, and XD reduced SNAP-25 cleavage in neurons. One of two independent experiments is shown. (FIG. 11E) DAS assays were carried out with BoNT/A (6 pg). The indicated concentrations of A8-^{ci}BoNT/XC were injected into the same muscle 18 h later. DAS scores were recorded and plotted over time. (n=8 per group). (FIG. 11F) DAS assays were carried out with BoNT/A (6 pg). The indicated concentrations of A8-^{ci}BoNT/XD were injected into the same muscle 18 h later. DAS scores were recorded and plotted over time. (vehicle and A8-^{ci}BoNT/XD at 60 μg, n=8; A8-^{ci}BoNT/XA at 6 μg; n=3).

FIGS. 12A-12B show production of ^{ci}BoNT/C and A8-^{ci}BoNT/C. (FIG. 12A) Schematic drawing of A8-^{ci}BoNT/C fusion protein. (FIG. 12B) ^{ci}BoNT/C and A8-^{ci}BoNT/C were expressed and purified as His6-tagged proteins in E. coli, activated by thrombin, and analyzed by SDS-PAGE gels with or without DTT.

FIGS. 13A-13D show generating active BoNT/XA, A8-BoNT/XA, and A8-J10-BoNT/XA using sortase-mediated ligation. (FIG. 13A) Schematic drawing of sortase-mediated ligation to generate BoNT/XA, A8-BoNT/XA, and A8-J10-BoNT/XA containing the active form of LC/X. (FIG. 13B) The active form of BoNT/XA and A8-BoNT/XA were generated via sortase-mediated ligation and analyzed on SDS-PAGE gels. One of three independent experiments is shown. (FIG. 13C) The indicated concentrations of LCH_{N}/X, A8-LCH_{N}/X, and A8-J10-LCH_{N}/X were activated with thrombin and then incubated with recombinantly purified GST-tagged VAMP2 in the presence of DTT. Cleavage of VAMP2 was analyzed by SDS-PAGE gels and Coomassie blue staining. Fusion with nanobodies did not affect cleavage of VAMP2 by LC/X. (FIG. 13D) The active form of A8-J10-BoNT/XA was generated via sortase-mediated ligation and analyzed on SDS-PAGE gels. The arrows indicate the ligated full-length toxins. One of three independent experiments is shown.

FIGS. 14A-14B show A8-^{ci}BoNT/XA utilizes the same receptors to target neurons as BoNT/A. (FIG. 14A) A8-^{ci}BoNT/XA and ^{ci}BoNT/A were pre-incubated with GST-tagged the 4^{th} luminal domain of SV2C (SV2C-L4), and then applied to cultured rat cortical neurons for 8 min in culture medium. Neurons were washed, fixed, and subjected to immunostaining to detect A8-^{ci}BoNT/XA and ^{ci}BoNT/A using a human monoclonal antibody (RAZ-1) that recognizes BoNT/A-Hc. Synapsin was detected as a marker for synaptic terminals. Pre-incubation with SV2C-L4 reduced binding of both A8-^{ci}BoNT/XA and ^{ci}BoNT/A to neurons. One of two independent experiments is shown. (FIG. 14B) BoNT/A (20 pM) was pre-incubated with either A8 or ^{ci}BoNT/XA (50 nM). The mixture was then added to the medium and exposed to cultured rat cortical neurons. Cell lysates were analyzed by immunoblot to detect SNAP-25 and actin. Pre-incubation with A8 did not affect cleavage of SNAP-25, indicating that A8 is incapable of inhibiting toxins prior to its entry into neurons. ^{ci}BoNT/XA was able to reduce cleavage of SNAP-25 when applied at the same time as BoNT/A, suggesting that it can compete with BoNT/A for binding to neurons. One of two independent experiments is shown.

FIGS. 15A-15E show A8-^{ci}BoNT/XA reduced BoNT/A-induced local leg muscle paralysis in vivo. (FIG. 15A) Experiments were carried out as described in FIG. 8B. The representative images of mice are presented to show that A8-^{ci}BoNT/XA allowed complete recovery of toe spreading by day 3, while injection of A8 or ^{ci}BoNT/XA alone did not reduce paralysis. (FIG. 15B) DAS assays were carried out using BoNT/B (3.5 pg). A8-^{ci}BoNT/XA was injected 18 h later into the same muscle. DAS scores were recorded and plotted over time (right panel) and representative images on day 3 are shown in the left panel. A8-^{ci}BoNT/XA did not affect BoNT/B-induced paralysis. Data are pooled from two independent experiments (n=10 each group). (FIG. 15C) The indicated mixture of A8 and ^{ci}BoNT/XA were injected as controls for experiments described in FIG. 8D (n=8). (FIG. 15D) The indicated mixture of A8 and ^{ci}BoNT/XA were injected as controls for experiments described in FIG. 8E (vehicle, n=18; A8 (6 μg)/^{ci}BoNT/XA (54 μg), n=10; A8 (60 μg)/^{ci}BoNT/XA (540 μg), n=8). (FIG. 15E) The indicated mixture of A8 and ^{ci}BoNT/XA were injected as controls for experiments described in FIG. 8F (vehicle, n=9; A8 (0.6 μg)/^{ci}BoNT/XA (5.4 μg), n=8).

FIGS. 16A-16E show characterization of A8-J10-^{ci}BoNT/XA in vitro and on cultured neurons. (FIG. 16A) A8-J10-^{ci}BoNT/XA was expressed and purified from E. coli, activated by thrombin, and analyzed on SDS-PAGE gels with or without DTT. (FIG. 16B) LC/B was incubated with BDE in the presence of A8-J10-^{ci}BoNT/XA or A8-J10. Cleavage of VAMP2 by LC/B was analyzed by immunoblot. A8-J10-^{ci}BoNT/XA and A8-J10 inhibited LC/B activity to a similar degree. One of two independent experiments is shown. (FIG. 16C) LC/A was incubated with BDE in the presence of A8-J10, A8-^{ci}BoNT/XA, or A8-J10-^{ci}BoNT/XA. Cleavage of SNAP-25 by LC/A was analyzed by immunoblot. A8-J10-^{ci}BoNT/XA and A8-J10 inhibited LC/A activity to a similar degree. One of two independent experiments is shown. (FIG. 16D) Active forms of BoNT/XA, A8-BoNT/XA, and A8-J10-BoNT/XA were generated via sortase-mediated ligation of the LCH_{N}/X, A8-LCH_{N}/X, or A8-J10-LCH_{N}/X with Hc/A. Cultured rat cortical neurons were exposed to these toxins, and cleavage of VAMP2 was analyzed by immunoblot. SNAP-25 served as a loading control. A8-J10-BoNT/XA and A8-BoNT/XA delivered the LC/X into neurons with similar efficacy. One of three independent experiments is shown. (FIG. 16E) Cultured cortical neurons were exposed to BoNT/A for 12 h, washed, and further incubated with toxin-free medium for 24 h. A8-^{ci}BoNT/XA or A8-J10-^{ci}BoNT/XA was then added to the medium for 48 h. Neuro lysates were harvested and analyzed by immunoblot to detect the three SNARE proteins and actin. Adding A8-J10-^{ci}BoNT/XA or A8-^{ci}BoNT/XA both reduced cleavage of SNAP-25, with A8-^{ci}BoNT/XA showing more protection of SNAP-25 than A8-J10-^{ci}BoNT/XA. One of three independent experiments is shown.

FIG. 17 shows in vivo toxicity analysis of the indicated proteins. The indicated proteins were purified from E. coli with endotoxin removed. They were injected IP into mice. Surviving mice were observed for 21 days.

FIG. 18 shows clinical scores for botulism in mice. The humane endpoint is defined as a combined clinical score (from all categories) 5.

FIG. 19 shows schematic illustration of the indicated constructs.

FIGS. 20A-20E show delivery of two nanobodies (J10-A8) using ^{ci}BoNT/XA for post-exposure treatment of BoNT/A and BoNT/B intoxication. (FIG. 20A) Schematic drawing of ^{ci}BoNT/XA with two nanobodies (J10 and A8) fused to its N-terminus. The fusion protein is termed J10-A8-^{ci}BoNT/XA. (FIG. 20B) DAS assays were carried out with BoNT/A (6 pg). The indicated concentrations of J10-A8-^{ci}BoNT/XA were injected into the same leg muscle 18 h later. Representative image showing that J10-A8-^{ci}BoNT/XA treatment shortened the duration of muscle paralysis induced by BoNT/A. (FIG. 20C) DAS assays were carried out with BoNT/A (6 pg) and the indicated concentrations of J10-A8-^{ci}BoNT/XA were injected into the same leg muscle 18 h later. DAS scores were plotted over time. (FIG. 20D) DAS assays were carried out with BoNT/B (3.5 pg). The indicated concentrations of J10-A8-^{ci}BoNT/XA were injected into the same leg muscle 18 h later. Representative image showing that J10-A8-^{ci}BoNT/XA treatment shortened the duration of muscle paralysis induced by BoNT/B. (FIG. 20E) DAS assays were carried out with BoNT/B (3.5 pg) and the indicated concentrations of J10-A8-^{ci}BoNT/XA were injected into the same leg muscle 18 h later. DAS scores were plotted over time.

FIGS. 21A-21B show neutralization activity of A8-^{ci}BoNT/PMP1-A via IM injection in vivo in mouse. (FIG. 21A) Schematic drawing of ^{ci}BoNT/PMP1-A with A8 fused to its N-terminus. The fusion protein is termed A8-^{ci}BoNT/PmA. (FIG. 21B) 6 pg of BoNT/A were injected in mouse hind limb muscle. After 18 hours, the mice showed paralysis (DAS score 2-3). The indicated concentrations of A8-^{ci}BoNT/PmA or A8-^{ci}BoNT/XA were injected in the same muscle and DAS score were recorded over time.

### DETAILED DESCRIPTION OF CERTAIN EMBODIMENTS

Clostridium Botulinum neurotoxins (BoNTs) are a family of bacterial toxins produced by clostridium bacteria, with seven well-established serotypes (BoNT/A-G). Recently a new BoNT serotype, serotype H (BoNT/H) has been reported (e.g., as described in Maslanka et al., J Infect Dis. 2016 Feb. 1; 213(3):379-85, incorporated herein by reference). BoNTs are one of the most dangerous potential bio-terrorism agents, classified as a “Category A” select agent by Center for Disease Control (CDC) of United States. These toxins are produced as a single polypeptide and can be separated by bacterial or host proteases into a light chain (LC, ˜50 kDa) and a heavy chain (H_{C}, ˜100 kDa). The two chains remain connected via an inter-chain disulfide bond. The H_{c} contains two sub-domains: the N-terminal H_{N} domain that mediates translocation of the LC across endosomal membranes, and the C-terminal Hc domain that mediates binding to receptors on neurons. The inter-chain disulfide bond is reduced once the LC translocates into the cytosol. Released LC acts as a protease to specifically cleave a set of neuronal proteins: BoNT/A, C, and E cleave at distinct sites on a protein known as SNAP-25; BoNT/B, D, F, and G cleave at different sites on a vesicle protein VAMP; and BoNT/C also cleaves a transmembrane protein syntaxin 1. These three proteins form a complex, known as SNARE complex, which is essential for release of neurotransmitters. Cleavage of any one of these three SNARE proteins blocks neurotransmitters release from neurons, thus paralyzing muscles.

BoNTs are the most potent toxins known and cause the human and animal disease known as botulism. The major form of botulism is caused by ingesting food contaminated with BoNTs (food botulism). Other forms also exist such as infant botulism, which is due to colonization of the intestine by toxin-producing bacteria in infants.

Because local injections of minute amounts of toxins can attenuate neuronal activity in targeted regions, BoNTs have been used to treat a growing list of medical conditions, including muscle spasms, chronic pain, overactive bladder problems, as well as for cosmetic applications. The market for BoNTs has already surpassed $1.5 billion in 2011 and is projected to reach 2.9 billion by 2018.

Clostridium botulinum neurotoxin (BoNT)-based delivery system have been previously developed and showed to be able to deliver proteins into neurons (e.g., as described in Bade et al., J Neurochem 91, 1461-1472, 2004, incorporated herein by reference). To serve as a delivery tool, BoNTs must be “de-toxified” by introducing point mutations into its protease domain (light chain) to abolish its protease activity inside neurons. However, currently reported “inactive” form of BoNTs still showed a level of toxicity when injected into mice, causing paralysis and even death of mice (e.g., as shown in Vazquez-Cintron et al., Sci Rep 7, 42923, 2017 and Webb et al., Toxins (Basel) 9, 2017, incorporated herein by reference). While the reason for the residual toxicity remains unknown, it is a major barrier for developing a useful delivery system using catalytically inactive BoNTs.

It was surprisingly found herein that, two recently discovered new BoNT-like toxins, one from Clostridium botulinum, designated serotype X (BoNT/X, as described in described in Zhang et al., Nat Commun 8, 14130, 2017, incorporated herein by reference) and one from Enterococcus faecium (BoNT/En, as described in as described in Zhang et al., Cell Host Microbe 23, 169-176 e166, 2018, incorporated herein by reference) overcome the residual toxicity challenge posed by traditional BoNT-based delivery systems. Additionally, another recently discovered new BoNT-like toxin from Paraclostridium bifermentans herein termed BoNT/PMP1 (e.g., as described in Contraras et al., Nature Communications volume 10, Article number: 2869 (2019), incorporated herein by reference) can also be used as a delivery vehicle described herein without residual toxicity issues.

It was demonstrated herein that, in contrast to delivery tools based on traditional BoNTs (BoNT/A-H), catalytically inactive BoNT/X (as well as fragments or chimeric toxins derived from BoNT/X such as catalytically inactive BoNT/XA) showed no detectable toxicity in vivo in mouse models, thus representing a safe and effective delivery system for delivering cargo/therapeutics into cells (e.g., neurons). Two other BoNT-like neurotoxins that are highly similar to BoNT/X, BoNT/En and BoNT/PMP1 are expected also have no toxicity in vivo in their inactive form. Accordingly, the present disclosure, in some aspects, provide catalytically inactive botulinum neurotoxin-like toxins from Clostridium botulinum, serotype X (BoNT/X), from Enterococcus faecium (BoNT/En), or from Paraclostridium bifermentans (BoNT/PMP1) and their uses as delivery vehicles to deliver agents (e.g., therapeutic agents or diagnostic agents) to a cell (e.g., a neuron).

A “Clostridium Botulinum neurotoxin (BoNT),” as used herein encompasses broadly any BoNT polypeptides, variants, or fragments from Clostridium botulinum (e.g., from Clostridium botulinum serotypes A, B, C, D, E, F, G, and H). In some embodiments, the term BoNT also encompasses a BoNT-like toxin from Clostridium botulinum serotype X (BoNT/X), a BoNT-like toxin from Enterococcus faecium (BoNT/En), or a BoNT-like toxin from Paraclostridium bifermentans (BoNT/PMP1) and variants and fragments thereof.

In some embodiments, a BoNT refers to a full-length BoNT. A full-length BoNT comprises a light chain (LC) and a heavy chain (HC). The light chain of a BoNT comprises the protease domain, and the heavy chain of a BoNT contains a translocation domain at the N-terminus and a receptor binding domain at the C-terminus. The heavy chain and light chain are translated as a single polypeptide chain, wherein the LC and the HC are linked via a linker region. The linker region is cleaved by a protease and the LC and HC remain linked via a disulfide bond between two cysteine residues, producing a mature BoNT or BoNT-like toxin. In some embodiments, a BoNT refers to a fragment of a full length BoNT, e.g., a BoNT fragment that comprises only the LC (protease domain), a BoNT fragment that comprises the LC (protease domain) and the N-terminus of the HC (referred to herein as “LC-Hn”), or a BoNT fragment that comprises the C-terminus of the HC (receptor binding domain, referred to herein as “Hc”).

“BoNT/X” refers to a BoNT-like toxin from Clostridium botulinum, serotype X. BoNT/X has been described in Zhang et al., Nat Commun 8, 14130, 2017, incorporated herein by reference. The full length BoNT/X wild type protein sequence (GenBank No. BAQ12790.1) is provided in Table 2 as SEQ ID NO: 1.

“BoNT/En” refers to a BoNT-like toxin from Enterococcus faecium. BoNT/En has been described in Zhang et al., Cell Host Microbe 23, 169-176 e166, 2018, incorporated herein by reference. The full length BoNT/En wild type protein sequence (GenBank No. OTO22244.1) is provided in Table 2 as SEQ ID NO: 2.

“BoNT/PMP1” refers to a BoNT-like toxin from Paraclostridium bifermentans. PMP1 is also referred to in short as “Pm” in some of the figures and examples. Similarly, BoNT/PMP1 is also referred to as “BoNT/Pm” herein. BoNT/PMP1 has been described in Contraras et al., Nature Communications volume 10, Article number: 2869 (2019), incorporated herein by reference. The full length BoNT/PMP1 wild type protein sequence is provided in Table 2 as SEQ ID NO: 85.

The light chain of a BoNT (e.g., a BoNT-like toxin such as BoNT/X, BoNT/EN or BoNT/PMP1) comprises a protease domain, which cleaves natural BoNT substrates (e.g., certain SNARE proteins and VAMP proteins). The protease domain or the LC of BoNT/X is considered to correspond to about amino acid 1-439 of full length BoNT/X as set forth in SEQ ID NO: 1. The domain boundary may vary by about 25 amino acids. For example, the protease domain of BoNT/X may correspond to amino acids 1-414 or 1-464 of full length BoNT/X as set forth in SEQ ID NO: 1. In some embodiments, the protease domain corresponds to amino acids 1-414, 1-415, 1-416, 1-417, 1-418, 1-419, 1-420, 1-421, 1-422, 1-423, 1-424, 1-425, 1-426, 1-427, 1-428, 1-429, 1-430, 1-431, 1-432, 1-433, 1-434, 1-435, 1-436, 1-437, 1-438, 1-439, 1-440, 1-441, 1-442, 1-443, 1-444, 1-445, 1-446, 1-447, 1-448, 1-449, 1-450, 1-451, 1-452, 1-453, 1-454, 1-455, 1-456, 1-457, 1-458, 1-459, 1-460, 1-461, 1-462, 1-463, or 1-464 of full length BoNT/X as set forth in SEQ ID NO: 1. In some embodiments, the protease domain of BoNT/X corresponds to amino acids 1-422 of full-length BoNT/X as set forth in SEQ ID NO: 1.

The protease domain or the LC of BoNT/En corresponds to about amino acid 1-423 of full length BoNT/En as set forth in SEQ ID NO: 2. The domain boundary may vary by about 25 amino acids. For example, the protease domain corresponds to amino acids 1-398 or 1-448 of full length BoNT/En as set forth in SEQ ID NO: 2. In some embodiments, the protease domain may correspond to amino acids 1-398, 1-399, 1-400, 1-401, 1-402, 1-403, 1-404, 1-405, 1-406, 1-407, 1-408, 1-409, 1-410, 1-411, 1-412, 1-413, 1-414, 1-415, 1-416, 1-417, 1-418, 1-419, 1-420, 1-421, 1-422, 1-423, 1-424, 1-425, 1-426, 1-427, 1-428, 1-429, 1-430, 1-431, 1-432, 1-433, 1-434, 1-435, 1-436, 1-437, 1-438, 1-439, 1-440, 1-441, 1-442, 1-443, 1-444, 1-445, 1-446, 1-447, or 1-448 of full length BoNT/En as set forth in SEQ ID NO: 2. In some embodiments, the protease domain of BoNT/En corresponds to amino acids 1-423 of full length BoNT/En as set forth in SEQ ID NO: 2.

The protease domain or the LC of BoNT/PMP1 corresponds to about amino acid 1-394 of full length BoNT/PMP1 as set forth in SEQ ID NO: 85. The domain boundary may vary by about 25 amino acids. For example, the protease domain corresponds to amino acids 1-369 or 1-419 of full length BoNT/PMP1 as set forth in SEQ ID NO: 85. In some embodiments, the protease domain may correspond to amino acids 1-369, 1-370, 1-371, 1-372, 1-373, 1-374, 1-375, 1-376, 1-377, 1-378, 1-379, 1-380, 1-381, 1-382, 1-383, 1-384, 1-385, 1-386, 1-387, 1-388, 1-389, 1-390, 1-391, 1-392, 1-393, 1-394, 1-395, 1-396, 1-397, 1-398, 1-399, 1-400, 1-401, 1-402, 1-403, 1-404, 1-405, 1-406, 1-407, 1-408, 1-409, 1-410, 1-411, 1-412, 1-413, 1-414, 1-415, 1-416, 1-417, 1-418, or 1-419 of full length BoNT/PMP1 as set forth in SEQ ID NO: 85. In some embodiments, the protease domain of BoNT/PMP1 corresponds to amino acids 1-394 of full length BoNT/PMP1 as set forth in SEQ ID NO: 85.

A “catalytically inactive BoNT,” refers to a provide modified BoNT polypeptide comprising an inactive protease domain. Catalytically inactive BoNT polypeptides cannot cleave BoNT substrate proteins (e.g., a SNARE protein) due to the inactivation of the protease domain. In some embodiments, a catalytically inactive BoNT is a full length BoNT that is catalytically inactive or a BoNT fragment (e.g., the LC or LC-Hn fragment) that is catalytically inactive. In some embodiments, a catalytically inactive BoNT is a chimeric BoNT comprising a catalytically inactive LC-Hn fused to a receptor binding domain (Hc) from a different BoNT serotype or a BoNT from a different bacterial species.

In some embodiments, the catalytically inactive BoNT is an engineered chimeric BoNT comprising (a) a light chain comprising an inactive protease domain, and (b) a heavy chain comprising: (i) a translocation domain, and (ii) a receptor binding domain, wherein (a) and (b)(i) are from a BoNT/X, from a BoNT/En, or from a BoNT/PMP1, and wherein (b)(ii) is from a BoNT in Clostridium botulinum, serotype A, B, C, D, E, F, G or H (i.e., BoNT/A, BoNT/B, BoNT/C, BoNT/D, BoNT/E, BoNT/F, BoNT/G, or BoNT/H). It is to be understood that when a BoNT serotype is referred to herein (e.g., BoNT/A), all sub-types of the serotype (e.g., BoNT/A1, BoNT/A2, BoNT/A3, BoNT/A4, BoNT/A5, BoNT/A7, or BoNT/A8) is contemplated. All subtypes of BoNT/B, BoNT/C, BoNT/D, BoNT/E, BoNT/F, BoNT/G, or BoNT/H are also encompassed by the present disclosure. In some embodiments, the chimeric BoNT of the present disclosure comprises a light chain comprising an inactive protease domain of BoNT/X or BoNT/En, and a heavy chain comprising a translocation domain of BoNT/X or BoNT/En and a receptor binding domain of any one of BoNT/A, BoNT/B, BoNT/C, BoNT/D, BoNT/E, BoNT/F, BoNT/G, and BoNT/H.

In some embodiments, the catalytically inactive BoNT is a catalytically inactive BoNT/X. In some embodiments, the catalytically inactive BoNT/X is a catalytically inactive BoNT/X fragment comprising a catalytically inactive protease domain and a translocation domain (herein referred to as ^{ci}LC-Hn/X). In some embodiments, the catalytically inactive BoNT/X is a chimeric BoNT comprises a catalytically inactive LC-Hn/X and a Hc from a BoNT in Clostridium botulinum, serotype A, B, C, D, E, F, G or H (herein referred to as Hc/A, Hc/B, Hc/C, Hc/D, Hc/E, Hc/F, Hc/G, or Hc/F, respectively). Such chimeric BoNTs are referred to herein as ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH, respectively. It is to be understood that the ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH encompasses chimeric BoNTs comprising receptor binding domains from any subtypes of BoNT/A, BoNT/B, BoNT/C, BoNT/D, BoNT/E, BoNT/F, BoNT/G, or BoNT/H.

In some embodiments, the inactive protease domain of the catalytically inactive BoNT described herein (e.g., ^{ci}LC-Hn/X, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH) comprises one or more (e.g., 1, 2, 3, 4, 5) substitution mutation(s) in a position corresponding to R360, Y363, H227, E228, or H231 in SEQ ID NO: 1. In some embodiments, the inactive protease domain of the catalytically inactive BoNT described herein (e.g., ^{ci}LC-Hn/X, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH) comprises three amino acid substitutions in a position corresponding to E228, R360, and Y363 in SEQ ID NO: 1. In some embodiments, the inactive protease domain of the catalytically inactive BoNT described herein (e.g., ^{ci}LC-Hn/X, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH) comprises amino acid substitutions corresponding to E228Q, R360A, and Y363F in SEQ ID NO: 1.

In some embodiments, the catalytically inactive BoNT is a ^{ci}LC-Hn/X comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 3 and comprises one or more substitution mutation(s) in a position corresponding to R360, Y363, H227, E228, or H231 in SEQ ID NO: 1. In some embodiments, the catalytically inactive BoNT is a ^{ci}LC-Hn/X comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 3 and comprises amino acid substitutions corresponding to E228Q, R360A, and Y363F in SEQ ID NO: 1. In some embodiments, the catalytically inactive BoNT is a ^{ci}LC-Hn/X comprising the amino acid sequence of SEQ ID NO: 3. In some embodiments, the catalytically inactive BoNT is a ^{ci}LC-Hn/X consisting of the amino acid sequence of SEQ ID NO: 3.

In some embodiments, the catalytically inactive BoNT is a chimeric BoNT (e.g., ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 5-12 and comprises one or more substitution mutation(s) in a position corresponding to R360, Y363, H227, E228, or H231 in SEQ ID NO: 1. In some embodiments, the catalytically inactive BoNT is a chimeric BoNT (e.g., ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 5-12 and comprises amino acid substitutions corresponding to E228Q, R360A, and Y363F in SEQ ID NO: 1. In some embodiments, the catalytically inactive BoNT is a chimeric BoNT (e.g., ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH) comprising the amino acid sequence of any one of SEQ ID NOs: 5-12. In some embodiments, the catalytically inactive BoNT is a chimeric BoNT (e.g., ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH) consisting of the amino acid sequence of any one of SEQ ID NOs: 5-12.

In some embodiments, the catalytically inactive BoNT is a catalytically inactive BoNT/En. In some embodiments, the catalytically inactive BoNT/En is a catalytically inactive BoNT/En fragment comprising a catalytically inactive protease domain and a translocation domain (herein referred to as a ^{ci}LC-Hn/En). In some embodiments, the catalytically inactive BoNT/En is a chimeric BoNT comprises a catalytically inactive LC-Hn/En and a Hc from a BoNT in Clostridium botulinum, serotype A, B, C, D, E, F, G or H (herein referred to as Hc/A, Hc/B, Hc/C, Hc/D, Hc/E, Hc/F, Hc/G, or Hc/F, respectively). Such chimeric BoNTs are referred to herein as ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH, respectively. It is to be understood that the ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH encompasses chimeric BoNTs comprising receptor binding domains from any subtypes of BoNT/A, BoNT/B, BoNT/C, BoNT/D, BoNT/E, BoNT/F, BoNT/G, or BoNT/H.

In some embodiments, the inactive protease domain of the catalytically inactive BoNT described herein (e.g., ^{ci}LC-Hn/En, as ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) comprises one or more (e.g., 1, 2, or 3) substitution mutation(s) in a position corresponding to H225, E226, H229, R364, or Y367 in SEQ ID NO: 2. In some embodiments, the inactive protease domain of the catalytically inactive BoNT described herein (e.g., ^{ci}LC-Hn/En, as ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) comprises three amino acid substitutions in a position corresponding to E226, R364, or Y367 in SEQ ID NO: 2. In some embodiments, the inactive protease domain of the catalytically inactive BoNT described herein (e.g., ^{ci}LC-Hn/En, as ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) comprises amino acid substitutions corresponding to E226Q, R364A, and Y367F in SEQ ID NO: 2.

In some embodiments, the catalytically inactive BoNT is a ^{ci}LC-Hn/En comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 4 and comprises one or more substitution mutation(s) in a position corresponding to H225, E226, H229, R364, or Y367 in SEQ ID NO: 2. In some embodiments, the catalytically inactive BoNT is a ^{ci}LC-Hn/En comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 4 and comprises amino acid substitutions corresponding to E226Q, R364A, and Y367F in SEQ ID NO: 2. In some embodiments, the catalytically inactive BoNT is a ^{ci}LC-Hn/En comprising the amino acid sequence of SEQ ID NO: 4. In some embodiments, the catalytically inactive BoNT is a ^{ci}LC-Hn/En consisting of the amino acid sequence of SEQ ID NO: 4. In some embodiments, the catalytically inactive BoNT is a chimeric BoNT (e.g., ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 13-20 and comprises one or more substitution mutation(s) in a position corresponding to H225, E226, H229, R364, or Y367 in SEQ ID NO: 2. In some embodiments, the catalytically inactive BoNT is a chimeric BoNT (e.g., ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 13-20 and comprises amino acid substitutions corresponding to E226Q, R364A, and Y367F in SEQ ID NO: 2. In some embodiments, the catalytically inactive BoNT is a chimeric BoNT (e.g., ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) comprising the amino acid sequence of any one of SEQ ID NOs: 13-20. In some embodiments, the catalytically inactive BoNT is a chimeric BoNT (e.g., ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) consisting of the amino acid sequence of any one of SEQ ID NOs: 13-20.

In some embodiments, the catalytically inactive BoNT is a catalytically inactive BoNT/PMP1. In some embodiments, the catalytically inactive BoNT/PMP1 is a catalytically inactive BoNT/PMP1 fragment comprising a catalytically inactive protease domain and a translocation domain (herein referred to as ^{ci}LC-Hn/PMP1). In some embodiments, the catalytically inactive BoNT/PMP1 is a chimeric BoNT comprising a catalytically inactive LC-Hn/PMP1 and a Hc from a BoNT in Clostridium botulinum, serotype A, B, C, D, E, F, G or H (herein referred to as Hc/A, Hc/B, Hc/C, Hc/D, Hc/E, Hc/F, Hc/G, or Hc/F, respectively). Such chimeric BoNTs are referred to herein as ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H, respectively. It is to be understood that the ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H encompasses chimeric BoNTs comprising receptor binding domains from any subtypes of BoNT/A, BoNT/B, BoNT/C, BoNT/D, BoNT/E, BoNT/F, BoNT/G, or BoNT/H.

In some embodiments, the inactive protease domain of the catalytically inactive BoNT described herein (e.g., ^{ci}LC-Hn/PMP1, ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) comprises one or more (e.g., 1, 2, 3, 4, 5) substitution mutation(s) in a position corresponding to H208, E209, H212, R344, or Y347 in SEQ ID NO: 85. In some embodiments, the inactive protease domain of the catalytically inactive BoNT described herein (e.g., ^{ci}LC-Hn/PMP1, ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) comprises three amino acid substitutions in a position corresponding to E209, R344, and Y347 in SEQ ID NO: 85. In some embodiments, the inactive protease domain of the catalytically inactive BoNT described herein (e.g., ^{ci}LC-Hn/PMP1, ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) comprises amino acid substitutions corresponding to E209Q, R344A, and Y347F in SEQ ID NO: 85.

In some embodiments, the catalytically inactive BoNT is a ^{ci}LC-Hn/PMP1 comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 86 and comprises one or more substitution mutation(s) in a position corresponding to H208, E209, H212, R344, or Y347 in SEQ ID NO: 85. In some embodiments, the catalytically inactive BoNT is a ^{ci}LC-Hn/PMP1 comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 86 and comprises amino acid substitutions corresponding to E209Q, R344A, and Y347F in SEQ ID NO: 85. In some embodiments, the catalytically inactive BoNT is a ^{ci}LC-Hn/PMP1 comprising the amino acid sequence of SEQ ID NO: 86. In some embodiments, the catalytically inactive BoNT is a ^{ci}LC-Hn/PMP1 consisting of the amino acid sequence of SEQ ID NO: 86.

In some embodiments, the catalytically inactive BoNT is a chimeric BoNT (e.g., ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 87-94 and comprises one or more substitution mutation(s) in a position corresponding to H208, E209, H212, R344, or Y347 in SEQ ID NO: 85. In some embodiments, the catalytically inactive BoNT is a chimeric BoNT (e.g., ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 87-94 and comprises amino acid substitutions corresponding to E209Q, R344A, and Y347F in SEQ ID NO: 85. In some embodiments, the catalytically inactive BoNT is a chimeric BoNT (e.g., ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) comprising the amino acid sequence of any one of SEQ ID NOs: 87-94. In some embodiments, the catalytically inactive BoNT is a chimeric BoNT (e.g., ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) consisting of the amino acid sequence of any one of SEQ ID NOs: 87-94.

In the natural BoNTs, including the and BoNT-like toxins BoNT/X and BoNT/En, a linker is present between the LC and the N-terminus of the HC (i.e., between LC and Hn). Once a BoNT is translated, the linker is cleaved and the LC and HC are linked via a disulfide bond to produce a mature BoNT. In some embodiments, the catalytically inactive BoNT described herein (e.g., ^{ci}LC-Hn/X, ^{ci}LC-Hn/En, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, ^{ci}BoNT/EnH, ^{ci}LC-Hn/PMP1, ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) comprises a modified linker, which replaces the natural linker between the inactive protease domain (LC) and the translocation domain (Hn). A “modified linker” refers to a designed linker that is different from the natural linker between the LC and the Hn in BoNT/X, BoNT/E, or BoNT/PMP1). In some embodiments, the modified linker comprises a protease cleave site. A “protease cleavage site” refers to an amino acid sequence that is recognized and cleaved by a protease. Protease cleavage results in the breakage of an amino bond, producing two peptides. Exemplary protease cleavage sites that may be used in the modified linker of the present disclosure include, without limitation, cleavage sites for thrombin (LVPRIGS, SEQ ID NO: 77), TEV (ENLYFQIG, SEQ ID NO: 78), PreScission (3C protease, LEVLFQIGP, SEQ ID NO: 79), Factor Xa (IEGRI, SEQ ID NO: 80; or IEGRI, SEQ ID NO: 81), MMP-12, MMP-13, MMP-17, MMP-20, Granzyme-B, SUMO protease (AHREQIGGI, SEQ ID NO: 82), Furin (RXXRI) and Enterokinase and Enterokinase (DDDDKI, SEQ ID NO: 83). “I” indicates the position cleaved by the protease. In some embodiments, the linker comprises the amino acid sequence of any of SEQ ID NOs: 77-83), and they are used to replace the original linker sequences in BoNT/X (residues P424 to G466) and in BoNT/En (P425 to S437). In some embodiments, the linker contains a thrombin cleavage site. In some embodiments, the linker containing the thrombin cleave site comprises the amino acid sequence of CHKAIDGRSLGGSLVPRGSGGSAAAYNKTLDC (SEQ ID NO: 84). When the linker CHKAIDGRSLGGSLVPRGSGGSAAAYNKTLDC (SEQ ID NO: 84) is used, the disulfide bond between the LC and the HC of the processed BoNT is formed between the cysteine at position 1 and the cysteine at position 32 of the linker. In some embodiments, the catalytically inactive BoNT is a ^{ci}LC-Hn/X comprising a modified linker, and comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 21 and comprises one or more substitution mutation(s) in a position corresponding to R360, Y363, H227, E228, or H231 in SEQ ID NO: 1. In some embodiments, the catalytically inactive BoNT is a ^{ci}LC-Hn/X comprising a modified linker, and comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 20 and comprises amino acid substitutions corresponding to E228Q, R360A, and Y363F in SEQ ID NO: 1. In some embodiments, the catalytically inactive BoNT is a ^{ci}LC-Hn/X comprising the amino acid sequence of SEQ ID NO: 21. In some embodiments, the catalytically inactive BoNT is a ^{ci}LC-Hn/X consisting of the amino acid sequence of SEQ ID NO: 21.

In some embodiments, the catalytically inactive BoNT is a chimeric BoNT (e.g., ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH with a modified linker) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 23-30, and comprises one or more substitution mutation(s) in a position corresponding to R360, Y363, H227, E228, or H231 in SEQ ID NO: 1. In some embodiments, the catalytically inactive BoNT is a chimeric BoNT (e.g., ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH with a modified linker) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 23-30 and comprises amino acid substitutions corresponding to E228Q, R360A, and Y363F in SEQ ID NO: 1. In some embodiments, the catalytically inactive BoNT is a chimeric BoNT (e.g., ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH with a modified linker) comprising the amino acid sequence of any one of SEQ ID NOs: 23-30. In some embodiments, the catalytically inactive BoNT is a chimeric BoNT ((e.g., ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, or ^{ci}BoNT/XH with a modified linker) consisting of the amino acid sequence of any one of SEQ ID NOs: 23-30.

In some embodiments, the catalytically inactive BoNT is a ^{ci}LC-Hn/En comprising a modified linker, and comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 22 and comprises one or more substitution mutation(s) in a position corresponding to H225, E226, H229, R364, or Y367 in SEQ ID NO: 2. In some embodiments, the catalytically inactive BoNT is a ^{ci}LC-Hn/En comprising a modified linker, and comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 4 and comprises amino acid substitutions corresponding to E226Q, R364A, and Y367F in SEQ ID NO: 2. In some embodiments, the catalytically inactive BoNT is a ^{ci}LC-Hn/En comprising the amino acid sequence of SEQ ID NO: 22. In some embodiments, the catalytically inactive BoNT is a ^{ci}LC-Hn/En consisting of the amino acid sequence of SEQ ID NO: 22.

In some embodiments, the catalytically inactive BoNT is a chimeric BoNT (e.g., ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH with a modified linker) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 31-38, and comprises one or more substitution mutation(s) in a position corresponding to H225, E226, H229, R364, or Y367 in SEQ ID NO: 2. In some embodiments, the catalytically inactive BoNT is a chimeric BoNT (e.g., ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH with a modified linker) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 31-38 and comprises amino acid substitutions corresponding to E226Q, R364A, and Y367F in SEQ ID NO: 2. In some embodiments, the catalytically inactive BoNT is a chimeric BoNT (e.g., ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH with a modified linker) comprising the amino acid sequence of any one of SEQ ID NOs: 31-38. In some embodiments, the catalytically inactive BoNT is a chimeric BoNT (e.g., ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH with a modified linker) consisting of the amino acid sequence of any one of SEQ ID NOs: 31-38.

In some embodiments, the catalytically inactive BoNT is a ^{ci}LC-Hn/PMP1 comprising a modified linker, and comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 95 and comprises one or more substitution mutation(s) in a position corresponding to H208, E209, H212, R344, or Y347 in SEQ ID NO: 85. In some embodiments, the catalytically inactive BoNT is a ^{ci}LC-Hn/PMP1 comprising a modified linker, and comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 94 and comprises amino acid substitutions corresponding to E209Q, R344A, and Y347F in SEQ ID NO: 85. In some embodiments, the catalytically inactive BoNT is a ^{ci}LC-Hn/PMP1 comprising the amino acid sequence of SEQ ID NO: 95. In some embodiments, the catalytically inactive BoNT is a ^{ci}LC-Hn/PMP1 consisting of the amino acid sequence of SEQ ID NO: 95.

In some embodiments, the catalytically inactive BoNT is a chimeric BoNT (e.g., ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H with a modified linker) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 96-103, and comprises one or more substitution mutation(s) in a position corresponding to H208, E209, H212, R344, or Y347 in SEQ ID NO: 85. In some embodiments, the catalytically inactive BoNT is a chimeric BoNT (e.g., ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H with a modified linker) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 96-103 and comprises amino acid substitutions corresponding to E209Q, R344A, and Y347F in SEQ ID NO: 85. In some embodiments, the catalytically inactive BoNT is a chimeric BoNT (e.g., ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H with a modified linker) comprising the amino acid sequence of any one of SEQ ID NOs: 96-103. In some embodiments, the catalytically inactive BoNT is a chimeric BoNT ((e.g., ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H with a modified linker) consisting of the amino acid sequence of any one of SEQ ID NOs: 96-103.

In some embodiments, the catalytically inactive BoNT is in its processed form, wherein the light chain (e.g., any one of the inactive LC/X and LC/En described herein) and heavy chain (either Hn or the full heavy chain containing Hn and Hc) is linked by a disulfide bond. In some embodiments, the catalytically inactive BoNT comprises (a) a light chain comprising an inactive LC/X, and (b) a heavy chain comprising: (i) a translocation domain from BoNT/X, and (ii) a receptor binding domain from any one of BoNT/A, BoNT/B, BoNT/C, BoNT/D, BoNT/E, BoNT/F, BoNT/G, BoNT/H, and wherein the light chain and the heavy chain are linked via a disulfide bond. In some embodiments, the catalytically inactive BoNT comprises (a) a light chain comprising an inactive LC/En, and (b) a heavy chain comprising: (i) a translocation domain from BoNT/X, and (ii) a receptor binding domain from any one of BoNT/A, BoNT/B, BoNT/C, BoNT/D, BoNT/E, BoNT/F, BoNT/G, BoNT/H, and wherein the light chain and the heavy chain are linked via a disulfide bond.

In some embodiments, the catalytically inactive BoNT comprises: (a) a catalytically inactive light chain (^{ci}LC/X) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO:39, and comprises amino acid substitutions corresponding to E228Q, R360A, and Y363F in SEQ ID NO: 1; and (b) a heavy chain (Hn/X-Hc/A, B, C, D, E, F, G, or H) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 40-47, wherein the light chain and the heavy chain are linked via a disulfide bond. In some embodiments, the catalytically inactive BoNT comprises: (a) a light chain comprising an amino acid sequence of SEQ ID NO: 39; and (b) a heavy chain comprising the amino acid sequence of any one of SEQ ID NOs: 40-47, wherein the light chain and the heavy chain are linked via a disulfide bond.

In some embodiments, the catalytically inactive BoNT comprises: (a) a light chain (^{ci}LC/En) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 48, and comprises amino acid substitutions corresponding to amino acid substitutions corresponding to E226Q, R364A, and Y367F in SEQ ID NO: 2; and (b) a heavy chain (Hn/En-Hc/A, B, C, D, E, F, G, or H) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 49-56, wherein the light chain and the heavy chain are linked via a disulfide bond. In some embodiments, the catalytically inactive BoNT comprises: (a) a light chain comprising an amino acid sequence of SEQ ID NO: 48; and (b) a heavy chain comprising the amino acid sequence of any one of SEQ ID NOs: 49-56, wherein the light chain and the heavy chain are linked via a disulfide bond.

In some embodiments, the catalytically inactive BoNT comprises: (a) a catalytically inactive light chain (^{ci}LC/PMP1) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 104, and comprises amino acid substitutions corresponding to E209Q, R344A, and Y347F in SEQ ID NO: 85; and (b) a heavy chain (Hn/PMP1-Hc/A, B, C, D, E, F, G, or H) comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 105-112, wherein the light chain and the heavy chain are linked via a disulfide bond. In some embodiments, the catalytically inactive BoNT comprises: (a) a light chain comprising an amino acid sequence of SEQ ID NO: 104; and (b) a heavy chain comprising the amino acid sequence of any one of SEQ ID NOs: 105-112, wherein the light chain and the heavy chain are linked via a disulfide bond.

Other aspects of the present disclosure provide nucleic acids encoding any one of the BoNTs described herein. The nucleic acids may be DNA or RNA, double-stranded or single stranded. In some embodiments, the nucleic acid is within a vector, such as an expression vector. In some embodiments, the vector comprises a promoter operably linked to the nucleic acid. Also provided are cells comprising the nucleic acids or vectors, and cells expressing the BoNTs.

Variant nucleotide sequences include sequences that differ by one or more nucleotide substitutions, additions or deletions, such as allelic variants. In some embodiments, the isolated nucleic acid molecule of the present disclosure comprises a polynucleotide encoding a polypeptide comprising an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identity of any one of SEQ ID NOs: 1-121, and 124-150. In some embodiments, the isolated nucleic acid molecule of the present disclosure comprises a polynucleotide encoding a polypeptide comprising an amino acid sequence that has 85%, 86%, 87%, 88%, 89%, 90%, 91%, 92%, 93%, 94%, 95%, 96%, 97%, 98%, 99%, or 100% identity of any one of SEQ ID NOs: 1-121, and 124-150.

A variety of promoters can be used for expression of the polypeptides described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex virus promoter. Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)].

Other systems include FK506 dimer, VP16 or p65 using astradiol, RU486, diphenol murislerone, or rapamycin. Inducible systems are available from Invitrogen, Clontech and Ariad. Regulatable promoters that include a repressor with the operon can be used. In one embodiment, the lac repressor from Escherichia coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (HCMV) major immediate-early promoter to create a tetR-tet operator system to control gene expression in mammalian cells. In one embodiment, a tetracycline inducible switch is used (Yao et al., Human Gene Therapy; Gossen et al., Natl. Acad. Sci. USA, 89:5547-5551 (1992); Shockett et al., Proc. Natl. Acad. Sci. USA, 92:6522-6526 (1995)).

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

An expression vector comprising the nucleic acid can be transferred to a host cell by conventional techniques (e.g., electroporation, liposomal transfection, and calcium phosphate precipitation) and the transfected cells are then cultured by conventional techniques to produce the BoNTs described herein. In some embodiments, the expression of the BoNTs described herein is regulated by a constitutive, an inducible or a tissue-specific promoter.

The host cells used to express BoNTs described herein may be either bacterial cells such as Escherichia coli, or eukaryotic cells (e.g., mammalian cells, such as Chinese hamster ovary cells (CHO), in conjunction with a vector such as the major intermediate early gene promoter element from human cytomegalovirus). A variety of host-expression vector systems may be utilized to express the BoNTs described herein. Such host-expression systems represent vehicles by which the coding sequences of BoNTs described herein may be produced and subsequently purified, but also represent cells which may, when transformed or transfected with the appropriate nucleotide coding sequences, express the BoNTs described herein in situ. These include, but are not limited to, microorganisms such as bacteria (e.g., E. coli and B. subtilis) transformed with recombinant bacteriophage DNA, plasmid DNA or cosmid DNA expression vectors containing coding sequences for the BoNTs described herein; yeast (e.g., Saccharomyces pichia) transformed with recombinant yeast expression vectors containing sequences encoding the BoNTs described herein; insect cell systems infected with recombinant virus expression vectors (e.g., baclovirus) containing the sequences encoding the BoNTs described herein; plant cell systems infected with recombinant virus expression vectors (e.g., cauliflower mosaic virus (CaMV) and tobacco mosaic virus (TMV) or transformed with recombinant plasmid expression vectors (e.g., Ti plasmid) containing sequences encoding the BoNTs described herein; or mammalian cell systems (e.g., COS, CHO, BHK, 293, 293T, 3T3 cells, lymphotic cells (see U.S. Pat. No. 5,807,715), Per C.6 cells (human retinal cells developed by Crucell) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the BoNTs being expressed. For example, when a large quantity of such a protein is to be produced, for the generation of pharmaceutical compositions of BoNTs described herein, vectors which direct the expression of high levels of protein products that are readily purified may be desirable. Such vectors include, but are not limited, to the E. coli expression vector pUR278 (Rüther et al. (1983) “Easy Identification Of cDNA Clones,” EMBO J. 2:1791-1794), in which the coding sequence may be ligated individually into the vector in frame with the lac Z coding region so that a fusion protein is produced; pIN vectors (Inouye et al. (1985) “Up-Promoter Mutations In The lpp Gene Of Escherichia Coli,” Nucleic Acids Res. 13:3101-3110; Van Heeke et al. (1989) “Expression Of Human Asparagine Synthetase In Escherichia Coli,” J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to a matrix glutathione-agarose beads followed by elution in the presence of free glutathione.

The pGEX vectors are designed to include thrombin or factor Xa protease cleavage sites so that the cloned target gene product can be released from the GST moiety. In an insect system, Autographa californica nuclear polyhedrosis virus (AcNPV) is used as a vector to express foreign genes. The virus grows in Spodoptera frugiperda cells. The coding sequence may be cloned individually into non-essential regions (e.g., the polyhedrin gene) of the virus and placed under control of an AcNPV promoter (e.g., the polyhedrin promoter).

In mammalian host cells, a number of viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the immunoglobulin molecule in infected hosts (e.g., see Logan et al. (1984) “Adenovirus Tripartite Leader Sequence Enhances Translation Of mRNAs Late After Infection,” Proc. Natl. Acad. Sci. USA 81:3655-3659). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic.

The efficiency of expression may be enhanced by the inclusion of appropriate transcription enhancer elements, transcription terminators, etc. (see Bitter et al. (1987) “Expression And Secretion Vectors For Yeast,” Methods in Enzymol. 153:516-544). In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. For example, in certain embodiments, the BoNTs described herein may be expressed as a single gene product (e.g., as a single BoNT chain, i.e., as a polyprotein precursor), requiring proteolytic cleavage by native or recombinant cellular mechanisms to form separate LC and HC as described herein.

The disclosure thus encompasses engineering a nucleic acid sequence to encode a polyprotein precursor molecule comprising the BoNTs described herein, which includes coding sequences capable of directing post translational cleavage of said polyprotein precursor. Post-translational cleavage of the polyprotein precursor results in the BoNTs described herein. The post translational cleavage of the precursor molecule comprising the BoNTs described herein may occur in vivo (i.e., within the host cell by native or recombinant cell systems/mechanisms, e.g. furin cleavage at an appropriate site) or may occur in vitro (e.g. incubation of said BoNT chain in a composition comprising proteases or peptidases of known activity and/or in a composition comprising conditions or reagents known to foster the desired proteolytic action).

Purification and modification of recombinant proteins is well known in the art such that the design of the polyprotein precursor could include a number of embodiments readily appreciated by a skilled worker. Any known proteases or peptidases known in the art can be used for the described modification of the precursor molecule, e.g., thrombin or factor Xa (Nagai et al. (1985) “Oxygen Binding Properties Of Human Mutant Hemoglobins Synthesized In Escherichia Coli,” Proc. Nat. Acad. Sci. USA 82:7252-7255, and reviewed in Jenny et al. (2003) “A Critical Review Of The Methods For Cleavage Of Fusion Proteins With Thrombin And Factor Xa,” Protein Expr. Purif. 31:1-11, each of which is incorporated by reference herein in its entirety)), enterokinase (Collins-Racie et al. (1995) “Production Of Recombinant Bovine Enterokinase Catalytic Subunit In Escherichia Coli Using The Novel Secretory Fusion Partner DsbA,” Biotechnology 13:982-987 hereby incorporated by reference herein in its entirety)), furin, and AcTEV (Parks et al. (1994) “Release Of Proteins And Peptides From Fusion Proteins Using A Recombinant Plant Virus Proteinase,” Anal. Biochem. 216:413-417 hereby incorporated by reference herein in its entirety)) and the Foot and Mouth Disease Virus Protease C3.

Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, HeLa, COS, MDCK, 293, 293T, 3T3, WI38, BT483, Hs578T, HTB2, BT20 and T47D, CRL7030 and Hs578Bst.

For long-term, high-yield production of recombinant proteins, stable expression is preferred. For example, cell lines which stably express BoNTs described herein may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the BoNTs described herein.

A number of selection systems may be used, including but not limited to the herpes simplex virus thymidine kinase (Wigler et al. (1977) “Transfer Of Purified Herpes Virus Thymidine Kinase Gene To Cultured Mouse Cells,” Cell 11: 223-232), hypoxanthine-guanine phosphoribosyltransferase (Szybalska et al. (1992) “Use Of The HPRT Gene And The HAT Selection TecH_{N}ique In DNA-Mediated Transformation Of Mammalian Cells First Steps Toward Developing Hybridoma Tecchiques And Gene Therapy,” Bioessays 14: 495-500), and adenine phosphoribosyltransferase (Lowy et al. (1980) “Isolation Of Transforming DNA: Cloning The Hamster aprt Gene,” Cell 22: 817-823) genes can be employed in tk-, hgprt- or aprt-cells, respectively. Also, antimetabolite resistance can be used as the basis of selection for the following genes: dhfr, which confers resistance to methotrexate (Wigler et al. (1980) “Transformation Of Mammalian Cells With An Amplifiable Dominant-Acting Gene,” Proc. Natl. Acad. Sci. USA 77:3567-3570; O'Hare et al. (1981) “Transformation Of Mouse Fibroblasts To Methotrexate Resistance By A Recombinant Plasmid Expressing A Prokaryotic Dihydrofolate Reductase,” Proc. Natl. Acad. Sci. USA 78: 1527-1531); gpt, which confers resistance to mycophenolic acid (Mulligan et al. (1981) “Selection For Animal Cells That Express The Escherichia coli Gene Coding For Xanthine-Guanine Phosphoribosyltransferase,” Proc. Natl. Acad. Sci. USA 78: 2072-2076); neo, which confers resistance to the aminoglycoside G-418 (Tolstoshev (1993) “Gene Therapy, Concepts, Current Trials And Future Directions,” Ann. Rev. Pharmacol. Toxicol. 32:573-596; Mulligan (1993) “The Basic Science Of Gene Therapy,” Science 260:926-932; and Morgan et al. (1993) “Human Gene Therapy,” Ann. Rev. Biochem. 62:191-217) and hygro, which confers resistance to hygromycin (Santerre et al. (1984) “Expression Of Prokaryotic Genes For Hygromycin B And G418 Resistance As Dominant-Selection Markers In Mouse L Cells,” Gene 30:147-156). Methods commonly known in the art of recombinant DNA technology which can be used are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al. (1981) “A New Dominant Hybrid Selective Marker For Higher Eukaryotic Cells,” J. Mol. Biol. 150:1-14.

The expression levels of BoNTs described herein can be increased by vector amplification (for a review, see Bebbington and Hentschel, The use of vectors based on gene amplification for the expression of cloned genes in mammalian cells in DNA cloning, Vol. 3 (Academic Press, New York, 1987). When a marker in the vector system expressing a BoNT described herein is amplifiable, increase in the level of inhibitor present in culture of host cell will increase the number of copies of the marker gene. Since the amplified region is associated with the nucleotide sequence of a BoNT described herein, production of the BoNT will also increase (Crouse et al. (1983) “Expression And Amplification Of Engineered Mouse Dihydrofolate Reductase Minigenes,” Mol. Cell. Biol. 3:257-266).

Once a BoNT described herein has been recombinantly expressed, it may be purified by any method known in the art for purification of polypeptides, polyproteins or antibodies (e.g., analogous to antibody purification schemes based on antigen selectivity) for example, by chromatography (e.g., ion exchange, affinity, particularly by affinity for the specific antigen (optionally after Protein A selection where the polypeptide comprises an Fc domain (or portion thereof)), and sizing column chromatography), centrifugation, differential solubility, or by any other standard techₙique for the purification of polypeptides or antibodies. Other aspects of the present disclosure relate to a cell comprising a nucleic acid described herein or a vector described herein.

The cell may be a prokaryotic or eukaryotic cell. In some embodiments, the cell in a mammalian cell. Exemplary cell types are described herein. Other aspects of the present disclosure related to a cell expressing the BoNT described herein. The cell may be a prokaryotic or eukaryotic cell. In some embodiments, the cell in a mammalian cell. The cell can be for propagation of the nucleic acid or for expression of the nucleic acid, or both. Such cells include, without limitation, prokaryotic cells including, without limitation, strains of aerobic, microaerophilic, capnophilic, facultative, anaerobic, gram-negative and gram-positive bacterial cells such as those derived from, e.g., Escherichia coli, Bacillus subtilis, Bacillus licheniformis, Bacteroides fragilis, Clostridia perfringens, Clostridia difficile, Caulobacter crescentus, Lactococcus lactis, Methylobacterium extorquens, Neisseria meningirulls, Neisseria meningitidis, Pseudomonas fluorescens and Salmonella typhimurium; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; insect cells and cell lines derived from insects, such as, e.g., those derived from Spodoptera frugiperda, Trichoplusia ni, Drosophila melanogaster and Manduca sexta; and mammalian cells and cell lines derived from mammalian cells, such as, e.g., those derived from mouse, rat, hamster, porcine, bovine, equine, primate and human. Cell lines may be obtained from the American Type Culture Collection, European Collection of Cell Cultures and the German Collection of Microorganisms and Cell Cultures. Non-limiting examples of specific protocols for selecting, making and using an appropriate cell line are described in e.g., INSECT CELL CULTURE ENGINEERING (Mattheus F. A. Goosen et al. eds., Marcel Dekker, 1993); INSECT CELL CULTURES: FUNDAMENTAL AND APPLIED ASPECTS (J. M. Vlak et al. eds., Kluwer Academic Publishers, 1996); Maureen A. Harrison & Ian F. Rae, GENERAL TECH_{N}IQUES OF CELL CULTURE (Cambridge University Press, 1997); CELL AND TISSUE CULTURE: LABORATORY PROCEDURES (Alan Doyle et al eds., JOH_{N} Wiley and Sons, 1998); R. Ian FresH_{N}ey, CULTURE OF ANIMAL CELLS: A MANUAL OF BASIC TECH_{N}IQUE (Wiley-Liss, 4.sup.th ed. 2000); ANIMAL CELL CULTURE: A PRACTICAL APPROACH (JOH_{N} R. W. Masters ed., Oxford University Press, 3.sup.rd ed. 2000); MOLECULAR CLONING A LABORATORY MANUAL, supra, (2001); BASIC CELL CULTURE: A PRACTICAL APPROACH (JOH_{N} M. Davis, Oxford Press, 2.sup.nd ed. 2002); and CURRENT PROTOCOLS IN MOLECULAR BIOLOGY, supra, (2004).

These protocols are routine procedures within the scope of one skilled in the art and from the teaching herein. Yet other aspects of the present disclosure relate to a method of producing a BoNT described herein, the method comprising obtaining a cell described herein and expressing nucleic acid described herein in said cell. In some embodiments, the method further comprises isolating and purifying a BoNT described herein.

The term “identity” refers to the overall relatedness between polymeric molecules, for example, between polynucleotide molecules (e.g. DNA molecules and/or RNA molecules) and/or between polypeptide molecules. Calculation of the percent identity of two polypeptide sequences, for example, can be performed by aligning the two sequences for optimal comparison purposes (e.g., gaps can be introduced in one or both of a first and a second polypeptide sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding positions are then compared. When a position in the first sequence is occupied by the same amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm, which have been described and are available to those skilled in the art.

The catalytically inactive BoNTs described herein (e.g., ^{ci}LC-Hn/X, ^{ci}LC-Hn/En, ^{ci}LC-Hn/PMP1, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, ^{ci}BoNT/EnH, ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) can enter a cell (e.g., a neuron) but cannot cleave its substrates in the cell. The present disclosure provides the use of the catalytically inactive BoNTs described herein (e.g., ^{ci}LC-Hn/X, ^{ci}LC-Hn/En, ^{ci}LC-Hn/PMP1, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, ^{ci}BoNT/EnH, ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) as a delivery vehicle. In some embodiments, the catalytically inactive BoNT described herein (e.g., ^{ci}LC-Hn/X, ^{ci}LC-Hn/En, ^{ci}LC-Hn/PMP1, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, ^{ci}BoNT/EnH, ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) is used to deliver an agent (e.g., a therapeutic agent or diagnostic agent) into a cell (e.g., a neuron). The agent (e.g., a therapeutic agent or diagnostic agent) may be associated (e.g., covalently or non-covalently) with the catalytically inactive BoNTs described herein (e.g., ^{ci}LC-Hn/X, ^{ci}LC-Hn/En, ^{ci}LC-Hn/PMP1, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, ^{ci}BoNT/EnH, ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H).

Accordingly, other aspects of the present disclosure provide complexes comprising a catalytically inactive BoNT-like toxin described herein (e.g., ^{ci}LC-Hn/X, ^{ci}LC-Hn/En, ^{ci}LC-Hn/PMP1, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, ^{ci}BoNT/EnH, ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) associated with an agent. In some embodiments, the agent is associated with the catalytically inactive BoNT-like toxin covalently (e.g., via a chemical bondage or a chemical linker, or a peptide linker as a protein fusion). In some embodiments, the agent is associated with the catalytically inactive BoNT-like toxin non-covalently (e.g., via hydrogen bonding or van der waals interaction). In some embodiments, the agent is associated with the light chain of the catalytically inactive BoNT-like toxin. In some embodiments, the agent is associated with the heavy chain of the catalytically inactive BoNT-like toxin.

Being “covalently” associated means the two molecules are linked via a form of chemical bonding that is characterized by the sharing of one or more pairs of electrons between atoms. A covalent bond formed between two molecules may, for example, be an amide bond, an acyl bond, a disulfide bond, an alkyl bond, an ether bond, or an ester bond. A covalent bond formed between two molecules may be, for example, a carbon-carbon bond, a carbon-oxygen bond, a carbon-nitrogen bond, a carbon-sulfur bond, a sulfur-sulfur bond, a carbon-phosphorus bond, a phosphorus-oxygen bond, or a phosphorus-nitrogen bond. When two molecules are covalently associated, they are also referred to herein as being “conjugated” or “fused.” The covalent association can be, for example, via a direct or indirect (e.g., via a linker) covalent linkage. For example, in some embodiments, where two proteins are conjugated to each other, to form a protein fusion, the two proteins may be conjugated via a polypeptide linker, e.g., an amino acid sequence connecting the C-terminus of one protein to the N-terminus of the other protein.

In some embodiments, the catalytically inactive BoNT-like toxin and/or the agent may be functionalized with a reactive chemical group. One example of such reactive group is a “click chemistry handle.” Click chemistry is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “Click chemistry”) include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azidealkyne Huisgon cycloaddition; thiolyne addition; imine formation; and Michael additions (e.g., maleimide addition). Non-limiting examples of a click chemistry handle include an azide handle, an alkyne handle, or an aziridine handle. Azide is the anion with the formula N3. It is the conjugate base of hydrazoic acid (HN3). N3 is a linear anion that is isoelectronic with CO2, NCO—, N2O, NO2+ and NCF. Azide can be described by several resonance structures, an important one being —N═N+=N—. An alkyne is an unsaturated hydrocarbon containing at least one carboncarbon triple bond. The simplest acyclic alkynes with only one triple bond and no other functional groups form a homologous series with the general chemical formula CnH2n−2. Alkynes are traditionally known as acetylenes, although the name acetylene also refers specifically to C2H2, known formally as ethyne using IUPAC nomenclature. Like other hydrocarbons, alkynes are generally hydrophobic but tend to be more reactive. Aziridines are organic compounds containing the aziridine functional group, a three-membered heterocycle with one amine group (—NH—) and two methylene bridges (—CH2-).

Other non-limiting, exemplary reactive groups include: acetals, ketals, hemiacetals, and hemiketals, carboxylic acids, strong non-oxidizing acids, strong oxidizing acids, weak acids, acrylates and acrylic acids, acyl halides, sulfonyl halides, chloroformates, alcohols and polyols, aldehydes, alkynes with or without acetylenic hydrogen amides and imides, amines, aromatic, amines, phosphines, pyridines, anhydrides, aryl halides, azo, diazo, azido, hydrazine, and azide compounds, strong bases, weak bases, carbamates, carbonate salts, chlorosilanes, conjugated dienes, cyanides, inorganic, diazonium salts, epoxides, esters, sulfate esters, phosphate esters, thiophosphate esters borate esters, ethers, soluble fluoride salts, fluorinated organic compounds, halogenated organic compounds, halogenating agents, aliphatic saturated hydrocarbons, aliphatic unsaturated hydrocarbons, hydrocarbons, aromatic, insufficient information for classification, isocyanates and isothiocyanates, ketones, metal hydrides, metal alkyls, metal aryls, and silanes, alkali metals, nitrate and nitrite compounds, inorganic, nitrides, phosphides, carbides, and silicides, nitriles, nitro, nitroso, nitrate, nitrite compounds, organic, non-redox-active inorganic compounds, organometallics, oximes, peroxides, organic, phenolic salts, phenols and cresols, polymerizable compounds, quaternary ammonium and phosphonium salts, strong reducing agents, weak reducing agents, acidic salts, basic salts, siloxanes, inorganic sulfides, organic sulfides, sulfite and thiosulfate salts, sulfonates, phosphonates, organic thiophosphonates, thiocarbamate esters and salts, and dithiocarbamate esters and salts.

When one molecule of the complex (e.g., the catalytically inactive BoNT-like toxin or the agent) is functionalized with a chemically reactive group, the other molecule of the complex (e.g., the agent or the catalytically inactive BoNT-like toxin) may contain a corresponding chemical group that reacts with the chemically reactive group, thus resulting in covalent attachment. In some embodiments, the agent is a protein or peptide and one or more of the amino acids of the protein or peptide may be modified to include a chemical entity such as a carbohydrate group, a hydroxyl group, a phosphate group, a farnesyl group, an isofarnesyl group, a fatty acid group, a linker for attaching to the catalytically inactive BoNT-like toxin.

In some embodiments, the agent is linked to catalytically inactive toxins via sortase-mediated protein ligation, e.g., as described in Antos et al., Current Opinion in Structural Biology, 2016, 38:111-118, incorporated herein by reference.

Being “non-covalently” associated means two molecules are associated via a type of interaction that does not involve the sharing of electrons between the molecules, but involves variations of electromagnetic, electrostatic, or hydrophobic interactions. “Associated with” includes both covalent or non-covalent associate. When the associate is non-covalent, in some embodiments, the interactions between two molecules have a K_{D} of <10⁻⁵ M, <10⁻⁶ M, <10⁻⁷ M, <10⁻⁸ M, <10⁻⁹ M, <10⁻¹⁰ M, <10⁻¹¹ M, or <10⁻¹² M.

In some embodiments, the complex described herein comprises a catalytically inactive BoNT-like toxin (e.g., ^{ci}LC-Hn/X, ^{ci}LC-Hn/En, ^{ci}LC-Hn/PMP1, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, ^{ci}BoNT/EnH, ^{ci}BoNT/PMP1A, ^{ci}BoNT/PMP1B, ^{ci}BoNT/PMP1C, ^{ci}BoNT/PMP1D, ^{ci}BoNT/PMP1E, ^{ci}BoNT/PMP1F, ^{ci}BoNT/PMP1G, or ^{ci}BoNT/PMP1H) associated with an agent, wherein the catalytically inactive BoNT-like toxin is in its processed form (i.e., when the heavy chain and light chain are linked via a disulfide bond).

In some embodiments, the complex comprises ^{ci}BoNT/XA associated with an agent, wherein the ^{ci}BoNT/XA comprises: (a) a light chain comprising an inactive LC/X, (b) a heavy chain comprising: (i) a translocation domain from BoNT/X (Hn/X), and (ii) a receptor binding domain from BoNT/A (Hc/A), wherein the light chain and the heavy chain are linked via a disulfide bond. In some embodiments, the agent is a polypeptide and is fused to the N-terminus of the light chain (LC/X).

In some embodiments, the complex comprises ^{ci}BoNT/EnA associated with an agent, wherein the ^{ci}BoNT/EnA comprises: (a) a light chain comprising an inactive LC/En, (b) a heavy chain comprising: (i) a translocation domain from BoNT/En (Hn/En), and (ii) a receptor binding domain from BoNT/A (Hc/A), wherein the light chain and the heavy chain are linked via a disulfide bond. In some embodiments, the agent is a polypeptide and is fused to the N-terminus of the light chain (LC/En).

In some embodiments, the complex comprises ^{ci}BoNT/PMP1A associated with an agent, wherein the ^{ci}BoNT/PMP1A comprises: (a) a light chain comprising an inactive LC/PMP1, (b) a heavy chain comprising: (i) a translocation domain from BoNT/PMP1 (Hn/PMP1), and (ii) a receptor binding domain from BoNT/A (Hc/A), wherein the light chain and the heavy chain are linked via a disulfide bond. In some embodiments, the agent is a polypeptide and is fused to the N-terminus of the light chain (LC/PMP1).

In some embodiments, the agent is a nucleic acid. A “nucleic acid” is at least two nucleotides covalently linked together, and in some instances, may contain phosphodiester bonds (e.g., a phosphodiester “backbone”). A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine. Nucleic acids of the present disclosure may be produced using standard molecular biology methods (see, e.g., Green and Sambrook, Molecular Cloning, A Laboratory Manual, 2012, Cold Spring Harbor Press). None limiting examples of nucleic acid molecules that may be used as the agent of the present disclosure include: DNA or messenger RNA (mRNA) encoding a protein effector (e.g., without limitation, enzymes, antigens, antibodies, immune modulators, transcriptional activators, and transcriptional repressors), RNAi molecules (e.g., microRNA, siRNA, or shRNA), guide RNA (gRNAs), and DNA/RNA based aptamers.

In some embodiments, the agent is a protein or peptide. The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. The proteins/peptides can also be linked to inactive BoNT-like toxins via a disulfide bond, which would be able to release the delivered protein from the toxin once it reaches the cytosol of cells. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

In some embodiments, the agent is a small molecule. A “small molecule” refers to an organic compound, whether naturally-occurring or artificially created (e.g., via chemical synthesis) that has a relatively low molecular weight. Typically, an organic compound contains carbon. An organic compound may contain multiple carbon-carbon bonds, stereocenters, and other functional groups (e.g., amines, hydroxyl, carbonyls, or heterocyclic rings). In some embodiments, small molecules are monomeric organic compounds that have a molecular weight of less than about 1500 g/mol. In certain embodiments, the molecular weight of the small molecule is less than about 1000 g/mol or less than about 500 g/mol. In certain embodiments, the small molecule is a drug, for example, a drug that has already been deemed safe and effective for use in humans or animals by the appropriate governmental agency or regulatory body.

In some embodiments, the agent is a therapeutic agent. A “therapeutic agent” refers to an agent that has therapeutic effects to a disease or disorder. A therapeutic agent may be, without limitation, proteins, peptides, nucleic acids, polysaccharides and carbohydrates, lipids, glycoproteins, small molecules, synthetic organic and inorganic drugs exerting a biological effect when administered to a subject, a proteolysis targeting chimera molecule (PROTAC) and combinations thereof. In some embodiments, the therapeutic agent is an anti-inflammatory agent, a vaccine antigen, a vaccine adjuvant, an antibody, and enzyme, an anti-cancer drug or chemotherapeutic drug, a clotting factor, a hormone, a steroid, a cytokine, an antibiotic, or a drug for the treatment of cardiovascular disease, an infectious disease, an autoimmune disease, allergy, a blood disorder, a metabolic disorder, a skin disease, or a neurological disease. In some embodiments, the therapeutic agent is a drug for treating botulism, e.g., an antibody that can neutralize a BoNT.

In some embodiments, the therapeutic agent is an antibody or an antibody fragment. An “antibody” or “immunoglobulin (Ig)” is a large, Y-shaped protein produced mainly by plasma cells that is used by the immune system to neutralize an exogenous substance (e.g., a pathogens such as bacteria and viruses). Antibodies are classified as IgA, IgD, IgE, IgG, and IgM. “Antibodies” and “antibody fragments” include whole antibodies and any antigen binding fragment (i.e., “antigen-binding portion”) or single chain thereof. In some embodiments, an antibody is a glycoprotein comprising two or more heavy (H) chains and two or more light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody may be a polyclonal antibody or a monoclonal antibody.

The basic 4-chain antibody unit is a heterotetrameric glycoprotein composed of two identical L chains and two H chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to a H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site. For the structure and properties of the different classes of antibodies, (e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6, incorporated herein by reference).

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated α, δ, ε, γ and μ, respectively. The γ and a classes are further divided into subclasses on the basis of relatively minor differences in CH sequence and function, e.g., humans express the following subclasses: IgG1, IgG2, IgG3, IgG4, IgA1, and IgA2.

The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a (3-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, Md. (1991), incorporated herein by reference). The constant domains are not involved directly in binding an antibody to an antigen, but exhibit various effector functions, such as participation of the antibody in antibody dependent cellular cytotoxicity (ADCC).

In some embodiments, the antibody is a monoclonal antibody. A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present invention may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567). Monoclonal antibodies may also be isolated from phage antibody libraries, e.g., using the techniques described in Clackson et al., Nature, 352:624-628 (1991) and Marks et al., J. Mol. Biol., 222:581-597 (1991), incorporated herein by reference.

The monoclonal antibodies described herein encompass “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc.), and human constant region sequences.

In some embodiments, the antibodies are “humanized” for use in human (e.g., as therapeutics). “Humanized” forms of non-human (e.g., rodent) antibodies are chimeric antibodies that contain minimal sequence derived from the non-human antibody. Humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

In some embodiments, the therapeutic agent is an antibody fragment containing the antigen-binding portion of an antibody. The antigen-binding portion of an antibody refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (e.g., as described in Ward et al., (1989) Nature 341:544-546, incorporated herein by reference), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883, incorporated herein by reference). Such single chain antibodies are also intended to be encompassed within the term “antigen-binding portion” of an antibody. These antibody fragments are obtained using conventional techniques known to those with skill in the art, and the fragments are screened for utility in the same manner as are full-length antibodies.

In some embodiments, the therapeutic agent of the present disclosure is a Fc fragment, a Fv fragment, or a single-change Fv fragment. The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The Fv fragment is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

In some embodiments, the therapeutic agent is an antigen binding fragment. An “antigen binding fragment (Fab)” is the region on an antibody that binds antigens. The Fab is composed of one constant and one variable domain from each of the heavy and light chain polypeptides of the antibody. The antigen binding site is formed by the variable domains of the heavy and light chain antibodies.

In some embodiments, the therapeutic agent is a single chain variable fragment (ScFv). A “single-chain variable fragment (scFv)” is a fusion protein of the variable regions of the heavy (VH) and light chains (VL) of immunoglobulins, connected with a short peptide linker comprising 10-25 amino acids. The linker peptide is usually rich in glycine for flexibility, as well as serine or threonine for solubility, and connects the N-terminus of the VH chain with the C-terminus of the VL chain, or vice versa. The scFv retains the specificity of the original immunoglobulin, despite the addition of the linker and removal of the constant regions. In some embodiments, the sFv polypeptide further comprises a polypeptide linker between the VH and VL domains which enables the sFv to form the desired structure for antigen binding (e.g., as described in Pluckthun in The Pharmacology of Monoclonal Antibodies, vol. 113, Rosenburg and Moore eds., Springer-Verlag, New York, pp. 269-315 (1994); Borrebaeck 1995, incorporated herein by reference).

In some embodiments, the therapeutic agent is a diabody. A diabody is a dimeric antibody fragment designed to form two antigen binding sites. Diabodies are composed of two single-chain variable fragments (scFvs) in the same polypeptide connected by a linker peptide which is too short (˜3-6 amino acids) to allow pairing between the two domains on the same chain, forcing the domains to pair with complementary domains of another chain to form two antigen binding sites. Alternately, the two scFvs can also be connected with longer linkers, such as leucine zippers.

In some embodiments, the therapeutic agent is an affibody. An “affibody” is an antibody mimetics engineered to bind to a large number of target proteins or peptides with high affinity, imitating monoclonal antibodies. These molecules can be used for molecular recognition in diagnostic and therapeutic applications.

In some embodiments, the therapeutic agent of the present disclosure is single chain antibody (e.g., a heavy chain-only antibody). It is known that Camilids produce heavy chain-only antibodies (e.g., as described in Hamers-Casterman et al., 1992, incorporated herein by reference). The single-domain variable fragments of these heavy chain-only antibodies are termed VHHs or nanobodies. VHHs retain the immunoglobulin fold shared by antibodies, using three hypervariable loops, CDR1, CDR2 and CDR3, to bind to their targets. Many VHHs bind to their targets with affinities similar to conventional full-size antibodies, but possess other properties superior to them. Therefore, VHHs are attractive tools for use in biological research and therapeutics. VHHs are usually between 10 to 15 kDa in size, and can be recombinantly expressed in high yields, both in the cytosol and in the periplasm in E. coli. VHHs can bind to their targets in mammalian cytosol. A VHH fragment (e.g., NANOBODY®) is a recombinant, antigen-specific, single-domain, variable fragment derived from camelid heavy chain antibodies. Although they are small, VHH fragments retain the full antigen-binding capacity of the full antibody. VHHs are small in size, highly soluble and stable, and have greater set of accessible epitopes, compared to traditional antibodies. They are also easy to use as the extracellular target-binding moiety of the chimeric receptor described herein, because no reformatting is required. In the some embodiments, the therapeutic agent is a series of antibodies (e.g., VHHs) that target different targets.

In some embodiments, the therapeutic agent is an antibody that can neutralize a BoNT. Such BoNT-neutralizing antibodies can be delivered into a neuron using the catalytically inactive BoNT and the methods described herein. BoNT-neutralizing antibodies can be used to treat botulism, e.g., in subjects that have been exposed to a BoNT. In some embodiments, the BoNT-neutralizing antibody is a full length antibody, a FAB, a ScFv, a VHH, a diabody, or an affibody. BoNT-neutralizing antibodies are known in the art, e.g., as described in Tremblay et al., 2010, Toxicon, 56:990-998, incorporated herein by reference.

In some embodiments, the BoNT-neutralizing antibody is a BoNT/A antibody. In some embodiments, the BoNT/A antibody is an anti-BoNT/A VHH. In some embodiments, the anti-BoNT/A VHH targets the LC of BoNT/A. In some embodiments, the anti-BoNT/A VHH comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 57 or SEQ ID NO: 58. In some embodiments, the anti-BoNT/A VHH comprises the amino acid sequence of SEQ ID NO: 57 or SEQ ID NO: 58. In some embodiments, the anti-BoNT/A VHH consists of the amino acid sequence of SEQ ID NO: 57 or SEQ ID NO: 58.

In some embodiments, the BoNT-neutralizing antibody is a BoNT/B antibody. In some embodiments, the BoNT/A antibody is an anti-BoNT/B VHH. In some embodiments, the anti-BoNT/B VHH targets the LC of BoNT/B. In some embodiments, the anti-BoNT/B VHH comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 67, SEQ ID NO: 113, SEQ ID NO: 114, or SEQ ID NO: 130. In some embodiments, the anti-BoNT/B VHH comprises the amino acid sequence of SEQ ID NO: 67, SEQ ID NO: 113, SEQ ID NO: 114, or SEQ ID NO: 130. In some embodiments, the anti-BoNT/B VHH consists of the amino acid sequence of SEQ ID NO: 67, SEQ ID NO: 113, or SEQ ID NO: 114, or SEQ ID NO: 130.

In some embodiments, the BoNT-neutralizing antibody is a VHH fusion polypeptide (e.g., with a VHH targeting BoNT/A fused to a VHH targeting BoNT/B). In some embodiments, the VHH fusion polypeptide comprises a VHH as set forth in SEQ ID NO: 57 or SEQ ID NO: 58, or any variants thereof, fused to a VHH as set forth in SEQ ID NO 67, SEQ ID NO: 113, or SEQ ID NO: 114, or any variants thereof.

In some embodiments, the therapeutic agent for use in accordance with the present disclosure is a BoNT/A antibody (e.g., an anti-BoNT/A VHH) fused to a E. coli Thioredoxin 1 (TrxA), i.e., a TrxA-anti-BoNT/A VHH fusion protein or TrxA-anti-BoNT/B VHH fusion protein. In some embodiments, TrxA facilitates the folding of VHH protein and increase the yield of VHH in E. coli. In some embodiments, the TrxA is fused to the C-terminus of the anti-BoNT/A VHH. In some embodiments, the TrxA is fused to the N-terminus of the anti-BoNT/A VHH. In some embodiments, the TrxA is fused to the anti-BoNT/A VHH via a peptide linker (e.g., a linker that contains a protease cleavage site). The linker may contain any of the protease cleavage sites provided herein (e.g., SEQ ID NOs:77-84). In some embodiments, the TrxA-anti-BoNT/A VHH fusion protein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 59, 60, and 68. In some embodiments, the TrxA-anti-BoNT/A VHH fusion protein comprises the amino acid sequence of any one of SEQ ID NOs: 59, 60, and 68. In some embodiments, the TrxA-anti-BoNT/A VHH fusion protein consists of the amino acid sequence of any one of SEQ ID NOs: 59, 60, and 68. In some embodiments, a TrxA is fused to a VHH fusion protein (e.g., as exemplified in SEQ ID NOs: 116, 117, and 118). In some embodiments, the fusion polypeptide comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NOs: 116, 117, and 118. In some embodiments, fusion polypeptide comprises the amino acid sequence of SEQ ID NOs: 116, 117, and 118.

In some embodiments, the anti-BoNT/A VHH or the TrxA-anti-BoNT/A VHH fusion protein is attached to the LC of the catalytically inactive BoNT/X, BoNT/En, or BoNT/PMP1 described herein. For example, the anti-BoNT/A VHH or the TrxA-anti-BoNT/A VHH fusion protein may be fused to the N-terminus of the LC of the catalytically inactive BoNT/X, BoNT/En, or BoNT/PMP1 described herein. In some embodiments, the anti-BoNT/A VHH or the TrxA-anti-BoNT/A VHH fusion protein is fused to the N-terminus of the LC of ^{ci}BoNT/XA. In some embodiments, the anti-BoNT/A VHH or the TrxA-anti-BoNT/A VHH fusion protein is fused to the N-terminus of the LC of ^{ci}BoNT/EnA. In some embodiments, the anti-BoNT/A VHH or the TrxA-anti-BoNT/A VHH fusion protein is fused to the N-terminus of the LC of ^{ci}BoNT/PMP1A. In some embodiments, the anti-BoNT/A VHH or the TrxA-anti-BoNT/A VHH fusion protein is fused to the N-terminus of the LC of ^{ci}BoNT/XA, wherein the ^{ci}BoNT/XA is in its processed form (i.e., wherein the LC and the HC are linked via a disulfide bond). In some embodiments, the anti-BoNT/A VHH or the TrxA-anti-BoNT/A VHH fusion protein is fused to the N-terminus of the LC of ^{ci}BoNT/EnA, wherein the ^{ci}BoNT/EnA is in its processed form (i.e., wherein the LC and the HC are linked via a disulfide bond). In some embodiments, the anti-BoNT/A VHH or the TrxA-anti-BoNT/A VHH fusion protein is fused to the N-terminus of the LC of ^{ci}BoNT/PMP1A, wherein the ^{ci}BoNT/PMP1A is in its processed form (i.e., wherein the LC and the HC are linked via a disulfide bond).

In some embodiments, the complex described herein comprises a first polypeptide comprising an anti-BoNT/A VHH or TrxA-anti-BoNT/A VHH fusion protein fused to the N-terminus of a catalytically inactive LC/X and a second polypeptide comprising a Hn/X and Hc/A, wherein the first polypeptide and the second polypeptide are linked via a disulfide bond. In some embodiments, the complex described herein comprises a first polypeptide comprising an anti-BoNT/A VHH or TrxA-anti-BoNT/A VHH fusion protein fused to the N-terminus of a catalytically inactive LC/En and a second polypeptide comprising a Hn/En and Hc/A, wherein the first polypeptide and the second polypeptide are linked via a disulfide bond. In some embodiments, the complex described herein comprises a first polypeptide comprising an anti-BoNT/A VHH or TrxA-anti-BoNT/A VHH fusion protein fused to the N-terminus of a catalytically inactive LC/PMP1 and a second polypeptide comprising a Hn/PMP1 and Hc/A, wherein the first polypeptide and the second polypeptide are linked via a disulfide bond.

In some embodiments, the anti-BoNT/B VHH or the TrxA-anti-BoNT/B VHH fusion protein is attached to the LC of the catalytically inactive BoNT/X or BoNT/En described herein. For example, the anti-BoNT/B VHH or the TrxA-anti-BoNT/B VHH fusion protein may be fused to the N-terminus of the LC of the catalytically inactive BoNT/X or BoNT/En described herein. In some embodiments, the anti-BoNT/A VHH or the TrxA-anti-BoNT/B VHH fusion protein is fused to the N-terminus of the LC of ^{ci}BoNT/XA. In some embodiments, the anti-BoNT/B VHH or the TrxA-anti-BoNT/B VHH fusion protein is fused to the N-terminus of the LC of ^{ci}BoNT/EnA. In some embodiments, the anti-BoNT/A VHH or the TrxA-anti-BoNT/B VHH fusion protein is fused to the N-terminus of the LC of ^{ci}BoNTPMP1A. In some embodiments, the anti-BoNT/B VHH or the TrxA-anti-BoNT/B VHH fusion protein is fused to the N-terminus of the LC of ^{ci}BoNT/XA, wherein the ^{ci}BoNT/XA is in its processed form (i.e., wherein the LC and the HC are linked via a disulfide bond). In some embodiments, the anti-BoNT/B VHH or the TrxA-anti-BoNT/B VHH fusion protein is fused to the N-terminus of the LC of ^{ci}BoNT/EnA, wherein the ^{ci}BoNT/EnA is in its processed form (i.e., wherein the LC and the HC are linked via a disulfide bond). In some embodiments, the anti-BoNT/B VHH or the TrxA-anti-BoNT/B VHH fusion protein is fused to the N-terminus of the LC of ^{ci}BoNT/PMP1A, wherein the ^{ci}BoNT/PMP1A is in its processed form (i.e., wherein the LC and the HC are linked via a disulfide bond).

In some embodiments, the complex described herein comprises a first polypeptide comprising an anti-BoNT/B VHH or TrxA-anti-BoNT/B VHH fusion protein fused to the N-terminus of a catalytically inactive LC/X and a second polypeptide comprising a Hn/X and Hc/A, wherein the first polypeptide and the second polypeptide are linked via a disulfide bond. In some embodiments, the complex described herein comprises a first polypeptide comprising an anti-BoNT/B VHH or TrxA-anti-BoNT/B VHH fusion protein fused to the N-terminus of a catalytically inactive LC/En and a second polypeptide comprising a Hn/En and Hc/A, wherein the first polypeptide and the second polypeptide are linked via a disulfide bond. In some embodiments, the complex described herein comprises a first polypeptide comprising an anti-BoNT/B VHH or TrxA-anti-BoNT/B VHH fusion protein fused to the N-terminus of a catalytically inactive LC/PMP1 and a second polypeptide comprising a Hn/PMP1 and Hc/A, wherein the first polypeptide and the second polypeptide are linked via a disulfide bond.

In some embodiments, the complex described herein comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 61, 62, and 69, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 40-47, wherein the first polypeptide and the second polypeptide are linked via disulfide bond. In some embodiments, the complex described herein comprises a first polypeptide comprising the amino acid sequence of SEQ ID NO: 61 and a second polypeptide comprising the amino acid sequence of SEQ ID NO: 40, wherein the first polypeptide and the second polypeptide are linked via disulfide bond. In some embodiments, the complex described herein comprises a first polypeptide comprising the amino acid sequence of SEQ ID NO: 62 and a second polypeptide comprising the amino acid sequence of SEQ ID NO: 40, wherein the first polypeptide and the second polypeptide are linked via disulfide bond. In some embodiments, the complex described herein comprises a first polypeptide comprising the amino acid sequence of SEQ ID NO: 69 and a second polypeptide comprising the amino acid sequence of SEQ ID NO: 40, wherein the first polypeptide and the second polypeptide are linked via disulfide bond.

In some embodiments, the complex described herein comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 124, 125, and 131, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 40-47, wherein the first polypeptide and the second polypeptide are linked via disulfide bond. In some embodiments, the complex described herein comprises a first polypeptide comprising the amino acid sequence of SEQ ID NO: 124 and a second polypeptide comprising the amino acid sequence of SEQ ID NO: 40, wherein the first polypeptide and the second polypeptide are linked via disulfide bond. In some embodiments, the complex described herein comprises a first polypeptide comprising the amino acid sequence of SEQ ID NO: 125 and a second polypeptide comprising the amino acid sequence of SEQ ID NO: 40, wherein the first polypeptide and the second polypeptide are linked via disulfide bond. In some embodiments, the complex described herein comprises a first polypeptide comprising the amino acid sequence of SEQ ID NO: 131 and a second polypeptide comprising the amino acid sequence of SEQ ID NO: 40, wherein the first polypeptide and the second polypeptide are linked via disulfide bond.

In some embodiments, the complex described herein comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 63, 64, and 70, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 49-56, wherein the first polypeptide and the second polypeptide are linked via disulfide bond. In some embodiments, the complex described herein comprises a first polypeptide comprising the amino acid sequence of SEQ ID NO: 63 and a second polypeptide comprising the amino acid sequence of SEQ ID NO: 49, wherein the first polypeptide and the second polypeptide are linked via disulfide bond. In some embodiments, the complex described herein comprises a first polypeptide comprising the amino acid sequence of SEQ ID NO: 64 and a second polypeptide comprising the amino acid sequence of SEQ ID NO: 49, wherein the first polypeptide and the second polypeptide are linked via disulfide bond. In some embodiments, the complex described herein comprises a first polypeptide comprising the amino acid sequence of SEQ ID NO: 70 and a second polypeptide comprising the amino acid sequence of SEQ ID NO: 49, wherein the first polypeptide and the second polypeptide are linked via disulfide bond.

In some embodiments, the complex described herein comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 126, 127, and 132, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 49-56, wherein the first polypeptide and the second polypeptide are linked via disulfide bond. In some embodiments, the complex described herein comprises a first polypeptide comprising the amino acid sequence of SEQ ID NO: 126 and a second polypeptide comprising the amino acid sequence of SEQ ID NO: 49, wherein the first polypeptide and the second polypeptide are linked via disulfide bond. In some embodiments, the complex described herein comprises a first polypeptide comprising the amino acid sequence of SEQ ID NO: 127 and a second polypeptide comprising the amino acid sequence of SEQ ID NO: 49, wherein the first polypeptide and the second polypeptide are linked via disulfide bond. In some embodiments, the complex described herein comprises a first polypeptide comprising the amino acid sequence of SEQ ID NO: 132 and a second polypeptide comprising the amino acid sequence of SEQ ID NO: 49, wherein the first polypeptide and the second polypeptide are linked via disulfide bond.

In some embodiments, the complex described herein comprise an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 65, 66, 71, 72, 128, 129, 133, and 134. In some embodiments, the complex described herein comprises the amino acid sequence of any one of SEQ ID NOs: 65, 66, 71, 72, 128, 129, 133, and 134.

In some embodiments, the complex described herein comprise an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 128, 129, 133, and 134. In some embodiments, the complex described herein comprises the amino acid sequence of any one of SEQ ID NOs: 128, 129, 133, and 134.

In some embodiments, the complex described herein comprises two BoNT targeting VHHs (an anti-BoNT/A VHH and an anti-BoNT/B VHH) fused to a catalytically inactive BoNT described herein. VHH fusion polypeptides comprising two VHHs are exemplified in SEQ ID NOs: 116, 117, and 118. In some embodiments, the VHH fusion polypeptide comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NOs: 116, 117, and 118. In some embodiments, VHH fusion polypeptide comprises the amino acid sequence of SEQ ID NOs: 116, 117, and 118.

In some embodiments, the complex described herein comprises two BoNT targeting VHHs (an anti-BoNT/A VHH and an anti-BoNT/B VHH) fused to a catalytically inactive BoNT described herein. VHH fusion polypeptides comprising two VHHs are exemplified in SEQ ID NOs: 139, 140, and 141. In some embodiments, the VHH fusion polypeptide comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NOs: 139, 140, and 141. In some embodiments, VHH fusion polypeptide comprises the amino acid sequence of SEQ ID NOs: 139, 140, and 141.

In some embodiments, the complex comprising two BoNT-targeting VHHs comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 73, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 40-47, wherein the first polypeptide and the second polypeptide are linked via disulfide bond. In some embodiments, the complex comprising two BoNT-targeting VHHs comprises a first polypeptide comprising the amino acid sequence of SEQ ID NO: 73 and a second polypeptide comprising the amino acid sequence of SEQ ID NO: 40, wherein the first polypeptide and the second polypeptide are linked via disulfide bond.

In some embodiments, the complex comprising two BoNT-targeting VHHs comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 135, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 40-47, wherein the first polypeptide and the second polypeptide are linked via disulfide bond. In some embodiments, the complex comprising two BoNT-targeting VHHs comprises a first polypeptide comprising the amino acid sequence of SEQ ID NO: 135 and a second polypeptide comprising the amino acid sequence of SEQ ID NO: 40, wherein the first polypeptide and the second polypeptide are linked via disulfide bond.

In some embodiments, the complex comprising two BoNT-targeting VHHs comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 74, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 49-56, wherein the first polypeptide and the second polypeptide are linked via disulfide bond. In some embodiments, the complex comprising two BoNT-targeting VHHs comprises a first polypeptide comprising the amino acid sequence of SEQ ID NO: 74 and a second polypeptide comprising the amino acid sequence of SEQ ID NO: 49, wherein the first polypeptide and the second polypeptide are linked via disulfide bond.

In some embodiments, the complex comprising two BoNT-targeting VHHs comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 136, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 49-56, wherein the first polypeptide and the second polypeptide are linked via disulfide bond. In some embodiments, the complex comprising two BoNT-targeting VHHs comprises a first polypeptide comprising the amino acid sequence of SEQ ID NO: 136 and a second polypeptide comprising the amino acid sequence of SEQ ID NO: 49, wherein the first polypeptide and the second polypeptide are linked via disulfide bond.

In some embodiments, the complex comprising two BoNT-targeting VHHs comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 75, 76, 119, 120, 137, 138, and 142-150 In some embodiments, the complex comprising two BoNT-targeting VHHs comprises the amino acid sequence of any one of 75, 76, 119, 120, 137, 138, and 142-150.

Other antibodies that may be used in accordance with the present disclosure target antigens including, without limitation: (a) anti-cluster of differentiation antigen CD-1 through CD-166 and the ligands or counter receptors for these molecules; (b) anti-cytokine antibodies, e.g., anti-IL-1 through anti-IL-18 and the receptors for these molecules; (c) anti-immune receptor antibodies, antibodies against T cell receptors, major histocompatibility complexes I and II, B cell receptors, selectin killer inhibitory receptors, killer activating receptors, OX-40, MadCAM-1, Gly-CAM1, integrins, cadherens, sialoadherens, Fas, CTLA-4, Fc.gamma.-receptors, Fcalpha-receptors, Fc.epsilon.-receptors, Fc.mu.-receptors, and their ligands; (d) anti-metalloproteinase antibodies, e.g., collagenase, MMP-1 through MMP-8, TIMP-1, TIMP-2; anti-cell lysis/proinflammatory molecules, e.g., perforin, complement components, prostanoids, nitron oxide, thromboxanes; and (e) anti-adhesion molecules, e.g., carcioembryonic antigens, lamins, fibronectins.

Other non-limiting, exemplary antibodies and fragments thereof that may be used in accordance with the present disclosure include: bevacizumab (AVASTIN®), trastuzumab (HERCEPTIN®), alemtuzumab (CAMPATH®, indicated for B cell chronic lymphocytic leukemia,), gemtuzumab (MYLOTARG®, hP67.6, anti-CD33, indicated for leukemia such as acute myeloid leukemia), rituximab (RITUXAN®), tositumomab (BEXXAR®, anti-CD20, indicated for B cell malignancy), MDX-210 (bispecific antibody that binds simultaneously to HER-2/neu oncogene protein product and type I Fc receptors for immunoglobulin G (IgG) (Fc gamma RI)), oregovomab (OVAREX®, indicated for ovarian cancer), edrecolomab (PANOREX®), daclizumab (ZENAPAX®), palivizumab (SYNAGIS®, indicated for respiratory conditions such as RSV infection), ibritumomab tiuxetan (ZEVALIN®, indicated for Non-Hodgkin's lymphoma), cetuximab (ERBITUX®), MDX-447, MDX-22, MDX-220 (anti-TAG-72), IOR-05, IOR-T6 (anti-CD1), IOR EGF/R3, celogovab (ONCOSCINT® OV103), epratuzumab (LYMPHOCIDE®), pemtumomab (THERAGYN®) and Gliomab-H (indicated for brain cancer, melanoma). Other antibodies and antibody fragments are contemplated and may be used in accordance with the disclosure.

In some embodiments, the therapeutic agent is a vaccine antigen. A “vaccine antigen” is a molecule or moiety that, when administered to a subject, activates or increases the production of antibodies that specifically bind the antigen. In some embodiments, an antigen is a protein or a polysaccharide. Antigens of pathogens are well known to those of skill in the art and include, but are not limited to parts (coats, capsules, cell walls, flagella, fimbriae, and toxins) of bacteria, viruses, and other microorganisms. A vaccine typically comprises an antigen, and is intentionally administered to a subject to induce an immune response in the recipient subject. The antigen may be from a pathogenic virus, bacteria, or fungi.

Examples of pathogenic virus include, without limitation: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses’); Poxviridae (variola viruses, vaccinia viruses, pox viruses); and Iridoviridae (e.g., African swine fever virus); and unclassified viruses (e.g., the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Examples of pathogenic bacteria include, without limitation: Helicobacter pyloris, Borelia burgdorferi, Legionella pneumophilia, Mycobacteria spp. (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic spp.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidum, Treponema pertenue, Leptospira, and Actinomyces israelli.

Examples of pathogenic fungi include, without limitation: Cryptococcus neoformans, Histoplasma capsulatum, Coccidioides immitis, Blastomyces dermatitidis, Chlamydia trachomatis, Candida albicans. Other infectious organisms (i.e., protists) include: Plasmodium falciparum and Toxoplasma gondii.

Other non-limiting examples of agents that may be delivered using the glycosphingolipids described herein are provided.

Non-limiting, exemplary chemopharmaceutically compositions that may be used in the liposome drug delivery systems of the present disclosure include, Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine. In some embodiments, the chemotherapeutic agent is Doxorubicin.

Examples of antineoplastic compounds include, without limitation: nitrosoureas, e.g., carmustine, lomustine, semustine, strepzotocin; Methylhydrazines, e.g., procarbazine, dacarbazine; steroid hormones, e.g., glucocorticoids, estrogens, progestins, androgens, tetrahydrodesoxycaricosterone, cytokines and growth factors; Asparaginase.

Examples of immunoactive compounds include, without limitation: immunosuppressives, e.g., pyrimethamine, trimethopterin, penicillamine, cyclosporine, azathioprine; immunostimulants, e.g., levamisole, diethyl dithiocarbamate, enkephalins, endorphins.

Examples of antimicrobial compounds include, without limitation: antibiotics, e.g., beta lactam, penicillin, cephalosporins, carbapenims and monobactams, beta-lactamase inhibitors, aminoglycosides, macrolides, tetracyclins, spectinomycin; Antimalarials, Amebicides, Antiprotazoal, Antifungals, e.g., amphotericin beta, antiviral, e.g., acyclovir, idoxuridine, ribavirin, trifluridine, vidarbine, gancyclovir.

Examples of parasiticides include, without limitation: antihalmintics, Radiopharmaceutics, gastrointestinal drugs.

Examples of hematologic compounds include, without limitation: immunoglobulins; blood clotting proteins; e.g., antihemophilic factor, factor IX complex; anticoagulants, e.g., dicumarol, heparin Na; fibrolysin inhibitors, tranexamic acid.

Examples of cardiovascular drugs include, without limitation: peripheral antiadrenergic drugs, centrally acting antihypertensive drugs, e.g., methyldopa, methyldopa HCl; antihypertensive direct vasodilators, e.g., diazoxide, hydralazine HCl; drugs affecting renin-angiotensin system; peripheral vasodilators, phentolamine; antianginal drugs; cardiac glycosides; inodilators; e.g., amrinone, milrinone, enoximone, fenoximone, imazodan, sulmazole; antidysrhythmic; calcium entry blockers; drugs affecting blood lipids; ranitidine, bosentan, rezulin.

Examples of respiratory drugs include, without limitation: sypathomimetic drugs: albuterol, bitolterol mesylate, dobutamine HCl, dopamine HCl, ephedrine SO, epinephrine, fenfluramine HCl, isoproterenol HCl, methoxamine HCl, norepinephrine bitartrate, phenylephrine HCl, ritodrine HCl; cholinomimetic drugs, e.g., acetylcholine Cl; anticholinesterases, e.g., edrophonium Cl; cholinesterase reactivators; adrenergic blocking drugs, e.g., acebutolol HCl, atenolol, esmolol HCl, labetalol HCl, metoprolol, nadolol, phentolamine mesylate, propanolol HCl; antimuscarinic drugs, e.g., anisotropine methylbromide, atropine SO4, clinidium Br, glycopyrrolate, ipratropium Br, scopolamine HBr.

Examples of neuromuscular blocking drugs include, without limitation: depolarizing, e.g., atracurium besylate, hexafluorenium Br, metocurine iodide, succinylcholine Cl, tubocurarine Cl, vecuronium Br; centrally acting muscle relaxants, e.g., baclofen.

Examples of neurotransmitters and neurotransmitter agents include, without limiation: acetylcholine, adenosine, adenosine triphosphate, amino acid neurotransmitters, e.g., excitatory amino acids, GABA, glycine; biogenic amine neurotransmitters, e.g., dopamine, epinephrine, histamine, norepinephrine, octopamine, serotonin, tyramine; neuropeptides, nitric oxide, K+ channel toxins,

Examples of antiparkinson drugs include, without limiation: amaltidine HCl, benztropine mesylate, e.g., carbidopa.

Examples of diuretic drugs include, without limitation: dichlorphenamide, methazolamide, bendroflumethiazide, polythiazide.

Examples of uterine, antimigraine drugs include, without limitation: carboprost tromethamine, mesylate, methysergide maleate.

Examples of hormones include, without limitation: pituitary hormones, e.g., chorionic gonadotropin, cosyntropin, menotropins, somatotropin, iorticotropin, protirelin, thyrotropin, vasopressin, lypressin; adrenal hormones, e.g., beclomethasone dipropionate, betamethasone, dexamethasone, triamcinolone; pancreatic hormones, e.g., glucagon, insulin; parathyroid hormone, e.g., dihydrochysterol; thyroid hormones, e.g., calcitonin etidronate disodium, levothyroxine Na, liothyronine Na, liotrix, thyroglobulin, teriparatide acetate; antithyroid drugs; estrogenic hormones; progestins and antagonists, hormonal contraceptives, testicular hormones; gastrointestinal hormones: cholecystokinin, enteroglycan, galanin, gastric inhibitory polypeptide, epidermal growth factor-urogastrone, gastric inhibitory polypeptide, gastrin-releasing peptide, gastrins, pentagastrin, tetragastrin, motilin, peptide YY, secretin, vasoactive intestinal peptide, sincalide.

Examples of enzymes include, without limitation: hyaluronidase, streptokinase, tissue plasminogen activator, urokinase, PGE-adenosine deaminase, oxidoreductases, transferases, polymerases, hydrolases, lyases, synthases, isomerases, and ligases, digestive enzymes (e.g., proteases, lipases, carbohydrases, and nucleases). In some embodiments, the enzyme is selected from the group consisting of lactase, beta-galactosidase, a pancreatic enzyme, an oil-degrading enzyme, mucinase, cellulase, isomaltase, alginase, digestive lipases (e.g., lingual lipase, pancreatic lipase, phospholipase), amylases, cellulases, lysozyme, proteases (e.g., pepsin, trypsin, chymotrypsin, carboxypeptidase, elastase), esterases (e.g. sterol esterase), disaccharidases (e.g., sucrase, lactase, beta-galactosidase, maltase, isomaltase), DNases, and RNases.

Examples of intravenous anesthetics include, without limitation: droperidol, etomidate, fetanyl citrate/droperidol, hexobarbital, ketamine HCl, methohexital Na, thiamylal Na, thiopental Na.

Examples of antiepileptics include, without limitation, carbamazepine, clonazepam, divalproex Na, ethosuximide, mephenytoin, paramethadione, phenytoin, primidone.

Examples of peptides and proteins that may be used as therapeutic agents include, without limiation: ankyrins, arrestins, bacterial membrane proteins, clathrin, connexins, dystrophin, endothelin receptor, spectrin, selectin, cytokines; chemokines; growth factors, insulin, erythropoietin (EPO), tumor necrosis factor (TNF), neuropeptides, neuropeptide Y, neurotensin, transforming growth factor alpha, transforming growth factor beta, interferon (IFN), and hormones, growth inhibitors, e.g., genistein, steroids etc; glycoproteins, e.g., ABC transporters, platelet glycoproteins, GPIb-IX complex, GPIIb-IIIa complex, vitronectin, thrombomodulin, CD4, CD55, CD58, CD59, CD44, lymphocye function-associated antigen, intercellular adhesion molecule, vascular cell adhesion molecule, Thy-1, antiporters, CA-15-3 antigen, fibronectins, laminin, myelin-associated glycoprotein, GAP, GAP-43, Exendin-4, and GLP-1.

Examples of cytokines and cytokine receptors include, without limitation: interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-1 receptor, IL-2 receptor, IL-3 receptor, IL-4 receptor, IL-5 receptor, IL-6 receptor, IL-7 receptor, IL-8 receptor, IL-9 receptor, IL-10 receptor, IL-11 receptor, IL-12 receptor, IL-13 receptor, IL-14 receptor, IL-15 receptor, IL-16 receptor, IL-17 receptor, IL-18 receptor, lymphokine inhibitory factor, macrophage colony stimulating factor, platelet derived growth factor, stem cell factor, tumor growth factor beta, tumor necrosis factor, lymphotoxin, Fas, granulocyte colony stimulating factor, granulocyte macrophage colony stimulating factor, interferon-alpha, interferon-beta, interferon-gamma.

Examples of growth factors and protein hormones include, without limitation: erythropoietin, angiogenin, hepatocyte growth factor, fibroblast growth factor, keratinocyte growth factor, nerve growth factor, tumor growth factor-alpha, thrombopoietin, thyroid stimulating factor, thyroid releasing hormone, neurotrophin, epidermal growth factor, VEGF, ciliary neurotrophic factor, LDL, somatomedin, insulin growth factor, insulin-like growth factor I and II.

Examples of chemokines include, without limitation: ENA-78, ELC, GRO-alpha, GRO-beta, GRO-gamma, HRG, LIF, IP-10, MCP-1, MCP-2, MCP-3, MCP-4, MIP-lalpha, MIP-lbeta, MIG, MDC, NT-3, NT-4, SCF, LIF, leptin, RANTES, lymphotactin, eotaxin-1, eotaxin-2, TARC, TECK, WAP-1, WAP-2, GCP-1, GCP-2; alpha-chemokine receptors: CXCR1, CXCR2, CXCR3, CXCR4, CXCR5, CXCR6, CXCR7; beta-chemokine receptors: CCR1, CCR2, CCR3, CCR4, CCR5, CCR6, CCR7.

In some embodiments, the therapeutic agent is a regulatory protein. A regulatory protein may be, in some embodiments, a transcription factor or a immunoregulatory protein. Non-limiting, exemplary transcriptional factors include: those of the NFkB family, such as Rel-A, c-Rel, Rel-B, p50 and p52; those of the AP-1 family, such as Fos, FosB, Fra-1, Fra-2, Jun, JunB and JunD; ATF; CREB; STAT-1, -2, -3, -4, -5 and -6; NFAT-1, -2 and -4; MAF; Thyroid Factor; IRF; Oct-1 and -2; NF-Y; Egr-1; and USF-43, EGR1, Spl, and E2F1.

In some embodiments, the therapeutic agent is an antiviral agent. Examples of antiviral agents include, without limitation: reverse transcriptase inhibitors and nucleoside analogs, e.g. ddI, ddC, 3TC, ddA, AZT; protease inhibitors, e.g., Invirase, ABT-538; inhibitors of in RNA processing, e.g., ribavirin.

Other non-limiting examples of known therapeutics which may be delivered by coupling to a glycosphingolipid a ceramide structure described herein include:

(a) Capoten, Monopril, Pravachol, Avapro, Plavix, Cefzil, Duricef/Ultracef, Azactam, Videx, Zerit, Maxipime, VePesid, Paraplatin, Platinol, Taxol, UFT, Buspar, Serzone, Stadol NS, Estrace, Glucophage (Bristol-Myers Squibb);

(b) Ceclor, Lorabid, Dynabac, Prozac, Darvon, Permax, Zyprexa, Humalog, Axid, Gemzar, Evista (Eli Lily);

(c) Vasotec/Vaseretic, Mevacor, Zocor, Prinivil/Prinizide, Plendil, Cozaar/Hyzaar, Pepcid, Prilosec, Primaxin, Noroxin, Recombivax HB, Varivax, Timoptic/XE, Trusopt, Proscar, Fosamax, Sinemet, Crixivan, Propecia, Vioxx, Singulair, Maxalt, Ivermectin (Merck & Co.);

(d) Diflucan, Unasyn, Sulperazon, Zithromax, Trovan, Procardia XL, Cardura, Norvasc, Dofetilide, Feldene, Zoloft, Zeldox, Glucotrol XL, Zyrtec, Eletriptan, Viagra, Droloxifene, Aricept, Lipitor (Pfizer);

(e) Vantin, Rescriptor, Vistide, Genotropin, Micronase/Glyn./Glyb., Fragmin, Total Medrol, Xanax/alprazolam, Sermion, Halcion/triazolam, Freedox, Dostinex, Edronax, Mirapex, Pharmorubicin, Adriamycin, Camptosar, Remisar, Depo-Provera, Caverject, Detrusitol, Estring, Healon, Xalatan, Rogaine (Pharmacia & Upjohn);

(f) Lopid, Accrupil, Dilantin, Cognex, Neurontin, Loestrin, Dilzem, Fempatch, Estrostep, Rezulin, Lipitor, Omnicef, FemHRT, Suramin, Clinafloxacin (Warner Lambert).

Further non-limiting examples of therapeutic agents which may be delivered by the glycosphingolipid-therapeutic agent complex of the present invention may be found in: Goodman and Gilman's The Pharmacological Basis of Therapeutics. 9th ed. McGraw-Hill 1996, incorporated herein by reference.

In some embodiments, the agent is a diagnostic agent. A “diagnostic agent” refers to an agent that is used for diagnostic purpose, e.g., by detecting another molecule in a cell or a tissue. In some embodiments, the diagnostic agent is an agent that targets (e.g., binds) a biomarker known to be associated with a disease (e.g., a nucleic acid biomarker, protein biomarker, or a metabolite biomarker) in a subject and produces a detectable signal, which can be used to determine the presence/absence of the biomarker, thus to diagnose a disease. For example, the diagnostic agent may be, without limitation, an antibody or an antisense nucleic acid.

In some embodiments, the diagnostic agent contains a detectable molecule. A detectable molecule refers to a moiety that has at least one element, isotope, or a structural or functional group incorporated that enables detection of a molecule, e.g., a protein or polypeptide, or other entity, to which the diagnostic agent binds. In some embodiments, a detectable molecule falls into any one (or more) of five classes: a) an agent which contains isotopic moieties, which may be radioactive or heavy isotopes, including, but not limited to, 2H, 3H, 13C, 14C, 15N, 18F, 31P, 32P, 35S, 67Ga, 76Br, 99mTc (Tc-99m), 111In, 123I, 125I, 131I, 153Gd, 169Yb, and 186Re; b) an agent which contains an immune moiety, which may be an antibody or antigen, which may be bound to an enzyme (e.g., such as horseradish peroxidase); c) an agent comprising a colored, luminescent, phosphorescent, or fluorescent moiety (e.g., such as the fluorescent label fluoresceinisothiocyanat (FITC); d) an agent which has one or more photo affinity moieties; and e) an agent which is a ligand for one or more known binding partners (e.g., biotin-streptavidin, His-NiTNAFK506-FKBP). In some embodiments, a detectable molecule comprises a radioactive isotope. In some embodiments, a detection agent comprises a fluorescent moiety. In some embodiments, the detectable molecule comprises a dye, e.g., a fluorescent dye, e.g., fluorescein isothiocyanate, Texas red, rhodamine, Cy3, Cy5, Cy5.5, Alexa 647 and derivatives. In some embodiments, the detectable molecule comprises biotin. In some embodiments, the detectable molecule is a fluorescent polypeptide (e.g., GFP or a derivative thereof such as enhanced GFP (EGFP)) or a luciferase (e.g., a firefly, Renilla, or Gaussia luciferase). In some embodiments, a detectable molecule may react with a suitable substrate (e.g., a luciferin) to generate a detectable signal. Non-limiting examples of fluorescent proteins include GFP and derivatives thereof, proteins comprising chromophores that emit light of different colors such as red, yellow, and cyan fluorescent proteins, etc. Exemplary fluorescent proteins include, e.g., Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, T-Sapphire, mAmetrine, mKeima. See, e.g., Chalfie, M. and Kain, S R (eds.) Green fluorescent protein: properties, applications, and protocols (Methods of biochemical analysis, v. 47, Wiley-Interscience, and Hoboken, N.J., 2006, and/or Chudakov, D M, et al., Physiol Rev. 90(3):1103-63, 2010, incorporated herein by reference, for discussion of GFP and numerous other fluorescent or luminescent proteins. In some embodiments, a detectable molecule comprises a dark quencher, e.g., a substance that absorbs excitation energy from a fluorophore and dissipates the energy as heat.

Other aspects of the present disclosure provide compositions comprising any of the catalytically inactive BoNT-like toxin described herein (e.g., ^{ci}LC-Hn/X, ^{ci}LC-Hn/En, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) or any the complexes comprising any of the catalytically inactive BoNT-like toxin described herein associated with an agent (e.g., a BoNT neutralizing antibody such as an anti-BoNT/A VHH). In some embodiments, the composition is a pharmaceutical composition. In some embodiments, the pharmaceutical composition further comprises other therapeutic agents suitable for the specific disease such composition is designed to treat. In some embodiments, the pharmaceutically composition of the present disclosure further comprises a pharmaceutically-acceptable carrier.

The term “pharmaceutically-acceptable carrier”, as used herein, means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the polypeptide from one site (e.g., the delivery site) of the body, to another site (e.g., organ, tissue or portion of the body).

A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethylcellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, a BoNT polypeptide of the present disclosure in a composition is administered by injection, by means of a catheter, by means of a suppository, or by means of an implant, the implant being of a porous, non-porous, or gelatinous material, including a membrane, such as a sialastic membrane, or a fiber.

Typically, when administering the composition, materials to which the catalytically inactive BoNT-like toxin or the complex of the disclosure does not absorb are used. In other embodiments, the BoNT polypeptides of the present disclosure are delivered in a controlled release system. Such compositions and methods for administration are provides in U.S. Patent publication No. 2007/0020295, the contents of which are herein incorporated by reference. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

The catalytically inactive BoNT-like toxin or the complex of the present disclosure can be administered as pharmaceutical compositions comprising a therapeutically effective amount of a binding agent and one or more pharmaceutically compatible ingredients. In typical embodiments, the pharmaceutical composition is formulated in accordance with routine procedures as a pharmaceutical composition adapted for intravenous or subcutaneous administration to a subject, e.g., a human being.

Typically, compositions for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical is administered by injection, an ampoule of sterile water for injection or saline can be provided so that the ingredients can be mixed prior to administration. A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein.

The catalytically inactive BoNT or the compolex of the present disclosure can be entrapped in ‘stabilized plasmid-lipid particles’ (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757. The pharmaceutical compositions of the present disclosure may be administered or packaged as a unit dose, for example.

The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle. In some embodiments, the BoNT/X polypeptides described herein may be conjugated to a therapeutic moiety, e.g., an antibiotic. TecH_{N}iques for conjugating such therapeutic moieties to polypeptides, including e.g., Fc domains, are well known; see, e.g., Amon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc.); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al. (1982) “The Preparation And Cytotoxic Properties Of Antibody-Toxin Conjugates,” Immunol. Rev., 62:119-158. Further, the pharmaceutical composition can be provided as a pharmaceutical kit comprising (a) a container containing a polypeptide of the disclosure in lyophilized form and (b) a second container containing a pharmaceutically acceptable diluent (e.g., sterile water) for injection. The pharmaceutically acceptable diluent can be used for reconstitution or dilution of the lyophilized polypeptide of the disclosure. Optionally associated with such container(s) can be a notice in the form prescribed by a governmental agency regulating the manufacture, use or sale of pharmaceuticals or biological products, which notice reflects approval by the agency of manufacture, use or sale for human administration. In another aspect, an article of manufacture containing materials useful for the treatment of the diseases described above is included. In some embodiments, the article of manufacture comprises a container and a label.

Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is an isolated polypeptide of the disclosure. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

The catalytically inactive BoNT-like toxin described herein can enter cells. For example, the ^{ci}LC-Hn/X or ^{ci}LC-Hn/En can enter cells non-specifically (i.e., not targeting a certain cell type). In some embodiments, the chimeric BoNT-like toxins (e.g., ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) targets neurons via its receptor binding domain. The present disclosure provide the use of the catalytically inactive BoNT-like toxin described herein (e.g., ^{ci}LC-Hn/X, ^{ci}LC-Hn/En, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) for delivering an agent (e.g., a therapeutic agent or a diagnostic agent) to a cell (e.g., a neuron). The present disclosure further provides the use of the complex comprising the catalytically inactive BoNT-like toxin described herein (e.g., ^{ci}LC-Hn/X, ^{ci}LC-Hₙ/En, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) associated with the agent (e.g., a therapeutic agent or a diagnostic agent) for treating or diagnosing a disease.

In some embodiments, methods of delivering an agent (e.g., a therapeutic agent or a diagnostic agent) to a cell (e.g., a neuron) comprises contacting the cell (e.g., a neuron) with the complex comprising the catalytically inactive BoNT-like toxin described herein (e.g., ^{ci}LC-Hn/X, ^{ci}LC-Hn/En, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) associated with the agent (e.g., a therapeutic agent or a diagnostic agent). In some embodiments, the contacting is in vitro (e.g., in cultured cells). In some embodiments, the contacting is ex vivo (e.g., in cells isolated from a subject). In some embodiments, the contacting is in vivo (e.g., in cells in a subject).

The catalytically inactive BoNT-like toxins described herein (e.g., ^{ci}LC-Hn/X, ^{ci}LC-Hn/En, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) are particularly suitable for use as a delivery vehicle to deliver agents in to cells (e.g., neurons) because it shows minimal residual toxicity, which is a major challenger in all other existing BoNT-mediated delivery methods (e.g., using catalytically inactive BoNT/A, BoNT/B, BoNT/C, BoNT/D, BoNT/E, BoNT/F, BoNT/G, or BoNT/H).

A “neuron” refers to an electrically excitable cell that communicates with other cells via specialized connections called synapses. A neuron may be a sensory neuron or a motor neuron. Sensory neurons respond to stimulus such as touch, sound, or light that affect the cells of the sensory organs and sends signals to the spinal cord or brain. Motor neurons receive signals from the brain and spinal cord to control everything from muscle contractions to glandular output. Interneurons connect neurons to other neurons within the same region of the brain or spinal cord in neural networks. A typical neuron consists of a cell body (soma), dendrites, and a single axon.

Other aspects of the present disclosure provide methods of diagnosing/treating a disease. In some embodiments, a method of diagnosing a disease comprises administering to a subject in need thereof an effective amount of the complex comprising the catalytically inactive BoNT-like toxin described herein (e.g., ^{ci}LC-Hn/X, ^{ci}LC-Hn/En, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) associated with a diagnostic agent. In some embodiments, the method of diagnosing a disease further comprise detecting a signal produced by the diagnostic agent, thus to diagnose the disease.

In some embodiments, a method of treating a disease comprises administering to a subject in need thereof an effective amount of the complex comprising the catalytically inactive BoNT-like toxins described herein (e.g., ^{ci}LC-Hn/X, ^{ci}LC-Hn/En, ^{ci}BoNT/XA, ^{ci}BoNT/XB, ^{ci}BoNT/XC, ^{ci}BoNT/XD, ^{ci}BoNT/XE, ^{ci}BoNT/XF, ^{ci}BoNT/XG, ^{ci}BoNT/XH, ^{ci}BoNT/EnA, ^{ci}BoNT/EnB, ^{ci}BoNT/EnC, ^{ci}BoNT/EnD, ^{ci}BoNT/EnE, ^{ci}BoNT/EnF, ^{ci}BoNT/EnG, or ^{ci}BoNT/EnH) associated with a therapeutic agent.

The terms “treatment,” “treat,” and “treating” refer to reversing, alleviating, delaying the onset of, or inhibiting the progress of a disease described herein (e.g., cancer or an autoimmune disease). In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence. Prophylactic treatment refers to the treatment of a subject who is not and was not with a disease but is at risk of developing the disease or who was with a disease, is not with the disease, but is at risk of regression of the disease. In some embodiments, the subject is at a higher risk of developing the disease or at a higher risk of regression of the disease than an average healthy member of a population.

An “effective amount” refers to an amount sufficient to elicit the desired biological response. An effective amount may vary depending on such factors as the desired biological endpoint, the pharmacokinetics of the compound, the condition being treated, the mode of administration, and the age and health of the subject. In some embodiments, an effective amount is a therapeutically effective amount. In some embodiments, an effective amount is a prophylactic treatment. In some embodiments, an effective amount is the amount of an agent in a single dose. In some embodiments, an effective amount is the combined amounts of an agent described herein in multiple doses. When an effective amount is referred to herein, it means the amount is prophylactically and/or therapeutically effective, depending on the subject and/or the disease to be treated. Determining the effective amount or dosage is within the abilities of one skilled in the art.

The terms “administer,” “administering,” or “administration” refers to implanting, absorbing, ingesting, injecting, inhaling, or otherwise introducing a compound described herein, or a composition thereof, in or on a subject. The complexes described herein, or composition(s) containing such complexes may be administered systemically (e.g., via intravenous injection) or locally (e.g., via local injection). In some embodiments, the complex or the composition comprising such complex described herein is administered via injection, e.g., intravenously, or sublingually. Parenteral administration is also contemplated. The term “parenteral” as used herein includes subcutaneous, intracutaneous, intravenous, intramuscular, intraarticular, intraarterial, intrasynovial, intrasternal, intrathecal, intralesional, intradermally, and intracranial injection or infusion techniques.

Empirical considerations, such as the half-life, generally will contribute to the determination of the dosage. For example, therapeutic agents that are compatible with the human immune system, such as polypeptides comprising regions from humanized antibodies or fully human antibodies, may be used to prolong half-life of the polypeptide and to prevent the polypeptide being attacked by the host's immune system. Frequency of administration may be determined and adjusted over the course of therapy, and is generally, but not necessarily, based on treatment and/or suppression and/or amelioration and/or delay of a disease. Alternatively, sustained continuous release formulations of a polypeptide may be appropriate. Various formulations and devices for achieving sustained release are known in the art.

In some embodiments, dosage is daily, every other day, every three days, every four days, every five days, or every six days. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the polypeptide used) can vary over time.

In some embodiments, for an adult subject of normal weight, doses ranging from about 0.01 to 1000 mg/kg may be administered. In some embodiments, the dose is between 1 to 200 mg. The particular dosage regimen, i.e., dose, timing and repetition, will depend on the particular subject and that subject's medical history, as well as the properties of the polypeptide (such as the half-life of the polypeptide, and other considerations well known in the art).

For the purpose of the present disclosure, the appropriate dosage of will depend on the specific agent (or compositions thereof) employed, the formulation and route of administration, the type and severity of the disease, whether the polypeptide is administered for preventive or therapeutic purposes, previous therapy, the subject's clinical history and response to the antagonist, and the discretion of the attending physician. Typically the clinician will administer a polypeptide until a dosage is reached that achieves the desired result. Administration of one or more polypeptides can be continuous or intermittent, depending, for example, upon the recipient's physiological condition, whether the purpose of the administration is therapeutic or prophylactic, and other factors known to skilled practitioners. The administration of an agent may be essentially continuous over a preselected period of time or may be in a series of spaced dose, e.g., either before, during, or after developing a disease.

“A subject in need thereof”, refers to an individual who has a disease, a symptom of the disease, or a predisposition toward the disease, with the purpose to cure, heal, alleviate, relieve, alter, remedy, ameliorate, improve, or affect the disease, the symptom of the disease, or the predisposition toward the disease.

A “subject” to which administration is contemplated refers to a human (i.e., male or female of any age group, e.g., pediatric subject (e.g., infant, child, or adolescent) or adult subject (e.g., young adult, middleaged adult, or senior adult)) or nonhuman animal. In some embodiments, the nonhuman animal is a mammal (e.g., rodent (e.g., mouse or rat), primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). The non-human animal may be a male or female at any stage of development. The non-human animal may be a transgenic animal or genetically engineered animal.

In some embodiments, the subject is a companion animal (a pet). “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include: rodents (e.g., rats, mice, guinea pigs, and hamsters), rabbits, or non-human primates.

Alleviating a disease includes delaying the development or progression of the disease, or reducing disease severity. Alleviating the disease does not necessarily require curative results. As used therein, “delaying” the development of a disease means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease includes initial onset and/or recurrence.

In some embodiments, the disease treated using the complex comprising the catalytically inactive BoNT-like toxin (e.g., ^{ci}BoNT/XA or ^{ci}BoNT/EnA as described herein) associated with a therapeutic agent is botulism, and the therapeutic agent is an BoNT-neutralizing antibody (e.g., an anti-BoNT/A VHH as described herein). “Botulism” is a serious illness caused by a BoNT that is active or having residual activity. The toxin causes paralysis. Paralysis starts in the face and spreads to the limbs. If it reaches the breathing muscles, respiratory failure can result. In some embodiments, the subject who has botulism has been administered a BoNT for treatment of another condition, or have been in contact with a BoNT (e.g., in contact with a substance contaminated with Clostridium botulinum. The strategies described herein are advantageous because the complex can enter neurons and neutralize BoNTs in the neurons that are causing botulism, and the catalytically inactive BoNTs used as delivery vehicles do not have residual toxicity.

In some embodiments, for treating botulism, the complex administered to the subject in need thereof comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 61 or SEQ ID NO: 62, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 40-47, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 61 and a second polypeptide comprising the amino acid sequence of any one of SEQ ID NOs: 40-47, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 62 and a second polypeptide comprising the amino acid sequence of any one of SEQ ID NOs: 40-47, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 61 and a second polypeptide comprising the amino acid sequence of SEQ ID NO: 40, wherein the first polypeptide and the second polypeptide are linked via disulfide bond.

In some embodiments, for treating botulism, the complex administered to the subject in need thereof comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 124 or SEQ ID NO: 125, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 40-47, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 124 and a second polypeptide comprising the amino acid sequence of any one of SEQ ID NOs: 40-47, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 125 and a second polypeptide comprising the amino acid sequence of any one of SEQ ID NOs: 40-47, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 124 and a second polypeptide comprising the amino acid sequence of SEQ ID NO: 40, wherein the first polypeptide and the second polypeptide are linked via disulfide bond.

In some embodiments, for treating botulism, the complex administered to the subject in need thereof comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 63 or SEQ ID NO: 64, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 49-56, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 63 and a second polypeptide comprising the amino acid sequence of any one of SEQ ID NOs: 49-56, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 64 and a second polypeptide comprising the amino acid sequence of any one of SEQ ID NOs: 49-56, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 63 and a second polypeptide comprising the amino acid sequence of SEQ ID NO: 49, wherein the first polypeptide and the second polypeptide are linked via disulfide bond.

In some embodiments, for treating botulism, the complex administered to the subject in need thereof comprises a first polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of SEQ ID NO: 126 or SEQ ID NO: 127, and a second polypeptide comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 49-56, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 126 and a second polypeptide comprising the amino acid sequence of any one of SEQ ID NOs: 49-56, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 127 and a second polypeptide comprising the amino acid sequence of any one of SEQ ID NOs: 49-56, wherein the first polypeptide and the second polypeptide are linked via disulfide bond. In some embodiments, for treating botulism, the complex administered to the subject in need thereof comprises a first polypeptide comprising the amino acid sequence of SEQ ID NO: 126 and a second polypeptide comprising the amino acid sequence of SEQ ID NO: 49, wherein the first polypeptide and the second polypeptide are linked via disulfide bond.

In some embodiments, the BoNT-neutralizing antibody (e.g., an anti-BoNT/A VHH as described herein) neutralizes the BoNT that is causing the botulism (e.g., reduces the activity of the BoNT causing the botulism by at least 20%). In some embodiments, the BoNT-neutralizing antibody (e.g., an anti-BoNT/A VHH as described herein) reduces the activity of the BoNT causing the botulism by at least 20%, at least 30%, at least 40%, at least 50%, at least 60%, at least 70%, at least 80%, at least 90%, or 100%.

In some embodiments, the disease treated using the complex comprising the catalytically inactive BoNT associated with a therapeutic agent is a neurological condition, and the therapeutic agent is a therapeutic agent for neurological conditions. Exemplary neurological conditions include, without limitation, spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions as well as other secretory disorders, pain from muscle spasms, headache pain.

In some embodiments, the condition is spinal muscular atrophy (SMA) and the therapeutic agent being delivered is functional SMN1 and/or SMN2 proteins, or small molecules and oligonucleotide that adjust expression of SMN1 and SMN2.

In some embodiments, the condition is Amyotrophic lateral sclerosis (ALS), and the therapeutic agent being delivered is antibodies or small molecules that target aggregated SOD1 proteins.

In some embodiments, the condition is an inherited form of motor neuron degeneration diseases and the agent being delivered is a gene editing agent for correcting genomic mutations in relevate genes (e.g., a Cas9 protein and a sgRNAs targeting the relevant genes, or zinc-finger nuclease for genetic editing).

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1. Delivery of Therapeutic Agents Using Catalytically Inactive Botulium Neurotoxins

Botulinum neurotoxins are a family of bacterial toxins, including seven major serotypes (BoNT/A-G)¹. These toxins target motor nerve terminals with extreme specificity and blocks neurotransmitter release from motor neurons, thus paralyzing animals and humans and resulting in a disease known as botulism. These toxins have been widely used for treating a variety of human diseases, and they are also classified as one of the six most dangerous potential bioterrorism agents.

The major issue with treating botulism is that some toxins have extremely long half-life in human neurons, for instance, maintaining its activity and blocking synaptic transmission for 6 months. This posts a formidable challenge for developing effective small molecular inhibitors. BoNTs can be neutralized by neutralizing antibodies. However, this only works for toxins that still have not entered motor neurons. There are currently no available toxin inhibitors that can block toxin activity inside neurons or shorten the duration of toxin half-life inside neurons.

If toxin neutralizing antibodies can be delivered into motor neurons, these antibodies can then bind to toxins and block toxin activity inside neurons. However, there are two major challenges: (1) be able to target motor neurons specifically; (2) be able to penetrate the cell membrane and deliver the antibody into the cytosol of the neuron. Ironically, these two challenges have been fully addressed by BoNTs themselves, as these toxins target motor neurons specifically and can deliver its functional domain, which is 50 kDa, across endosomal membranes into the cytosol of the cells. Therefore, BoNTs can be potentially utilized as an ideal delivery tool targeting motor neurons. Furthermore, such delivery tools would be useful for delivering a variety of cargoes/therapeutics into motor neurons for modulating motor neuron activities and for treating motor neuron related diseases including motor neuron degenerative diseases such as amyotrophic lateral sclerosis (ALS).

Such a BoNT-based delivery system have been previously developed in the laboratory and showed to be able to deliver proteins into neurons. Of course, to serve as a delivery tool, BoNTs must be “de-toxified” to fully get rid of its toxicity. BoNTs are composed of three functional domains: (1) the light chain (LC), which is a protease domain that is delivered into the cytosol of cells; (2) translocation domain (H_{N}), which helps the LC across the endosomal membrane in cells into the cytosol; (3) receptor-binding domain (Hc), which is responsible for targeting motor neurons⁴. Inactive form of toxin is usually generated by introducing point mutations into its LC to abolish its protease activity inside neurons. However, all currently reported “inactive” form of BoNTs still showed a low level of toxicity when injected into mice^{3,5}. The reason for this residual toxicity remains unknown, but it forms the major barrier for developing a useful delivery system.

Three new BoNT-like toxins, termed BoNT/X, BoNT/En and BoNT/PMP1^{6,7,63} were recently identified. These toxins share the same overall structure and function as other BoNTs, but they have significant divergence on sequences from BoNTs and form a separated branch from BoNTs. Their LCs and translocation domains display the same function as these domains in BoNTs, but their Hcs do not specifically target motor neurons in mice, thus BoNT/X, BoNT/En, and BoNT/PMP1 do not target mammalian motor neurons. Here it was examined whether a safe delivery tool can be developed based on creating novel chimeric proteins containing the LC and translocation domain of BoNT/X or BoNT/En and the receptor-binding domain of a BoNT, which confers specific binding to moto neurons. It was found that, in contrast to delivery tools based on traditional BoNTs (BoNT/A-G), these designed chimeric toxin (for instance: BoNT/XA (LC-Hn of BoNT/X fused with the Hc of BoNT/A)) showed no detectable toxicity in vivo in mouse models, thus representing a safe and effective delivery system for deliver cargo/therapeutics into motor neurons.

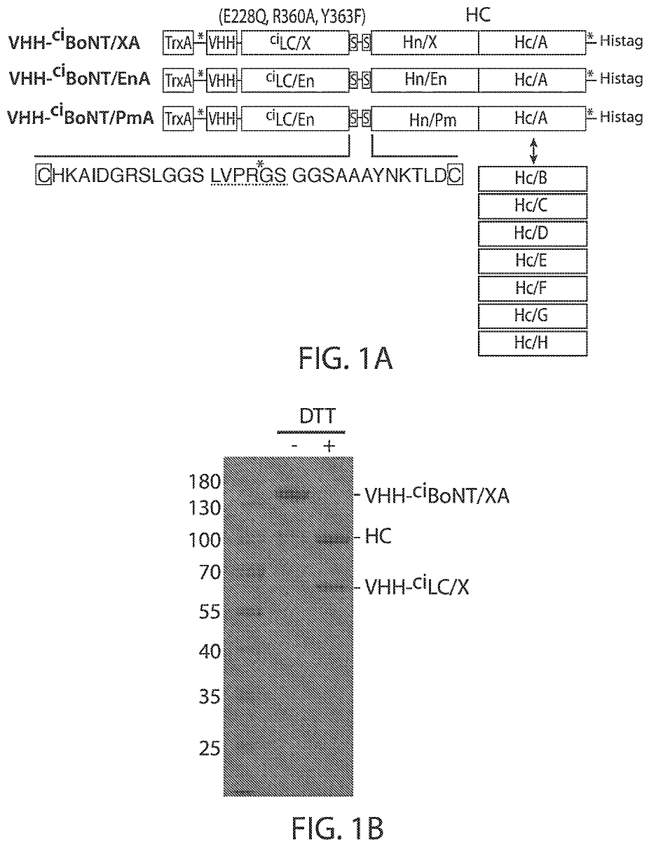

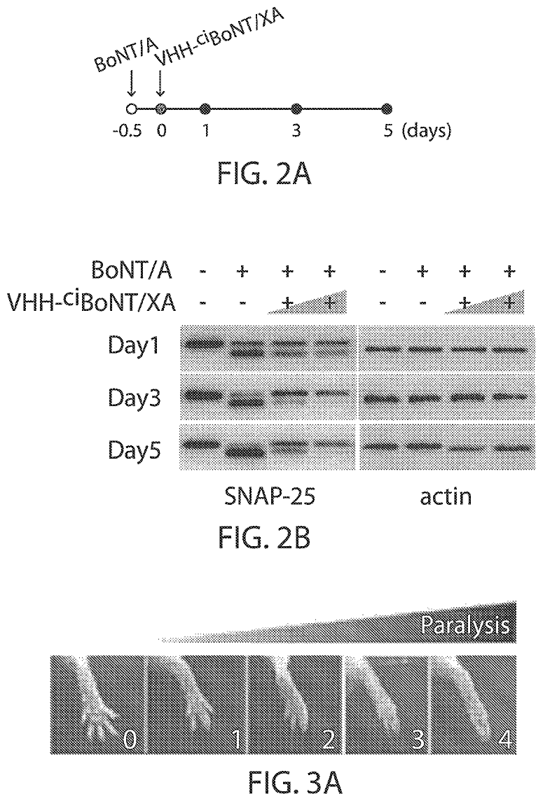

A VHH antibody⁸ fused with catalytically inactive BoNT (VHH-^{ci}BoNT) was generated (FIG. 1A). The Hc of a BoNT (from BoNT/A-H) can be utilized to replace the Hc of these inactive BoNT-like toxins in order to confer the specificity toward mammalian motor neurons. LCHn/X and Hc/A was used as an example. LCHn/X was fused to Hc/A (BoNT/XA) because Hc/X had shown no specific binding to mouse motor neurons. To abolish the catalytic activity, three amino acids at the active site were mutated (E228Q, R360A, Y363F for LC/X) using site-directed mutation. The VHHB8 clone, which binds to LC/A with high affinity (K_{D}=1.06 nM) and is able to neutralize LC/A as described in previous publication Tremblay J M et al., 2010, Toxicon, 56:990-998, was conjugated at the N-terminal of LC/X to be delivered into the neuron. It was first examined whether ^{ci}BoNT/XA can deliver the VHH antibody into neuronal cytosol and neutralize LC/A on the cultured neuron. Rat cultured cortical neurons were exposed to 20 pM BoNT/A. After 12 hours, the BoNT/A in the medium was washed out, and intoxicated neurons were further incubated with VHH-^{ci}BoNT/XA for up to 5 days (FIG. 2A). Cleavage SNAP-25 were detected via immunoblotting analysis. As shown in FIG. 2B, VHH-^{ci}BoNT/XA decrease the number of cleavage SNAP-25 at dose-dependent manner at day 1. VHH-^{ci}BoNT/XA almost completely blocked the cleavage of SNAP25 after 3 days, but not VHH-^{ci}BoNT/C. This indicates that ^{ci}BoNT/XA successfully delivers the VHHB8 antibody into neuronal cytosol and ^{ci}BoNT/XA showed a high level of efficacy in delivering VHHB8 than inactive ^{ci}BoNT/C.

Next, the toxicity levels of various constructs, including VHH fused with catalytically inactive BoNT/XA, XC (LC-Hn of BoNT/X fused with the Hc of BoNT/C) and XD (LC-Hn of BoNT/X fused with the Hc of BoNT/D) were evaluated, and compared with VHH fused with inactive BoNT/C (VHH-^{ci}BoNT/C) and BoNT/D (VHH-^{ci}BoNT/D), as well as isolated VHH and ^{ci}BoNT/C. As shown in Table 1, the indicated amount of proteins were injected into mice via IP injection and the health of each mouse was monitored for 5 days. VHH alone showed no toxicity at the dose tested (10 mg/kg). ^{ci}BoNT/C at 4 mg/kg), VHH-^{ci}BoNT/C at 0.8 mg/kg, and VHH-^{ci}BoNT/D at 0.2 mg/kg caused ‘botulism-like’ paralysis and these mice died within 10 h after injection. In contrast, VHH-^{ci}BoNT/XA, /XC, and/XD did not show any adverse effect at even 100 mg/kg.

These results further confirmed that delivery tools based on inactive traditional BoNTs have residue toxicity, while the novel delivery tool based on inactive chimeric toxin such as ^{ci}BoNT/XA showed no toxicity, thus it represents a feasible drug delivery tool targeting neurons.

Table 1 shows toxicity of VHH-fused catalytically inactive BoNT/XA, /XC, and /XD (VHH-^{d}BoNT/XA, /XC, and/XD) in vivo in mouse. The indicated dose of VHH-^{ci}BoNT/XA, /XC, /XD, /C, /D, VHH B8, and ^{ci}BoNT/C was administrated to mouse by IP injection. The mice were observed up to 5 days after injection. ^{ci}BoNT (4 mg/kg), VHH-^{ci}BoNT/C (0.8 mg/kg) and VHH-^{ci}BoNT/D (0.2 mg/kg) caused ‘botulism-like’ paralysis and these mice died within 10 h after injection, but no adverse effect was observed by VHH-^{ci}BoNT/XA, /XC, and/XD (100 mg/kg).

| | | Number | |
|---|---|---|---|
| | | survived/ | |
| | | Number | |
| | Dose | on the | Survival |
| Protein | [mg/kg] | study | (%) |
| VHH-^{ci}BoNT/X-A | 2 | 3/3 | 100 |
| | 6 | 6/6 | 100 |
| | 30 | 6/6 | 100 |
| | 60 | 6/6 | 100 |
| | 100 | 6/6 | 100 |
| VHH-^{ci}BoNT/X-C | 0.2 | 2/2 | 100 |
| | 2 | 6/6 | 100 |
| | 6 | 6/6 | 100 |
| | 100 | 6/6 | 100 |
| VHH-^{ci}BoNT/X-D | 100 | 4/4 | 100 |
| VHH B8 | 10 | 6/6 | 100 |
| ^{ci}BoNT/C | 2 | 8/12 | 67 |
| | 4 | 1/6 | 17 |
| | 10 | 0/3 | 0 |
| VHH-^{ci}BoNT/C | 0.2 | 6/6 | 100 |
| | 0.4 | 6/6 | 100 |
| | 0.8 | 1/6 | 17 |
| | 2 | 1/6 | 17 |
| Boiled VHHB8-^{ci}BoNT/C | 2 | 6/6 | 100 |
| VHH-^{ci}BoNT/D | 0.02 | 9/9 | 100 |
| | 0.2 | 4/13 | 31 |

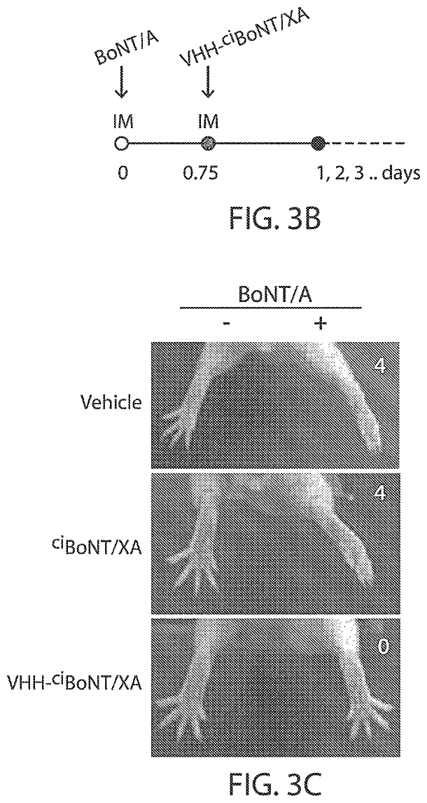

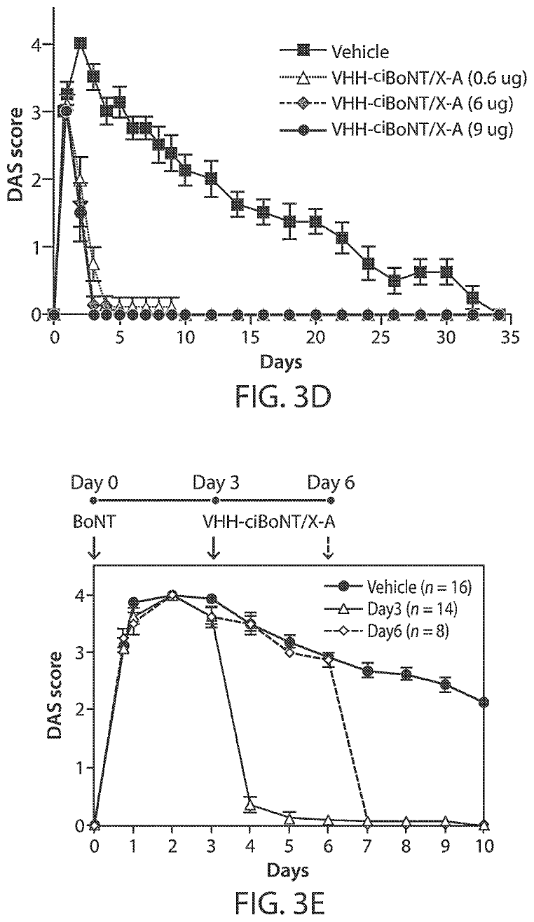

The therapeutic potential of intramuscular administrated VHH-^{ci}BoNT/XA to mice that showed leg muscle paralysis by BoNT/A was next investigated. The neutralization activity of VHH-^{ci}BoNT/XA was evaluated using the Digit Abduction Score (DAS) assay, which is a well-established non-lethal assay. Mice were injected with 5.8 pg of BoNT/A in the right hind limb muscle. Muscle paralysis was assessed according to the DAS scale, as previously reported (FIG. 3A). After 18 hours, the BoNTA-injected mice showed score ‘2-3,’ indicating that BoNT/A entered into the neuron and cleaved the substrate. Then the indicated concentration of VHH-^{ci}BoNT/XA were injected into the same muscle (FIG. 3B). As shown FIG. 3C, VHH-^{ci}BoNT/XA-injected mice showed score ‘0’ after 3 days, while ^{ci}BoNT/XA that is not fused with VHHB8 antibody did not show any recovery. It was next examined how VHH-^{ci}BoNT/XA led to shortening the paralysis by BoNT/A. BoNT/A induced muscle paralysis was recovered after 35 days. In contrast, 9 ug of VHH-^{ci}BoNT/XA showed complete recovery from the paralysis within 3 days (FIG. 3D). 0.6 and 6 ug of VHH-^{ci}BoNT/XA also showed similar results. Finally, treatment with VHH-^{ci}BoNT/XA 3-days and 6-days after exposure to BoNT/A was tested. As shown in FIG. 3E, injection of VHH-^{ci}BoNT/XA was able to quickly restore the muscle contraction ability and reduce the DAS score to 0 within 15 hours at both 3-days and 6-days post-exposure to BoNT/A. Together, these results demonstrate that the ^{ci}BoNT/XA can deliver the VHH into neuronal cytosol and neutralize BoNT/A.

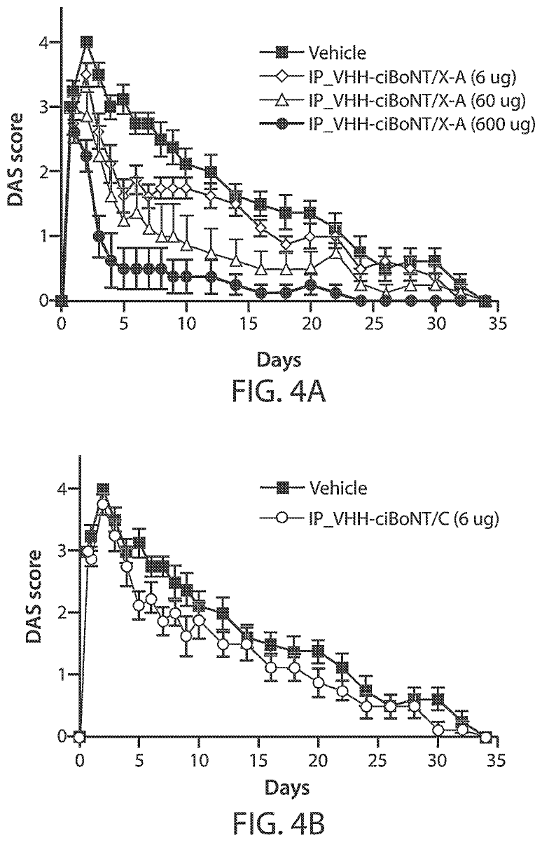

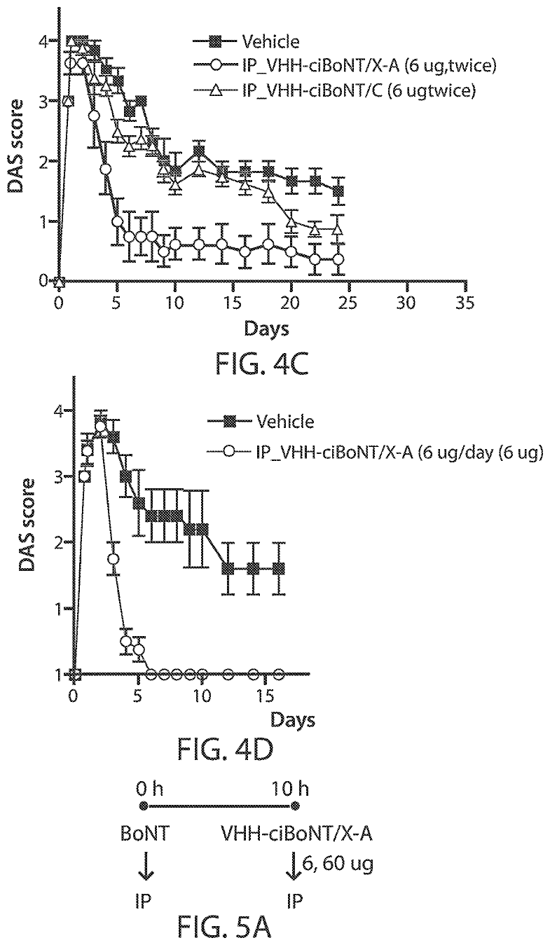

The therapeutic potential of VHH-^{ci}BoNT/XA was further examined using intraperitoneal (IP) injection. For this test, the mouse was administrated BoNT/A in the hind limb muscle first. VHH-^{ci}BoNT/XA was administrated by IP injection 18 hours later. 6 ug of VHH-^{ci}BoNT/XA and VHH-^{ci}BoNT/C were slightly effective, but did not shorten the duration of paralysis (FIGS. 4A-4B). The dose of VHH-^{ci}BoNT/XA was further increased to 60 and 600 ug. High-doses of VHH-^{ci}BoNT/C were not tested due to the toxicity issue (Table 1). Increasing the dose of VHH-^{ci}BoNT/XA showed better neutralization (FIG. 4A). The effect of multiple injections of VHH-BoNT/XA was tested next (FIGS. 4C-4D). Two consecutive injection of VHH-BoNT/XA (6 μg) showed a similar result of a single injection of 600 ug. Injection of VHH-BoNT/XA (6 μg) once per day for 6 days resulted in complete recovery within 6 days.

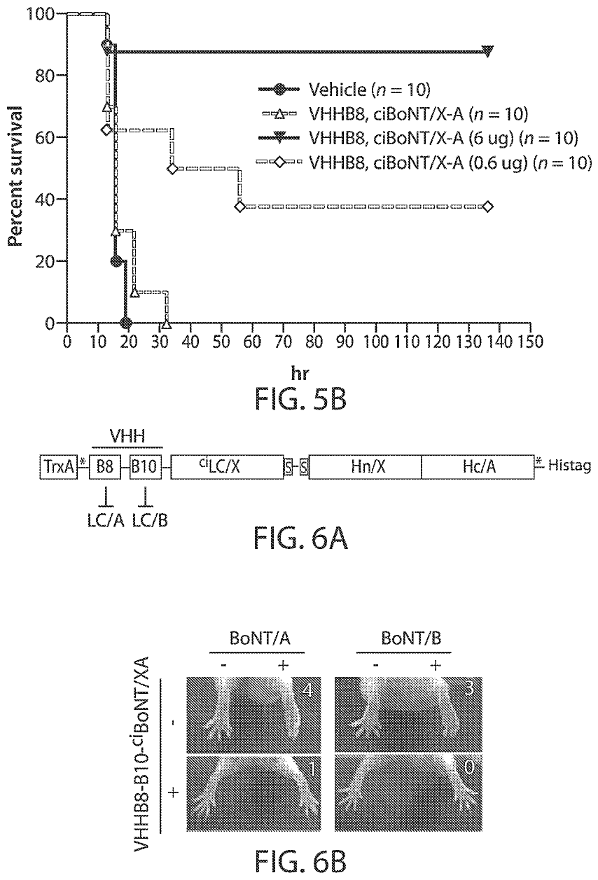

Next, whether VHHB8-^{ci}BoNT/XA can neutralize BoNT/A was analyzed in a systematic lethality assay. Briefly, 20 pg of BoNT/A (˜4 LD50 value) were injected into mouse via IP injection. 10 hours later, the mice that showed typical systematic botulism phenotype were randomly separated into four groups and subjected to IP injection of the indicated proteins: group 1; vehicle, 0.2% gelatin-saline, group 2; VHH and ^{ci}BoNT/XA mixture, group 3; 6 ug of VHH-^{ci}BoNT/XA, group 4; 0.6 ug of VHH-^{ci}BoNT/XA. Mice were further observed for 5 more days (FIG. 5A). While 100% of mice in the group 1 and group 2 died, 90% of mice in the group 3 survived and showed a complete restoration of their activity (FIG. 5B). Group 4, which was injected with 10-fold less VHH-^{ci}BoNT/XA compared to the group 3, showed ˜40% survival rate (FIG. 5B). These results demonstrate that VHH-^{ci}BoNT/XA can be utilized to treat systematic botulism.

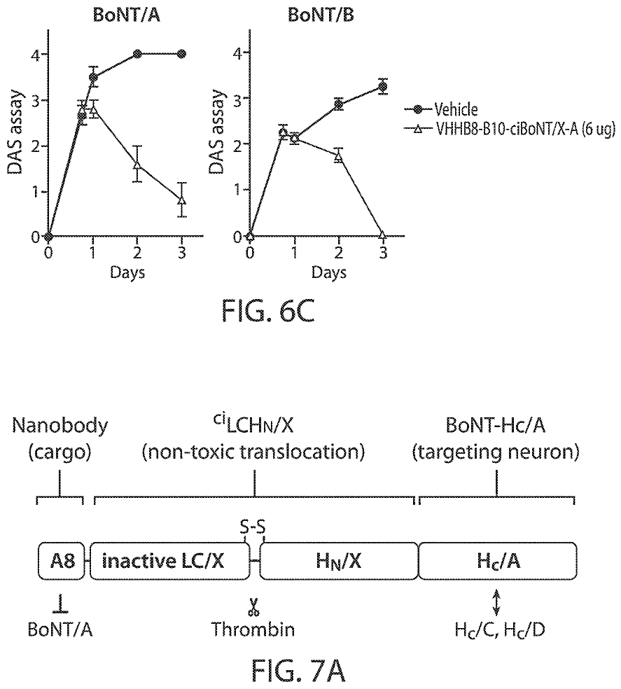

Next, whether multiple VHHs can be tethered and delivered together into neurons were examined. A VHH known as B10 that binds to LC of BoNT/B (LC/B) and neutralizes LC/B activity⁸ was chosen. A new construct that contains both VHHB8 (targeting LC/A) and VHH-B10 (targeting LC/B) was fused to ^{ci}BoNT/XA (FIG. 6A). BoNT/A (5.8 pg) or BoNT/B (3.5 pg) were injected in hind limb muscles in different mice. After 18 hours, 6 μg of VHH B8-B10-^{ci}BoNT/XA were injected in the same muscle and DAS score were recorded. As shown in FIG. 6B, VHH-B8-B10-^{ci}BoNT/XA treatment shortened the duration of muscle paralysis induced by BoNT/A (left panel) and BoNT/B (right panel). The DAS scores over time further demonstrate that VHH B8-B10-^{ci}BoNT/XA was effective in shortening the duration of paralysis induced by BoNT/A (left panel) and BoNT/B (right panel). These data demonstrate that multiple VHHs can be successfully delivered into the cytosol of neurons using ^{ci}BoNT/XA-based delivery tool. VHH B8-B10-^{ci}BoNT/XA also represent a single unique therapeutic agent that can be utilized to treat botulism caused by two different toxins (BoNT/A and BoNT/B, which are responsible for majority of human botulism cases). Such a multi-target agent will provide significant reduction in drug development cost, and can be utilize to treat patients prior to knowing the serotype of the toxins, which could take a few days to determine.

Herein it is demonstrated that catalytically inactive BoNT/XA (^{ci}BoNT/XA) protein can deliver the VHH antibody into motor neuron with high efficiency. Furthermore, VHH-^{ci}BoNT/XA, XC, and XD does not have toxicity against mouse model thus ^{ci}BoNT/XA, XC, and XD representing a new generation of delivery tools that is effective and safe for targeting neurons. The results also established a novel way to treat botulism in a post-exposure manner, by delivering VHHs into the cytosol of neurons and neutralizing LC of BoNTs inside neurons.

### Materials and Methods

Antibodies: The following antibodies were purchased from indicated vendors: mouse monoclonal anti-SNAP-25 (71.1, Synaptic Systems), mouse monoclonal Anti-β-Actin (Sigma).

Protein expression and purification: VHH-^{ci}BoNT/XA, ^{ci}BoNT/XA, VHH-^{ci}BoNT/C and ^{ci}BoNT/C were expressed as His6 tagged recombinant proteins in E. coli BL21 (DE3) cells using autoinduction medium. Expression was allowed to proceed incubated at 16-18° C. overnight with vicious shaking. Cell pellets were resuspended in binding buffer for Ni-affinity [20 mM Tris-HCl pH7.5, 500 mM NaCl, 20 mM Imidazole, 10% glycerol with 0.1 mM of PMSF. Cells were disrupted by sonication on ice. Lysates were clarified by centrifugation at 20,000 rpm for 30 min at 4° C. The proteins were purified via HisTrap HP (GE Healthcare). Purified proteins were treated with thrombin (2 U/mg of protein) at 4° C. overnight. The activated proteins were further purified by Superdex 200 pg 16/600 gel-filtration column (GE Healthcare). To remove LPS, proteins were passed through the Pierce™ High Capacity Endotoxin Removal Resin column (ThermoFisher Scientific). Proteins were sterile by 0.22 um filter and store at −80° C.

Neuron culture and toxin neutralization assay: Rat cortical neurons were prepared from E19 embryos as described previously⁹. Neurons were exposed to 20 pM of BoNT/A in 300 uL medium for 12 h. The cells were washed by cultured medium three times and further incubated with 10 and 50 nM of VHH-^{ci}BoNT/XA up to 5 days. Immunoblot analysis was carried out to detect SNAP-25. Actin was used as a loading control.

Digit abduction score (DAS) assay and administration of VHH-^{ci}BoNT/XA; mice were injected in the left hind limb muscle with 5.8 pg of BoNT/A (META biology, Inc.) diluted in saline with 0.2% gelatin. After 18 hours, the score reached “2-3” and VHH-^{ci}BoNT/XA was administrated by IM injection at the same muscle or by IP injection. Muscle paralysis was assessed one time per one to two days according to the DAS assay scale.

### Example 2 Targeted Intracellular Delivery of Nanobodies Inhibits Botulinum Neurotoxins in Neurons and Achieves Effective Treatment of Botulism

BoNTs are a family of bacterial toxins with seven major serotypes (BoNT/A-G)¹⁰⁻¹⁵. They are the most potent toxins known and classified in the United States as one of the six most dangerous potential bioterrorism agents (Category A and Tier 1)¹⁶. These toxins target and enter motor neurons and block neurotransmitter release, causing the disease known as botulism, whose defining symptom is flaccid paralysis (losing the ability to contract muscles). Among the seven serotypes, BoNT/A, B, and E (and rarely F) are associated with human botulism, with BoNT/A and B responsible for most cases. Although rare, botulism cases persist in human populations with a death rate of ˜3-5%^{17,18}

A major challenge for addressing the threats posed by BoNT/A and B is their extraordinary long half-life within the cytosol of neurons¹⁹⁻²³, leading to persistent nerve blockade and muscle paralysis that lasts for months in humans. Patients must rely on intensive care and mechanical ventilation for weeks to months to stay alive, which renders the treatment expensive and could easily overwhelm the health care system during a large-scale outbreak¹⁶. BoNT-neutralizing antibodies have been developed²⁴⁻²⁷, but they are useful only before toxins enter neurons, and there are no inhibitors available that can block toxin action within neurons.

BoNT/A and B are also the two serotypes approved for treating a multitude of medical conditions as well as for reducing wrinkles, benefiting millions of people every year^{10,11,28} BoNT/A is the dominant form in clinical use. Local injection of tiny amounts of BoNT/A provides persistent muscle relaxation that lasts 4-6 months. However, if the patient is dissatisfied with the effect or there is unwanted diffusion of BoNT/A, there are no available post-exposure remedies that can reverse paralysis.

BoNTs are composed of two chains and three functional domains¹⁰⁻¹⁵: a light chain (LC, 50 kDa) which is a protease domain; and a heavy chain (HC) that can be further divided into a membrane translocation domain (H_{N}, ˜50 kDa) and a receptor-binding domain (H_{C}, ˜50 kDa). BoNTs are initially synthesized as a single polypeptide. The linker region between LC and HC needs to be proteolytically cleaved in order to generate the active di-chain form, in which the LC remains covalently connected to the HC via an inter-chain disulfide bond. These toxins target motor neurons with extraordinary specificity by binding to neuronal receptors through the Hc and enter neurons via receptor-mediated endocytosis. A drop in pH within endosomes then triggers conformational changes in the HN, leading to translocation of the LC across endosomal membranes into the cytosol. The inter-chain disulfide bond is reduced once the LC reaches the cytosol, thus releasing the LC. The LC then cleaves a specific set of neuronal proteins belonging to the SNARE protein family, including SNAP-25 (cleaved by BoNT/A, E, and C), Syntaxin 1 (cleaved by BoNT/C), and three homologous vesicle membrane proteins VAMP1, 2, and 3 (targets for BoNT/B, D, F, and G). Syntaxin 1 and SNAP-25 are localized on plasma membranes and form a complex with VAMPs, known as the SNARE complex, which is the core machinery mediating fusion of synaptic vesicle membranes to the plasma membranes^{29,30}. Cleavage of any one of these three SNARE proteins disrupts vesicle membrane fusion to plasma membranes, thus blocking the release of neurotransmitters.

It has been shown that the LC of BoNT/A (LC/A) maintains its activity within neurons for months, which is the reason for its ability to induce persistent paralysis that lasts 4-6 months in humans¹⁹⁻²³. Intoxicated neurons fully recover their function once the toxin LC loses its activity. Thus, successful treatment of botulism requires targeting and inhibiting LCs within neurons. However, it has been challenging to develop small-molecular inhibitors that work effectively in neurons. Many neutralizing antibodies against LCs have been developed, but they cannot target motor neurons and penetrate the cell membrane into the cytosol of neurons.

As BoNTs are naturally capable of targeting motor neurons and delivering their LCs into neurons, they were explored as a carrier for targeted delivery of protein cargoes in 2004 by Bade et al³¹. They fused different proteins to the N-terminus of full-length active form of BoNT/D and tested these fusion proteins on cultured neurons, utilizing cleavage of VAMP2 by BoNT/D-LC (LC/D) in neurons as a sensitive readout for successful translocation of the cargo into the cytosol. They found that fusion of a dihydrofolate reductase (DHFR) or a LC/A did not affect overall translocation efficacy, while fusion of firefly luciferase or green fluorescent protein (GFP) reduced translocation efficacy. These studies demonstrate that a protein cargo can be delivered into the cytosol of neurons through direct fusion to a BoNT, and the translocation efficacy varies depending on cargo proteins.

Before use as a delivery tool, BoNTs must be “de-toxified”, which turns out to be challenging. Simply deleting the LC often creates solubility issues due to disrupting native interactions between LC and H_{N}³²⁻³⁴. An alternative approach is to abolish LC protease activity by mutating key residues. LCs are zinc-dependent proteases with a conserved HEXXH motif¹⁹⁻³⁵. Mutations are usually introduced to one or two residues in this motif plus two residues (e.g. R363A and Y366F in BoNT/A) that are conserved in all BoNTs and critical for their protease activity³⁶. Such catalytically inactive forms of BoNT/A and BoNT/C containing three designed point mutations have been developed and shown to have no protease activity. However, both still induced flaccid paralysis and death at μg-per-mouse levels³⁷⁻³⁸. This residual toxicity has been independently reported for inactive full-length BoNT/A, B, C, E, and F containing three point-mutations in their LCs³⁹. The source of this residual toxicity remains unknown, but it could be due to the translocation process, which may disrupt endosomes at μg-per-mouse levels. Although this toxicity is much lower compared with active toxins (μg versus pg per mouse), it nevertheless is a safety concern and a major barrier to the development of inactive BoNTs as delivery tools.

A BoNT-like toxin was recently identified, termed BoNT/X⁴⁰, which has the same conserved domain structure as other BoNTs, with certain distinct features. For instance, the LC of BoNT/X (LC/X) cleaves not only the canonical substrates VAMP1/2/3, but also additional VAMP family members VAMP4, VAMPS, and Ykt6⁴⁰. Furthermore, the Hc of BoNT/X (H_{C}/X) does not target motor neurons in mice; the host species targeted by BoNT/X remains to be established. Interestingly, the fragment containing the LC-H_{N} portion of BoNT/X (LCH_{N}/X) can translocate its LC more efficiently into neurons than the corresponding fragments of BoNT/A and BoNT/B⁴⁰. Utilizing the LCH_{N}/X, a chimeric inactive toxin-based neuron-specific drug-delivery platform was developed by de-activating its protease activity through mutations and by fusing it to the H_{C} of a BoNT. Therapeutic proteins targeting BoNT-LCs were then created by fusion of the chimeric inactive toxin platform with nanobodies (also known as VHH antibody), which are 12-15 kDa proteins derived from the single variable domain of the heavy-chain-only antibodies in Camelidae such as alpacas and llamas. Such therapeutic proteins showed no toxicity even at 100 mg/Kg dose in vivo in mice and successfully neutralized BoNT-LC activity in neurons, shorten the duration of muscle paralysis, and rescue mice from lethal dose of BoNT/A and BoNT/B after the onset of botulism.

### Results

### Chimeric Inactive Toxin-Based Delivery Platform Shows No Toxicity In Vivo

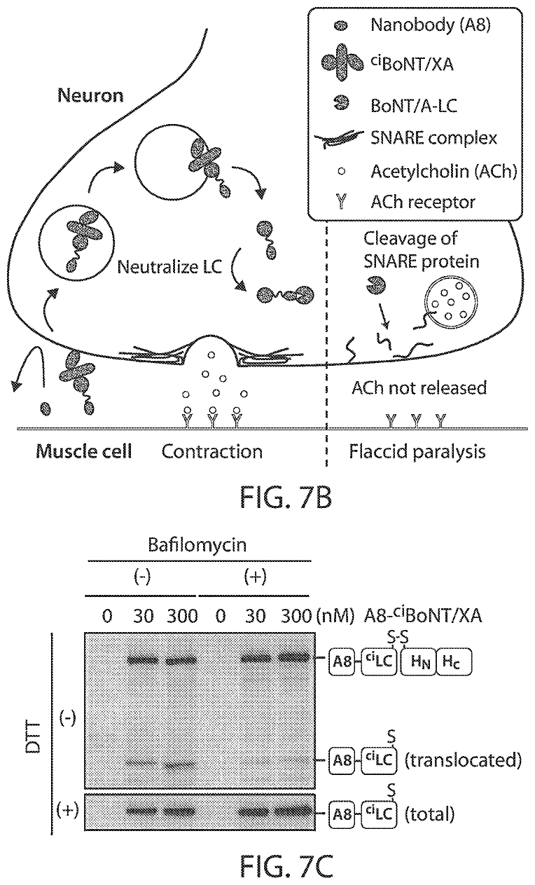

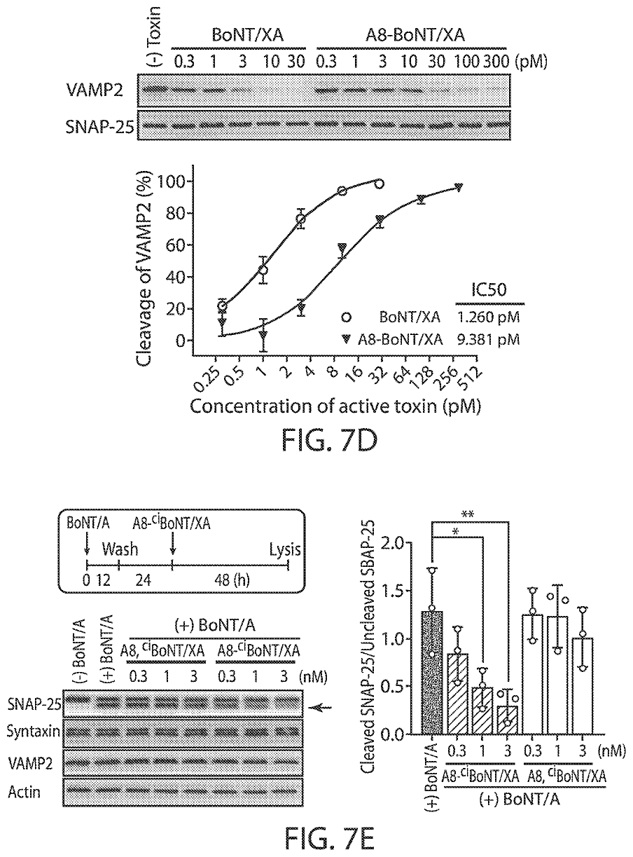

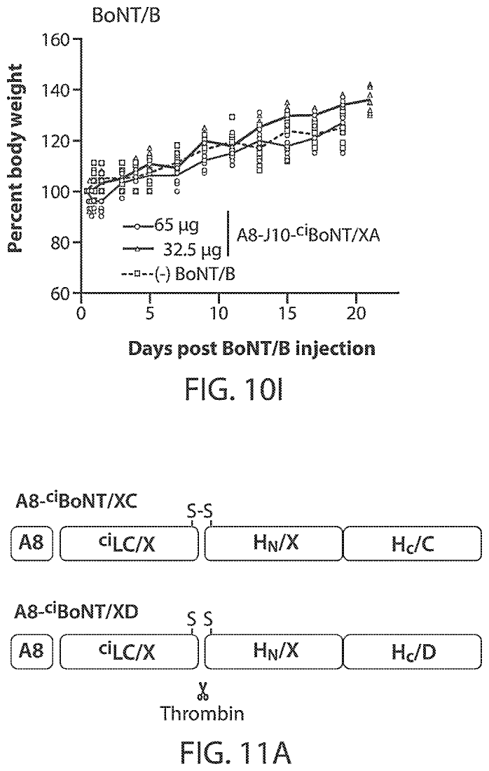

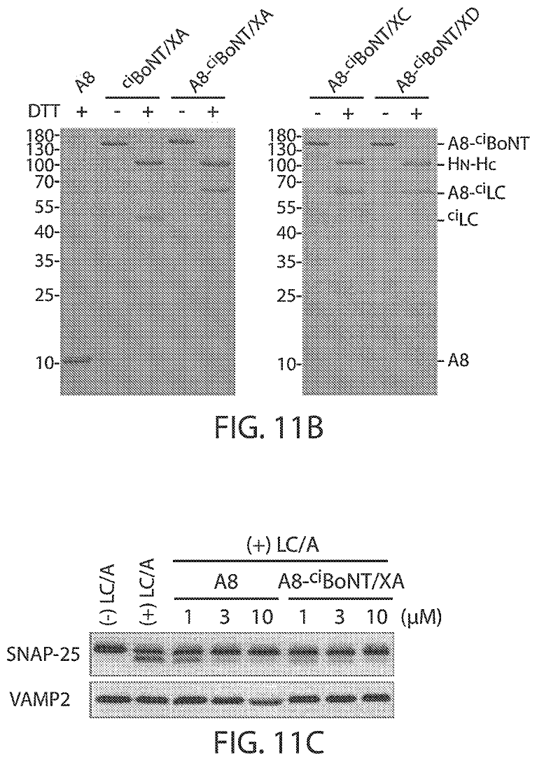

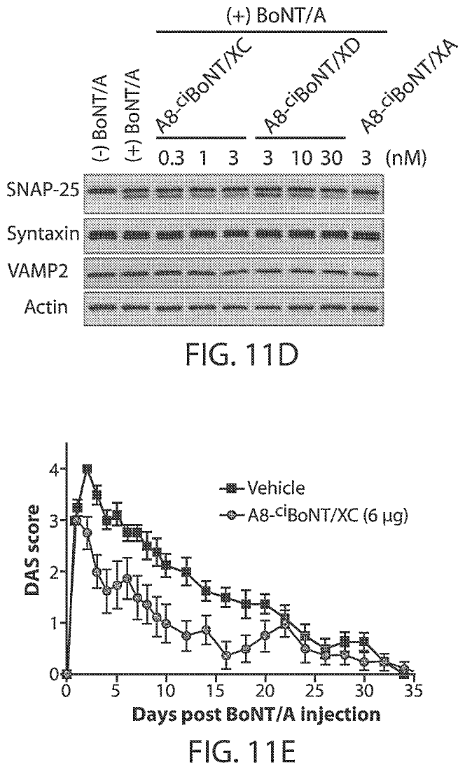

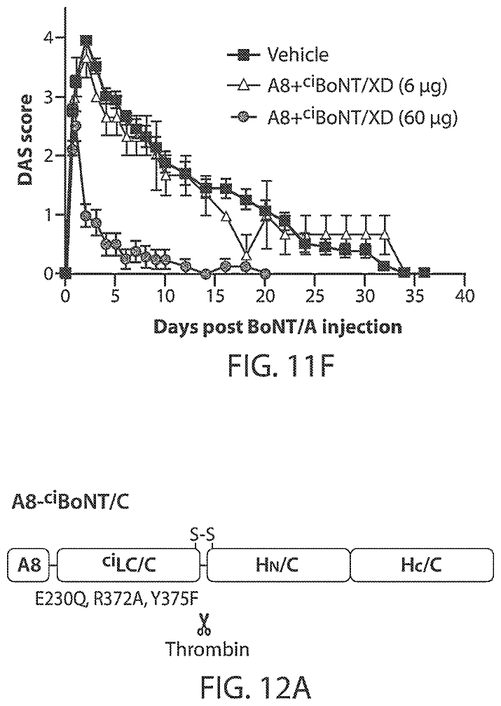

To explore whether LCH_{N}/X might provide a safer delivery tool than inactive BoNTs, three chimeric inactive toxins were created by: (1) fusing LCH_{N}/X with the Hc of BoNT/A (H_{C}/A), BoNT/C (H_{C}/C), or BoNT/D (H_{C}/D). These H_{C}s essentially replace Hc/X and confer specificity toward mammalian motor neurons (FIGS. 7A and 7B, and FIG. 11A); (2) introducing three point-mutations (E228Q/R360A/Y363F) to key residues in LC/X to abolish its protease activity (designated catalytic inactive form, ^{ci}LCH_{N}); and (3) modifying the linker region between the LC and H_{N} to include a thrombin cleavage site, which enables us to specifically convert the chimeric toxin from a single chain into a di-chain form using thrombin. In addition, a thrombin cleavage site is also introduced before the C-terminal His6 tag to cleave off the His6 tag after protein purification.

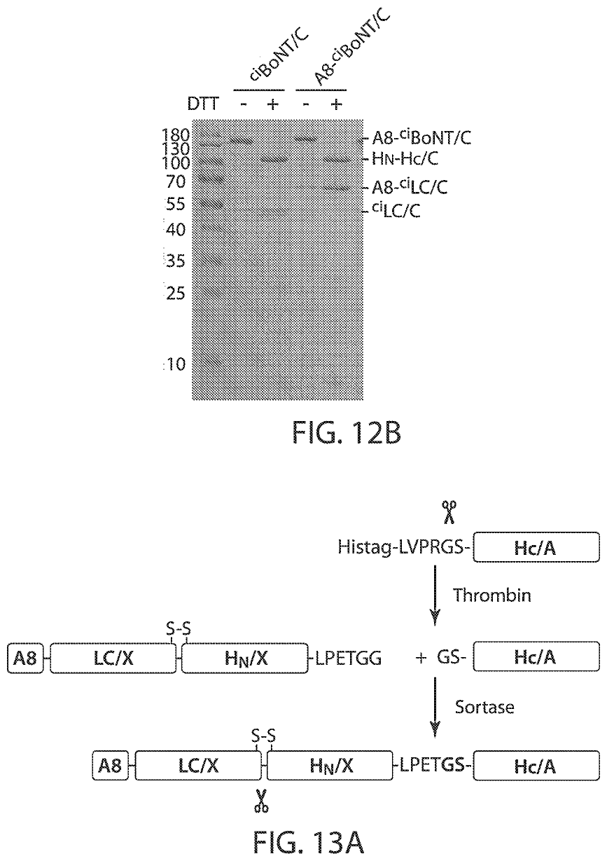

These chimeric inactive toxins are termed ^{ci}BoNT/XA, ^{ci}BoNT/XC, and ^{ci}BoNT/XD. A previously reported nanobody was then selected (known as VHH-ALc-B8, abbreviated “A8” here), which was raised against recombinantly purified LC/A in alpaca and has been demonstrated to inhibit LC/A in vitro and in cells^{41,42}. A8 served as a cargo and was fused directly to the N-terminus of chimeric inactive toxins, generating A8-^{ci}BoNT/XA (FIGS. 7A and 7B), A8-^{ci}BoNT/XC, and A8-^{ci}BoNT/XD (FIG. 11A). For comparison, a catalytically inactive form of BoNT/C (^{ci}BoNT/C) was also constructed, containing the same set of three-point mutations in its LC as in ^{ci}LC/X, as well as A8-^{ci}BoNT/C fusion protein (FIG. 12).

These fusion proteins were expressed and purified in E. coli, and readily converted into a di-chain form by thrombin treatment (FIGS. 11B and 12B). The inter-chain disulfide bond is formed as the reducing agent DTT treatment separates these proteins into two parts: one is ^{ci}LC (˜50 kDa) or A8-^{ci}LC (˜65 kDa), and the other is H_{N}-H_{C} (˜100 kDa, FIGS. 11B and 12B). A8-^{ci}LC showed the same level of potency as A8 alone in inhibiting LC/A activity in vitro (FIG. 11C).

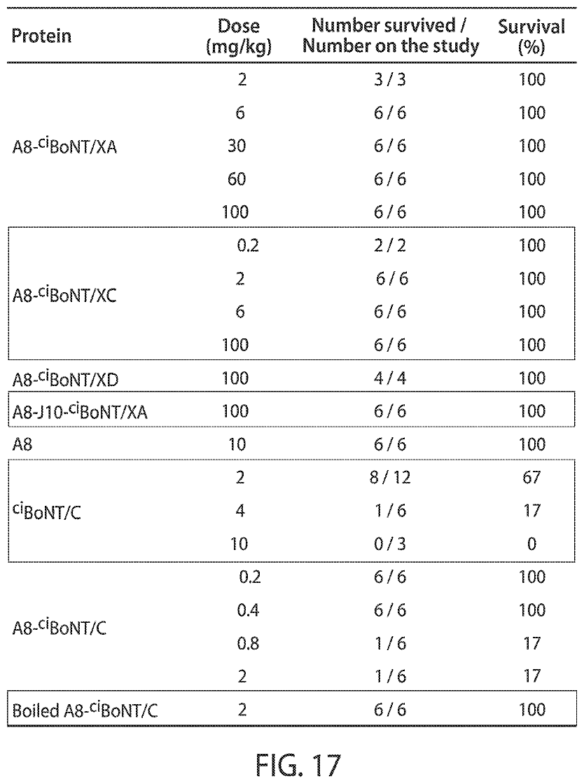

It was first examined whether these fusion proteins have any toxicity in vivo in mice. Intraperitoneal (IP) injection of A8-^{ci}BoNT/C caused death of most mice at 0.8 mg/Kg range (FIG. 17). Injection of A8 alone or boiled A8-^{ci}BoNT/C showed no toxicity, while ^{ci}BoNT/C alone induced death in the 2 mg/Kg (FIG. 17). These findings are consistent with previous reports that BoNTs with inactive LCs still show residual toxicity^{37,39}. In contrast, IP injection of A8-^{ci}BoNT/XA, A8-^{ci}BoNT/XC, or A8-^{ci}BoNT/XD all showed no detectable toxicity, even at 100 mg/Kg (FIG. 17).

A8-LC/X is Delivered into the Cytosol of Cultured Neurons

It was then analyzed whether the chimeric inactive toxin platform can deliver the A8-^{ci}LC/X fragment into the cytosol of cultured neurons. The experimental design took advantage of the fact that A8-^{ci}LC/X is connected via a disulfide bond to the H_{N}-H_{C}. Acidification of endosomes induces translocation, which would deliver the A8-^{ci}LC/X across the endosomal membrane into the cytosol (FIG. 7B). Once reaching the cytosol side, the disulfide bond is reduced, which has been shown to be assisted by the thioredoxin reductase-thioredoxin protein disulfide-reducing system⁴³, and A8-^{ci}LC/X is separated from the H_{N}-H_{C} (FIG. 7B). If the translocation is not successful, A8-^{ci}LC/X would still be connected with the H_{N}-H_{C}. Thus, the appearance of isolated A8-^{ci}LC/X in neuron lysates when samples were analyzed under non-reducing conditions indicates that translocation was successful.

Cultured rat cortical neurons were exposed to A8-^{ci}BoNT/XA at 30 and 300 nM concentrations for 12 h. Neuron lysates were harvested and subjected to immunoblot analysis under non-reducing conditions. A8 could be detected using an antibody against the constant region of nanobodies. The isolated A8-^{ci}LC/X band was detected in neuron lysates (FIG. 7C). To further demonstrate that the A8-^{ci}LC/X bands were generated by translocation, the same experiment was carried out in the presence of bafilomycin, a small molecule inhibitor that blocks acidification of endosomes. This treatment did not affect the overall binding of A8-^{ci}BoNT/XA to neurons, as the full-length band at 165 kDa showed intensity similar to that of neurons not treated with bafilomycin, yet bafilomycin treatment greatly reduced the isolated A8-^{ci}LC/X band (FIG. 7C). Together, these experiments demonstrate that A8-^{ci}LC/X has been delivered into the cytosol of neurons.

### Delivered A8 and LC/X are Functional in the Cytosol of Neurons

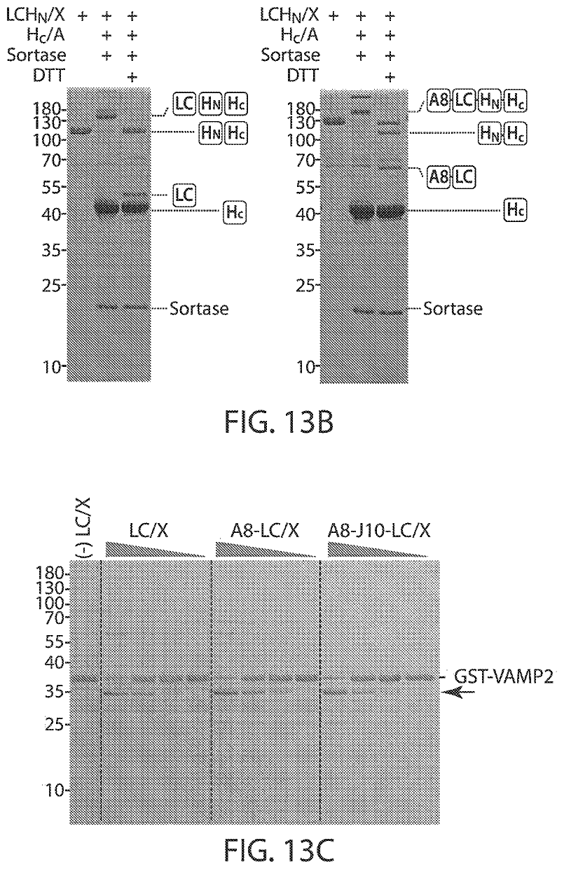

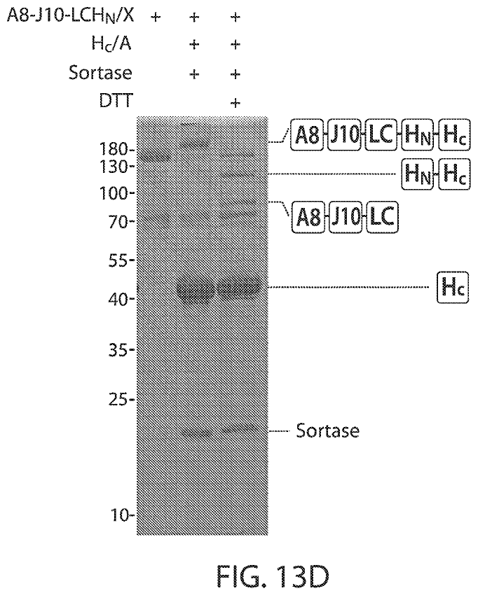

To further estimate the translocation efficacy and determine whether translocated proteins are functional in cells, a new construct was built that expresses A8 fused with the active LCH_{N}/X containing no mutations in its LC. There is an additional short sortase recognition tag (residues LPETGG) added to the C-terminus. This tag can be recognized by the bacterial transpeptidase sortase, which can ligate the fusion protein covalently to the N-terminus of a second protein containing a free glycine on the N-terminus (FIG. 13A)⁴⁰⁻⁴⁴. A8-LCH_{N}/X was ligated with the H_{C}/A, yielding an A8 fused with an active full-length BoNT/XA (termed A8-BoNT/XA, FIGS. 13A and 13B). As a control, the active BoNT/XA was also generated by ligating LCH_{N}/X and H_{C}/A (FIG. 13B). This approach allows us to produce limited amounts of active toxin without creating the coding sequence for full-length toxins to ensure biosafety. A8-BoNT/XA allows us to examine whether the translocated A8-LC/X is functional in cultured neurons by analyzing cleavage of VAMP2. As shown in FIG. 7D, incubation of cultured neurons with picomolar levels of A8-BoNT/XA or BoNT/XA both resulted in cleavage of VAMP2, demonstrating that the A8-LC are functional after translocation. Compared with BoNT/XA, A8-BoNT/XA showed 7.4-fold reduction in efficacy based on assessing VAMP2 cleavage in neurons (FIG. 7D). A8-LCH_{N}/X and LCH_{N}/X showed similar activity in cleaving recombinant VAMP2 protein in vitro, indicating that fusion with A8 does not affect LC activity (FIG. 13C). Together, these data suggest that A8-LC/X was delivered into neurons at ˜7.4-fold lower efficacy compared with LC/X.

It was then evaluated whether the delivered A8-^{ci}LC/X can neutralize LC/A within cultured neurons. Neurons were first exposed to BoNT/A for 12 h, washed, further incubated in toxin-free medium for another 24 h, followed by incubation with A8-^{ci}BoNT/XA for 48 h (FIG. 7E). Incubation with a mixture of separated A8 and ^{ci}BoNT/XA proteins was analyzed in parallel as a control. Cell lysates were harvested and analyzed by immunoblot, revealing persistent cleavage of SNAP-25 by LC/A^{45,46}. Incubation with separated A8 and ^{ci}BoNT/XA did not affect cleavage of SNAP-25, whereas incubation with A8-^{ci}BoNT/XA reduced SNAP-25 cleavage in neurons (FIG. 7E). Similarly, incubation with A8-^{ci}BoNT/XC or A8-^{ci}BoNT/XD also reduced SNAP-25 cleavage in neurons in this post-exposure model (FIG. 11D). These data demonstrate that ^{ci}BoNT/XA, XC, and XD were able to deliver a functional A8 into the cytosol of neurons.

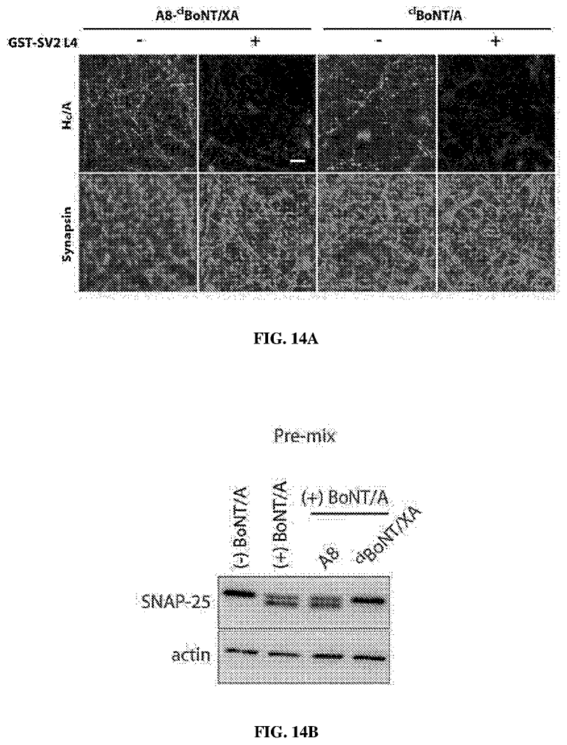

The receptor-binding property of A8-^{ci}BoNT/XA was also validated and it was confirmed that its binding to neurons was reduced by a recombinant protein containing the 4th luminal domain fragment of SV2C, which is a protein receptor for BoNT/A (FIG. 14A)^{47,48}. Consistently, pre-mixing nanomolar A8-^{ci}BoNT/XA with picomolar BoNT/A and adding them together to cultured neurons reduced cleavage of SNAP-25 compared with BoNT/A alone, further suggesting that A8-^{ci}BoNT/XA utilizes the same receptors as BoNT/A and thus reduced binding and entry of BoNT/A into neurons (FIG. 14B). SV2 are a family of synaptic vesicle membrane proteins including SV2A, B, and C, and their exposure to the cell surface is reduced after synaptic vesicle exocytosis is blocked by BoNTs. However, SV2 still travels to cell surfaces during its nascent biogenesis before it is internalized and sorted into synaptic vesicles, and this constitutional secretory pathway is not affected by any BoNTs^{49,50}, which likely provides an entry pathway for A8-^{ci}BoNT/XA after synaptic vesicle exocytosis is blocked by pre-loaded BoNT/A.

### Intramuscular Injection of A8-^{ci}BoNT/XA Shortens BoNT/A-Induced Leg Muscle Paralysis

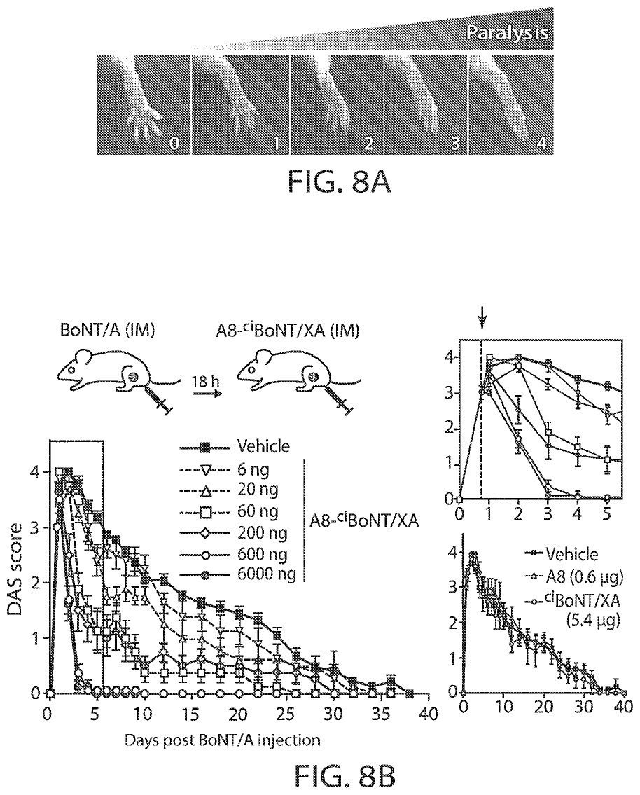

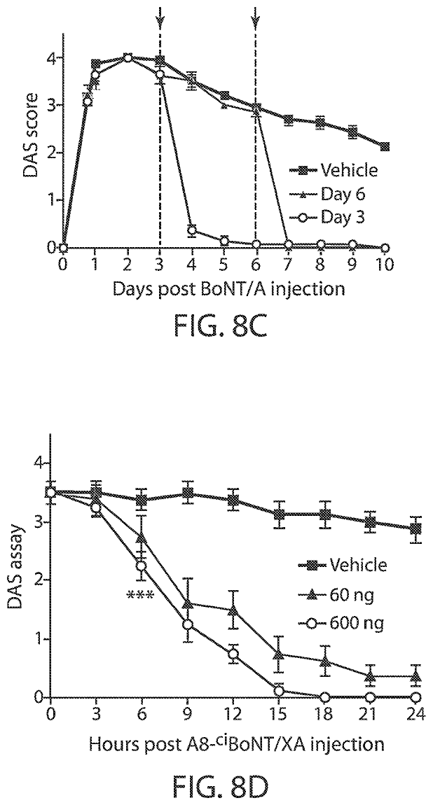

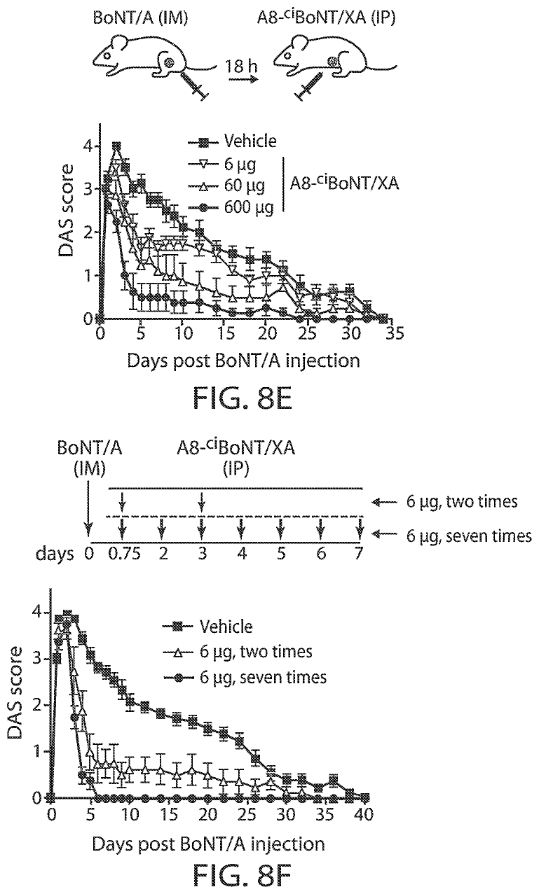

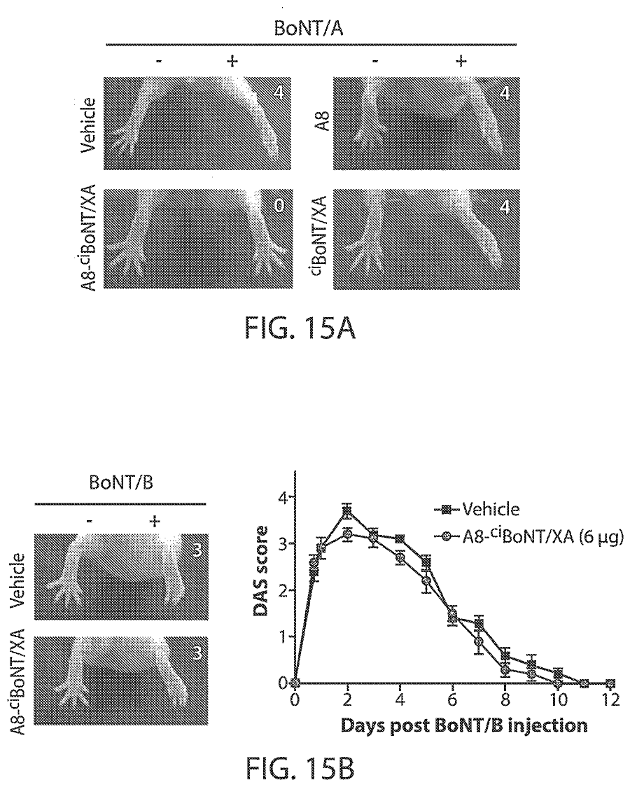

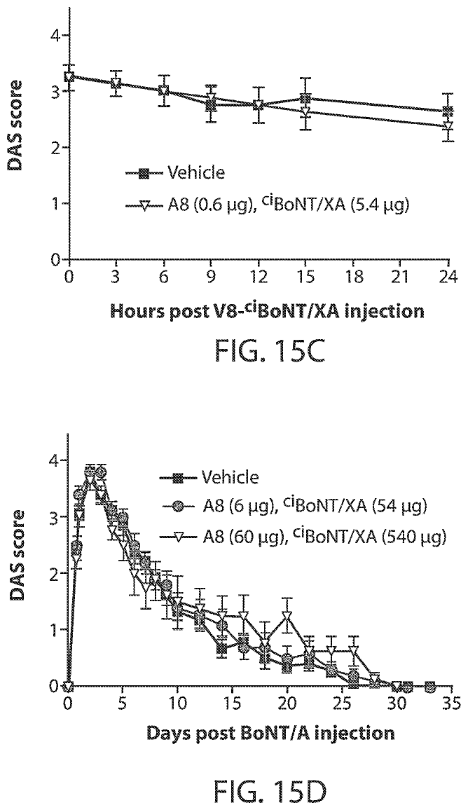

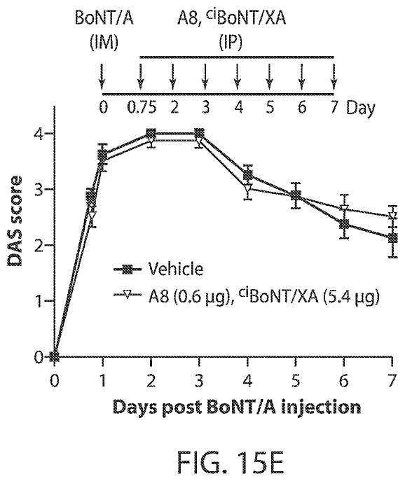

After validating these fusion proteins in cultured neurons, assessing their effectiveness in treating BoNT/A intoxication in vivo was then focused on. A local paralysis model known as the Digit Abduction Score (DAS) assay⁵¹ was first utilized. Sub-lethal doses of BoNT/A are injected intramuscularly (IM) into the hind legs of mice, which paralyzes the leg muscle and prevents toe spreading during the startle response. The degree of toe spreading is scored 0-4, reflecting the degree of muscle paralysis (FIG. 8A). Injection of BoNT/A at 6 pg induced the severest scores of 3-4. In mice, possibly due to their fast metabolism rates, BoNT/A induces paralysis that lasts ˜30-40 days (FIG. 8B). To develop a post-exposure model, BoNT/A was first injected to mice, and after a 18 h period, the leg is obviously paralyzed with scores 2-3. IM injection of A8-^{ci}BoNT/XA were then carried out to the same BoNT/A injection site (FIG. 8B). Separated A8 and ^{ci}BoNT/XA proteins were analyzed in parallel as controls: neither affected the degree or duration of muscle paralysis (FIG. 8B, right-lower panel). In contrast, injecting as little as 60 ng of A8-^{ci}BoNT/XA drastically reduced muscle paralysis (FIG. 8B). Injecting 600 ng A8-^{ci}BoNT/XA fully restored muscle function (reaching a score of 0) within three days, and increasing the dose to 6 μg yielded similar results (FIG. 8B). The effect is specific for BoNT/A, as A8-^{ci}BoNT/XA did not alter the degree and duration of paralysis induced by BoNT/B in DAS assays (FIGS. 15A and 15B). Furthermore, A8-^{ci}BoNT/XC and A8-^{ci}BoNT/XD reduced the degree or duration of BoNT/A-induced leg muscle paralysis, albeit requiring higher doses than A8-^{ci}BoNT/XA, suggesting that A8-^{ci}BoNT/XA is the most effective one in vivo (FIGS. 11D and 11E). A8-^{ci}BoNT/XA was thus focused on as a prototype.

To further confirm that A8-^{ci}BoNT/XA shortens muscle paralysis after toxin entry into motor neurons, A8-^{ci}BoNT/XA was injected on day 3 or day 6 after the initial BoNT/A injection, by which time paralysis is already decreasing (FIG. 8C, day 3 in red, day 6 in the triangle). Injecting 600 ng A8-^{ci}BoNT/XA to the same site where BoNT/A was injected restored muscle function within one day for both 3-days and 6-days post-injection of BoNT/A (FIG. 8C). More frequent monitoring of the degree of muscle paralysis revealed that the DAS score showed obvious decrease 6 h after injection of A8-^{ci}BoNT/XA, and muscle function was completely recovered by 15 h (FIG. 8D). Recovery of similar speed was achieved with 60 ng A8-^{ci}BoNT/XA (FIG. 8D), while the control mixture of separated V8 and ^{ci}BoNT/XA proteins showed no effect on the degree or duration of muscle paralysis (FIG. 15C).

### IP Injection of A8-^{ci}BoNT/XA Shortens BoNT/A-Induced Leg Muscle Paralysis

It was then analyzed whether A8-^{ci}BoNT/XA can effectively reach the paralyzed leg muscle through systemic circulation in vivo. BoNT/A (6 pg) was first injected to the hind leg muscle and waited 18 h for the muscle to be paralyzed. A8-^{ci}BoNT/XA was then injected via IP and the DAS scores were monitored (FIG. 8E). Injecting A8-^{ci}BoNT/XA reduced the local leg muscle paralysis and DAS scores, although a much higher dose (e.g. 600 μg) of A8-^{ci}BoNT/XA is required compared with the previous IM injection of A8-^{ci}BoNT/XA to the same BoNT/A injection site.

Interestingly, the effective dose can be lowered with multiple administrations of A8-^{ci}BoNT/XA. For instance, IP injection of 6 μg of A8-^{ci}BoNT/XA daily for two days elicited a recovery rate similar to a single dose of 600 μg, while dosing with 6 μg daily for seven days achieved an even faster recovery rate (FIG. 8F). As controls, injecting a total of 600 μg separated A8 and ^{ci}BoNT/XA, or 7 days of daily injections of A8 and ^{ci}BoNT/XA, did not affect the degree or duration of muscle paralysis (FIGS. 15D and 15E).

IP Injection of A8-^{ci}BoNT/XA Rescues Mice from Systemic BoNT/A Intoxication

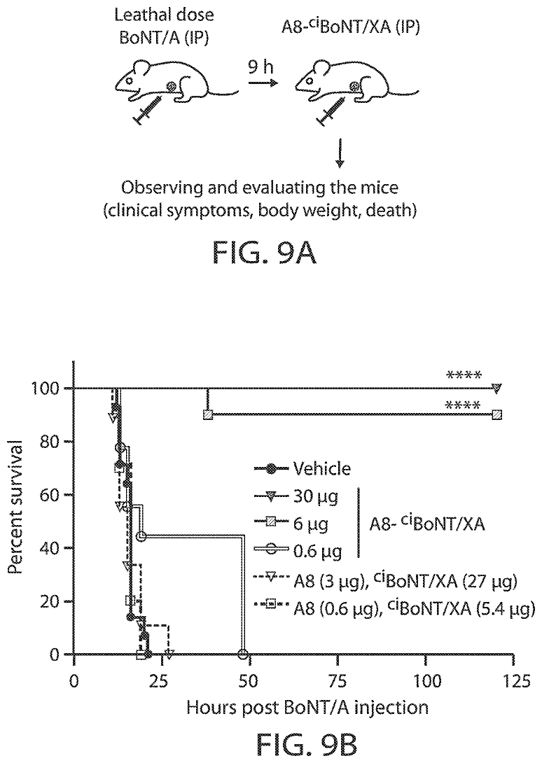

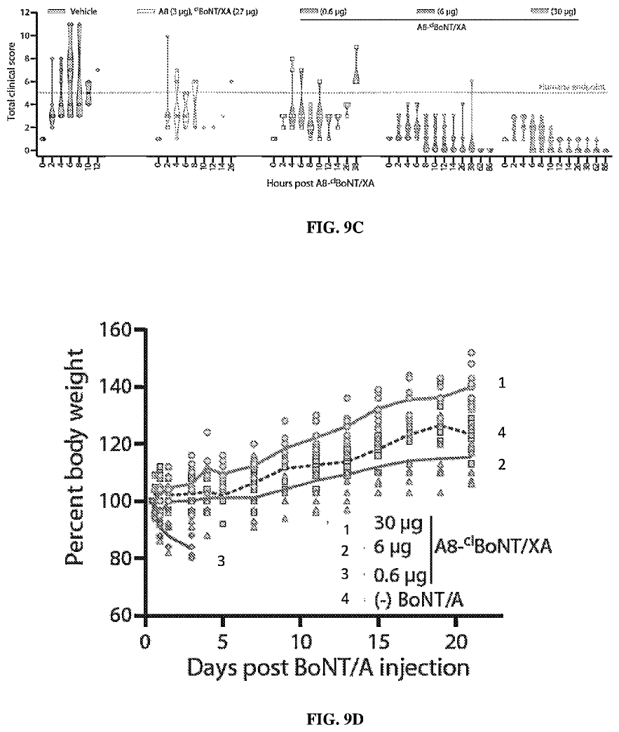

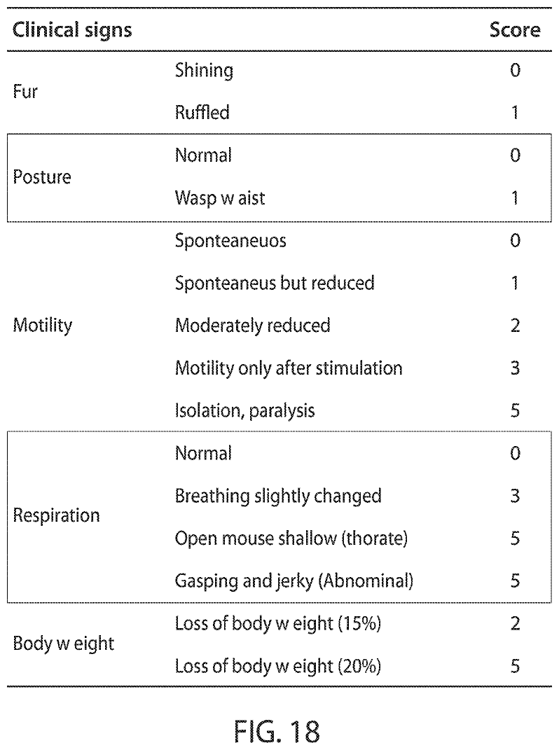

It was then examined whether A8-^{ci}BoNT/XA provides effective post-exposure treatment of systemic BoNT/A intoxication and rescues mice from botulism. IP injection of 19.5 pg BoNT/A induced typical systemic botulism symptoms of a “wasp” body shape and reduced mobility within 9 h in mice, and all mice further developed immobility and severe respiratory stress that required euthanization within a few hours. To quantify the disease progress, a scoring system was developed based on the appearance of the wasp shape, the degree of mobility/activity, respiratory distress, and body weight changes (FIG. 18). Mice were first injected with BoNT/A (19.5 pg, IP), and IP injection of A8-^{ci}BoNT/XA was then carried out 9 h later in animals that developed obvious botulism symptoms (FIG. 9A). Injecting 0.6 μg/mouse of A8-^{ci}BoNT/XA reduced the rate of increase in the clinical score, but these mice eventually developed severe symptoms and lost 20% body weight within 48 h; all were euthanized (FIGS. 9B to 9D). A8-^{ci}BoNT/XA at 6 μg/mouse reduced clinical scores within 8 h, but one mouse (of 10) relapsed by 36 h and was euthanized. Further increasing A8-^{ci}BoNT/XA to 30 μg/mouse reduced clinical score and restored mobility/activity within 6 h. Body weight gains were comparable with those in control mice and no mice relapsed, suggesting full and complete recovery (FIGS. 9B to 9D). As controls, mixtures of A8 and ^{ci}BoNT/XA proteins did not offer any protection in this post-exposure model (FIGS. 9B to 9D).

Simultaneous Delivery of Two Nanobodies into Neurons

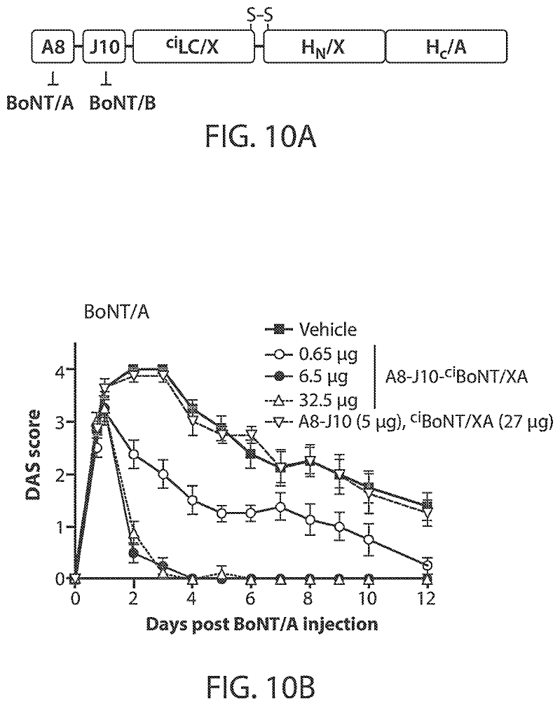

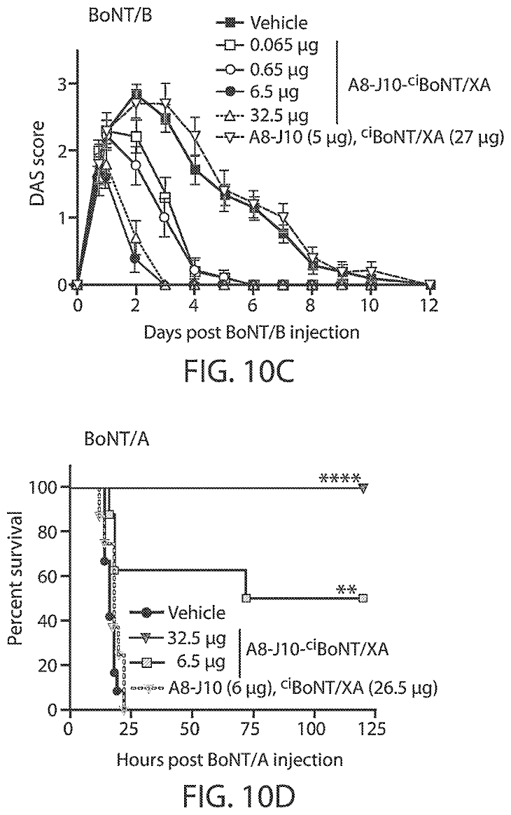

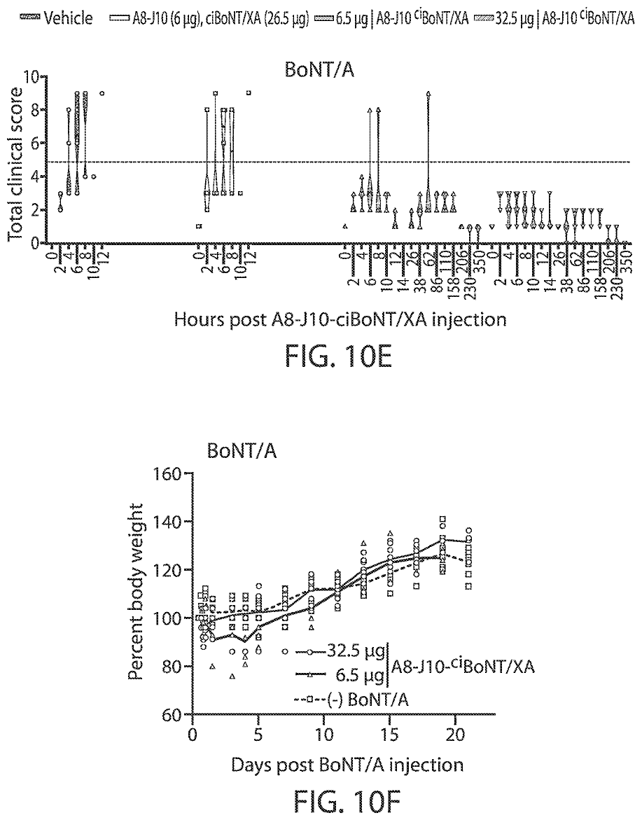

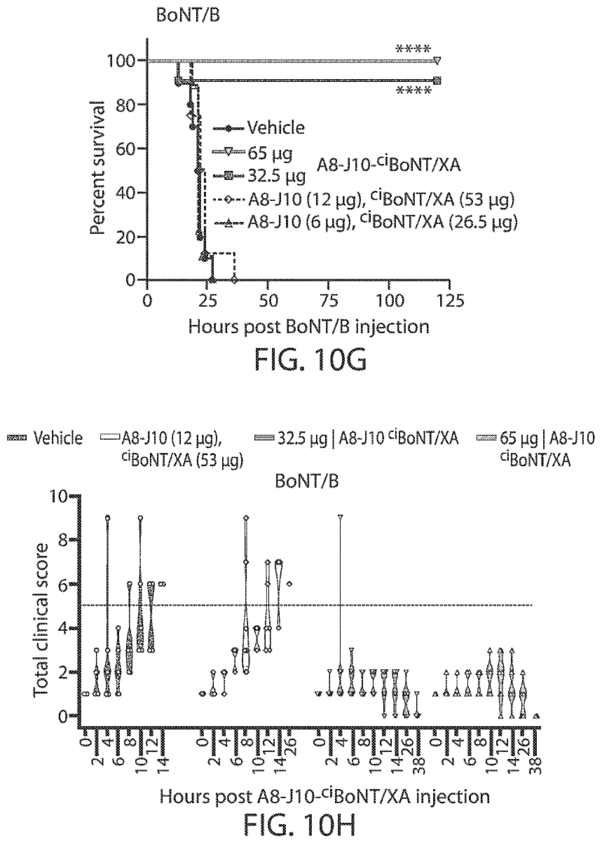

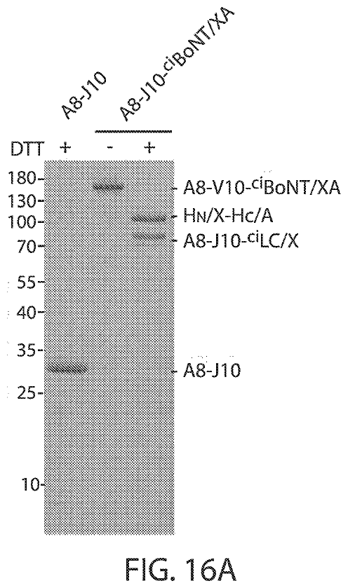

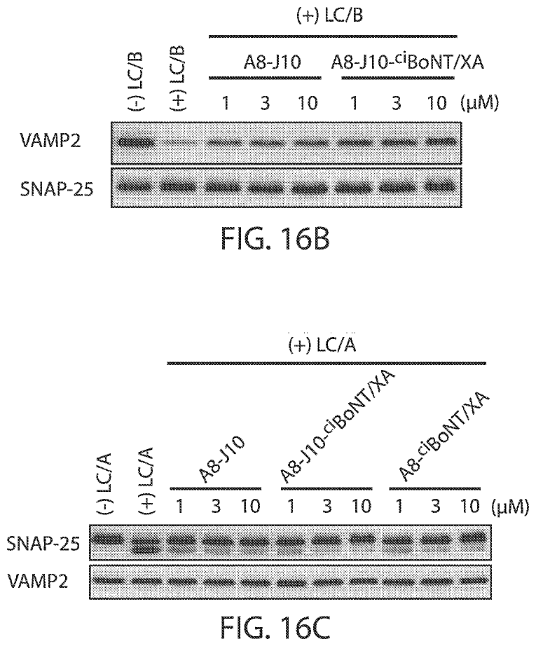

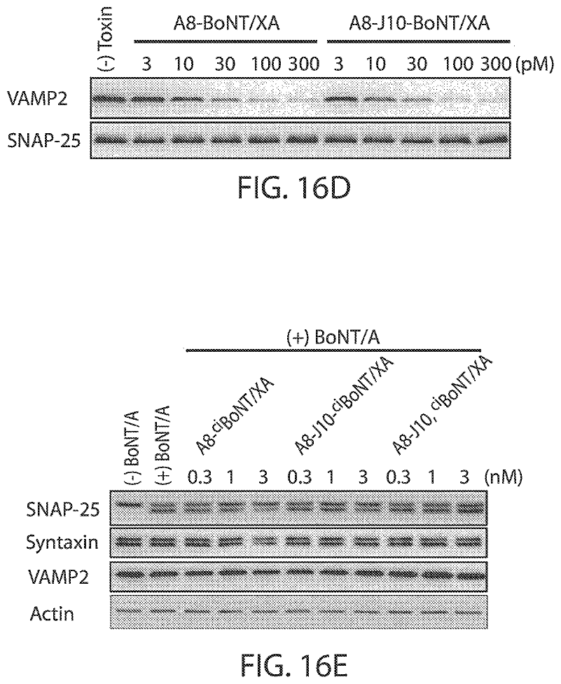

The studies expanded to target BoNT/B and evaluated whether multiple nanobodies can be delivered simultaneously by ^{ci}BoNT/XA. A nanobody raised against LC/B in alpaca (known as VHH-BL_{C}-JNE-B10, here abbreviated J10) was selected, which inhibits cleavage of VAMP2 by LC/B in vitro (FIGS. 16A and 16B). A8 and J10 were fused in tandem to the N-terminus of ^{ci}BoNT/XA and the fusion protein was expressed and purified in E. coli (FIG. 10A and FIG. 16A, termed A8-J10-^{ci}BoNT/XA). A8-J10-^{ci}BoNT/XA can be activated by thrombin and separated into two fragments, A8-J10-^{ci}LC/X and H_{N}—H_{C}, in the presence of DTT (FIG. 16A). Separated A8-J10-^{ci}LC/X was able to inhibit cleavage of VAMP2 by LC/B and cleavage of SNAP-25 by LC/A in the rat brain lysates with a potency similar to A8-J10 (FIGS. 16B and 16C). These results confirmed that A8 and J10 maintained their ability to inhibit LC/A and LC/B, respectively, within the A8-J10-^{ci}LC/X fusion protein.

The translocation efficacy of two nanobodies (A8-J10) was then compared with a single nanobody (A8). A new construct expressing A8-J10 fused with the active form of LCH_{N}/X was generated (A8-J10-LCH_{N}/X). LC/X within this A8-J10-LCH_{N}/X cleaved VAMP2 with efficacy similar to isolated LC/X in vitro, indicating that fusion with A8-J10 does not affect the activity of LC/X (FIG. 13C). A8-J10-LCH_{N}/X was then ligated with Hc/A using sortase to generate the active form A8-J10-BoNT/XA (FIG. 13D). Translocation efficacy was compared by examining cleavage of VAMP2 in cultured neurons exposed to ligated active toxins. Exposure to picomolar levels of A8-J10-BoNT/XA resulted in cleavage of VAMP2, and the degree of cleavage was similar to that of neurons exposed to the same concentrations of A8-BoNT/XA (FIG. 16D), suggesting that LC/X fused with two nanobodies was delivered into the cytosol of neurons as efficiently as the one fused with a single nanobody.

The ability of A8-J10-^{ci}BoNT/XA to inhibit LC/A within neurons was further assessed. Neurons were exposed to BoNT/A for 12 h, washed, incubated for another 24 h, and then incubated with V8-J10-^{ci}BoNT/XA for 48 h. A8-^{ci}BoNT/XA and a mixture of separated A8-J10 and ^{ci}BoNT/XA were analyzed in parallel as controls. Cell lysates were analyzed by immunoblot, detecting cleavage of SNAP-25 by LC/A. Incubation with A8-J10-^{ci}BoNT/XA reduced cleavage of SNAP-25, while the control mixture of A8-J10 and ^{ci}BoNT/XA did not affect cleavage of SNAP-25 (FIG. 16E). Incubation with A8-^{ci}BoNT/XA resulted in a larger reduction in cleavage of SNAP-25 compared with the same concentrations of A8-J10-^{ci}BoNT/XA (FIG. 16E), suggesting that A8-J10-^{ci}BoNT/XA showed overall lower efficacy in inhibiting LC/A in the cytosol of neurons compared with A8-^{ci}BoNT/XA.

### A8-J10-^{ci}BoNT/XA can Treat Both BoNT/A and BoNT/B Intoxication In Vivo

A8-J10-^{ci}BoNT/XA was then tested in vivo in mice. Like A8-^{ci}BoNT/XA, A8-J10-^{ci}BoNT/XA showed no toxicity after IP injection at 100 mg/Kg (FIG. 17). DAS assays were first carried out with injection of BoNT/A (FIG. 10B) or BoNT/B (FIG. 10C, BoNT/B-induced paralysis lasts ˜10-14 days in mice) to the hind leg. IM injection of A8-J10-^{ci}BoNT/XA to the same site 18 h later reduced DAS scores and shortened the duration of paralysis in a concentration-dependent manner for mice injected with either BoNT/A or BoNT/B. Muscle function was completely restored within 3 days for BoNT/A and within 2 days for BoNT/B after injection of A8-J10-^{ci}BoNT/XA, while the control mixture of A8-J10 and ^{ci}BoNT/XA did not affect the degree or duration of paralysis (FIGS. 10B and 10C). Notably, A8-J10-^{ci}BoNT/XA appeared to be less potent than A8-^{ci}BoNT/XA, as 6.5 μg is required to reduce DAS score to a similar degree as 60 ng of A8-^{ci}BoNT/XA (FIG. 8B and FIG. 10B). Further optimization of the A8-J10-^{ci}BoNT/XA protein might be needed to enhance its efficacy in neurons and in vivo.

The capability of A8-J10-^{ci}BoNT/XA was next examined to rescue mice from systemic toxicity of BoNT/A and BoNT/B, using the post-exposure IP injection model described in FIG. 9. IP administration of A8-J10-^{ci}BoNT/XA at 32.5 μg/mouse, 9 h after pre-injection of lethal doses of BoNT/A, rescued mice from death (FIG. 10D), reduced clinical scores (FIG. 10E), and eliminated body weight reduction (FIG. 10F). Lower concentrations (6.5 μg/mouse) elicited partial effects, while the control mixture of A8-J10 and ^{ci}BoNT/XA showed no effect (FIGS. 10D to 10F). Similar experiments were carried out with a lethal dose of BoNT/B (10 pg). Injection of A8-J10-^{ci}BoNT/XA showed concentration-dependent rescue from death (FIG. 10G), reduction in botulism phenotypes (FIG. 10H), and elimination of body weight reduction (FIG. 10I), with complete rescue achieved at 65 μg/mouse, while the control mixture of A8-J10 and ^{ci}BoNT/XA showed no effect (FIGS. 10G to 10I).

### Discussion

Development of biological drugs (biologics) such as proteins and antibodies has revolutionized many therapeutic areas. However, current generations of biologics are largely limited to acting on cell-surface targets. Intracellular proteins and processes represent vast untapped drug targets, yet the cell membrane forms a formidable barrier to both biologics and membrane impermeable small-molecule drugs. In addition, the capability to target a specific cell type is another major challenge for enhancing therapeutic efficacy and minimizing side effects. A protein-based drug delivery platform was developed that achieves both highly specific targeting of neurons and successful delivery of therapeutics into the cytosol of cells.

The effectiveness of this platform has been fully validated using BoNT intoxication models in vivo in mice. An anti-BoNT/A therapeutic protein was created by fusing a nanobody (A8) against LC/A to the N-terminus of the delivery protein. Using cultured neurons, it was demonstrated that A8 was delivered into the cytosol of neurons and neutralized LC/A. IM injection of this therapeutic protein 3-days or 6-days after the initial injection of BoNT/A restored muscle activity within 15 h in a local leg muscle paralysis model in mice. IP administration of this therapeutic protein rescued mice completely from systemic toxicity of BoNT/A after botulism symptoms developed. A second therapeutic protein was further developed containing two different nanobodies, one against LC/A and the other against LC/B. This single agent neutralized BoNT/A and BoNT/B in both local paralysis and systemic toxicity models, demonstrating that multiple nanobodies can be delivered simultaneously using this platform, and a single agent can thus be created to target multiple toxins.

The delivery platform is a 150 kDa chimeric protein, with one third derived from the H_{C} of a BoNT, and two thirds derived from the recently discovered BoNT-like toxin BoNT/X. The Hc of BoNT confers specificity toward neurons. The BoNT/X fragment includes an inactive form of LCH_{N} with LC catalytic activity abolished through mutations. The key finding here is that the chimeric protein containing ^{ci}LCH_{N}/X showed no toxicity in mice even at 100 mg/Kg, which allowed us to create a safe and effective protein-based delivery platform.

The molecular basis for the lack of toxicity of ^{ci}LCH_{N}/X in mice remains to be determined. BoNT/X is a newly identified BoNT-like toxin, sharing ˜28-30% sequence identity with other BoNTs and the overall conserved domain arrangement⁴⁰. Besides BoNT/X, two other BoNT-like toxins have been recently reported: one is BoNT/En, identified in an Enterococcus faecium strain^{52,53}, which shares 24-27% protein sequence identity to other BoNTs and 37% identity to BoNT/X. BoNT/En showed no toxicity in mice, and replacing its Hc with H_{C}/A resulted in a chimeric toxin that potently induced muscle paralysis in mice, suggesting that mice lack the proper receptor for BoNT/En. The other BoNT-like toxin is designated PMP1 (paraclostridial mosquitocidal protein 1), identified by screening bacteria that kill anopheles mosquito larvae⁵⁴. PMP1 shares 36% protein sequence identity with BoNT/X and 34% with BoNT/En, and the three of them form a distinct cluster within the BoNT superfamily. The natural hosts targeted by BoNT/X and BoNT/En remain unknown, while PMP1 appears to target mosquito larvae. It will be interesting to characterize ^{ci}LCH_{N} of BoNT/En and PMP1 to determine whether they share this characteristic of no toxicity in mice with ^{ci}LCH_{N}/X.

The constructed and tested fusion of ^{ci}LCH_{N}/X to three different H_{C}s: H_{C}/A, H_{C}/C, and H_{C}/D. A8-^{ci}BoNT/XA and A8-^{ci}BoNT/XC showed similar levels of efficacy in reducing SNAP-25 cleavage in cultured neurons, but 60 ng of A8-^{ci}BoNT/XA achieved better reduction in paralysis in DAS assays than 6 μg of A8-^{ci}BoNT/XC in vivo. These data suggest that A8-^{ci}BoNT/XC is less effective (or less stable) in vivo compared with A8-^{ci}BoNT/XA. A8-^{ci}BoNT/XD showed lower efficacy in reducing SNAP-25 cleavage than A8-^{ci}BoNT/XA or A8-^{ci}BoNT/XC, and its in vivo efficacy is lower than A8-^{ci}BoNT/XC. These data indicate that the choice of H_{C} affects delivery and in vivo efficacy. Potential structural conflicts between ^{ci}LCH_{N} and Hc might contribute to instability of the protein, reducing efficacy. It will be interesting to further optimize the choice of H_{C}s, as an alternative Hc other than Hc/A has the benefit of not immunizing patients against BoNT/A, thus preserving the possibility of treating patients with BoNT/A in the future.

These studies demonstrate that nanobodies can be effectively delivered into motor neurons in their functional form using this delivery platform. Interestingly, at least two tandemly fused nanobodies can be translocated into the cytosol of neurons as efficiently as a single nanobody. This allows us to develop a single agent that can simultaneously target two distinct toxins. Furthermore, dimers of two nanobodies targeting the same toxin may also be utilized to enhance the binding and inhibition of the target toxin as previously reported⁵⁵.

Nanobodies are one of the most versatile small antibody-derived protein binders that can be readily developed against any protein of interest. Besides binding and inhibiting the target protein directly, the therapeutic potential of nanobodies might be further enhanced by promoting degradation of the target protein via fusion with a protein degradation signal (degron) or a moiety that recruits E3-ubiquitin ligase. This is similar to the proteolysis-targeting chimeras (PROTACs) approach⁵⁶⁻⁵⁷, but using nanobodies rather than chemical probes for targeting the protein of interest. It has been shown that expression of A8 fused with a 15 kDa F-box domain, which recruits E3-ubiquitin ligase, accelerated degradation of LC/A in cells⁴¹. More generally, the approach of fusing of a nanobody to a protein domain recruiting E3-ubiquitin ligase to induce degradation of the target protein has been well established in cells and in model organisms⁵⁸⁻⁶². However, these previous studies lacked a way to deliver the fusion protein into cells and relied on transfection or transgenic approaches. The delivery platform reported here will enable the use of nanobody fusion proteins or nanobody conjugated with a chemical ligand to induce degradation of intracellular targets.

The range of proteins that can be efficiently delivered by this delivery platform remains to be explored experimentally. Bade et al. showed that the translocation efficacy of proteins fused to BoNT/D are influenced not only by size, but more importantly by structural rigidity³¹. For instance, firefly luciferase (62 kDa) fused to BoNT/D was translocated into neurons at a higher rate than GFP (27 kDa). Furthermore, fusion of DHFR (25 kDa) to BoNT/D did not affect translocation of BoNT/D, but when DHFR is stabilized by binding to folate analogue Mtx, it reduced translocation of the fusion protein 10-fold. It is interestingly to note that firefly luciferase fused with BoNT/D was translocated at 14-fold lower efficacy compared with BoNT/D alone, while A8 fusion to BoNT/XA showed a similar range of reduction (˜7.4-fold) in translocation efficacy compared with BoNT/XA. The major limiting factor for any protein-based delivery platform is likely the generation of neutralizing antibodies over time, which renders repeated usage less effective. This issue could be ameliorated through additional protein engineering of deimmunization. This however is not an issue for treating botulism, which likely involves only a single treatment event.

In summary, a neuron-specific delivery platform was created based on a chimeric toxin approach by combining the neuronal specificity of the BoNT-Hc and the unique non-toxic property of the de-activated LCH_{N} of BoNT/X. Based on this platform, a safe and effective post-exposure treatment was developed for BoNT/A and BoNT/B. The modular nature of these wechimeric toxins offers a general approach to targeting distinct cell types through changing the receptor-binding domain. Furthermore, different types of cargoes, such as therapeutic peptide/proteins, small molecules, and potentially DNA/RNA, can be conjugated to the delivery system, with the potential to target and modulate previously hard-to-reach cytosolic targets.

### Methods

### Study Design

The objective of this study is to establish a drug delivery platform to target and inhibit botulinum neurotoxins (BoNTs) within the cytosol of neurons to provide a post-exposure treatment for BoNT intoxication and botulism. A catalytically inactive chimeric toxin-based delivery vehicle was created and utilized nanobodies against BoNTs as therapeutic cargoes. The nanobody-delivery vehicle fusion proteins were expressed in E. coli and purified as His6-tagged proteins. The toxicity to mice via IP injections was first evaluated, and the delivery of nanobodies into the cytosol of cultured rat cortical neurons was then examined, followed by assessing the therapeutic effect in vivo using both a local muscle paralysis model and a systemic toxicity model in mice. Experiments were carried out three times independently. The sample sizes were selected based on previous literature and noted in each figure. The humane endpoint was defined based on clinical scores and body weight reduction (FIG. 18). Mice were randomly assigned to either treatment or control groups. For all animal experiments, investigators were not blinded to the treatment/control groups or the data analysis. All procedures using mice were conducted in accordance with the guidelines approved by the Institute Animal Care and Use Committee at Boston Children's Hospital (#18-10-3794R).

### Materials

Goat Anti-Llama IgG H&L (HRP) (ab112786, 1:500) was purchased from Abcam (Cambridge, United Kingdom). Mouse monoclonal antibodies for Syntaxin 1 (Cl 78.2, 1:3,000), SNAP-25 (C171.1, 1:2,000), and VAMP2 (Cl 69.1, 1:1,000) were purchased from Synaptic Systems (Gottingen, Germany). The following antibodies were purchased from the indicated vendors: rabbit polyclonal antibody for Synapsin (Millipore); mouse monoclonal antibody for actin (AC-15, Sigma, 1:1,000). The human monoclonal antibody against BoNT/A (Raz-1, 1:1,000) was generously provided by Jianlong Lou and James Marks (San Francisco, Calif.). BoNT/A and BoNT/B were purchased from Metabiologics (Madison, Wis., USA).

### Plasmid Construction

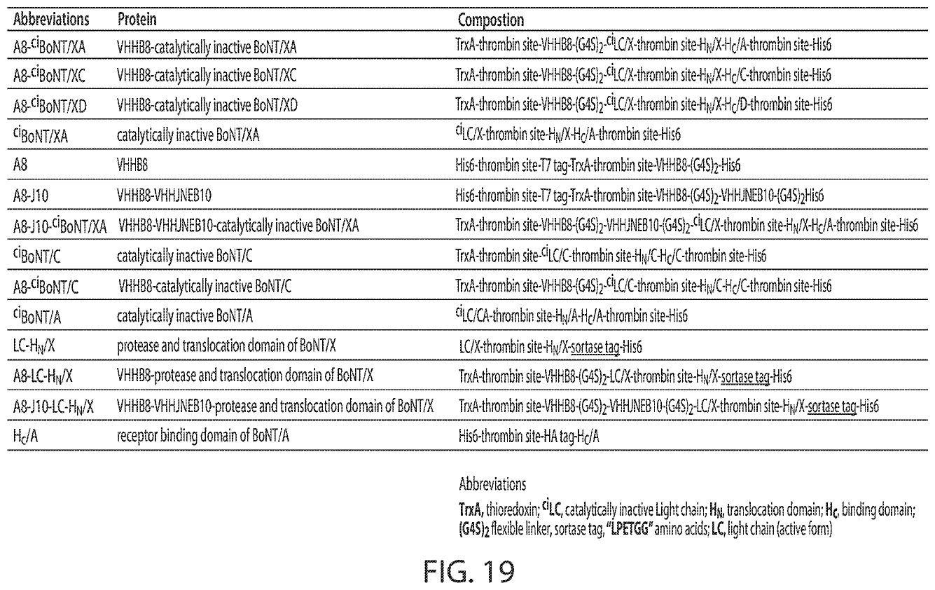

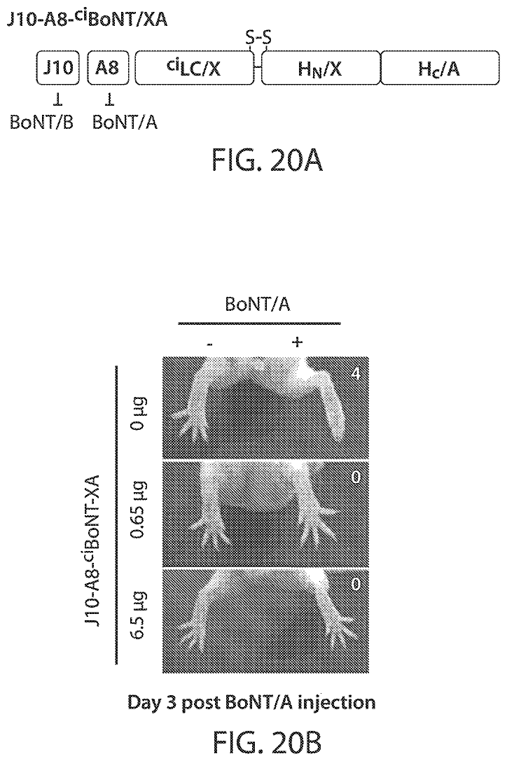

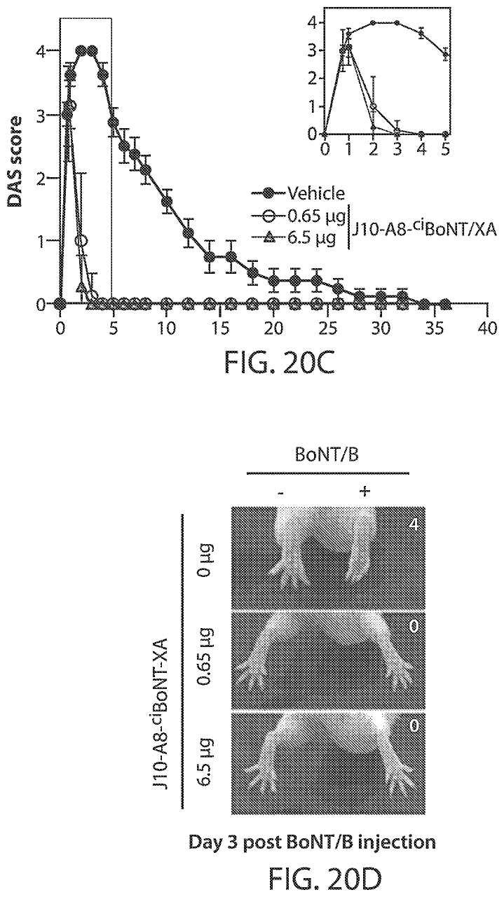

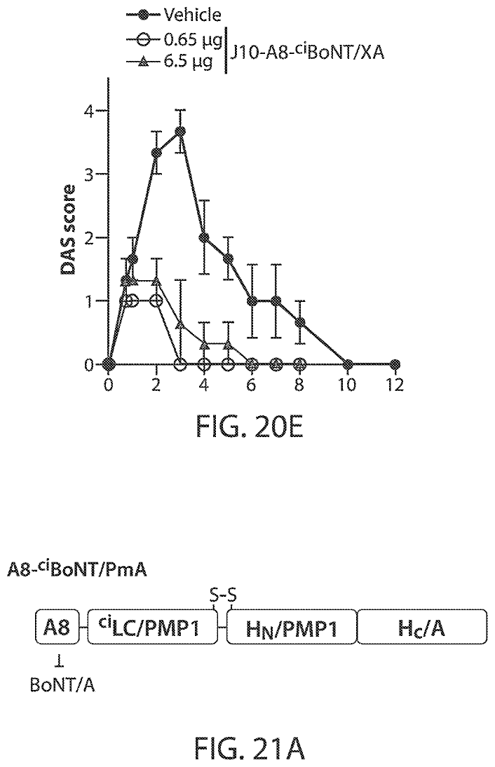

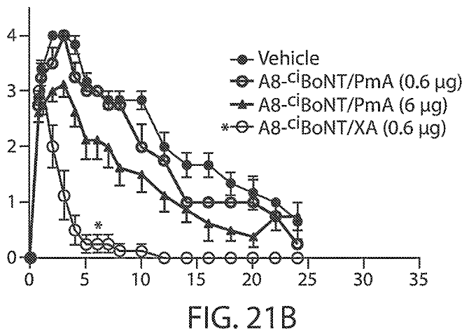

The cDNA encoding A8 (GenBank: FJ643070.1) and J10 were synthesized by IDT (Coralville, Iowa). Plasmids were constructed using PCR and NEBuilder® HiFi DNA Assembly Master Mix (New England Biolabs, Beverly, Mass.). The composition of all constructs in this study is summarized in FIG. 19. The cDNAs encoding ^{ci}BoNT/XA (LC/X, residues 1-422; H_{N}/X, residues 468-924; H_{C}/A, residues 873-1296) were cloned into pET28a vector with a His6-tag fused to its C-terminus. Three amino acids in LC/X were mutated (residues E228Q, R360A, and Y363F) by site-directed mutagenesis. Three thrombin cleavage sites were introduced to the locations between LC/X and H_{N}/X, between Hc and the His6-tag, and between the N-terminal thioredoxin tag (TrxA) and LC/X. A8-^{ci}BoNT/XA, XC, and XD chimera (Hc/C, residues 868-1291; Hc/D, residues 864-1276) were cloned into pET28a vectors with His6-tag on their N-termini. Flexible 10-amino acid linker (Gly4Ser)₂ was introduced between A8 and LC/X. ^{ci}BoNT/C (E230Q, R372A, and Y375F), A8-^{ci}BoNT/C, and ^{ci}BoNT/A (E224Q, R363A, and Y366F) were sub-cloned into pET28a vector. A8, J10, and A8-J10 were cloned into pET28a vector with TrxA tag at N-termini and His6-tag at C-termini. A8-J10-^{ci}BoNT/XA was cloned into pET28a vector. Catalytically active LCH_{N}/X, A8-LCH_{N}/X, and A8-J10-LCH_{N}/X were cloned into pET28a vector with the sortase tag sequence “LPETGG” fused to their C-termini, followed by a His6-tag. Rat SV2C-L4 (residues 473-567) and Rat VAMP2 (1-93) was cloned into pGEX-4T-1. The construct encoding His6-tagged sortase (SrtA*) was generously provided by B. Pentelute (Boston, Mass., USA).

### Protein Expression, Purification, and Activation

Plasmids were transformed into E. coli BL21 (DE3). Cells were cultured at 37° C. and 300 rpm shaking in 2 L baffled flasks containing 400 mL of autoinduction medium (Formedium). Once the OD₆₀₀ reached 0.4-0.6, the temperature was decreased to 16° C., and further incubated for 18-24 h. The cells were harvested at 4,000 rpm for 30 min and stored at −80° C. For A8-J10-^{ci}BoNT/XA expression, the plasmid was transformed into SHuffle T7 Express E. coli (NEB). Cells were cultured at 30° C. and 250 rpm shaking in 2 L baffled flasks containing 1,000 mL of Terrific Broth medium. Expression was induced with 0.4 mM IPTG when OD₆₀₀ reached 0.5-0.8, then the temperature was decreased to 16° C., and further cultured for 18 h. The cells were harvested at 4,000 rpm for 30 min and stored at −80° C.

All protein purification steps were performed at 4° C. Bacterial cells for ^{ci}BoNT or nanobody-^{ci}BoNT were disrupted by sonication in the binding buffer (20 mM Tris-HCl pH 7.5, 500 mM NaCl, 10% glycerol, 20 mM imidazole, 1 mM PMSF). Lysates were centrifuged at 20,000 rpm for 30 min at 4° C. The supernatant was loaded on to a HisTrap HP (5 mL, GE) and washed with the binding buffer. Proteins were eluted by a linear gradient of 20-250 mM of imidazole over 50 mL. Target proteins were collected based on molecular weight and concentrated using Vivaspin (GE Healthcare, cut-off 100 kDa). To generate the di-chain form of ^{ci}BoNT, proteins were proteolytically cleaved with thrombin (2 U/mg protein, Millipore) at 4° C. overnight. The proteins were further purified using size-exclusion column (Superdex 200 pg 16/60, GE Health care) in 20 mM Tris-HCl pH 7.5, 150 mM NaCl. The elution peak was collected and concentrated using Vivaspin (100 kDa MWCO). Proteins were passed through an endotoxin removal resin (Thermo scientific) and sterilized using 0.22 μm filters (Millipore). Purified proteins were aliquoted (50-100 μL/tube) and stored at −80° C.

A8 and A8-J10 were purified using a HisTrap column. For removing the TrxA tag, the elution was treated with thrombin at 4° C. overnight and passed through a PD-10 column (GE Healthcare) equilibrated in the binding buffer. Elutions were incubated with Ni-NTA beads at room temperature (RT) for 30 min and washed three times using the binding buffer. The A8 and A8-J10 were eluted using 250 mM imidazole and concentrated using Vivaspin (10 kDa MWCO). The proteins were further purified using size-exclusion column (Superdex 75 10/30, GE Health care) in 20 mM Tris-HCl pH 7.5, 150 mM NaCl. LCH_{N}/X, A8-LCH_{N}/X, A8-J10-LCH_{N}/X, and H_{C}/A were purified using HisTrap column. The proteins were further purified using size-exclusion column (Superdex 200 pg 16/60) in 20 mM Tris-HCl pH 7.5, 150 mM NaCl, and 10% glycerol.

### Neuron Cultures

Pregnant rats were purchased from Charles River. 24-well plates were coated with poly-D-lysine (0.5 mg/mL in deionized water) at 37° C. for 3 h and washed three times with deionized water. Primary rat cortical neurons were prepared from E18-19 embryos using a papain dissociation kit (Worthington Biochemical). Pregnant rats were euthanized by CO₂ asphyxiation and embryos removed. Dissected cortical tissue was dissociated in papain solution at 37° C. for 60 min. Cortical neurons were plated on poly-D-lysine coated 24-well plates at a density of 250,000 cells/well (for western-blot) or 150,000 cells/well (for immunostaining) in 1 mL of culture medium (Neurobasal medium containing 1× B27, 0.5% FBS).

### Detection of Nanobody in the Cytosol of Neuron

Neurons were exposed to A8-^{ci}BoNT/XA with or without 100 nM of bafilomycin A1 in medium for 12 h. Cells were washed with PBS three times and lysed with 100 μL of lysis buffer (PBS containing 1% Triton X-100, 0.05% SDS, protease inhibitor cocktail tablet (Thermo scientific)). Lysates were centrifuged for 10 min at 4° C. The supernatant was mixed with SDS-sample buffer (62.5 mM Tris-HCl pH 6.8, 2% SDS, 10% glycerol, 0.005% bromophenol blue) without DTT and subjected to immunoblot analysis under non-reducing conditions to detect translocated A8-^{ci}LC/X. The A8-^{ci}LC/X and A8-^{ci}BoNT/XA were detected using HRP-conjugated goat anti-llama IgG via enhanced chemiluminescence (Thermo Scientific Pierce, #32106).

### Post-Exposure Inhibition of BoNT/A in Cultured Cortical Neurons

Neurons were cultured in 1.5 mL cultured medium. After 11 days in vitro, the 1,200 μL of culture medium were collected and used as a conditioned medium. Neurons were exposed to 20 pM BoNT/A in 300 μL of the conditional medium at 37° C. for 12 h. Cells were washed two times with the medium to remove residual BoNTs, and incubated in 300 μL of the medium for 24 h. Neurons were further exposed to A8-^{ci}BoNT/XA in 400 μL of the medium for 48 h, and then lysed with 200 μL of lysis buffer. Lysates were centrifuged for 10 min at 4° C. Supernatants were subjected to SDS-PAGE and immunoblot analysis.

### Immunostaining

A8-^{ci}BoNT/XA (200 nM) and GST-SV2C-L4 (2 μM) were incubated for 20 min at 37° C. Neurons were exposed to the mixture in medium for 8 min at 37° C. Cells were washed with ice-cold PBS and fixed with PBS containing 4% paraformaldehyde for 20 min at RT. Cells were treated with PBS containing 10% goat normal serum for 45 min and exposed to human anti-BoNT/A antibody (1:500) and rabbit anti-Synapsin antibody (1:600) at 4° C. overnight. Cells were washed with PBS and incubated with Alexa-488 goat anti-human IgG and Alexa-546 goat anti-rabbit IgG (1:800) for 1 h. The coverslip was then mounted on a slide and images were collected using a fluorescence microscope (Olympus IX81).

### Sortase-Mediated Ligation and Assessing Translocation Efficacy

HA-tagged H_{C}/A was cleaved overnight at 4° C. by thrombin to expose the glycine residue at the N-terminus. The ligation reaction was set up in 50 μL Tris buffer pH 7.5, with H_{C}/A (40 μM), LCH_{N}/X, A8-LCH_{N}/X or A8-J10-LCH_{N}/X (4 μM), Ca²⁺ (10 mM), and sortase (0.5 μM) for 45 min at RT. The Ca²⁺ and sortase were removed using a Vivaspin concentrator (100 kDa MWCO, GE). The ligation products were activated by thrombin treatment (0.4 U) for 30 min at RT. Ligated products were subjected to SDS-PAGE and the concentration was quantified using ImageJ software. Neurons were exposed to ligated products in 300 μL cultured medium for 12 h at 37° C. Cell lysates were subjected to immunoblot analysis detecting cleavage of VAMP2.

### Cleavage of Recombinant VAMP2 by LC/X, A8-LC/X, and A8-J10-LC/X

VAMP2 (1-93) was expressed and purified as a GST-tagged protein. LCH_{N}/X, A8-LCH_{N}/X, and A8-J10-LCH_{N}/X were activated with thrombin treatment and incubated with DTT to generate LC/X, A8-LC/X, and A8-J10-LC/X. GST-VAMP2 (4 μM) were incubated with LC/X, A8-LC/X, or A8-J10-LC/X (300, 100, 30 or 10 nM) for 2 min at 37° C. Samples were analyzed by SDS-PAGE and Coomassie blue staining.

### Brain Detergent Extract Preparation and In Vitro Toxin Neutralization Assay

Rat brain detergent extracts (BDE) were prepared as previously described⁴⁰. The LC/A (1 μM, final concentration), or LC/B (1 μM) was pre-incubated with A8, A8-^{ci}BoNT/XA, or A8-J10-^{ci}BoNT/XA in 15 μL Tris buffer pH7.5 for 30 min at RT. The mixtures were then added to 15 μL BDE (2 mg/mL) and incubated for 1 h at 37° C. Samples were subjected to SDS-PAGE and immunoblot analysis.

### Digit Abduction Score (DAS) Assay

Male mice (CD-1 strain, 20-30 g) were purchased from Envigo. BoNTs were diluted in 0.2% gelatin-phosphate buffer pH 6.3. Mice were anesthetized with isoflurane and administered 10 μL BoNT/A (6 pg) or BoNT/B (3.6 pg) by IM injection into the gastrocnemius muscle of the right hind limb using a 30-gauge needle attached to a Hamilton syringe. Mice were scored for DAS response by muscle paralysis and the spread of hind toe digit abduction starting 18 h following BoNT injection. The degree of digit abduction was scored on a five-point scale (0; normal, to 4; maximal paralysis, FIG. 8A). Mice were monitored once per day for 10 days, then further monitored once every other day until fully recovered from the paralysis.

### Mouse Lethality Assay and Systemic Post-Exposure Treatment Model

Mice were administered a lethal dose of BoNT/A (19.5 pg) or BoNT/B (10 pg) in 100 μL of 0.2% gelatin-Phosphate buffer pH 6.3 through IP injection. After 9 h, mice that developed typical botulism phenotypes such as wasp waist were selected and randomly assigned to either treatment or control groups. These mice were then administered vehicle control (0.2% gelatin-PBS), a mixture of A8 and ^{ci}BoNT/XA, A8-J10-^{ci}BoNT/XA, A8-^{ci}BoNT/XA or A8-J10-^{ci}BoNT/XA in 0.2% gelatin-PBS by IP injection. Mice were monitored once per every 2 h for 14 h, followed by three times per day for 5 days, and then once every other day for 21 days. Survival rates, clinical scores (FIG. 18), and body weight were recorded. The humane endpoint was set as total clinical score above 5.

### Statistical Analysis

Statistical analysis was performed using GraphPad Prism 8.3 software. The statistical significance of the observed differences was calculated using one- or two-way ANOVA with Dunnett post-hoc tests. Survival curves were analyzed using Log-rank (Mantel-Cox) test. Results were considered significant when P<0.05.

TABLE 2
Amino Acid Sequences

| | | SEQ ID |
|---|---|---|
| Name | Amino Acid Sequence | NO |
| BoNT/X (GenBank | MKLEINKFNYNDPIDGINVITMRPPRHSDKINKGKGPFKAFQVIKNIWIVP | 1 |
| No. BAQ12790.1) | ERYNFTNNTNDLNIPSEPIMEADAIYNPNYLNTPSEKDEFLQGVIKVLERI | |
| | KSKPEGEKLLELISSSIPLPLVSNGALTLSDNETIAYQENNNIVSNLQANLV | |
| | IYGPGPDIANNATYGLYSTPISNGEGTLSEVSFSPFYLKPFDESYGNYRSL | |
| | VNIVNKFVKREFAPDPASTLMHELVHVTHNLYGISNRNFYYNFDTGKIET | |
| | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVR | |
| | KHYLKERPIDPIYVNILDDNSYSTLEGFNISSQGSNDFQGQLLESSYFEKIE | |
| | SNALRAFIKICPRNGLLYNAIYRNSKNYLNNIDLEDKKTTSKTNVSYPCSL | |
| | LNGCIEVENKDLFLISNKDSLNDINLSEEKIKPETTVFFKDKLPPQDITLSN | |
| | YDFTEANSIPSISQQNILERNEELYEPIRNSLFEIKTIYVDKLTTFHFLEAQN | |
| | IDESIDSSKIRVELTDSVDEALSNPNKVYSPFKNMSNTINSIETGITSTYIFY | |
| | QWLRSIVKDFSDETGKIDVIDKSSDTLAIVPYIGPLLNIGNDIRHGDFVGAI | |
| | ELAGITALLEYVPEFTIPILVGLEVIGGELAREQVEAIVNNALDKRDQKW | |
| | AEVYNITKAQWWGTIHLQINTRLAHTYKALSRQANAIKMNMEFQLANY | |
| | KGNIDDKAKIKNAISETEILLNKSVEQAMKNTEKFMIKLSNSYLTKEMIPK | |
| | VQDNLKNFDLETKKTLDKFIKEKEDILGTNLSSSLRRKVSIRLNKNIAFDI | |
| | NDIPFSEFDDLINQYKNEIEDYEVLNLGAEDGKIKDLSGTTSDINIGSDIEL | |
| | ADGRENKAIKIKGSENSTIKIAMNKYLRFSATDNFSISFWIKHPKPTNLLN | |
| | NGIEYTLVENFNQRGWKISIQDSKLIWYLRDHNNSIKIVTPDYIAFNGWN | |
| | LITITNNRSKGSIVYVNGSKIEEKDISSIWNTEVDDPIIFRLKNNRDTQAFTL | |
| | LDQFSIYRKELNQNEVVKLYNYYFNSNYIRDIWGNPLQYNKKYYLQTQD | |
| | KPGKGLIREYWSSFGYDYVILSDSKTITFPNNIRYGALYNGSKVLIKNSKK | |
| | LDGLVRNKDFIQLEIDGYNMGISADRFNEDTNYIGTTYGTTHDLTTDFEII | |
| | QRQEKYRNYCQLKTPYNIFHKSGLMSTETSKPTFHDYRDWVYSSAWYF | |
| | QNYENLNLRKHTKTNWYFIPKDEGWDED | |
| BoNT/En | MVTINDLHYSDPIDEDNIINMRIPLYDLEVDDQFINHNVPDLKAFQVFPNV | 2 |
| (GenBank No. | WVVPERYTFYSTMKNLDAPANPSRSSYYDPTYLQSDAEKEVFLQQMILL | |
| OTO22244.1) | FKRINSTQEGQQFLNLLSRSIPVPYESNGDVAMGTTQVIKQMDDKGNVL | |
| | KHRRAHIIIYGPGPDLMAKGSKALTKSRETGRGCMAEIYFSPMYHKTYST | |
| | KLTNKNSLVDKSVQEFVPDPAVTLIHELCHGLHALYGIDLGNVGSWEFN | |
| | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTRARYLVPKVENIVQVDILSPNVYTIDKGFNHLSKGFK | |
| | GQSVSQSYFRKISALARGAVVRACPNPHFSSQRGLSSCIEILEDDLFIMSS | |
| | KDSFTDTDFSEPSVGPVSYKAKKGADTILDSTLSNYDFSKEINFTSTVPIIT | |
| | VEDPLETDEDVPVISEDRTVYVDDYTTFHFLEAQKIGKEVVPTQTKVVFT | |
| | TNMEEALFDSKKVYTVFENTASRINEAGTGIANGMMFYQWLKGIVQDFT | |
| | EEATQKDTFDKISDVTMIVPYLGNILNIGNDIRKGDFMGAVELGGVTILLE | |
| | AIPELTLPVLIGLTIIEDELEKEQVSQTVYNVLDKRDEKWEEVYGFVKQQ | |
| | WWWMVHTQFETRILHAYQALNHQVEAIKANMTYQLANYRGNQEDKEL | |
| | LEKAIDDTLQSLYYAVDQAMHNIKRFLIQSSKSYLLNQMLPKTKEQLLAF | |
| | DQQTLRNVNDFINKNQGVLGESLAKDLKKKVEKRLTSLPVFNLEDLPISE | |
| | FEDLIHSHEIDIQDSEVLNIGVNNGKIQDLSGENTPLTLGENLHIVNGRDN | |
| | QAVRLNNQLDSKLEIQSRPNIHFTAFEDFSISIWIRCSMLRNNRNRGQKYT | |
| | IIQQFNKYGWQLAIQDSVFVWTLHDTFNNQIQLTSGSALTNKNYLLQNF | |
| | WLHITVTNKRSEKSRLYINGVLQDQKDISVLGNCHPKEPILFSIQDNSDPN | |
| | YFVRFEQFNVYRKALTDSEVNRLYWKYFEGSYLRDVWGERLTYNRDYY | |
| | MQLSTLPGRGIKREYRTWSGFDYIILSELGTQKIPTHEVTYPKLYQGQKIT | |
| | IHSDGKNLEPHVKSNKNIRLKIDDFYIGVVNPFKLPEWRPESGAYVVTTY | |
| | NHAEDLCLYFRTRSSSQSLYYGQLIMNDGRNKSLLNYTLKGSTYWIWSS | |
| | AWYYENYNTSSKTAGNWYFIPVDEGWKED | |
| BoNT/PMP1 | MLQIRVFNYNDPIDGENIVELRYHNRSPVKAFQIVDGIWIIPERYNFTNDT | 85 |
| | KKVPDDRALTILEDEVFAVRENDYLTTDVNEKNSFLNNITKLFKRINSSNI | |
| | GNQLLNYISTSVPYPVVSTNSIKARDYNTIKFDSIDGRRITKSANVLIYGPS | |
| | MKNLLDKQTRAINGEEAKNGIGCLSDIIFSPNYLSVQTVSSSRFVEDPASS | |
| | LTHELIHALHNLYGIQYPGEEKFKFGGFIDKLLGTRECIDYEEVLTYGGK | |
| | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVRKHYITKQINP | |
| | KYTELTNDVYTEDKGFVNGQSIDNQNFKIIDDLISKKVKLCSITSKNRVNI | |
| | CIDVNKEDLYFISDKEGFENIDFSEPEIRYDSNVTTATTSSFTDHFLVNRTF | |
| | NDSDRFPPVELEYAIEPAEIVDNTIMPDIDQKSEISLDNLTTFHYLNAQKM | |
| | DLGFDSSKEQLKMVTSIEESLLDSKKVYTPFTRTAHSVNERISGIAESYLF | |
| | YQWLKTVINDFTDELNQKSNTDKVADISWIIPYVGPALNIGLDLSHGDFT | |
| | KAFEDLGVSILFAIAPEFATISLVALSIYENIEEDSQKEKVINKVENTLARRI | |
| | EKWHQVYAFMVAQWWGMVHTQIDTRIHQMYESLSHQIIAIKANMEYQ | |
| | LSHYKGPDNDKLLLKDYIYEAEIALNTSANRAMKNIERFMIESSISYLKN | |
| | NLIPSVVENLKKFDADTKKNLDQFIDKNSSVLGSDLHILKSQVDLELNPT | |
| | TKVAFNIQSIPDFDINALIDRLGIQLKDNLVFSLGVESDKIKDLSGNNTNLE | |
| | VKTGVQIVDGRDSKTIRLNSNENSSIIVQKNESINFSYFSDFTISFWIRVPRL | |
| | NKNDFIDLGIEYDLVNNMDNQGWKISLKDGNLVWRMKDRFGKIIDIITSL | |
| | TFSNSFIDKYISSNIWRHITITVNQLKDCTLYINGDKIDSKSINELRGIDNNS | |
| | PIIFKLEGNRNKNQFIRLDQFNIYQRALNESEVEMLFNSYFNSNILRDFWG | |
| | EPLEYNKSYYMINQAILGGPLRSTYKSWYGEYYPYISRMRTFNVSSFILIP | |
| | YLYHKGSDVEKVKIINKNNVDKYVRKNDVADVKFENYGNLILTLPMYS | |
| | KIKERYMVLNEGRNGDLKLIQLQSNDKYYCQIRIFEMYRNGLLSIADDEN | |
| | WLYSSGWYLYSSGWYLDNYKTLDLKKHTKTNWYFVSEDEGWKE | |
| ^{8ci}LC-Hn/X | MKLEINKFNYNDPIDGINVITMRPPRHSDKINKGKGPFKAFQVIKNIWIVP | 3 |
| | ERYNFTNNTNDLNIPSEPIMEADAIYNPNYLNTPSEKDEFLQGVIKVLERI | |
| | KSKPEGEKLLELISSSIPLPLVSNGALTLSDNETIAYQENNNIVSNLQANLV | |
| | IYGPGPDIANNATYGLYSTPISNGEGTLSEVSFSPFYLKPFDESYGNYRSL | |
| | VNIVNKFVKREFAPDPASTLMHQLVHVTHNLYGISNRNFYYNFDTGKIE | |
| | TSRQQNSLIFEELLTFGGIDSKAISSLIIKKIIETAKNNYTTLISERLNTVTVE | |
| | NDLLKYIKNKIPVQGRLGNFKLDTAEFEKKLNTILFVLNESNLAQRFSILV | |
| | AKHFLKERPIDPIYVNILDDNSYSTLEGFNISSQGSNDFQGQLLESSYFEKI | |
| | ESNALRAFIKICPRNGLLYNAIYRNSKNYLNNIDLEDKKTTSKTNVSYPCS | |
| | LLNGCIEVENKDLFLISNKDSLNDINLSEEKIKPETTVFFKDKLPPQDITLS | |
| | NYDFTEANSIPSISQQNILERNEELYEPIRNSLFEIKTIYVDKLTTFHFLEAQ | |
| | NIDESIDSSKIRVELTDSVDEALSNPNKVYSPFKNMSNTINSIETGITSTYIF | |
| | YQWLRSIVKDFSDETGKIDVIDKSSDTLAIVPYIGPLLNIGNDIRHGDFVG | |
| | AIELAGITALLEYVPEFTIPILVGLEVIGGELAREQVEAIVNNALDKRDQK | |
| | WAEVYNITKAQWWGTIHLQINTRLAHTYKALSRQANAIKMNMEFQLAN | |
| | YKGNIDDKAKIKNAISETEILLNKSVEQAMKNTEKFMIKLSNSYLTKEMIP | |
| | KVQDNLKNFDLETKKTLDKFIKEKEDILGTNLSSSLRRKVSIRLNKNIAFD | |
| | INDIPFSEFDDLINQYKNEIEDYEVLNLGAEDGKIKDLSGTTSDINIGSDIE | |
| ^{ci}LC-Hn/En | MVTINDLHYSDPIDEDNIINMRIPLYDLEVDDQFINHNVPDLKAFQVFPNV | 4 |
| | WVVPERYTFYSTMKNLDAPANPSRSSYYDPTYLQSDAEKEVFLQQMILL | |
| | FKRINSTQEGQQFLNLLSRSIPVPYESNGDVAMGTTQVIKQMDDKGNVL | |
| | KHRRAHIIIYGPGPDLMAKGSKALTKSRETGRGCMAEIYFSPMYHKTYST | |
| | KLTNKNSLVDKSVQEFVPDPAVTLIHQLCHGLHALYGIDLGNVGSWEFN | |
| | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACPNPHFSSQRGLSSCIEILEDDLFIMSS | |
| | KDSFTDTDFSEPSVGPVSYKAKKGADTILDSTLSNYDFSKEINFTSTVPIIT | |
| | VEDPLETDEDVPVISEDRTVYVDDYTTFHFLEAQKIGKEVVPTQTKVVFT | |
| | TNMEEALFDSKKVYTVFENTASRINEAGTGIANGMMFYQWLKGIVQDFT | |
| | EEATQKDTFDKISDVTMIVPYLGNILNIGNDIRKGDFMGAVELGGVTILLE | |
| | AIPELTLPVLIGLTIIEDELEKEQVSQTVYNVLDKRDEKWEEVYGFVKQQ | |
| | WWWMVHTQFETRILHAYQALNHQVEAIKANMTYQLANYRGNQEDKEL | |
| | LEKAIDDTLQSLYYAVDQAMHNIKRFLIQSSKSYLLNQMLPKTKEQLLAF | |
| | DQQTLRNVNDFINKNQGVLGESLAKDLKKKVEKRLTSLPVFNLEDLPISE | |
| | FEDLIHSHEIDIQDSEVLNIGVNNGKIQDLSGENTPLTLGENLH | |
| ^{ci}LC-Hn/PMP1 | MLQIRVFNYNDPIDGENIVELRYHNRSPVKAFQIVDGIWIIPERYNFTNDT | 86 |
| | KKVPDDRALTILEDEVFAVRENDYLTTDVNEKNSFLNNITKLFKRINSSNI | |
| | GNQLLNYISTSVPYPVVSTNSIKARDYNTIKFDSIDGRRITKSANVLIYGPS | |
| | MKNLLDKQTRAINGEEAKNGIGCLSDIIFSPNYLSVQTVSSSRFVEDPASS | |
| | LTHQLIHALHNLYGIQYPGEEKFKFGGFIDKLLGTRECIDYEEVLTYGGK | |
| | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCSITSKNRVNICI | |
| | DVNKEDLYFISDKEGFENIDFSEPEIRYDSNVTTATTSSFTDHFLVNRTFN | |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQ | |
| ^{ci}BoNT/XA | MKLEINKFNYNDPIDGINVITMRPPRHSDKINKGKGPFKAFQVIKNIWIVP | 5 |
| | ERYNFTNNTNDLNIPSEPIMEADAIYNPNYLNTPSEKDEFLQGVIKVLERI | |
| | KSKPEGEKLLELISSSIPLPLVSNGALTLSDNETIAYQENNNIVSNLQANLV | |
| | IYGPGPDIANNATYGLYSTPISNGEGTLSEVSFSPFYLKPFDESYGNYRSL | |
| | VNIVNKFVKREFAPDPASTLMHQLVHVTHNLYGISNRNFYYNFDTGKIET | |
| | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICPRNGLLYNAIYRNSKNYLNNIDLEDKKTTSKTNVSYPCSL | |
| | LNGCIEVENKDLFLISNKDSLNDINLSEEKIKPETTVFFKDKLPPQDITLSN | |
| | YDFTEANSIPSISQQNILERNEELYEPIRNSLFEIKTIYVDKLTTFHFLEAQN | |
| | IDESIDSSKIRVELTDSVDEALSNPNKVYSPFKNMSNTINSIETGITSTYIFY | |
| | QWLRSIVKDFSDETGKIDVIDKSSDTLAIVPYIGPLLNIGNDIRHGDFVGAI | |
| | ELAGITALLEYVPEFTIPILVGLEVIGGELAREQVEAIVNNALDKRDQKW | |
| | AEVYNITKAQWWGTIHLQINTRLAHTYKALSRQANAIKMNMEFQLANY | |
| | KGNIDDKAKIKNAISETEILLNKSVEQAMKNTEKFMIKLSNSYLTKEMIPK | |
| | VQDNLKNFDLETKKTLDKFIKEKEDILGTNLSSSLRRKVSIRLNKNIAFDI | |
| | NDIPFSEFDDLINQYKNEIEDYEVLNLGAEDGKIKDLSGTTSDINIGSDIEII | |
| | NTSILNLRYESNHLIDLSRYASKINIGSKVNFDPIDKNQIQLFNLESSKIEVI | |
| | LKNAIVYNSMYENFSTSFWIRIPKYFNSISLNNEYTIINCMENNSGWKVSL | |
| | NYGEIIWTLQDTQEIKQRVVFKYSQMINISDYINRWIFVTITNNRLNNSKI | |
| | YINGRLIDQKPISNLGNIHASNNIMFKLDGCRDTHRYIWIKYFNLFDKELN | |
| | EKEIKDLYDNQSNSGILKDFWGDYLQYDKPYYMLNLYDPNKYVDVNNV | |
| | GIRGYMYLKGPRGSVMTTNIYLNSSLYRGTKFIIKKYASGNKDNIVRNND | |
| | RVYINVVVKNKEYRLATNASQAGVEKILSALEIPDVGNLSQVVVMKSKN | |
| | DQGITNKCKMNLQDNNGNDIGFIGFHQFNNIAKLVASNWYNRQIERSSR | |
| | TLGCSWEFIPVDDGWGERPLQ | |
| ^{ci}BoNT/XB | MKLEINKFNYNDPIDGINVITMRPPRHSDKINKGKGPFKAFQVIKNIWIVP | 6 |
| | ERYNFTNNTNDLNIPSEPIMEADAIYNPNYLNTPSEKDEFLQGVIKVLERI | |
| | KSKPEGEKLLELISSSIPLPLVSNGALTLSDNETIAYQENNNIVSNLQANLV | |
| | IYGPGPDIANNATYGLYSTPISNGEGTLSEVSFSPFYLKPFDESYGNYRSL | |
| | VNIVNKFVKREFAPDPASTLMHQLVHVTHNLYGISNRNFYYNFDTGKIET | |
| | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICPRNGLLYNAIYRNSKNYLNNIDLEDKKTTSKTNVSYPCSL | |
| | LNGCIEVENKDLFLISNKDSLNDINLSEEKIKPETTVFFKDKLPPQDITLSN | |
| | YDFTEANSIPSISQQNILERNEELYEPIRNSLFEIKTIYVDKLTTFHFLEAQN | |
| | IDESIDSSKIRVELTDSVDEALSNPNKVYSPFKNMSNTINSIETGITSTYIFY | |
| | QWLRSIVKDFSDETGKIDVIDKSSDTLAIVPYIGPLLNIGNDIRHGDFVGAI | |
| | ELAGITALLEYVPEFTIPILVGLEVIGGELAREQVEAIVNNALDKRDQKW | |
| | AEVYNITKAQWWGTIHLQINTRLAHTYKALSRQANAIKMNMEFQLANY | |
| | KGNIDDKAKIKNAISETEILLNKSVEQAMKNTEKFMIKLSNSYLTKEMIPK | |
| | VQDNLKNFDLETKKTLDKFIKEKEDILGTNLSSSLRRKVSIRLNKNIAFDI | |
| | NDIPFSEFDDLINQYKNEIEDYEVLNLGAEDGKIKDLSGTTSDINIGSDIEIL | |
| | NNIILNLRYKDNNLIDLSGYGAKVEVYDGVELNDKNQFKLTSSANSKIRV | |
| | TQNQNIIFNSVFLDFSVSFWIRIPKYKNDGIQNYIHNEYTIINCMKNNSGW | |
| | KISIRGNRIIWTLIDINGKTKSVFFEYNIREDISEYINRWFFVTITNNLNNAK | |
| | IYINGKLESNTDIKDIREVIANGEIIFKLDGDIDRTQFIWMKYFSIFNTELSQ | |
| | SNIEERYKIQSYSEYLKDFWGNPLMYNKEYYMFNAGNKNSYIKLKKDSP | |
| | VGEILTRSKYNQNSKYINYRDLYIGEKFIIRRKSNSQSINDDIVRKEDYIYL | |
| | DFFNLNQEWRVYTYKYFKKEEEKLFLAPISDSDEFYNTIQIKEYDEQPTY | |
| | SCQLLFKKDEESTDEIGLIGIHRFYESGIVFEEYKDYFCISKWYLKEVKRK | |
| | PYNLKLGCNWQFIPKDEGWTE | |
| ^{ci}BoNT/XC | MKLEINKFNYNDPIDGINVITMRPPRHSDKINKGKGPFKAFQVIKNIWIVP | 7 |
| | ERYNFTNNTNDLNIPSEPIMEADAIYNPNYLNTPSEKDEFLQGVIKVLERI | |
| | KSKPEGEKLLELISSSIPLPLVSNGALTLSDNETIAYQENNNIVSNLQANLV | |
| | IYGPGPDIANNATYGLYSTPISNGEGTLSEVSFSPFYLKPFDESYGNYRSL | |
| | VNIVNKFVKREFAPDPASTLMHQLVHVTHNLYGISNRNFYYNFDTGKIET | |
| | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICPRNGLLYNAIYRNSKNYLNNIDLEDKKTTSKTNVSYPCSL | |
| | LNGCIEVENKDLFLISNKDSLNDINLSEEKIKPETTVFFKDKLPPQDITLSN | |
| | YDFTEANSIPSISQQNILERNEELYEPIRNSLFEIKTIYVDKLTTFHFLEAQN | |
| | IDESIDSSKIRVELTDSVDEALSNPNKVYSPFKNMSNTINSIETGITSTYIFY | |
| | QWLRSIVKDFSDETGKIDVIDKSSDTLAIVPYIGPLLNIGNDIRHGDFVGAI | |
| | ELAGITALLEYVPEFTIPILVGLEVIGGELAREQVEAIVNNALDKRDQKW | |
| | AEVYNITKAQWWGTIHLQINTRLAHTYKALSRQANAIKMNMEFQLANY | |
| | KGNIDDKAKIKNAISETEILLNKSVEQAMKNTEKFMIKLSNSYLTKEMIPK | |
| | VQDNLKNFDLETKKTLDKFIKEKEDILGTNLSSSLRRKVSIRLNKNIAFDI | |
| | NDIPFSEFDDLINQYKNEIEDYEVLNLGAEDGKIKDLSGTTSDINIGSDIEIN | |
| | DSKILSLQNRKNTLVDTSGYNAEVSEEGDVQLNPIFPFDFKLGSSGEDRG | |
| | KVIVTQNENIVYNSMYESFSISFWIRINKWVSNLPGYTIIDSVKNNSGWSI | |
| | GIISNFLVFTLKQNEDSEQSINFSYDISNNAPGYNKWFFVTVTNNMMGNM | |
| | KIYINGKLIDTIKVKELTGINFSKTITFEINKIPDTGLITSDSDNINMWIRDFY | |
| | IFAKELDGKDINILFNSLQYTNVVKDYWGNDLRYNKEYYMVNIDYLNR | |
| | YMYANSRQIVFNTRRNNNDFNEGYKIIIKRIRGNTNDTRVRGGDILYFDM | |
| | TINNKAYNLFMKNETMYADNHSTEDIYAIGLREQTKDINDNIIFQIQPMN | |
| | NTYYYASQIFKSNFNGENISGICSIGTYRFRLGGDWYRHNYLVPTVKQGN | |
| | YASLLESTSTHWGFVPVSE | |
| ^{ci}BoNT/XD | MKLEINKFNYNDPIDGINVITMRPPRHSDKINKGKGPFKAFQVIKNIWIVP | 8 |
| | ERYNFTNNTNDLNIPSEPIMEADAIYNPNYLNTPSEKDEFLQGVIKVLERI | |
| | KSKPEGEKLLELISSSIPLPLVSNGALTLSDNETIAYQENNNIVSNLQANLV | |
| | IYGPGPDIANNATYGLYSTPISNGEGTLSEVSFSPFYLKPFDESYGNYRSL | |
| | VNIVNKFVKREFAPDPASTLMHQLVHVTHNLYGISNRNFYYNFDTGKIET | |
| | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICPRNGLLYNAIYRNSKNYLNNIDLEDKKTTSKTNVSYPCSL | |
| | LNGCIEVENKDLFLISNKDSLNDINLSEEKIKPETTVFFKDKLPPQDITLSN | |
| | YDFTEANSIPSISQQNILERNEELYEPIRNSLFEIKTIYVDKLTTFHFLEAQN | |
| | IDESIDSSKIRVELTDSVDEALSNPNKVYSPFKNMSNTINSIETGITSTYIFY | |
| | QWLRSIVKDFSDETGKIDVIDKSSDTLAIVPYIGPLLNIGNDIRHGDFVGAI | |
| | ELAGITALLEYVPEFTIPILVGLEVIGGELAREQVEAIVNNALDKRDQKW | |
| | AEVYNITKAQWWGTIHLQINTRLAHTYKALSRQANAIKMNMEFQLANY | |
| | KGNIDDKAKIKNAISETEILLNKSVEQAMKNTEKFMIKLSNSYLTKEMIPK | |
| | VQDNLKNFDLETKKTLDKFIKEKEDILGTNLSSSLRRKVSIRLNKNIAFDI | |
| | NDIPFSEFDDLINQYKNEIEDYEVLNLGAEDGKIKDLSGTTSDINIGSDIEIN | |
| | DSKILSLQNKKNALVDTSGYNAEVRVGDNVQLNTIYTNDFKLSSSGDKII | |
| | VNLNNNILYSAIYENSSVSFWIKISKDLTNSHNEYTIINSIEQNSGWKLCIR | |
| | NGNIEWILQDVNRKYKSLIFDYSESLSHTGYTNKWFFVTITNNIMGYMKL | |
| | YINGELKQSQKIEDLDEVKLDKTIVFGIDENIDENQMLWIRDFNIFSKELS | |
| | NEDINIVYEGQILRNVIKDYWGNPLKFDTEYYIINDNYIDRYIAPESNVLV | |
| | LVQYPDRSKLYTGNPITIKSVSDKNPYSRILNGDNIILHMLYNSRKYMIIR | |
| | DTDTIYATQGGECSQNCVYALKLQSNLGNYGIGIFSIKNIVSKNKYCSQIF | |
| | SSFRENTMLLADIYKPWRFSFKNAYTPVAVTNYETKLLSTSSFWKFISRD | |
| | PGWVE | |
| ^{ci}BoNT/XE | MKLEINKFNYNDPIDGINVITMRPPRHSDKINKGKGPFKAFQVIKNIWIVP | 9 |
| | ERYNFTNNTNDLNIPSEPIMEADAIYNPNYLNTPSEKDEFLQGVIKVLERI | |
| | KSKPEGEKLLELISSSIPLPLVSNGALTLSDNETIAYQENNNIVSNLQANLV | |
| | IYGPGPDIANNATYGLYSTPISNGEGTLSEVSFSPFYLKPFDESYGNYRSL | |
| | VNIVNKFVKREFAPDPASTLMHQLVHVTHNLYGISNRNFYYNFDTGKIET | |
| | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICPRNGLLYNAIYRNSKNYLNNIDLEDKKTTSKTNVSYPCSL | |
| | LNGCIEVENKDLFLISNKDSLNDINLSEEKIKPETTVFFKDKLPPQDITLSN | |
| | YDFTEANSIPSISQQNILERNEELYEPIRNSLFEIKTIYVDKLTTFHFLEAQN | |
| | IDESIDSSKIRVELTDSVDEALSNPNKVYSPFKNMSNTINSIETGITSTYIFY | |
| | QWLRSIVKDFSDETGKIDVIDKSSDTLAIVPYIGPLLNIGNDIRHGDFVGAI | |
| | ELAGITALLEYVPEFTIPILVGLEVIGGELAREQVEAIVNNALDKRDQKW | |
| | AEVYNITKAQWWGTIHLQINTRLAHTYKALSRQANAIKMNMEFQLANY | |
| | KGNIDDKAKIKNAISETEILLNKSVEQAMKNTEKFMIKLSNSYLTKEMIPK | |
| | VQDNLKNFDLETKKTLDKFIKEKEDILGTNLSSSLRRKVSIRLNKNIAFDI | |
| | NDIPFSEFDDLINQYKNEIEDYEVLNLGAEDGKIKDLSGTTSDINIGSDIEIK | |
| | SSSVLNMRYKNDKYVDTSGYDSNININGDVYKYPTNKNQFGIYNDKLSE | |
| | VNISQNDYIIYDNKYKNFSISFWVRIPNYDNKIVNVNNEYTIINCMRDNNS | |
| | GWKVSLNHNEIIWTLQDNAGINQKLAFNYGNANGISDYINKWIFVTITND | |
| | RLGDSKLYINGNLIDQKSILNLGNIHVSDNILFKIVNCSYTRYIGIRYFNIFD | |
| | KELDETEIQTLYSNEPNTNILKDFWGNYLLYDKEYYLLNVLKPNNFIDRR | |
| | KDSTLSINNIRSTILLANRLYSGIKVKIQRVNNSSTNDNLVRKNDQVYINF | |
| | VASKTHLFPLYADTATTNKEKTIKISSSGNRFNQVVVMNSVGNNCTMNF | |
| | KNNNGNNIGLLGFKADTVVASTWYYTHMRDHTNSNGCFWNFISEEHG | |
| | WQEK | |
| ^{ci}BoNT/XF | MKLEINKFNYNDPIDGINVITMRPPRHSDKINKGKGPFKAFQVIKNIWIVP | 10 |
| | ERYNFTNNTNDLNIPSEPIMEADAIYNPNYLNTPSEKDEFLQGVIKVLERI | |
| | KSKPEGEKLLELISSSIPLPLVSNGALTLSDNETIAYQENNNIVSNLQANLV | |
| | IYGPGPDIANNATYGLYSTPISNGEGTLSEVSFSPFYLKPFDESYGNYRSL | |
| | VNIVNKFVKREFAPDPASTLMHQLVHVTHNLYGISNRNFYYNFDTGKIET | |
| | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICPRNGLLYNAIYRNSKNYLNNIDLEDKKTTSKTNVSYPCSL | |
| | LNGCIEVENKDLFLISNKDSLNDINLSEEKIKPETTVFFKDKLPPQDITLSN | |
| | YDFTEANSIPSISQQNILERNEELYEPIRNSLFEIKTIYVDKLTTFHFLEAQN | |
| | IDESIDSSKIRVELTDSVDEALSNPNKVYSPFKNMSNTINSIETGITSTYIFY | |
| | QWLRSIVKDFSDETGKIDVIDKSSDTLAIVPYIGPLLNIGNDIRHGDFVGAI | |
| | ELAGITALLEYVPEFTIPILVGLEVIGGELAREQVEAIVNNALDKRDQKW | |
| | AEVYNITKAQWWGTIHLQINTRLAHTYKALSRQANAIKMNMEFQLANY | |
| | KGNIDDKAKIKNAISETEILLNKSVEQAMKNTEKFMIKLSNSYLTKEMIPK | |
| | VQDNLKNFDLETKKTLDKFIKEKEDILGTNLSSSLRRKVSIRLNKNIAFDI | |
| | NDIPFSEFDDLINQYKNEIEDYEVLNLGAEDGKIKDLSGTTSDINIGSDIEIK | |
| | DNSILDMRYENNKFIDISGYGSNISINGDVYIYSTNRNQFGIYSSKPSEVNI | |
| | AQNNDIIYNGRYQNFSISFWVRIPKYFNKVNLNNEYTIIDCIRNNNSGWKI | |
| | SLNYNKIIWTLQDTAGNNQKLVFNYTQMISISDYINKWIFVTITNNRLGNS | |
| | RIYINGNLIDEKSISNLGDIHVSDNILFKIVGCNDTRYVGIRYFKVFDTELG | |
| | KTEIETLYSDEPDPSILKDFWGNYLLYNKRYYLLNLLRTDKSITQNSNFLN | |
| | INQQRGVYQKPNIFSNTRLYTGVEVIIRKNGSTDISNTDNFVRKNDLAYIN | |
| | VVDRDVEYRLYADISIAKPEKIIKLIRTSNSNNSLGQIIVMDSIGNNCTMNF | |
| | QNNNGGNIGLLGFHSNNLVASSWYYNNIRKNTSSNGCFWSFISKEHGWQ | |
| | EN | |
| ^{ci}BoNT/XG | MKLEINKFNYNDPIDGINVITMRPPRHSDKINKGKGPFKAFQVIKNIWIVP | 11 |
| | ERYNFTNNTNDLNIPSEPIMEADAIYNPNYLNTPSEKDEFLQGVIKVLERI | |
| | KSKPEGEKLLELISSSIPLPLVSNGALTLSDNETIAYQENNNIVSNLQANLV | |
| | IYGPGPDIANNATYGLYSTPISNGEGTLSEVSFSPFYLKPFDESYGNYRSL | |
| | VNIVNKFVKREFAPDPASTLMHQLVHVTHNLYGISNRNFYYNFDTGKIET | |
| | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICPRNGLLYNAIYRNSKNYLNNIDLEDKKTTSKTNVSYPCSL | |
| | LNGCIEVENKDLFLISNKDSLNDINLSEEKIKPETTVFFKDKLPPQDITLSN | |
| | YDFTEANSIPSISQQNILERNEELYEPIRNSLFEIKTIYVDKLTTFHFLEAQN | |
| | IDESIDSSKIRVELTDSVDEALSNPNKVYSPFKNMSNTINSIETGITSTYIFY | |
| | QWLRSIVKDFSDETGKIDVIDKSSDTLAIVPYIGPLLNIGNDIRHGDFVGAI | |
| | ELAGITALLEYVPEFTIPILVGLEVIGGELAREQVEAIVNNALDKRDQKW | |
| | AEVYNITKAQWWGTIHLQINTRLAHTYKALSRQANAIKMNMEFQLANY | |
| | KGNIDDKAKIKNAISETEILLNKSVEQAMKNTEKFMIKLSNSYLTKEMIPK | |
| | VQDNLKNFDLETKKTLDKFIKEKEDILGTNLSSSLRRKVSIRLNKNIAFDI | |
| | NDIPFSEFDDLINQYKNEIEDYEVLNLGAEDGKIKDLSGTTSDINIGSDIEIS | |
| | SNAILSLSYRGGRLIDSSGYGATMNVGSDVIFNDIGNGQFKLNNSENSNIT | |
| | AHQSKFVVYDSMFDNFSINFWVRTPKYNNNDIQTYLQNEYTIISCIKNDS | |
| | GWKVSIKGNRIIWTLIDVNAKSKSIFFEYSIKDNISDYINKWFSITITNDRL | |
| | GNANIYINGSLKKSEKILNLDRINSSNDIDFKLINCTDTTKFVWIKDFNIFG | |
| | RELNATEVSSLYWIQSSTNTLKDFWGNPLRYDTQYYLFNQGMQNIYIKY | |
| | FSKASMGETAPRTNFNNAAINYQNLYLGLRFIIKKASNSRNINNDNIVRE | |
| | GDYIYLNIDNISDESYRVYVLVNSKEIQTQLFLAPINDDPTFYDVLQIKKY | |
| | YEKTTYNCQILCEKDTKTFGLFGIGKFVKDYGYVWDTYDNYFCISQWYL | |
| | RRISENINKLRLGCNWQFIPVDEGWTE | |
| ^{ci}BoNT/XH | MKLEINKFNYNDPIDGINVITMRPPRHSDKINKGKGPFKAFQVIKNIWIVP | 12 |
| | ERYNFTNNTNDLNIPSEPIMEADAIYNPNYLNTPSEKDEFLQGVIKVLERI | |
| | KSKPEGEKLLELISSSIPLPLVSNGALTLSDNETIAYQENNNIVSNLQANLV | |
| | IYGPGPDIANNATYGLYSTPISNGEGTLSEVSFSPFYLKPFDESYGNYRSL | |
| | VNIVNKFVKREFAPDPASTLMHQLVHVTHNLYGISNRNFYYNFDTGKIET | |
| | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICPRNGLLYNAIYRNSKNYLNNIDLEDKKTTSKTNVSYPCSL | |
| | LNGCIEVENKDLFLISNKDSLNDINLSEEKIKPETTVFFKDKLPPQDITLSN | |
| | YDFTEANSIPSISQQNILERNEELYEPIRNSLFEIKTIYVDKLTTFHFLEAQN | |
| | IDESIDSSKIRVELTDSVDEALSNPNKVYSPFKNMSNTINSIETGITSTYIFY | |
| | QWLRSIVKDFSDETGKIDVIDKSSDTLAIVPYIGPLLNIGNDIRHGDFVGAI | |
| | ELAGITALLEYVPEFTIPILVGLEVIGGELAREQVEAIVNNALDKRDQKW | |
| | AEVYNITKAQWWGTIHLQINTRLAHTYKALSRQANAIKMNMEFQLANY | |
| | KGNIDDKAKIKNAISETEILLNKSVEQAMKNTEKFMIKLSNSYLTKEMIPK | |
| | VQDNLKNFDLETKKTLDKFIKEKEDILGTNLSSSLRRKVSIRLNKNIAFDI | |
| | NDIPFSEFDDLINQYKNEIEDYEVLNLGAEDGKIKDLSGTTSDINIGSDIEL | |
| | KYNCILNIKYEMDRDKLVDSSGYRSRINIGTGVKFSEIDKNQVQLSNLESS | |
| | KIEVILNNGVIYNSMYENFSTSFWIRIPKYFRNINNEYKIISCMQNNSGWE | |
| | VSLNFSNMNSKIIWTLQDTEGIKKTVVFQYTQNINISDYINRWIFVTITNN | |
| | RLSNSKIYINGRLINEESISDLGNIHASNNIMFKLDGCRDPHRYIWIKYFNL | |
| | FDKELNKKEIKDLYDNQSNSGILKDFWGDYLQYDKPYYMLNLYDPNKY | |
| | LDVNNVGIRGYMYLKGPRGRIVTTNIYLNSTLYMGTKFIIKKYASGNKD | |
| | NIVRNNDRVYINVVVKNKEYRLATNASQAGVEKILSAVEIPDVGNLSQV | |
| | VVMKSENDQGIRNKCKMNLQDNNGNDIGFIGFHQFNNIAKLVASNWYN | |
| | RQIGKASRTFGCSWEFIPVDDGWGESSL | |
| ^{ci}BoNT/EnA | MVTINDLHYSDPIDEDNIINMRIPLYDLEVDDQFINHNVPDLKAFQVFPNV | 13 |
| | WVVPERYTFYSTMKNLDAPANPSRSSYYDPTYLQSDAEKEVFLQQMILL | |
| | FKRINSTQEGQQFLNLLSRSIPVPYESNGDVAMGTTQVIKQMDDKGNVL | |
| | KHRRAHIIIYGPGPDLMAKGSKALTKSRETGRGCMAEIYFSPMYHKTYST | |
| | KLTNKNSLVDKSVQEFVPDPAVTLIHQLCHGLHALYGIDLGNVGSWEFN | |
| | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACPNPHFSSQRGLSSCIEILEDDLFIMSS | |
| | KDSFTDTDFSEPSVGPVSYKAKKGADTILDSTLSNYDFSKEINFTSTVPIIT | |
| | VEDPLETDEDVPVISEDRTVYVDDYTTFHFLEAQKIGKEVVPTQTKVVFT | |
| | TNMEEALFDSKKVYTVFENTASRINEAGTGIANGMMFYQWLKGIVQDFT | |
| | EEATQKDTFDKISDVTMIVPYLGNILNIGNDIRKGDFMGAVELGGVTILLE | |
| | AIPELTLPVLIGLTIIEDELEKEQVSQTVYNVLDKRDEKWEEVYGFVKQQ | |
| | WWWMVHTQFETRILHAYQALNHQVEAIKANMTYQLANYRGNQEDKEL | |
| | LEKAIDDTLQSLYYAVDQAMHNIKRFLIQSSKSYLLNQMLPKTKEQLLAF | |
| | DQQTLRNVNDFINKNQGVLGESLAKDLKKKVEKRLTSLPVFNLEDLPISE | |
| | FEDLIHSHEIDIQDSEVLNIGVNNGKIQDLSGENTPLTLGENLHIINTSILNL | |
| | RYESNHLIDLSRYASKINIGSKVNFDPIDKNQIQLFNLESSKIEVILKNAIVY | |
| | NSMYENFSTSFWIRIPKYFNSISLNNEYTIINCMENNSGWKVSLNYGEIIW | |
| | TLQDTQEIKQRVVFKYSQMINISDYINRWIFVTITNNRLNNSKIYINGRLID | |
| | QKPISNLGNIHASNNIMFKLDGCRDTHRYIWIKYFNLFDKELNEKEIKDL | |
| | YDNQSNSGILKDFWGDYLQYDKPYYMLNLYDPNKYVDVNNVGIRGYM | |
| | YLKGPRGSVMTTNIYLNSSLYRGTKFIIKKYASGNKDNIVRNNDRVYINV | |
| | VVKNKEYRLATNASQAGVEKILSALEIPDVGNLSQVVVMKSKNDQGITN | |
| | KCKMNLQDNNGNDIGFIGFHQFNNIAKLVASNWYNRQIERSSRTLGCSW | |
| | EFIPVDDGWGERPLQ | |
| ^{ci}BoNT/EnB | MVTINDLHYSDPIDEDNIINMRIPLYDLEVDDQFINHNVPDLKAFQVFPNV | 14 |
| | WVVPERYTFYSTMKNLDAPANPSRSSYYDPTYLQSDAEKEVFLQQMILL | |
| | FKRINSTQEGQQFLNLLSRSIPVPYESNGDVAMGTTQVIKQMDDKGNVL | |
| | KHRRAHIIIYGPGPDLMAKGSKALTKSRETGRGCMAEIYFSPMYHKTYST | |
| | KLTNKNSLVDKSVQEFVPDPAVTLIHQLCHGLHALYGIDLGNVGSWEFN | |
| | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACPNPHFSSQRGLSSCIEILEDDLFIMSS | |
| | KDSFTDTDFSEPSVGPVSYKAKKGADTILDSTLSNYDFSKEINFTSTVPIIT | |
| | VEDPLETDEDVPVISEDRTVYVDDYTTFHFLEAQKIGKEVVPTQTKVVFT | |
| | TNMEEALFDSKKVYTVFENTASRINEAGTGIANGMMFYQWLKGIVQDFT | |
| | EEATQKDTFDKISDVTMIVPYLGNILNIGNDIRKGDFMGAVELGGVTILLE | |
| | AIPELTLPVLIGLTIIEDELEKEQVSQTVYNVLDKRDEKWEEVYGFVKQQ | |
| | WWWMVHTQFETRILHAYQALNHQVEAIKANMTYQLANYRGNQEDKEL | |
| | LEKAIDDTLQSLYYAVDQAMHNIKRFLIQSSKSYLLNQMLPKTKEQLLAF | |
| | DQQTLRNVNDFINKNQGVLGESLAKDLKKKVEKRLTSLPVFNLEDLPISE | |
| | FEDLIHSHEIDIQDSEVLNIGVNNGKIQDLSGENTPLTLGENLHILNNIILNL | |
| | RYKDNNLIDLSGYGAKVEVYDGVELNDKNQFKLTSSANSKIRVTQNQNI | |
| | IFNSVFLDFSVSFWIRIPKYKNDGIQNYIHNEYTIINCMKNNSGWKISIRGN | |
| | RIIWTLIDINGKTKSVFFEYNIREDISEYINRWFFVTITNNLNNAKIYINGKL | |
| | ESNTDIKDIREVIANGEIIFKLDGDIDRTQFIWMKYFSIFNTELSQSNIEERY | |
| | KIQSYSEYLKDFWGNPLMYNKEYYMFNAGNKNSYIKLKKDSPVGEILTR | |
| | SKYNQNSKYINYRDLYIGEKFIIRRKSNSQSINDDIVRKEDYIYLDFFNLN | |
| | QEWRVYTYKYFKKEEEKLFLAPISDSDEFYNTIQIKEYDEQPTYSCQLLF | |
| | KKDEESTDEIGLIGIHRFYESGIVFEEYKDYFCISKWYLKEVKRKPYNLKL | |
| | GCNWQFIPKDEGWTE | |
| ^{ci}BoNT/EnC | MVTINDLHYSDPIDEDNIINMRIPLYDLEVDDQFINHNVPDLKAFQVFPNV | 15 |
| | WVVPERYTFYSTMKNLDAPANPSRSSYYDPTYLQSDAEKEVFLQQMILL | |
| | FKRINSTQEGQQFLNLLSRSIPVPYESNGDVAMGTTQVIKQMDDKGNVL | |
| | KHRRAHIIIYGPGPDLMAKGSKALTKSRETGRGCMAEIYFSPMYHKTYST | |
| | KLTNKNSLVDKSVQEFVPDPAVTLIHQLCHGLHALYGIDLGNVGSWEFN | |
| | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACPNPHFSSQRGLSSCIEILEDDLFIMSS | |
| | KDSFTDTDFSEPSVGPVSYKAKKGADTILDSTLSNYDFSKEINFTSTVPIIT | |
| | VEDPLETDEDVPVISEDRTVYVDDYTTFHFLEAQKIGKEVVPTQTKVVFT | |
| | TNMEEALFDSKKVYTVFENTASRINEAGTGIANGMMFYQWLKGIVQDFT | |
| | EEATQKDTFDKISDVTMIVPYLGNILNIGNDIRKGDFMGAVELGGVTILLE | |
| | AIPELTLPVLIGLTIIEDELEKEQVSQTVYNVLDKRDEKWEEVYGFVKQQ | |
| | WWWMVHTQFETRILHAYQALNHQVEAIKANMTYQLANYRGNQEDKEL | |
| | LEKAIDDTLQSLYYAVDQAMHNIKRFLIQSSKSYLLNQMLPKTKEQLLAF | |
| | DQQTLRNVNDFINKNQGVLGESLAKDLKKKVEKRLTSLPVFNLEDLPISE | |
| | FEDLIHSHEIDIQDSEVLNIGVNNGKIQDLSGENTPLTLGENLHINDSKILS | |
| | LQNRKNTLVDTSGYNAEVSEEGDVQLNPIFPFDFKLGSSGEDRGKVIVTQ | |
| | NENIVYNSMYESFSISFWIRINKWVSNLPGYTIIDSVKNNSGWSIGIISNFL | |
| | VFTLKQNEDSEQSINFSYDISNNAPGYNKWFFVTVTNNMMGNMKIYING | |
| | KLIDTIKVKELTGINFSKTITFEINKIPDTGLITSDSDNINMWIRDFYIFAKEL | |
| | DGKDINILFNSLQYTNVVKDYWGNDLRYNKEYYMVNIDYLNRYMYAN | |
| | SRQIVFNTRRNNNDFNEGYKIIIKRIRGNTNDTRVRGGDILYFDMTINNKA | |
| | YNLFMKNETMYADNHSTEDIYAIGLREQTKDINDNIIFQIQPMNNTYYYA | |
| | SQIFKSNFNGENISGICSIGTYRFRLGGDWYRHNYLVPTVKQGNYASLLE | |
| | STSTHWGFVPVSE | |
| ^{ci}BoNT/EnD | MVTINDLHYSDPIDEDNIINMRIPLYDLEVDDQFINHNVPDLKAFQVFPNV | 16 |
| | WVVPERYTFYSTMKNLDAPANPSRSSYYDPTYLQSDAEKEVFLQQMILL | |
| | FKRINSTQEGQQFLNLLSRSIPVPYESNGDVAMGTTQVIKQMDDKGNVL | |
| | KHRRAHIIIYGPGPDLMAKGSKALTKSRETGRGCMAEIYFSPMYHKTYST | |
| | KLTNKNSLVDKSVQEFVPDPAVTLIHQLCHGLHALYGIDLGNVGSWEFN | |
| | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACPNPHFSSQRGLSSCIEILEDDLFIMSS | |
| | KDSFTDTDFSEPSVGPVSYKAKKGADTILDSTLSNYDFSKEINFTSTVPIIT | |
| | VEDPLETDEDVPVISEDRTVYVDDYTTFHFLEAQKIGKEVVPTQTKVVFT | |
| | TNMEEALFDSKKVYTVFENTASRINEAGTGIANGMMFYQWLKGIVQDFT | |
| | EEATQKDTFDKISDVTMIVPYLGNILNIGNDIRKGDFMGAVELGGVTILLE | |
| | AIPELTLPVLIGLTIIEDELEKEQVSQTVYNVLDKRDEKWEEVYGFVKQQ | |
| | WWWMVHTQFETRILHAYQALNHQVEAIKANMTYQLANYRGNQEDKEL | |
| | LEKAIDDTLQSLYYAVDQAMHNIKRFLIQSSKSYLLNQMLPKTKEQLLAF | |
| | DQQTLRNVNDFINKNQGVLGESLAKDLKKKVEKRLTSLPVFNLEDLPISE | |
| | FEDLIHSHEIDIQDSEVLNIGVNNGKIQDLSGENTPLTLGENLHINDSKILS | |
| | LQNKKNALVDTSGYNAEVRVGDNVQLNTIYTNDFKLSSSGDKIIVNLNN | |
| | NILYSAIYENSSVSFWIKISKDLTNSHNEYTIINSIEQNSGWKLCIRNGNIE | |
| | WILQDVNRKYKSLIFDYSESLSHTGYTNKWFFVTITNNIMGYMKLYINGE | |
| | LKQSQKIEDLDEVKLDKTIVFGIDENIDENQMLWIRDFNIFSKELSNEDINI | |
| | VYEGQILRNVIKDYWGNPLKFDTEYYIINDNYIDRYIAPESNVLVLVQYP | |
| | DRSKLYTGNPITIKSVSDKNPYSRILNGDNIILHMLYNSRKYMIIRDTDTIY | |
| | ATQGGECSQNCVYALKLQSNLGNYGIGIFSIKNIVSKNKYCSQIFSSFREN | |
| | TMLLADIYKPWRFSFKNAYTPVAVTNYETKLLSTSSFWKFISRDPGWVE | |
| ^{ci}BoNT/EnE | MVTINDLHYSDPIDEDNIINMRIPLYDLEVDDQFINHNVPDLKAFQVFPNV | 17 |
| | WVVPERYTFYSTMKNLDAPANPSRSSYYDPTYLQSDAEKEVFLQQMILL | |
| | FKRINSTQEGQQFLNLLSRSIPVPYESNGDVAMGTTQVIKQMDDKGNVL | |
| | KHRRAHIIIYGPGPDLMAKGSKALTKSRETGRGCMAEIYFSPMYHKTYST | |
| | KLTNKNSLVDKSVQEFVPDPAVTLIHQLCHGLHALYGIDLGNVGSWEFN | |
| | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACPNPHFSSQRGLSSCIEILEDDLFIMSS | |
| | KDSFTDTDFSEPSVGPVSYKAKKGADTILDSTLSNYDFSKEINFTSTVPIIT | |
| | VEDPLETDEDVPVISEDRTVYVDDYTTFHFLEAQKIGKEVVPTQTKVVFT | |
| | TNMEEALFDSKKVYTVFENTASRINEAGTGIANGMMFYQWLKGIVQDFT | |
| | EEATQKDTFDKISDVTMIVPYLGNILNIGNDIRKGDFMGAVELGGVTILLE | |
| | AIPELTLPVLIGLTIIEDELEKEQVSQTVYNVLDKRDEKWEEVYGFVKQQ | |
| | WWWMVHTQFETRILHAYQALNHQVEAIKANMTYQLANYRGNQEDKEL | |
| | LEKAIDDTLQSLYYAVDQAMHNIKRFLIQSSKSYLLNQMLPKTKEQLLAF | |
| | DQQTLRNVNDFINKNQGVLGESLAKDLKKKVEKRLTSLPVFNLEDLPISE | |
| | FEDLIHSHEIDIQDSEVLNIGVNNGKIQDLSGENTPLTLGENLHIKSSSVLN | |
| | MRYKNDKYVDTSGYDSNININGDVYKYPTNKNQFGIYNDKLSEVNISQN | |
| | DYIIYDNKYKNFSISFWVRIPNYDNKIVNVNNEYTIINCMRDNNSGWKVS | |
| | LNHNEIIWTLQDNAGINQKLAFNYGNANGISDYINKWIFVTITNDRLGDS | |
| | KLYINGNLIDQKSILNLGNIHVSDNILFKIVNCSYTRYIGIRYFNIFDKELDE | |
| | TEIQTLYSNEPNTNILKDFWGNYLLYDKEYYLLNVLKPNNFIDRRKDSTL | |
| | SINNIRSTILLANRLYSGIKVKIQRVNNSSTNDNLVRKNDQVYINFVASKT | |
| | HLFPLYADTATTNKEKTIKISSSGNRFNQVVVMNSVGNNCTMNFKNNNG | |
| | NNIGLLGFKADTVVASTWYYTHMRDHTNSNGCFWNFISEEHGWQEK | |
| ^{ci}BoNT/EnF | MVTINDLHYSDPIDEDNIINMRIPLYDLEVDDQFINHNVPDLKAFQVFPNV | 18 |
| | WVVPERYTFYSTMKNLDAPANPSRSSYYDPTYLQSDAEKEVFLQQMILL | |
| | FKRINSTQEGQQFLNLLSRSIPVPYESNGDVAMGTTQVIKQMDDKGNVL | |
| | KHRRAHIIIYGPGPDLMAKGSKALTKSRETGRGCMAEIYFSPMYHKTYST | |
| | KLTNKNSLVDKSVQEFVPDPAVTLIHQLCHGLHALYGIDLGNVGSWEFN | |
| | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACPNPHFSSQRGLSSCIEILEDDLFIMSS | |
| | KDSFTDTDFSEPSVGPVSYKAKKGADTILDSTLSNYDFSKEINFTSTVPIIT | |
| | VEDPLETDEDVPVISEDRTVYVDDYTTFHFLEAQKIGKEVVPTQTKVVFT | |
| | TNMEEALFDSKKVYTVFENTASRINEAGTGIANGMMFYQWLKGIVQDFT | |
| | EEATQKDTFDKISDVTMIVPYLGNILNIGNDIRKGDFMGAVELGGVTILLE | |
| | AIPELTLPVLIGLTIIEDELEKEQVSQTVYNVLDKRDEKWEEVYGFVKQQ | |
| | WWWMVHTQFETRILHAYQALNHQVEAIKANMTYQLANYRGNQEDKEL | |
| | LEKAIDDTLQSLYYAVDQAMHNIKRFLIQSSKSYLLNQMLPKTKEQLLAF | |
| | DQQTLRNVNDFINKNQGVLGESLAKDLKKKVEKRLTSLPVFNLEDLPISE | |
| | FEDLIHSHEIDIQDSEVLNIGVNNGKIQDLSGENTPLTLGENLHIKDNSILD | |
| | MRYENNKFIDISGYGSNISINGDVYIYSTNRNQFGIYSSKPSEVNIAQNNDI | |
| | IYNGRYQNFSISFWVRIPKYFNKVNLNNEYTIIDCIRNNNSGWKISLNYNK | |
| | IIWTLQDTAGNNQKLVFNYTQMISISDYINKWIFVTITNNRLGNSRIYING | |
| | NLIDEKSISNLGDIHVSDNILFKIVGCNDTRYVGIRYFKVFDTELGKTEIET | |
| | LYSDEPDPSILKDFWGNYLLYNKRYYLLNLLRTDKSITQNSNFLNINQQR | |
| | GVYQKPNIFSNTRLYTGVEVIIRKNGSTDISNTDNFVRKNDLAYINVVDR | |
| | DVEYRLYADISIAKPEKIIKLIRTSNSNNSLGQIIVMDSIGNNCTMNFQNNN | |
| | GGNIGLLGFHSNNLVASSWYYNNIRKNTSSNGCFWSFISKEHGWQEN | |
| ^{ci}BoNT/EnG | MVTINDLHYSDPIDEDNIINMRIPLYDLEVDDQFINHNVPDLKAFQVFPNV | 19 |
| | WVVPERYTFYSTMKNLDAPANPSRSSYYDPTYLQSDAEKEVFLQQMILL | |
| | FKRINSTQEGQQFLNLLSRSIPVPYESNGDVAMGTTQVIKQMDDKGNVL | |
| | KHRRAHIIIYGPGPDLMAKGSKALTKSRETGRGCMAEIYFSPMYHKTYST | |
| | KLTNKNSLVDKSVQEFVPDPAVTLIHQLCHGLHALYGIDLGNVGSWEFN | |
| | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACPNPHFSSQRGLSSCIEILEDDLFIMSS | |
| | KDSFTDTDFSEPSVGPVSYKAKKGADTILDSTLSNYDFSKEINFTSTVPIIT | |
| | VEDPLETDEDVPVISEDRTVYVDDYTTFHFLEAQKIGKEVVPTQTKVVFT | |
| | TNMEEALFDSKKVYTVFENTASRINEAGTGIANGMMFYQWLKGIVQDFT | |
| | EEATQKDTFDKISDVTMIVPYLGNILNIGNDIRKGDFMGAVELGGVTILLE | |
| | AIPELTLPVLIGLTIIEDELEKEQVSQTVYNVLDKRDEKWEEVYGFVKQQ | |
| | WWWMVHTQFETRILHAYQALNHQVEAIKANMTYQLANYRGNQEDKEL | |
| | LEKAIDDTLQSLYYAVDQAMHNIKRFLIQSSKSYLLNQMLPKTKEQLLAF | |
| | DQQTLRNVNDFINKNQGVLGESLAKDLKKKVEKRLTSLPVFNLEDLPISE | |
| | FEDLIHSHEIDIQDSEVLNIGVNNGKIQDLSGENTPLTLGENLHISSNAILSL | |
| | SYRGGRLIDSSGYGATMNVGSDVIFNDIGNGQFKLNNSENSNITAHQSKF | |
| | VVYDSMFDNFSINFWVRTPKYNNNDIQTYLQNEYTIISCIKNDSGWKVSI | |
| | KGNRIIWTLIDVNAKSKSIFFEYSIKDNISDYINKWFSITITNDRLGNANIYI | |
| | NGSLKKSEKILNLDRINSSNDIDFKLINCTDTTKFVWIKDFNIFGRELNATE | |
| | VSSLYWIQSSTNTLKDFWGNPLRYDTQYYLFNQGMQNIYIKYFSKASMG | |
| | ETAPRTNFNNAAINYQNLYLGLRFIIKKASNSRNINNDNIVREGDYIYLNI | |
| | DNISDESYRVYVLVNSKEIQTQLFLAPINDDPTFYDVLQIKKYYEKTTYN | |
| | CQILCEKDTKTFGLFGIGKFVKDYGYVWDTYDNYFCISQWYLRRISENIN | |
| | KLRLGCNWQFIPVDEGWTE | |
| ^{ci}BoNT/EnH | MVTINDLHYSDPIDEDNIINMRIPLYDLEVDDQFINHNVPDLKAFQVFPNV | 20 |
| | WVVPERYTFYSTMKNLDAPANPSRSSYYDPTYLQSDAEKEVFLQQMILL | |
| | FKRINSTQEGQQFLNLLSRSIPVPYESNGDVAMGTTQVIKQMDDKGNVL | |
| | KHRRAHIIIYGPGPDLMAKGSKALTKSRETGRGCMAEIYFSPMYHKTYST | |
| | KLTNKNSLVDKSVQEFVPDPAVTLIHQLCHGLHALYGIDLGNVGSWEFN | |
| | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACPNPHFSSQRGLSSCIEILEDDLFIMSS | |
| | KDSFTDTDFSEPSVGPVSYKAKKGADTILDSTLSNYDFSKEINFTSTVPIIT | |
| | VEDPLETDEDVPVISEDRTVYVDDYTTFHFLEAQKIGKEVVPTQTKVVFT | |
| | TNMEEALFDSKKVYTVFENTASRINEAGTGIANGMMFYQWLKGIVQDFT | |
| | EEATQKDTFDKISDVTMIVPYLGNILNIGNDIRKGDFMGAVELGGVTILLE | |
| | AIPELTLPVLIGLTIIEDELEKEQVSQTVYNVLDKRDEKWEEVYGFVKQQ | |
| | WWWMVHTQFETRILHAYQALNHQVEAIKANMTYQLANYRGNQEDKEL | |
| | LEKAIDDTLQSLYYAVDQAMHNIKRFLIQSSKSYLLNQMLPKTKEQLLAF | |
| | DQQTLRNVNDFINKNQGVLGESLAKDLKKKVEKRLTSLPVFNLEDLPISE | |
| | FEDLIHSHEIDIQDSEVLNIGVNNGKIQDLSGENTPLTLGENLHLKYNCILN | |
| | IKYEMDRDKLVDSSGYRSRINIGTGVKFSEIDKNQVQLSNLESSKIEVILN | |
| | NGVIYNSMYENFSTSFWIRIPKYFRNINNEYKIISCMQNNSGWEVSLNFSN | |
| | MNSKIIWTLQDTEGIKKTVVFQYTQNINISDYINRWIFVTITNNRLSNSKIY | |
| | INGRLINEESISDLGNIHASNNIMFKLDGCRDPHRYIWIKYFNLFDKELNK | |
| | KEIKDLYDNQSNSGILKDFWGDYLQYDKPYYMLNLYDPNKYLDVNNVG | |
| | IRGYMYLKGPRGRIVTTNIYLNSTLYMGTKFIIKKYASGNKDNIVRNNDR | |
| | VYINVVVKNKEYRLATNASQAGVEKILSAVEIPDVGNLSQVVVMKSEND | |
| | QGIRNKCKMNLQDNNGNDIGFIGFHQFNNIAKLVASNWYNRQIGKASRT | |
| | FGCSWEFIPVDDGWGESSL | |
| ^{ci}BoNT/PMP1A | MLQIRVFNYNDPIDGENIVELRYHNRSPVKAFQIVDGIWIIPERYNFTNDT | 87 |
| | KKVPDDRALTILEDEVFAVRENDYLTTDVNEKNSFLNNITKLFKRINSSNI | |
| | GNQLLNYISTSVPYPVVSTNSIKARDYNTIKFDSIDGRRITKSANVLIYGPS | |
| | MKNLLDKQTRAINGEEAKNGIGCLSDIIFSPNYLSVQTVSSSRFVEDPASS | |
| | LTHQLIHALHNLYGIQYPGEEKFKFGGFIDKLLGTRECIDYEEVLTYGGK | |
| | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCSITSKNRVNICI | |
| | DVNKEDLYFISDKEGFENIDFSEPEIRYDSNVTTATTSSFTDHFLVNRTFN | |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQIINTSILNLRYESNHLIDLSRYASKINIGSKV | |
| | NFDPIDKNQIQLFNLESSKIEVILKNAIVYNSMYENFSTSFWIRIPKYFNSIS | |
| | LNNEYTIINCMENNSGWKVSLNYGEIIWTLQDTQEIKQRVVFKYSQMINI | |
| | SDYINRWIFVTITNNRLNNSKIYINGRLIDQKPISNLGNIHASNNIMFKLDG | |
| | CRDTHRYIWIKYFNLFDKELNEKEIKDLYDNQSNSGILKDFWGDYLQYD | |
| | KPYYMLNLYDPNKYVDVNNVGIRGYMYLKGPRGSVMTTNIYLNSSLYR | |
| | GTKFIIKKYASGNKDNIVRNNDRVYINVVVKNKEYRLATNASQAGVEKI | |
| | LSALEIPDVGNLSQVVVMKSKNDQGITNKCKMNLQDNNGNDIGFIGFHQ | |
| | FNNIAKLVASNWYNRQIERSSRTLGCSWEFIPVDDGWGERPLQ | |
| ^{ci}BoNT/PMP1B | MLQIRVFNYNDPIDGENIVELRYHNRSPVKAFQIVDGIWIIPERYNFTNDT | 88 |
| | KKVPDDRALTILEDEVFAVRENDYLTTDVNEKNSFLNNITKLFKRINSSNI | |
| | GNQLLNYISTSVPYPVVSTNSIKARDYNTIKFDSIDGRRITKSANVLIYGPS | |
| | MKNLLDKQTRAINGEEAKNGIGCLSDIIFSPNYLSVQTVSSSRFVEDPASS | |
| | LTHQLIHALHNLYGIQYPGEEKFKFGGFIDKLLGTRECIDYEEVLTYGGK | |
| | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCSITSKNRVNICI | |
| | DVNKEDLYFISDKEGFENIDFSEPEIRYDSNVTTATTSSFTDHFLVNRTFN | |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQILNNIILNLRYKDNNLIDLSGYGAKVEVYD | |
| | GVELNDKNQFKLTSSANSKIRVTQNQNIIFNSVFLDFSVSFWIRIPKYKND | |
| | GIQNYIHNEYTIINCMKNNSGWKISIRGNRIIWTLIDINGKTKSVFFEYNIR | |
| | EDISEYINRWFFVTITNNLNNAKIYINGKLESNTDIKDIREVIANGEIIFKLD | |
| | GDIDRTQFIWMKYFSIFNTELSQSNIEERYKIQSYSEYLKDFWGNPLMYN | |
| | KEYYMFNAGNKNSYIKLKKDSPVGEILTRSKYNQNSKYINYRDLYIGEKF | |
| | IIRRKSNSQSINDDIVRKEDYIYLDFFNLNQEWRVYTYKYFKKEEEKLFLA | |
| | PISDSDEFYNTIQIKEYDEQPTYSCQLLFKKDEESTDEIGLIGIHRFYESGIV | |
| | FEEYKDYFCISKWYLKEVKRKPYNLKLGCNWQFIPKDEGWTE | |
| ^{ci}BoNT/PMP1C | MLQIRVFNYNDPIDGENIVELRYHNRSPVKAFQIVDGIWIIPERYNFTNDT | 89 |
| | KKVPDDRALTILEDEVFAVRENDYLTTDVNEKNSFLNNITKLFKRINSSNI | |
| | GNQLLNYISTSVPYPVVSTNSIKARDYNTIKFDSIDGRRITKSANVLIYGPS | |
| | MKNLLDKQTRAINGEEAKNGIGCLSDIIFSPNYLSVQTVSSSRFVEDPASS | |
| | LTHQLIHALHNLYGIQYPGEEKFKFGGFIDKLLGTRECIDYEEVLTYGGK | |
| | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCSITSKNRVNICI | |
| | DVNKEDLYFISDKEGFENIDFSEPEIRYDSNVTTATTSSFTDHFLVNRTFN | |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQINDSKILSLQNRKNTLVDTSGYNAEVSEEG | |
| | DVQLNPIFPFDFKLGSSGEDRGKVIVTQNENIVYNSMYESFSISFWIRINK | |
| | WVSNLPGYTIIDSVKNNSGWSIGIISNFLVFTLKQNEDSEQSINFSYDISNN | |
| | APGYNKWFFVTVTNNMMGNMKIYINGKLIDTIKVKELTGINFSKTITFEI | |
| | NKIPDTGLITSDSDNINMWIRDFYIFAKELDGKDINILFNSLQYTNVVKDY | |
| | WGNDLRYNKEYYMVNIDYLNRYMYANSRQIVFNTRRNNNDFNEGYKIII | |
| | KRIRGNTNDTRVRGGDILYFDMTINNKAYNLFMKNETMYADNHSTEDIY | |
| | AIGLREQTKDINDNIIFQIQPMNNTYYYASQIFKSNFNGENISGICSIGTYRF | |
| | RLGGDWYRHNYLVPTVKQGNYASLLESTSTHWGFVPVSE | |
| ciBoNT/PMP1D | MLQIRVFNYNDPIDGENIVELRYHNRSPVKAFQIVDGIWIIPERYNFTNDT | 90 |
| | KKVPDDRALTILEDEVFAVRENDYLTTDVNEKNSFLNNITKLFKRINSSNI | |
| | GNQLLNYISTSVPYPVVSTNSIKARDYNTIKFDSIDGRRITKSANVLIYGPS | |
| | MKNLLDKQTRAINGEEAKNGIGCLSDIIFSPNYLSVQTVSSSRFVEDPASS | |
| | LTHQLIHALHNLYGIQYPGEEKFKFGGFIDKLLGTRECIDYEEVLTYGGK | |
| | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCSITSKNRVNICI | |
| | DVNKEDLYFISDKEGFENIDFSEPEIRYDSNVTTATTSSFTDHFLVNRTFN | |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQINDSKILSLQNKKNALVDTSGYNAEVRVG | |
| | DNVQLNTIYTNDFKLSSSGDKIIVNLNNNILYSAIYENSSVSFWIKISKDLT | |
| | NSHNEYTIINSIEQNSGWKLCIRNGNIEWILQDVNRKYKSLIFDYSESLSHT | |
| | GYTNKWFFVTITNNIMGYMKLYINGELKQSQKIEDLDEVKLDKTIVFGID | |
| | ENIDENQMLWIRDFNIFSKELSNEDINIVYEGQILRNVIKDYWGNPLKFDT | |
| | EYYIINDNYIDRYIAPESNVLVLVQYPDRSKLYTGNPITIKSVSDKNPYSRI | |
| | LNGDNIILHMLYNSRKYMIIRDTDTIYATQGGECSQNCVYALKLQSNLGN | |
| | YGIGIFSIKNIVSKNKYCSQIFSSFRENTMLLADIYKPWRFSFKNAYTPVA | |
| | VTNYETKLLSTSSFWKFISRDPGWVE | |
| ^{ci}BoNT/PMP1E | MLQIRVFNYNDPIDGENIVELRYHNRSPVKAFQIVDGIWIIPERYNFTNDT | 91 |
| | KKVPDDRALTILEDEVFAVRENDYLTTDVNEKNSFLNNITKLFKRINSSNI | |
| | GNQLLNYISTSVPYPVVSTNSIKARDYNTIKFDSIDGRRITKSANVLIYGPS | |
| | MKNLLDKQTRAINGEEAKNGIGCLSDIIFSPNYLSVQTVSSSRFVEDPASS | |
| | LTHQLIHALHNLYGIQYPGEEKFKFGGFIDKLLGTRECIDYEEVLTYGGK | |
| | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCSITSKNRVNICI | |
| | DVNKEDLYFISDKEGFENIDFSEPEIRYDSNVTTATTSSFTDHFLVNRTFN | |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQIKSSSVLNMRYKNDKYVDTSGYDSNINING | |
| | DVYKYPTNKNQFGIYNDKLSEVNISQNDYIIYDNKYKNFSISFWVRIPNY | |
| | DNKIVNVNNEYTIINCMRDNNSGWKVSLNHNEIIWTLQDNAGINQKLAF | |
| | NYGNANGISDYINKWIFVTITNDRLGDSKLYINGNLIDQKSILNLGNIHVS | |
| | DNILFKIVNCSYTRYIGIRYFNIFDKELDETEIQTLYSNEPNTNILKDFWGN | |
| | YLLYDKEYYLLNVLKPNNFIDRRKDSTLSINNIRSTILLANRLYSGIKVKIQ | |
| | RVNNSSTNDNLVRKNDQVYINFVASKTHLFPLYADTATTNKEKTIKISSS | |
| | GNRFNQVVVMNSVGNNCTMNFKNNNGNNIGLLGFKADTVVASTWYYT | |
| | HMRDHTNSNGCFWNFISEEHGWQEK | |
| ^{ci}BoNT/PMP1F | MLQIRVFNYNDPIDGENIVELRYHNRSPVKAFQIVDGIWIIPERYNFTNDT | 92 |
| | KKVPDDRALTILEDEVFAVRENDYLTTDVNEKNSFLNNITKLFKRINSSNI | |
| | GNQLLNYISTSVPYPVVSTNSIKARDYNTIKFDSIDGRRITKSANVLIYGPS | |
| | MKNLLDKQTRAINGEEAKNGIGCLSDIIFSPNYLSVQTVSSSRFVEDPASS | |
| | LTHQLIHALHNLYGIQYPGEEKFKFGGFIDKLLGTRECIDYEEVLTYGGK | |
| | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCSITSKNRVNICI | |
| | DVNKEDLYFISDKEGFENIDFSEPEIRYDSNVTTATTSSFTDHFLVNRTFN | |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQIKDNSILDMRYENNKFIDISGYGSNISINGD | |
| | VYIYSTNRNQFGIYSSKPSEVNIAQNNDIIYNGRYQNFSISFWVRIPKYFNK | |
| | VNLNNEYTIIDCIRNNNSGWKISLNYNKIIWTLQDTAGNNQKLVFNYTQ | |
| | MISISDYINKWIFVTITNNRLGNSRIYINGNLIDEKSISNLGDIHVSDNILFKI | |
| | VGCNDTRYVGIRYFKVFDTELGKTEIETLYSDEPDPSILKDFWGNYLLYN | |
| | KRYYLLNLLRTDKSITQNSNFLNINQQRGVYQKPNIFSNTRLYTGVEVIIR | |
| | KNGSTDISNTDNFVRKNDLAYINVVDRDVEYRLYADISIAKPEKIIKLIRT | |
| | SNSNNSLGQIIVMDSIGNNCTMNFQNNNGGNIGLLGFHSNNLVASSWYY | |
| | NNIRKNTSSNGCFWSFISKEHGWQEN | |
| ^{ci}BoNT/PMP1G | MLQIRVFNYNDPIDGENIVELRYHNRSPVKAFQIVDGIWIIPERYNFTNDT | 93 |
| | KKVPDDRALTILEDEVFAVRENDYLTTDVNEKNSFLNNITKLFKRINSSNI | |
| | GNQLLNYISTSVPYPVVSTNSIKARDYNTIKFDSIDGRRITKSANVLIYGPS | |
| | MKNLLDKQTRAINGEEAKNGIGCLSDIIFSPNYLSVQTVSSSRFVEDPASS | |
| | LTHQLIHALHNLYGIQYPGEEKFKFGGFIDKLLGTRECIDYEEVLTYGGK | |
| | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCSITSKNRVNICI | |
| | DVNKEDLYFISDKEGFENIDFSEPEIRYDSNVTTATTSSFTDHFLVNRTFN | |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQISSNAILSLSYRGGRLIDSSGYGATMNVGSD | |
| | VIFNDIGNGQFKLNNSENSNITAHQSKFVVYDSMFDNFSINFWVRTPKYN | |
| | NNDIQTYLQNEYTIISCIKNDSGWKVSIKGNRIIWTLIDVNAKSKSIFFEYSI | |
| | KDNISDYINKWFSITITNDRLGNANIYINGSLKKSEKILNLDRINSSNDIDF | |
| | KLINCTDTTKFVWIKDFNIFGRELNATEVSSLYWIQSSTNTLKDFWGNPL | |
| | RYDTQYYLFNQGMQNIYIKYFSKASMGETAPRTNFNNAAINYQNLYLGL | |
| | RFIIKKASNSRNINNDNIVREGDYIYLNIDNISDESYRVYVLVNSKEIQTQL | |
| | FLAPINDDPTFYDVLQIKKYYEKTTYNCQILCEKDTKTFGLFGIGKFVKD | |
| | YGYVWDTYDNYFCISQWYLRRISENINKLRLGCNWQFIPVDEGWTE | |
| ^{ci}BoNT/PMP1H | MLQIRVFNYNDPIDGENIVELRYHNRSPVKAFQIVDGIWIIPERYNFTNDT | 94 |
| | KKVPDDRALTILEDEVFAVRENDYLTTDVNEKNSFLNNITKLFKRINSSNI | |
| | GNQLLNYISTSVPYPVVSTNSIKARDYNTIKFDSIDGRRITKSANVLIYGPS | |
| | MKNLLDKQTRAINGEEAKNGIGCLSDIIFSPNYLSVQTVSSSRFVEDPASS | |
| | LTHQLIHALHNLYGIQYPGEEKFKFGGFIDKLLGTRECIDYEEVLTYGGK | |
| | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCSITSKNRVNICI | |
| | DVNKEDLYFISDKEGFENIDFSEPEIRYDSNVTTATTSSFTDHFLVNRTFN | |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQLKYNCILNIKYEMDRDKLVDSSGYRSRINI | |
| | GTGVKFSEIDKNQVQLSNLESSKIEVILNNGVIYNSMYENFSTSFWIRIPK | |
| | YFRNINNEYKIISCMQNNSGWEVSLNFSNMNSKIIWTLQDTEGIKKTVVF | |
| | QYTQNINISDYINRWIFVTITNNRLSNSKIYINGRLINEESISDLGNIHASNN | |
| | IMFKLDGCRDPHRYIWIKYFNLFDKELNKKEIKDLYDNQSNSGILKDFWG | |
| | DYLQYDKPYYMLNLYDPNKYLDVNNVGIRGYMYLKGPRGRIVTTNIYL | |
| | NSTLYMGTKFIIKKYASGNKDNIVRNNDRVYINVVVKNKEYRLATNASQ | |
| | AGVEKILSAVEIPDVGNLSQVVVMKSENDQGIRNKCKMNLQDNNGNDI | |
| | GFIGFHQFNNIAKLVASNWYNRQIGKASRTFGCSWEFIPVDDGWGESSL | |
| ^{ci}LC-Hn/X | MKLEINKFNYNDPIDGINVITMRPPRHSDKINKGKGPFKAFQVIKNIWIVP | 21 |
| w/ linker that | ERYNFTNNTNDLNIPSEPIMEADAIYNPNYLNTPSEKDEFLQGVIKVLERI | |
| contains thrombin | KSKPEGEKLLELISSSIPLPLVSNGALTLSDNETIAYQENNNIVSNLQANLV | |
| cleavage sites | IYGPGPDIANNATYGLYSTPISNGEGTLSEVSFSPFYLKPFDESYGNYRSL | |
| between LC and | VNIVNKFVKREFAPDPASTLMHQLVHVTHNLYGISNRNFYYNFDTGKIET | |
| Hn | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICHKAIDGRSLGGSLVPRGSGGSAAAYNKTLDCIEVENKDL | |
| | FLISNKDSLNDINLSEEKIKPETTVFFKDKLPPQDITLSNYDFTEANSIPSIS | |
| | QQNILERNEELYEPIRNSLFEIKTIYVDKLTTFHFLEAQNIDESIDSSKIRVE | |
| | LTDSVDEALSNPNKVYSPFKNMSNTINSIETGITSTYIFYQWLRSIVKDFSD | |
| | ETGKIDVIDKSSDTLAIVPYIGPLLNIGNDIRHGDFVGAIELAGITALLEYV | |
| | PEFTIPILVGLEVIGGELAREQVEAIVNNALDKRDQKWAEVYNITKAQW | |
| | WGTIHLQINTRLAHTYKALSRQANAIKMNMEFQLANYKGNIDDKAKIKN | |
| | AISETEILLNKSVEQAMKNTEKFMIKLSNSYLTKEMIPKVQDNLKNFDLE | |
| | TKKTLDKFIKEKEDILGTNLSSSLRRKVSIRLNKNIAFDINDIPFSEFDDLIN | |
| | QYKNEIEDYEVLNLGAEDGKIKDLSGTTSDINIGSDIE | |
| ^{ci}LC-Hn/En | MVTINDLHYSDPIDEDNIINMRIPLYDLEVDDQFINHNVPDLKAFQVFPNV | 22 |
| w/ linker that | WVVPERYTFYSTMKNLDAPANPSRSSYYDPTYLQSDAEKEVFLQQMILL | |
| contains thrombin | FKRINSTQEGQQFLNLLSRSIPVPYESNGDVAMGTTQVIKQMDDKGNVL | |
| cleavage sites | KHRRAHIIIYGPGPDLMAKGSKALTKSRETGRGCMAEIYFSPMYHKTYST | |
| between LC and | KLTNKNSLVDKSVQEFVPDPAVTLIHQLCHGLHALYGIDLGNVGSWEFN | |
| Hn | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACHKAIDGRSLGGSLVPRGSGGSAAAY | |
| | NKTLDCIEILEDDLFIMSSKDSFTDTDFSEPSVGPVSYKAKKGADTILDSTL | |
| | SNYDFSKEINFTSTVPIITVEDPLETDEDVPVISEDRTVYVDDYTTFHFLEA | |
| | QKIGKEVVPTQTKVVFTTNMEEALFDSKKVYTVFENTASRINEAGTGIAN | |
| | GMMFYQWLKGIVQDFTEEATQKDTFDKISDVTMIVPYLGNILNIGNDIRK | |
| | GDFMGAVELGGVTILLEAIPELTLPVLIGLTIIEDELEKEQVSQTVYNVLD | |
| | KRDEKWEEVYGFVKQQWWWMVHTQFETRILHAYQALNHQVEAIKAN | |
| | MTYQLANYRGNQEDKELLEKAIDDTLQSLYYAVDQAMHNIKRFLIQSSK | |
| | SYLLNQMLPKTKEQLLAFDQQTLRNVNDFINKNQGVLGESLAKDLKKK | |
| | VEKRLTSLPVFNLEDLPISEFEDLIHSHEIDIQDSEVLNIGVNNGKIQDLSG | |
| | ENTPLTLGENLH | |
| ^{ci}LC-Hn/PMP1 | MLQIRVFNYNDPIDGENIVELRYHNRSPVKAFQIVDGIWIIPERYNFTNDT | 95 |
| w/ linker that | KKVPDDRALTILEDEVFAVRENDYLTTDVNEKNSFLNNITKLFKRINSSNI | |
| contains thrombin | GNQLLNYISTSVPYPVVSTNSIKARDYNTIKFDSIDGRRITKSANVLIYGPS | |
| cleavage sites | MKNLLDKQTRAINGEEAKNGIGCLSDIIFSPNYLSVQTVSSSRFVEDPASS | |
| between LC and | LTHQLIHALHNLYGIQYPGEEKFKFGGFIDKLLGTRECIDYEEVLTYGGK | |
| Hn | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCHKAIDGRSLG | |
| | GSLVPRGSGGSAAAYNKTLDCIDVNKEDLYFISDKEGFENIDFSEPEIRYD | |
| | SNVTTATTSSFTDHFLVNRTFNDSDRFPPVELEYAIEPAEIVDNTIMPDIDQ | |
| | KSEISLDNLTTFHYLNAQKMDLGFDSSKEQLKMVTSIEESLLDSKKVYTP | |
| | FTRTAHSVNERISGIAESYLFYQWLKTVINDFTDELNQKSNTDKVADISWI | |
| | IPYVGPALNIGLDLSHGDFTKAFEDLGVSILFAIAPEFATISLVALSIYENIE | |
| | EDSQKEKVINKVENTLARRIEKWHQVYAFMVAQWWGMVHTQIDTRIH | |
| | QMYESLSHQIIAIKANMEYQLSHYKGPDNDKLLLKDYIYEAEIALNTSAN | |
| | RAMKNIERFMIESSISYLKNNLIPSVVENLKKFDADTKKNLDQFIDKNSSV | |
| | LGSDLHILKSQVDLELNPTTKVAFNIQSIPDFDINALIDRLGIQ | |
| ^{ci}BoNT/XA | MKLEINKFNYNDPIDGINVITMRPPRHSDKINKGKGPFKAFQVIKNIWIVP | 23 |
| w/ linker that | ERYNFTNNTNDLNIPSEPIMEADAIYNPNYLNTPSEKDEFLQGVIKVLERI | |
| contains thrombin | KSKPEGEKLLELISSSIPLPLVSNGALTLSDNETIAYQENNNIVSNLQANLV | |
| cleavage sites | IYGPGPDIANNATYGLYSTPISNGEGTLSEVSFSPFYLKPFDESYGNYRSL | |
| between LC and | VNIVNKFVKREFAPDPASTLMHQLVHVTHNLYGISNRNFYYNFDTGKIET | |
| Hn | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICHKAIDGRSLGGSLVPRGSGGSAAAYNKTLDCIEVENKDL | |
| | FLISNKDSLNDINLSEEKIKPETTVFFKDKLPPQDITLSNYDFTEANSIPSIS | |
| | QQNILERNEELYEPIRNSLFEIKTIYVDKLTTFHFLEAQNIDESIDSSKIRVE | |
| | LTDSVDEALSNPNKVYSPFKNMSNTINSIETGITSTYIFYQWLRSIVKDFSD | |
| | ETGKIDVIDKSSDTLAIVPYIGPLLNIGNDIRHGDFVGAIELAGITALLEYV | |
| | PEFTIPILVGLEVIGGELAREQVEAIVNNALDKRDQKWAEVYNITKAQW | |
| | WGTIHLQINTRLAHTYKALS RQANAIKMNMEFQLANYKGNIDDKAKIKN | |
| | AISETEILLNKSVEQAMKNTEKFMIKLSNSYLTKEMIPKVQDNLKNFDLE | |
| | TKKTLDKFIKEKEDILGTNLSSSLRRKVSIRLNKNIAFDINDIPFSEFDDLIN | |
| | QYKNEIEDYEVLNLGAEDGKIKDLSGTTSDINIGSDIEIINTSILNLRYESN | |
| | HLIDLSRYASKINIGSKVNFDPIDKNQIQLFNLESSKIEVILKNAIVYNSMY | |
| | ENFSTSFWIRIPKYFNSISLNNEYTIINCMENNSGWKVSLNYGEIIWTLQDT | |
| | QEIKQRVVFKYSQMINISDYINRWIFVTITNNRLNNSKIYINGRLIDQKPIS | |
| | NLGNIHASNNIMFKLDGCRDTHRYIWIKYFNLFDKELNEKEIKDLYDNQS | |
| | NSGILKDFWGDYLQYDKPYYMLNLYDPNKYVDVNNVGIRGYMYLKGP | |
| | RGSVMTTNIYLNSSLYRGTKFIIKKYASGNKDNIVRNNDRVYINVVVKN | |
| | KEYRLATNASQAGVEKILSALEIPDVGNLSQVVVMKSKNDQGITNKCKM | |
| | NLQDNNGNDIGFIGFHQFNNIAKLVASNWYNRQIERSSRTLGCSWEFIPV | |
| | DDGWGERPLQ | |
| ^{ci}BoNT/XB | MKLEINKFNYNDPIDGINVITMRPPRHSDKINKGKGPFKAFQVIKNIWIVP | 24 |
| w/ linker that | ERYNFTNNTNDLNIPSEPIMEADAIYNPNYLNTPSEKDEFLQGVIKVLERI | |
| contains thrombin | KSKPEGEKLLELISSSIPLPLVSNGALTLSDNETIAYQENNNIVSNLQANLV | |
| cleavage sites | IYGPGPDIANNATYGLYSTPISNGEGTLSEVSFSPFYLKPFDESYGNYRSL | |
| between LC and | VNIVNKFVKREFAPDPASTLMHQLVHVTHNLYGISNRNFYYNFDTGKIET | |
| Hn | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICHKAIDGRSLGGSLVPRGSGGSAAAYNKTLDCIEVENKDL | |
| | FLISNKDSLNDINLSEEKIKPETTVFFKDKLPPQDITLSNYDFTEANSIPSIS | |
| | QQNILERNEELYEPIRNSLFEIKTIYVDKLTTFHFLEAQNIDESIDSSKIRVE | |
| | LTDSVDEALSNPNKVYSPFKNMSNTINSIETGITSTYIFYQWLRSIVKDFSD | |
| | ETGKIDVIDKSSDTLAIVPYIGPLLNIGNDIRHGDFVGAIELAGITALLEYV | |
| | PEFTIPILVGLEVIGGELAREQVEAIVNNALDKRDQKWAEVYNITKAQW | |
| | WGTIHLQINTRLAHTYKALSRQANAIKMNMEFQLANYKGNIDDKAKIKN | |
| | AISETEILLNKSVEQAMKNTEKFMIKLSNSYLTKEMIPKVQDNLKNFDLE | |
| | TKKTLDKFIKEKEDILGTNLSSSLRRKVSIRLNKNIAFDINDIPFSEFDDLIN | |
| | QYKNEIEDYEVLNLGAEDGKIKDLSGTTSDINIGSDIEILNNIILNLRYKDN | |
| | NLIDLSGYGAKVEVYDGVELNDKNQFKLTSSANSKIRVTQNQNIIFNSVF | |
| | LDFSVSFWIRIPKYKNDGIQNYIHNEYTIINCMKNNSGWKISIRGNRIIWTL | |
| | IDINGKTKSVFFEYNIREDISEYINRWFFVTITNNLNNAKIYINGKLESNTDI | |
| | KDIREVIANGEIIFKLDGDIDRTQFIWMKYFSIFNTELSQSNIEERYKIQSYS | |
| | EYLKDFWGNPLMYNKEYYMFNAGNKNSYIKLKKDSPVGEILTRSKYNQ | |
| | NSKYINYRDLYIGEKFIIRRKSNSQSINDDIVRKEDYIYLDFFNLNQEWRV | |
| | YTYKYFKKEEEKLFLAPISDSDEFYNTIQIKEYDEQPTYSCQLLFKKDEES | |
| | TDEIGLIGIHRFYESGIVFEEYKDYFCISKWYLKEVKRKPYNLKLGCNWQ | |
| | FIPKDEGWTE | |
| ^{ci}BoNT/XC | MKLEINKFNYNDPIDGINVITMRPPRHSDKINKGKGPFKAFQVIKNIWIVP | 25 |
| w/ linker that | ERYNFTNNTNDLNIPSEPIMEADAIYNPNYLNTPSEKDEFLQGVIKVLERI | |
| contains thrombin | KSKPEGEKLLELISSSIPLPLVSNGALTLSDNETIAYQENNNIVSNLQANLV | |
| cleavage sites | IYGPGPDIANNATYGLYSTPISNGEGTLSEVSFSPFYLKPFDESYGNYRSL | |
| between LC and | VNIVNKFVKREFAPDPASTLMHQLVHVTHNLYGISNRNFYYNFDTGKIET | |
| Hn | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICHKAIDGRSLGGSLVPRGSGGSAAAYNKTLDCIEVENKDL | |
| | FLISNKDSLNDINLSEEKIKPETTVFFKDKLPPQDITLSNYDFTEANSIPSIS | |
| | QQNILERNEELYEPIRNSLFEIKTIYVDKLTTFHFLEAQNIDESIDSSKIRVE | |
| | LTDSVDEALSNPNKVYSPFKNMSNTINSIETGITSTYIFYQWLRSIVKDFSD | |
| | ETGKIDVIDKSSDTLAIVPYIGPLLNIGNDIRHGDFVGAIELAGITALLEYV | |
| | PEFTIPILVGLEVIGGELAREQVEAIVNNALDKRDQKWAEVYNITKAQW | |
| | WGTIHLQINTRLAHTYKALSRQANAIKMNMEFQLANYKGNIDDKAKIKN | |
| | AISETEILLNKSVEQAMKNTEKFMIKLSNSYLTKEMIPKVQDNLKNFDLE | |
| | TKKTLDKFIKEKEDILGTNLSSSLRRKVSIRLNKNIAFDINDIPFSEFDDLIN | |
| | QYKNEIEDYEVLNLGAEDGKIKDLSGTTSDINIGSDIEINDSKILSLQNRKN | |
| | TLVDTSGYNAEVSEEGDVQLNPIFPFDFKLGSSGEDRGKVIVTQNENIVY | |
| | NSMYESFSISFWIRINKWVSNLPGYTIIDSVKNNSGWSIGIISNFLVFTLKQ | |
| | NEDSEQSINFSYDISNNAPGYNKWFFVTVTNNMMGNMKIYINGKLIDTIK | |
| | VKELTGINFSKTITFEINKIPDTGLITSDSDNINMWIRDFYIFAKELDGKDIN | |
| | ILFNSLQYTNVVKDYWGNDLRYNKEYYMVNIDYLNRYMYANSRQIVFN | |
| | TRRNNNDFNEGYKIIIKRIRGNTNDTRVRGGDILYFDMTINNKAYNLFMK | |
| | NETMYADNHSTEDIYAIGLREQTKDINDNIIFQIQPMNNTYYYASQIFKSN | |
| | FNGENISGICSIGTYRFRLGGDWYRHNYLVPTVKQGNYASLLESTSTHW | |
| | GFVPVSE | |
| ^{ci}BoNT/XD | MKLEINKFNYNDPIDGINVITMRPPRHSDKINKGKGPFKAFQVIKNIWIVP | 26 |
| w/ linker that | ERYNFTNNTNDLNIPSEPIMEADAIYNPNYLNTPSEKDEFLQGVIKVLERI | |
| contains thrombin | KSKPEGEKLLELISSSIPLPLVSNGALTLSDNETIAYQENNNIVSNLQANLV | |
| cleavage sites | IYGPGPDIANNATYGLYSTPISNGEGTLSEVSFSPFYLKPFDESYGNYRSL | |
| between LC and | VNIVNKFVKREFAPDPASTLMHQLVHVTHNLYGISNRNFYYNFDTGKIET | |
| Hn | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICHKAIDGRSLGGSLVPRGSGGSAAAYNKTLDCIEVENKDL | |
| | FLISNKDSLNDINLSEEKIKPETTVFFKDKLPPQDITLSNYDFTEANSIPSIS | |
| | QQNILERNEELYEPIRNSLFEIKTIYVDKLTTFHFLEAQNIDESIDSSKIRVE | |
| | LTDSVDEALSNPNKVYSPFKNMSNTINSIETGITSTYIFYQWLRSIVKDFSD | |
| | ETGKIDVIDKSSDTLAIVPYIGPLLNIGNDIRHGDFVGAIELAGITALLEYV | |
| | PEFTIPILVGLEVIGGELAREQVEAIVNNALDKRDQKWAEVYNITKAQW | |
| | WGTIHLQINTRLAHTYKALSRQANAIKMNMEFQLANYKGNIDDKAKIKN | |
| | AISETEILLNKSVEQAMKNTEKFMIKLSNSYLTKEMIPKVQDNLKNFDLE | |
| | TKKTLDKFIKEKEDILGTNLSSSLRRKVSIRLNKNIAFDINDIPFSEFDDLIN | |
| | QYKNEIEDYEVLNLGAEDGKIKDLSGTTSDINIGSDIEINDSKILSLQNKKN | |
| | ALVDTSGYNAEVRVGDNVQLNTIYTNDFKLSSSGDKIIVNLNNNILYSAI | |
| | YENSSVSFWIKISKDLTNSHNEYTIINSIEQNSGWKLCIRNGNIEWILQDVN | |
| | RKYKSLIFDYSESLSHTGYTNKWFFVTITNNIMGYMKLYINGELKQSQKI | |
| | EDLDEVKLDKTIVFGIDENIDENQMLWIRDFNIFSKELSNEDINIVYEGQIL | |
| | RNVIKDYWGNPLKFDTEYYIINDNYIDRYIAPESNVLVLVQYPDRSKLYT | |
| | GNPITIKSVSDKNPYSRILNGDNIILHMLYNSRKYMIIRDTDTIYATQGGEC | |
| | SQNCVYALKLQSNLGNYGIGIFSIKNIVSKNKYCSQIFSSFRENTMLLADI | |
| | YKPWRFSFKNAYTPVAVTNYETKLLSTSSFWKFISRDPGWVE | |
| ^{ci}BoNT/XE | MKLEINKFNYNDPIDGINVITMRPPRHSDKINKGKGPFKAFQVIKNIWIVP | 27 |
| w/ linker that | ERYNFTNNTNDLNIPSEPIMEADAIYNPNYLNTPSEKDEFLQGVIKVLERI | |
| contains thrombin | KSKPEGEKLLELISSSIPLPLVSNGALTLSDNETIAYQENNNIVSNLQANLV | |
| cleavage sites | IYGPGPDIANNATYGLYSTPISNGEGTLSEVSFSPFYLKPFDESYGNYRSL | |
| between LC and | VNIVNKFVKREFAPDPASTLMHQLVHVTHNLYGISNRNFYYNFDTGKIET | |
| Hn | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICHKAIDGRSLGGSLVPRGSGGSAAAYNKTLDCIEVENKDL | |
| | FLISNKDSLNDINLSEEKIKPETTVFFKDKLPPQDITLSNYDFTEANSIPSIS | |
| | QQNILERNEELYEPIRNSLFEIKTIYVDKLTTFHFLEAQNIDESIDSSKIRVE | |
| | LTDSVDEALSNPNKVYSPFKNMSNTINSIETGITSTYIFYQWLRSIVKDFSD | |
| | ETGKIDVIDKSSDTLAIVPYIGPLLNIGNDIRHGDFVGAIELAGITALLEYV | |
| | PEFTIPILVGLEVIGGELAREQVEAIVNNALDKRDQKWAEVYNITKAQW | |
| | WGTIHLQINTRLAHTYKALSRQANAIKMNMEFQLANYKGNIDDKAKIKN | |
| | AISETEILLNKSVEQAMKNTEKFMIKLSNSYLTKEMIPKVQDNLKNFDLE | |
| | TKKTLDKFIKEKEDILGTNLSSSLRRKVSIRLNKNIAFDINDIPFSEFDDLIN | |
| | QYKNEIEDYEVLNLGAEDGKIKDLSGTTSDINIGSDIEIKSSSVLNMRYKN | |
| | DKYVDTSGYDSNININGDVYKYPTNKNQFGIYNDKLSEVNISQNDYIIYD | |
| | NKYKNFSISFWVRIPNYDNKIVNVNNEYTIINCMRDNNSGWKVSLNHNEI | |
| | IWTLQDNAGINQKLAFNYGNANGISDYINKWIFVTITNDRLGDSKLYING | |
| | NLIDQKSILNLGNIHVSDNILFKIVNCSYTRYIGIRYFNIFDKELDETEIQTL | |
| | YSNEPNTNILKDFWGNYLLYDKEYYLLNVLKPNNFIDRRKDSTLSINNIR | |
| | STILLANRLYSGIKVKIQRVNNSSTNDNLVRKNDQVYINFVASKTHLFPL | |
| | YADTATTNKEKTIKISSSGNRFNQVVVMNSVGNNCTMNFKNNNGNNIGL | |
| | LGFKADTVVASTWYYTHMRDHTNSNGCFWNFISEEHGWQEK | |
| ^{ci}BoNT/XF | MKLEINKFNYNDPIDGINVITMRPPRHSDKINKGKGPFKAFQVIKNIWIVP | 28 |
| w/ linker that | ERYNFTNNTNDLNIPSEPIMEADAIYNPNYLNTPSEKDEFLQGVIKVLERI | |
| contains thrombin | KSKPEGEKLLELISSSIPLPLVSNGALTLSDNETIAYQENNNIVSNLQANLV | |
| cleavage sites | IYGPGPDIANNATYGLYSTPISNGEGTLSEVSFSPFYLKPFDESYGNYRSL | |
| between LC and | VNIVNKFVKREFAPDPASTLMHQLVHVTHNLYGISNRNFYYNFDTGKIET | |
| Hn | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICHKAIDGRSLGGSLVPRGSGGSAAAYNKTLDCIEVENKDL | |
| | FLISNKDSLNDINLSEEKIKPETTVFFKDKLPPQDITLSNYDFTEANSIPSIS | |
| | QQNILERNEELYEPIRNSLFEIKTIYVDKLTTFHFLEAQNIDESIDSSKIRVE | |
| | LTDSVDEALSNPNKVYSPFKNMSNTINSIETGITSTYIFYQWLRSIVKDFSD | |
| | ETGKIDVIDKSSDTLAIVPYIGPLLNIGNDIRHGDFVGAIELAGITALLEYV | |
| | PEFTIPILVGLEVIGGELAREQVEAIVNNALDKRDQKWAEVYNITKAQW | |
| | WGTIHLQINTRLAHTYKALSRQANAIKMNMEFQLANYKGNIDDKAKIKN | |
| | AISETEILLNKSVEQAMKNTEKFMIKLSNSYLTKEMIPKVQDNLKNFDLE | |
| | TKKTLDKFIKEKEDILGTNLSSSLRRKVSIRLNKNIAFDINDIPFSEFDDLIN | |
| | QYKNEIEDYEVLNLGAEDGKIKDLSGTTSDINIGSDIEIKDNSILDMRYEN | |
| | NKFIDISGYGSNISINGDVYIYSTNRNQFGIYSSKPSEVNIAQNNDIIYNGR | |
| | YQNFSISFWVRIPKYFNKVNLNNEYTIIDCIRNNNSGWKISLNYNKIIWTL | |
| | QDTAGNNQKLVFNYTQMISISDYINKWIFVTITNNRLGNSRIYINGNLIDE | |
| | KSISNLGDIHVSDNILFKIVGCNDTRYVGIRYFKVFDTELGKTEIETLYSDE | |
| | PDPSILKDFWGNYLLYNKRYYLLNLLRTDKSITQNSNFLNINQQRGVYQ | |
| | KPNIFSNTRLYTGVEVIIRKNGSTDISNTDNFVRKNDLAYINVVDRDVEY | |
| | RLYADISIAKPEKIIKLIRTSNSNNSLGQIIVMDSIGNNCTMNFQNNNGGNI | |
| | GLLGFHSNNLVASSWYYNNIRKNTSSNGCFWSFISKEHGWQEN | |
| ^{ci}BoNT/XG | MKLEINKFNYNDPIDGINVITMRPPRHSDKINKGKGPFKAFQVIKNIWIVP | 29 |
| w/ linker that | ERYNFTNNTNDLNIPSEPIMEADAIYNPNYLNTPSEKDEFLQGVIKVLERI | |
| contains thrombin | KSKPEGEKLLELISSSIPLPLVSNGALTLSDNETIAYQENNNIVSNLQANLV | |
| cleavage sites | IYGPGPDIANNATYGLYSTPISNGEGTLSEVSFSPFYLKPFDESYGNYRSL | |
| between LC and | VNIVNKFVKREFAPDPASTLMHQLVHVTHNLYGISNRNFYYNFDTGKIET | |
| Hn | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICHKAIDGRSLGGSLVPRGSGGSAAAYNKTLDCIEVENKDL | |
| | FLISNKDSLNDINLSEEKIKPETTVFFKDKLPPQDITLSNYDFTEANSIPSIS | |
| | QQNILERNEELYEPIRNSLFEIKTIYVDKLTTFHFLEAQNIDESIDSSKIRVE | |
| | LTDSVDEALSNPNKVYSPFKNMSNTINSIETGITSTYIFYQWLRSIVKDFSD | |
| | ETGKIDVIDKSSDTLAIVPYIGPLLNIGNDIRHGDFVGAIELAGITALLEYV | |
| | PEFTIPILVGLEVIGGELAREQVEAIVNNALDKRDQKWAEVYNITKAQW | |
| | WGTIHLQINTRLAHTYKALSRQANAIKMNMEFQLANYKGNIDDKAKIKN | |
| | AISETEILLNKSVEQAMKNTEKFMIKLSNSYLTKEMIPKVQDNLKNFDLE | |
| | TKKTLDKFIKEKEDILGTNLSSSLRRKVSIRLNKNIAFDINDIPFSEFDDLIN | |
| | QYKNEIEDYEVLNLGAEDGKIKDLSGTTSDINIGSDIEISSNAILSLSYRGG | |
| | RLIDSSGYGATMNVGSDVIFNDIGNGQFKLNNSENSNITAHQSKFVVYDS | |
| | MFDNFSINFWVRTPKYNNNDIQTYLQNEYTIISCIKNDSGWKVSIKGNRII | |
| | WTLIDVNAKSKSIFFEYSIKDNISDYINKWFSITITNDRLGNANIYINGSLK | |
| | KSEKILNLDRINSSNDIDFKLINCTDTTKFVWIKDFNIFGRELNATEVSSLY | |
| | WIQSSTNTLKDFWGNPLRYDTQYYLFNQGMQNIYIKYFSKASMGETAPR | |
| | TNFNNAAINYQNLYLGLRFIIKKASNSRNINNDNIVREGDYIYLNIDNISDE | |
| | SYRVYVLVNSKEIQTQLFLAPINDDPTFYDVLQIKKYYEKTTYNCQILCE | |
| | KDTKTFGLFGIGKFVKDYGYVWDTYDNYFCISQWYLRRISENINKLRLG | |
| | CNWQFIPVDEGWTE | |
| ^{ci}BoNT/XH | MKLEINKFNYNDPIDGINVITMRPPRHSDKINKGKGPFKAFQVIKNIWIVP | 30 |
| w/ linker that | ERYNFTNNTNDLNIPSEPIMEADAIYNPNYLNTPSEKDEFLQGVIKVLERI | |
| contains thrombin | KSKPEGEKLLELISSSIPLPLVSNGALTLSDNETIAYQENNNIVSNLQANLV | |
| cleavage sites | IYGPGPDIANNATYGLYSTPISNGEGTLSEVSFSPFYLKPFDESYGNYRSL | |
| between LC and | VNIVNKFVKREFAPDPASTLMHQLVHVTHNLYGISNRNFYYNFDTGKIET | |
| Hn | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICHKAIDGRSLGGSLVPRGSGGSAAAYNKTLDCIEVENKDL | |
| | FLISNKDSLNDINLSEEKIKPETTVFFKDKLPPQDITLSNYDFTEANSIPSIS | |
| | QQNILERNEELYEPIRNSLFEIKTIYVDKLTTFHFLEAQNIDESIDSSKIRVE | |
| | LTDSVDEALSNPNKVYSPFKNMSNTINSIETGITSTYIFYQWLRSIVKDFSD | |
| | ETGKIDVIDKSSDTLAIVPYIGPLLNIGNDIRHGDFVGAIELAGITALLEYV | |
| | PEFTIPILVGLEVIGGELAREQVEAIVNNALDKRDQKWAEVYNITKAQW | |
| | WGTIHLQINTRLAHTYKALSRQANAIKMNMEFQLANYKGNIDDKAKIKN | |
| | AISETEILLNKSVEQAMKNTEKFMIKLSNSYLTKEMIPKVQDNLKNFDLE | |
| | TKKTLDKFIKEKEDILGTNLSSSLRRKVSIRLNKNIAFDINDIPFSEFDDLIN | |
| | QYKNEIEDYEVLNLGAEDGKIKDLSGTTSDINIGSDIELKYNCILNIKYEM | |
| | DRDKLVDSSGYRSRINIGTGVKFSEIDKNQVQLSNLESSKIEVILNNGVIY | |
| | NSMYENFSTSFWIRIPKYFRNINNEYKIISCMQNNSGWEVSLNFSNMNSKI | |
| | IWTLQDTEGIKKTVVFQYTQNINISDYINRWIFVTITNNRLSNSKIYINGRL | |
| | INEESISDLGNIHASNNIMFKLDGCRDPHRYIWIKYFNLFDKELNKKEIKD | |
| | LYDNQSNSGILKDFWGDYLQYDKPYYMLNLYDPNKYLDVNNVGIRGY | |
| | MYLKGPRGRIVTTNIYLNSTLYMGTKFIIKKYASGNKDNIVRNNDRVYIN | |
| | VVVKNKEYRLATNASQAGVEKILSAVEIPDVGNLSQVVVMKSENDQGIR | |
| | NKCKMNLQDNNGNDIGFIGFHQFNNIAKLVASNWYNRQIGKASRTFGCS | |
| | WEFIPVDDGWGESSL | |
| ^{ci}BoNT/EnA | MVTINDLHYSDPIDEDNIINMRIPLYDLEVDDQFINHNVPDLKAFQVFPNV | 31 |
| w/ linker that | WVVPERYTFYSTMKNLDAPANPSRSSYYDPTYLQSDAEKEVFLQQMILL | |
| contains thrombin | FKRINSTQEGQQFLNLLSRSIPVPYESNGDVAMGTTQVIKQMDDKGNVL | |
| cleavage sites | KHRRAHIIIYGPGPDLMAKGSKALTKSRETGRGCMAEIYFSPMYHKTYST | |
| between LC and | KLTNKNSLVDKSVQEFVPDPAVTLIHQLCHGLHALYGIDLGNVGSWEFN | |
| Hn | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACHKAIDGRSLGGSLVPRGSGGSAAAY | |
| | NKTLDCIEILEDDLFIMSSKDSFTDTDFSEPSVGPVSYKAKKGADTILDSTL | |
| | SNYDFSKEINFTSTVPIITVEDPLETDEDVPVISEDRTVYVDDYTTFHFLEA | |
| | QKIGKEVVPTQTKVVFTTNMEEALFDSKKVYTVFENTASRINEAGTGIAN | |
| | GMMFYQWLKGIVQDFTEEATQKDTFDKISDVTMIVPYLGNILNIGNDIRK | |
| | GDFMGAVELGGVTILLEAIPELTLPVLIGLTIIEDELEKEQVSQTVYNVLD | |
| | KRDEKWEEVYGFVKQQWWWMVHTQFETRILHAYQALNHQVEAIKAN | |
| | MTYQLANYRGNQEDKELLEKAIDDTLQSLYYAVDQAMHNIKRFLIQSSK | |
| | SYLLNQMLPKTKEQLLAFDQQTLRNVNDFINKNQGVLGESLAKDLKKK | |
| | VEKRLTSLPVFNLEDLPISEFEDLIHSHEIDIQDSEVLNIGVNNGKIQDLSG | |
| | ENTPLTLGENLHIINTSILNLRYESNHLIDLSRYASKINIGSKVNFDPIDKNQ | |
| | IQLFNLESSKIEVILKNAIVYNSMYENFSTSFWIRIPKYFNSISLNNEYTIINC | |
| | MENNSGWKVSLNYGEIIWTLQDTQEIKQRVVFKYSQMINISDYINRWIFV | |
| | TITNNRLNNSKIYINGRLIDQKPISNLGNIHASNNIMFKLDGCRDTHRYIWI | |
| | KYFNLFDKELNEKEIKDLYDNQSNSGILKDFWGDYLQYDKPYYMLNLY | |
| | DPNKYVDVNNVGIRGYMYLKGPRGSVMTTNIYLNSSLYRGTKFIIKKYA | |
| | SGNKDNIVRNNDRVYINVVVKNKEYRLATNASQAGVEKILSALEIPDVG | |
| | NLSQVVVMKSKNDQGITNKCKMNLQDNNGNDIGFIGFHQFNNIAKLVAS | |
| | NWYNRQIERSSRTLGCSWEFIPVDDGWGERPLQ | |
| ^{ci}BoNT/EnB | MVTINDLHYSDPIDEDNIINMRIPLYDLEVDDQFINHNVPDLKAFQVFPNV | 32 |
| w/ linker that | WVVPERYTFYSTMKNLDAPANPSRSSYYDPTYLQSDAEKEVFLQQMILL | |
| contains thrombin | FKRINSTQEGQQFLNLLSRSIPVPYESNGDVAMGTTQVIKQMDDKGNVL | |
| cleavage sites | KHRRAHIIIYGPGPDLMAKGSKALTKSRETGRGCMAEIYFSPMYHKTYST | |
| between LC and | KLTNKNSLVDKSVQEFVPDPAVTLIHQLCHGLHALYGIDLGNVGSWEFN | |
| Hn | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACHKAIDGRSLGGSLVPRGSGGSAAAY | |
| | NKTLDCIEILEDDLFIMSSKDSFTDTDFSEPSVGPVSYKAKKGADTILDSTL | |
| | SNYDFSKEINFTSTVPIITVEDPLETDEDVPVISEDRTVYVDDYTTFHFLEA | |
| | QKIGKEVVPTQTKVVFTTNMEEALFDSKKVYTVFENTASRINEAGTGIAN | |
| | GMMFYQWLKGIVQDFTEEATQKDTFDKISDVTMIVPYLGNILNIGNDIRK | |
| | GDFMGAVELGGVTILLEAIPELTLPVLIGLTIIEDELEKEQVSQTVYNVLD | |
| | KRDEKWEEVYGFVKQQWWWMVHTQFETRILHAYQALNHQVEAIKAN | |
| | MTYQLANYRGNQEDKELLEKAIDDTLQSLYYAVDQAMHNIKRFLIQSSK | |
| | SYLLNQMLPKTKEQLLAFDQQTLRNVNDFINKNQGVLGESLAKDLKKK | |
| | VEKRLTSLPVFNLEDLPISEFEDLIHSHEIDIQDSEVLNIGVNNGKIQDLSG | |
| | ENTPLTLGENLHILNNIILNLRYKDNNLIDLSGYGAKVEVYDGVELNDKN | |
| | QFKLTSSANSKIRVTQNQNIIFNSVFLDFSVSFWIRIPKYKNDGIQNYIHNE | |
| | YTIINCMKNNSGWKISIRGNRIIWTLIDINGKTKSVFFEYNIREDISEYINR | |
| | WFFVTITNNLNNAKIYINGKLESNTDIKDIREVIANGEIIFKLDGDIDRTQFI | |
| | WMKYFSIFNTELSQSNIEERYKIQSYSEYLKDFWGNPLMYNKEYYMFNA | |
| | GNKNSYIKLKKDSPVGEILTRSKYNQNSKYINYRDLYIGEKFIIRRKSNSQ | |
| | SINDDIVRKEDYIYLDFFNLNQEWRVYTYKYFKKEEEKLFLAPISDSDEFY | |
| | NTIQIKEYDEQPTYSCQLLFKKDEESTDEIGLIGIHRFYESGIVFEEYKDYF | |
| | CISKWYLKEVKRKPYNLKLGCNWQFIPKDEGWTE | |
| ^{ci}BoNT/EnC | MVTINDLHYSDPIDEDNIINMRIPLYDLEVDDQFINHNVPDLKAFQVFPNV | 33 |
| w/ linker that | WVVPERYTFYSTMKNLDAPANPSRSSYYDPTYLQSDAEKEVFLQQMILL | |
| contains thrombin | FKRINSTQEGQQFLNLLSRSIPVPYESNGDVAMGTTQVIKQMDDKGNVL | |
| cleavage sites | KHRRAHIIIYGPGPDLMAKGSKALTKSRETGRGCMAEIYFSPMYHKTYST | |
| between LC and | KLTNKNSLVDKSVQEFVPDPAVTLIHQLCHGLHALYGIDLGNVGSWEFN | |
| Hn | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACHKAIDGRSLGGSLVPRGSGGSAAAY | |
| | NKTLDCIEILEDDLFIMSSKDSFTDTDFSEPSVGPVSYKAKKGADTILDSTL | |
| | SNYDFSKEINFTSTVPIITVEDPLETDEDVPVISEDRTVYVDDYTTFHFLEA | |
| | QKIGKEVVPTQTKVVFTTNMEEALFDSKKVYTVFENTASRINEAGTGIAN | |
| | GMMFYQWLKGIVQDFTEEATQKDTFDKISDVTMIVPYLGNILNIGNDIRK | |
| | GDFMGAVELGGVTILLEAIPELTLPVLIGLTIIEDELEKEQVSQTVYNVLD | |
| | KRDEKWEEVYGFVKQQWWWMVHTQFETRILHAYQALNHQVEAIKAN | |
| | MTYQLANYRGNQEDKELLEKAIDDTLQSLYYAVDQAMHNIKRFLIQSSK | |
| | SYLLNQMLPKTKEQLLAFDQQTLRNVNDFINKNQGVLGESLAKDLKKK | |
| | VEKRLTSLPVFNLEDLPISEFEDLIHSHEIDIQDSEVLNIGVNNGKIQDLSG | |
| | ENTPLTLGENLHINDSKILSLQNRKNTLVDTSGYNAEVSEEGDVQLNPIFP | |
| | FDFKLGSSGEDRGKVIVTQNENIVYNSMYESFSISFWIRINKWVSNLPGYT | |
| | IIDSVKNNSGWSIGIISNFLVFTLKQNEDSEQSINFSYDISNNAPGYNKWFF | |
| | VTVTNNMMGNMKIYINGKLIDTIKVKELTGINFSKTITFEINKIPDTGLITS | |
| | DSDNINMWIRDFYIFAKELDGKDINILFNSLQYTNVVKDYWGNDLRYNK | |
| | EYYMVNIDYLNRYMYANSRQIVFNTRRNNNDFNEGYKIIIKRIRGNTNDT | |
| | RVRGGDILYFDMTINNKAYNLFMKNETMYADNHSTEDIYAIGLREQTKD | |
| | INDNIIFQIQPMNNTYYYASQIFKSNFNGENISGICSIGTYRFRLGGDWYRH | |
| | NYLVPTVKQGNYASLLESTSTHWGFVPVSE | |
| ^{ci}BoNT/EnD | MVTINDLHYSDPIDEDNIINMRIPLYDLEVDDQFINHNVPDLKAFQVFPNV | 34 |
| w/ linker that | WVVPERYTFYSTMKNLDAPANPSRSSYYDPTYLQSDAEKEVFLQQMILL | |
| contains thrombin | FKRINSTQEGQQFLNLLSRSIPVPYESNGDVAMGTTQVIKQMDDKGNVL | |
| cleavage sites | KHRRAHIIIYGPGPDLMAKGSKALTKSRETGRGCMAEIYFSPMYHKTYST | |
| between LC and | KLTNKNSLVDKSVQEFVPDPAVTLIHQLCHGLHALYGIDLGNVGSWEFN | |
| Hn | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACHKAIDGRSLGGSLVPRGSGGSAAAY | |
| | NKTLDCIEILEDDLFIMSSKDSFTDTDFSEPSVGPVSYKAKKGADTILDSTL | |
| | SNYDFSKEINFTSTVPIITVEDPLETDEDVPVISEDRTVYVDDYTTFHFLEA | |
| | QKIGKEVVPTQTKVVFTTNMEEALFDSKKVYTVFENTASRINEAGTGIAN | |
| | GMMFYQWLKGIVQDFTEEATQKDTFDKISDVTMIVPYLGNILNIGNDIRK | |
| | GDFMGAVELGGVTILLEAIPELTLPVLIGLTIIEDELEKEQVSQTVYNVLD | |
| | KRDEKWEEVYGFVKQQWWWMVHTQFETRILHAYQALNHQVEAIKAN | |
| | MTYQLANYRGNQEDKELLEKAIDDTLQSLYYAVDQAMHNIKRFLIQSSK | |
| | SYLLNQMLPKTKEQLLAFDQQTLRNVNDFINKNQGVLGESLAKDLKKK | |
| | VEKRLTSLPVFNLEDLPISEFEDLIHSHEIDIQDSEVLNIGVNNGKIQDLSG | |
| | ENTPLTLGENLHINDSKILSLQNKKNALVDTSGYNAEVRVGDNVQLNTIY | |
| | TNDFKLSSSGDKIIVNLNNNILYSAIYENSSVSFWIKISKDLTNSHNEYTIIN | |
| | SIEQNSGWKLCIRNGNIEWILQDVNRKYKSLIFDYSESLSHTGYTNKWFF | |
| | VTITNNIMGYMKLYINGELKQSQKIEDLDEVKLDKTIVFGIDENIDENQM | |
| | LWIRDFNIFSKELSNEDINIVYEGQILRNVIKDYWGNPLKFDTEYYIINDN | |
| | YIDRYIAPESNVLVLVQYPDRSKLYTGNPITIKSVSDKNPYSRILNGDNIIL | |
| | HMLYNSRKYMIIRDTDTIYATQGGECSQNCVYALKLQSNLGNYGIGIFSI | |
| | KNIVSKNKYCSQIFSSFRENTMLLADIYKPWRFSFKNAYTPVAVTNYETK | |
| | LLSTSSFWKFISRDPGWVE | |
| ^{ci}BoNT/EnE | MVTINDLHYSDPIDEDNIINMRIPLYDLEVDDQFINHNVPDLKAFQVFPNV | 35 |
| w/ linker that | WVVPERYTFYSTMKNLDAPANPSRSSYYDPTYLQSDAEKEVFLQQMILL | |
| contains thrombin | FKRINSTQEGQQFLNLLSRSIPVPYESNGDVAMGTTQVIKQMDDKGNVL | |
| cleavage sites | KHRRAHIIIYGPGPDLMAKGSKALTKSRETGRGCMAEIYFSPMYHKTYST | |
| between LC and | KLTNKNSLDKSVQEFVPDPAVTLIHQLCHGLHALYGIDLGNVGSWEFN | |
| Hn | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACHKAIDGRSLGGSLVPRGSGGSAAAY | |
| | NKTLDCIEILEDDLFIMSSKDSFTDTDFSEPSVGPVSYKAKKGADTILDSTL | |
| | SNYDFSKEINFTSTVPIITVEDPLETDEDVPVISEDRTVYVDDYTTFHFLEA | |
| | QKIGKEVVPTQTKVVFTTNMEEALFDSKKVYTVFENTASRINEAGTGIAN | |
| | GMMFYQWLKGIVQDFTEEATQKDTFDKISDVTMIVPYLGNILNIGNDIRK | |
| | GDFMGAVELGGVTILLEAIPELTLPVLIGLTIIEDELEKEQVSQTVYNVLD | |
| | KRDEKWEEVYGFVKQQWWWMVHTQFETRILHAYQALNHQVEAIKAN | |
| | MTYQLANYRGNQEDKELLEKAIDDTLQSLYYAVDQAMHNIKRFLIQSSK | |
| | SYLLNQMLPKTKEQLLAFDQQTLRNVNDFINKNQGVLGESLAKDLKKK | |
| | VEKRLTSLPVFNLEDLPISEFEDLIHSHEIDIQDSEVLNIGVNNGKIQDLSG | |
| | ENTPLTLGENLHIKSSSVLNMRYKNDKYVDTSGYDSNININGDVYKYPT | |
| | NKNQFGIYNDKLSEVNISQNDYIIYDNKYKNFSISFWVRIPNYDNKIVNV | |
| | NNEYTIINCMRDNNSGWKVSLNHNEIIWTLQDNAGINQKLAFNYGNANG | |
| | ISDYINKWIFVTITNDRLGDSKLYINGNLIDQKSILNLGNIHVSDNILFKIVN | |
| | CSYTRYIGIRYFNIFDKELDETEIQTLYSNEPNTNILKDFWGNYLLYDKEY | |
| | YLLNVLKPNNFIDRRKDSTLSINNIRSTILLANRLYSGIKVKIQRVNNSSTN | |
| | DNLVRKNDQVYINFVASKTHLFPLYADTATTNKEKTIKISSSGNRFNQVV | |
| | VMNSVGNNCTMNFKNNNGNNIGLLGFKADTVVASTWYYTHMRDHTNS | |
| | NGCFWNFISEEHGWQEK | |
| ^{ci}BoNT/EnF | MVTINDLHYSDPIDEDNIINMRIPLYDLEVDDQFINHNVPDLKAFQVFPNV | 36 |
| w/ linker that | WVVPERYTFYSTMKNLDAPANPSRSSYYDPTYLQSDAEKEVFLQQMILL | |
| contains thrombin | FKRINSTQEGQQFLNLLSRSIPVPYESNGDVAMGTTQVIKQMDDKGNVL | |
| cleavage sites | KHRRAHIIIYGPGPDLMAKGSKALTKSRETGRGCMAEIYFSPMYHKTYST | |
| between LC and | KLTNKNSLVDKSVQEFVPDPAVTLIHQLCHGLHALYGIDLGNVGSWEFN | |
| Hn | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACHKAIDGRSLGGSLVPRGSGGSAAAY | |
| | NKTLDCIEILEDDLFIMSSKDSFTDTDFSEPSVGPVSYKAKKGADTILDSTL | |
| | SNYDFSKEINFTSTVPIITVEDPLETDEDVPVISEDRTVYVDDYTTFHFLEA | |
| | QKIGKEVVPTQTKVVFTTNMEEALFDSKKVYTVFENTASRINEAGTGIAN | |
| | GMMFYQWLKGIVQDFTEEATQKDTFDKISDVTMIVPYLGNILNIGNDIRK | |
| | GDFMGAVELGGVTILLEAIPELTLPVLIGLTIIEDELEKEQVSQTVYNVLD | |
| | KRDEKWEEVYGFVKQQWWWMVHTQFETRILHAYQALNHQVEAIKAN | |
| | MTYQLANYRGNQEDKELLEKAIDDTLQSLYYAVDQAMHNIKRFLIQSSK | |
| | SYLLNQMLPKTKEQLLAFDQQTLRNVNDFINKNQGVLGESLAKDLKKK | |
| | VEKRLTSLPVFNLEDLPISEFEDLIHSHEIDIQDSEVLNIGVNNGKIQDLSG | |
| | ENTPLTLGENLHIKDNSILDMRYENNKFIDISGYGSNISINGDVYIYSTNRN | |
| | QFGIYSSKPSEVNIAQNNDIIYNGRYQNFSISFWVRIPKYFNKVNLNNEYTI | |
| | IDCIRNNNSGWKISLNYNKIIWTLQDTAGNNQKLVFNYTQMISISDYINK | |
| | WIFVTITNNRLGNSRIYINGNLIDEKSISNLGDIHVSDNILFKIVGCNDTRY | |
| | VGIRYFKVFDTELGKTEIETLYSDEPDPSILKDFWGNYLLYNKRYYLLNL | |
| | LRTDKSITQNSNFLNINQQRGVYQKPNIFSNTRLYTGVEVIIRKNGSTDISN | |
| | TDNFVRKNDLAYINVVDRDVEYRLYADISIAKPEKIIKLIRTSNSNNSLGQ | |
| | IIVMDSIGNNCTMNFQNNNGGNIGLLGFHSNNLVASSWYYNNIRKNTSS | |
| | NGCFWSFISKEHGWQEN | |
| ^{ci}BoNT/EnG | MVTINDLHYSDPIDEDNIINMRIPLYDLEVDDQFINHNVPDLKAFQVFPNV | 37 |
| w/ linker that | WVVPERYTFYSTMKNLDAPANPSRSSYYDPTYLQSDAEKEVFLQQMILL | |
| contains thrombin | FKRINSTQEGQQFLNLLSRSIPVPYESNGDVAMGTTQVIKQMDDKGNVL | |
| cleavage sites | KHRRAHIIIYGPGPDLMAKGSKALTKSRETGRGCMAEIYFSPMYHKTYST | |
| between LC and | KLTNKNSLVDKSVQEFVPDPAVTLIHQLCHGLHALYGIDLGNVGSWEFN | |
| Hn | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACHKAIDGRSLGGSLVPRGSGGSAAAY | |
| | NKTLDCIEILEDDLFIMSSKDSFTDTDFSEPSVGPVSYKAKKGADTILDSTL | |
| | SNYDFSKEINFTSTVPIITVEDPLETDEDVPVISEDRTVYVDDYTTFHFLEA | |
| | QKIGKEVVPTQTKVVFTTNMEEALFDSKKVYTVFENTASRINEAGTGIAN | |
| | GMMFYQWLKGIVQDFTEEATQKDTFDKISDVTMIVPYLGNILNIGNDIRK | |
| | GDFMGAVELGGVTILLEAIPELTLPVLIGLTIIEDELEKEQVSQTVYNVLD | |
| | KRDEKWEEVYGFVKQQWWWMVHTQFETRILHAYQALNHQVEAIKAN | |
| | MTYQLANYRGNQEDKELLEKAIDDTLQSLYYAVDQAMHNIKRFLIQSSK | |
| | SYLLNQMLPKTKEQLLAFDQQTLRNVNDFINKNQGVLGESLAKDLKKK | |
| | VEKRLTSLPVFNLEDLPISEFEDLIHSHEIDIQDSEVLNIGVNNGKIQDLSG | |
| | ENTPLTLGENLHISSNAILSLSYRGGRLIDSSGYGATMNVGSDVIFNDIGN | |
| | GQFKLNNSENSNITAHQSKFVVYDSMFDNFSINFWVRTPKYNNNDIQTY | |
| | LQNEYTIISCIKNDSGWKVSIKGNRIIWTLIDVNAKSKSIFFEYSIKDNISDY | |
| | INKWFSITITNDRLGNANIYINGSLKKSEKILNLDRINSSNDIDFKLINCTDT | |
| | TKFVWIKDFNIFGRELNATEVSSLYWIQSSTNTLKDFWGNPLRYDTQYYL | |
| | FNQGMQNIYIKYFSKASMGETAPRTNFNNAAINYQNLYLGLRFIIKKASN | |
| | SRNINNDNIVREGDYIYLNIDNISDESYRVYVLVNSKEIQTQLFLAPINDDP | |
| | TFYDVLQIKKYYEKTTYNCQILCEKDTKTFGLFGIGKFVKDYGYVWDTY | |
| | DNYFCISQWYLRRISENINKLRLGCNWQFIPVDEGWTE | |
| ^{ci}BoNT/EnH | MVTINDLHYSDPIDEDNIINMRIPLYDLEVDDQFINHNVPDLKAFQVFPNV | 38 |
| w/ linker that | WVVPERYTFYSTMKNLDAPANPSRSSYYDPTYLQSDAEKEVFLQQMILL | |
| contains thrombin | FKRINSTQEGQQFLNLLSRSIPVPYESNGDVAMGTTQVIKQMDDKGNVL | |
| cleavage sites | KHRRAHIIIYGPGPDLMAKGSKALTKSRETGRGCMAEIYFSPMYHKTYST | |
| between LC and | KLTNKNSLVDKSVQEFVPDPAVTLIHQLCHGLHALYGIDLGNVGSWEFN | |
| Hn | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRACHKAIDGRSLGGSLVPRGSGGSAAAY | |
| | NKTLDCIEILEDDLFIMSSKDSFTDTDFSEPSVGPVSYKAKKGADTILDSTL | |
| | SNYDFSKEINFTSTVPIITVEDPLETDEDVPVISEDRTVYVDDYTTFHFLEA | |
| | QKIGKEVVPTQTKVVFTTNMEEALFDSKKVYTVFENTASRINEAGTGIAN | |
| | GMMFYQWLKGIVQDFTEEATQKDTFDKISDVTMIVPYLGNILNIGNDIRK | |
| | GDFMGAVELGGVTILLEAIPELTLPVLIGLTIIEDELEKEQVSQTVYNVLD | |
| | KRDEKWEEVYGFVKQQWWWMVHTQFETRILHAYQALNHQVEAIKAN | |
| | MTYQLANYRGNQEDKELLEKAIDDTLQSLYYAVDQAMHNIKRFLIQSSK | |
| | SYLLNQMLPKTKEQLLAFDQQTLRNVNDFINKNQGVLGESLAKDLKKK | |
| | VEKRLTSLPVFNLEDLPISEFEDLIHSHEIDIQDSEVLNIGVNNGKIQDLSG | |
| | ENTPLTLGENLHLKYNCILNIKYEMDRDKLVDSSGYRSRINIGTGVKFSEI | |
| | DKNQVQLSNLESSKIEVILNNGVIYNSMYENFSTSFWIRIPKYFRNINNEY | |
| | KIISCMQNNSGWEVSLNFSNMNSKIIWTLQDTEGIKKTVVFQYTQNINISD | |
| | YINRWIFVTITNNRLSNSKIYINGRLINEESISDLGNIHASNNIMFKLDGCR | |
| | DPHRYIWIKYFNLFDKELNKKEIKDLYDNQSNSGILKDFWGDYLQYDKP | |
| | YYMLNLYDPNKYLDVNNVGIRGYMYLKGPRGRIVTTNIYLNSTLYMGT | |
| | KFIIKKYASGNKDNIVRNNDRVYINVVVKNKEYRLATNASQAGVEKILS | |
| | AVEIPDVGNLSQVVVMKSENDQGIRNKCKMNLQDNNGNDIGFIGFHQFN | |
| | NIAKLVASNWYNRQIGKASRTFGCSWEFIPVDDGWGESSL | |
| ^{ci}BoNT/PMP1A | MLQIRVFNYNDPIDGENIVELRYHNRSPVKAFQIVDGIWIIPERYNFTNDT | 96 |
| w/ linker that | KKVPDDRALTILEDEVFAVRENDYLTTDVNEKNSFLNNITKLFKRINSSNI | |
| contains thrombin | GNQLLNYISTSVPYPVVSTNSIKARDYNTIKFDSIDGRRITKSANVLIYGPS | |
| cleavage sites | MKNLLDKQTRAINGEEAKNGIGCLSDIIFSPNYLSVQTVSSSRFVEDPASS | |
| between LC and | LTHQLIHALHNLYGIQYPGEEKFKFGGFIDKLLGTRECIDYEEVLTYGGK | |
| Hn | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCHKAIDGRSLG | |
| | GSLVPRGSGGSAAAYNKTLDCIDVNKEDLYFISDKEGFENIDFSEPEIRYD | |
| | SNVTTATTSSFTDHFLVNRTFNDSDRFPPVELEYAIEPAEIVDNTIMPDIDQ | |
| | KSEISLDNLTTFHYLNAQKMDLGFDSSKEQLKMVTSIEESLLDSKKVYTP | |
| | FTRTAHSVNERISGIAESYLFYQWLKTVINDFTDELNQKSNTDKVADISWI | |
| | IPYVGPALNIGLDLSHGDFTKAFEDLGVSILFAIAPEFATISLVALSIYENIE | |
| | EDSQKEKVINKVENTLARRIEKWHQVYAFMVAQWWGMVHTQIDTRIH | |
| | QMYESLSHQIIAIKANMEYQLSHYKGPDNDKLLLKDYIYEAEIALNTSAN | |
| | RAMKNIERFMIESSISYLKNNLIPSVVENLKKFDADTKKNLDQFIDKNSSV | |
| | LGSDLHILKSQVDLELNPTTKVAFNIQSIPDFDINALIDRLGIQIINTSILNLR | |
| | YESNHLIDLSRYASKINIGSKVNFDPIDKNQIQLFNLESSKIEVILKNAIVY | |
| | NSMYENFSTSFWIRIPKYFNSISLNNEYTIINCMENNSGWKVSLNYGEIIW | |
| | TLQDTQEIKQRVVFKYSQMINISDYINRWIFVTITNNRLNNSKIYINGRLID | |
| | QKPISNLGNIHASNNIMFKLDGCRDTHRYIWIKYFNLFDKELNEKEIKDL | |
| | YDNQSNSGILKDFWGDYLQYDKPYYMLNLYDPNKYVDVNNVGIRGYM | |
| | YLKGPRGSVMTTNIYLNSSLYRGTKFIIKKYASGNKDNIVRNNDRVYINV | |
| | VVKNKEYRLATNASQAGVEKILSALEIPDVGNLSQVVVMKSKNDQGITN | |
| | KCKMNLQDNNGNDIGFIGFHQFNNIAKLVASNWYNRQIERSSRTLGCSW | |
| | EFIPVDDGWGERPLQ | |
| ^{ci}BoNT/PMP1B | MLQIRVFNYNDPIDGENIVELRYHNRSPVKAFQIVDGIWIIPERYNFTNDT | 97 |
| w/ linker that | KKVPDDRALTILEDEVFAVRENDYLTTDVNEKNSFLNNITKLFKRINSSNI | |
| contains thrombin | GNQLLNYISTSVPYPVVSTNSIKARDYNTIKFDSIDGRRITKSANVLIYGPS | |
| cleavage sites | MKNLLDKQTRAINGEEAKNGIGCLSDIIFSPNYLSVQTVSSSRFVEDPASS | |
| between LC and | LTHQLIHALHNLYGIQYPGEEKFKFGGFIDKLLGTRECIDYEEVLTYGGK | |
| Hn | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCHKAIDGRSLG | |
| | GSLVPRGSGGSAAAYNKTLDCIDVNKEDLYFISDKEGFENIDFSEPEIRYD | |
| | SNVTTATTSSFTDHFLVNRTFNDSDRFPPVELEYAIEPAEIVDNTIMPDIDQ | |
| | KSEISLDNLTTFHYLNAQKMDLGFDSSKEQLKMVTSIEESLLDSKKVYTP | |
| | FTRTAHSVNERISGIAESYLFYQWLKTVINDFTDELNQKSNTDKVADISWI | |
| | IPYVGPALNIGLDLSHGDFTKAFEDLGVSILFAIAPEFATISLVALSIYENIE | |
| | EDSQKEKVINKVENTLARRIEKWHQVYAFMVAQWWGMVHTQIDTRIH | |
| | QMYESLSHQIIAIKANMEYQLSHYKGPDNDKLLLKDYIYEAEIALNTSAN | |
| | RAMKNIERFMIESSISYLKNNLIPSVVENLKKFDADTKKNLDQFIDKNSSV | |
| | LGSDLHILKSQVDLELNPTTKVAFNIQSIPDFDINALIDRLGIQILNNIILNL | |
| | RYKDNNLIDLSGYGAKVEVYDGVELNDKNQFKLTSSANSKIRVTQNQNI | |
| | IFNSVFLDFSVSFWIRIPKYKNDGIQNYIHNEYTIINCMKNNSGWKISIRGN | |
| | RIIWTLIDINGKTKSVFFEYNIREDISEYINRWFFVTITNNLNNAKIYINGKL | |
| | ESNTDIKDIREVIANGEIIFKLDGDIDRTQFIWMKYFSIFNTELSQSNIEERY | |
| | KIQSYSEYLKDFWGNPLMYNKEYYMFNAGNKNSYIKLKKDSPVGEILTR | |
| | SKYNQNSKYINYRDLYIGEKFIIRRKSNSQSINDDIVRKEDYIYLDFFNLN | |
| | QEWRVYTYKYFKKEEEKLFLAPISDSDEFYNTIQIKEYDEQPTYSCQLLF | |
| | KKDEESTDEIGLIGIHRFYESGIVFEEYKDYFCISKWYLKEVKRKPYNLKL | |
| | GCNWQFIPKDEGWTE | |
| ^{ci}BoNT/PMP1C | MLQIRVFNYNDPIDGENIVELRYHNRSPVKAFQIVDGIWIIPERYNFTNDT | 98 |
| w/ linker that | KKVPDDRALTILEDEVFAVRENDYLTTDVNEKNSFLNNITKLFKRINSSNI | |
| contains thrombin | GNQLLNYISTSVPYPVVSTNSIKARDYNTIKFDSIDGRRITKSANVLIYGPS | |
| cleavage sites | MKNLLDKQTRAINGEEAKNGIGCLSDIIFSPNYLSVQTVSSSRFVEDPASS | |
| between LC and | LTHQLIHALHNLYGIQYPGEEKFKFGGFIDKLLGTRECIDYEEVLTYGGK | |
| Hn | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCHKAIDGRSLG | |
| | GSLVPRGSGGSAAAYNKTLDCIDVNKEDLYFISDKEGFENIDFSEPEIRYD | |
| | SNVTTATTSSFTDHFLVNRTFNDSDRFPPVELEYAIEPAEIVDNTIMPDIDQ | |
| | KSEISLDNLTTFHYLNAQKMDLGFDSSKEQLKMVTSIEESLLDSKKVYTP | |
| | FTRTAHSVNERISGIAESYLFYQWLKTVINDFTDELNQKSNTDKVADISWI | |
| | IPYVGPALNIGLDLSHGDFTKAFEDLGVSILFAIAPEFATISLVALSIYENIE | |
| | EDSQKEKVINKVENTLARRIEKWHQVYAFMVAQWWGMVHTQIDTRIH | |
| | QMYESLSHQIIAIKANMEYQLSHYKGPDNDKLLLKDYIYEAEIALNTSAN | |
| | RAMKNIERFMIESSISYLKNNLIPSVVENLKKFDADTKKNLDQFIDKNSSV | |
| | LGSDLHILKSQVDLELNPTTKVAFNIQSIPDFDINALIDRLGIQINDSKILSL | |
| | QNRKNTLVDTSGYNAEVSEEGDVQLNPIFPFDFKLGSSGEDRGKVIVTQN | |
| | ENIVYNSMYESFSISFWIRINKWVSNLPGYTIIDSVKNNSGWSIGIISNFLVF | |
| | TLKQNEDSEQSINFSYDISNNAPGYNKWFFVTVTNNMMGNMKIYINGKL | |
| | IDTIKVKELTGINFSKTITFEINKIPDTGLITSDSDNINMWIRDFYIFAKELD | |
| | GKDINILFNSLQYTNVVKDYWGNDLRYNKEYYMVNIDYLNRYMYANS | |
| | RQIVFNTRRNNNDFNEGYKIIIKRIRGNTNDTRVRGGDILYFDMTINNKA | |
| | YNLFMKNETMYADNHSTEDIYAIGLREQTKDINDNIIFQIQPMNNTYYYA | |
| | SQIFKSNFNGENISGICSIGTYRFRLGGDWYRHNYLVPTVKQGNYASLLE | |
| | STSTHWGFVPVSE | |
| ^{ci}BoNT/PMP1D | MLQIRVFNYNDPIDGENIVELRYHNRSPVKAFQIVDGIWIIPERYNFTNDT | 99 |
| w/ linker that | KKVPDDRALTILEDEVFAVRENDYLTTDVNEKNSFLNNITKLFKRINSSNI | |
| contains thrombin | GNQLLNYISTSVPYPVVSTNSIKARDYNTIKFDSIDGRRITKSANVLIYGPS | |
| cleavage sites | MKNLLDKQTRAINGEEAKNGIGCLSDIIFSPNYLSVQTVSSSRFVEDPASS | |
| between LC and | LTHQLIHALHNLYGIQYPGEEKFKFGGFIDKLLGTRECIDYEEVLTYGGK | |
| Hn | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCHKAIDGRSLG | |
| | GSLVPRGSGGSAAAYNKTLDCIDVNKEDLYFISDKEGFENIDFSEPEIRYD | |
| | SNVTTATTSSFTDHFLVNRTFNDSDRFPPVELEYAIEPAEIVDNTIMPDIDQ | |
| | KSEISLDNLTTFHYLNAQKMDLGFDSSKEQLKMVTSIEESLLDSKKVYTP | |
| | FTRTAHSVNERISGIAESYLFYQWLKTVINDFTDELNQKSNTDKVADISWI | |
| | IPYVGPALNIGLDLSHGDFTKAFEDLGVSILFAIAPEFATISLVALSIYENIE | |
| | EDSQKEKVINKVENTLARRIEKWHQVYAFMVAQWWGMVHTQIDTRIH | |
| | QMYESLSHQIIAIKANMEYQLSHYKGPDNDKLLLKDYIYEAEIALNTSAN | |
| | RAMKNIERFMIESSISYLKNNLIPSVVENLKKFDADTKKNLDQFIDKNSSV | |
| | LGSDLHILKSQVDLELNPTTKVAFNIQSIPDFDINALIDRLGIQINDSKILSL | |
| | QNKKNALVDTSGYNAEVRVGDNVQLNTIYTNDFKLSSSGDKIIVNLNNN | |
| | ILYSAIYENSSVSFWIKISKDLTNSHNEYTIINSIEQNSGWKLCIRNGNIEWI | |
| | LQDVNRKYKSLIFDYSESLSHTGYTNKWFFVTITNNIMGYMKLYINGELK | |
| | QSQKIEDLDEVKLDKTIVFGIDENIDENQMLWIRDFNIFSKELSNEDINIVY | |
| | EGQILRNVIKDYWGNPLKFDTEYYIINDNYIDRYIAPESNVLVLVQYPDRS | |
| | KLYTGNPITIKSVSDKNPYSRILNGDNIILHMLYNSRKYMIIRDTDTIYATQ | |
| | GGECSQNCVYALKLQSNLGNYGIGIFSIKNIVSKNKYCSQIFSSFRENTML | |
| | LADIYKPWRFSFKNAYTPVAVTNYETKLLSTSSFWKFISRDPGWVE | |
| ^{ci}BoNT/PMP1E | MLQIRVFNYNDPIDGENIVELRYHNRSPVKAFQIVDGIWIIPERYNFTNDT | 100 |
| w/ linker that | KKVPDDRALTILEDEVFAVRENDYLTTDVNEKNSFLNNITKLFKRINSSNI | |
| contains thrombin | GNQLLNYISTSVPYPVVSTNSIKARDYNTIKFDSIDGRRITKSANVLIYGPS | |
| cleavage sites | MKNLLDKQTRAINGEEAKNGIGCLSDIIFSPNYLSVQTVSSSRFVEDPASS | |
| between LC and | LTHQLIHALHNLYGIQYPGEEKFKFGGFIDKLLGTRECIDYEEVLTYGGK | |
| Hn | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCHKAIDGRSLG | |
| | GSLVPRGSGGSAAAYNKTLDCIDVNKEDLYFISDKEGFENIDFSEPEIRYD | |
| | SNVTTATTSSFTDHFLVNRTFNDSDRFPPVELEYAIEPAEIVDNTIMPDIDQ | |
| | KSEISLDNLTTFHYLNAQKMDLGFDSSKEQLKMVTSIEESLLDSKKVYTP | |
| | FTRTAHSVNERISGIAESYLFYQWLKTVINDFTDELNQKSNTDKVADISWI | |
| | IPYVGPALNIGLDLSHGDFTKAFEDLGVSILFAIAPEFATISLVALSIYENIE | |
| | EDSQKEKVINKVENTLARRIEKWHQVYAFMVAQWWGMVHTQIDTRIH | |
| | QMYESLSHQIIAIKANMEYQLSHYKGPDNDKLLLKDYIYEAEIALNTSAN | |
| | RAMKNIERFMIESSISYLKNNLIPSVVENLKKFDADTKKNLDQFIDKNSSV | |
| | LGSDLHILKSQVDLELNPTTKVAFNIQSIPDFDINALIDRLGIQIKSSSVLN | |
| | MRYKNDKYVDTSGYDSNININGDVYKYPTNKNQFGIYNDKLSEVNISQN | |
| | DYIIYDNKYKNFSISFWVRIPNYDNKIVNVNNEYTIINCMRDNNSGWKVS | |
| | LNHNEIIWTLQDNAGINQKLAFNYGNANGISDYINKWIFVTITNDRLGDS | |
| | KLYINGNLIDQKSILNLGNIHVSDNILFKIVNCSYTRYIGIRYFNIFDKELDE | |
| | TEIQTLYSNEPNTNILKDFWGNYLLYDKEYYLLNVLKPNNFIDRRKDSTL | |
| | SINNIRSTILLANRLYSGIKVKIQRVNNSSTNDNLVRKNDQVYINFVASKT | |
| | HLFPLYADTATTNKEKTIKISSSGNRFNQVVVMNSVGNNCTMNFKNNNG | |
| | NNIGLLGFKADTVVASTWYYTHMRDHTNSNGCFWNFISEEHGWQEK | |
| ^{ci}BoNT/PMP1F | MLQIRVFNYNDPIDGENIVELRYHNRSPVKAFQIVDGIWIIPERYNFTNDT | 101 |
| w/ linker that | KKVPDDRALTILEDEVFAVRENDYLTTDVNEKNSFLNNITKLFKRINSSNI | |
| contains thrombin | GNQLLNYISTSVPYPVVSTNSIKARDYNTIKFDSIDGRRITKSANVLIYGPS | |
| cleavage sites | MKNLLDKQTRAINGEEAKNGIGCLSDIIFSPNYLSVQTVSSSRFVEDPASS | |
| between LC and | LTHQLIHALHNLYGIQYPGEEKFKFGGFIDKLLGTRECIDYEEVLTYGGK | |
| Hn | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCHKAIDGRSLG | |
| | GSLVPRGSGGSAAAYNKTLDCIDVNKEDLYFISDKEGFENIDFSEPEIRYD | |
| | SNVTTATTSSFTDHFLVNRTFNDSDRFPPVELEYAIEPAEIVDNTIMPDIDQ | |
| | KSEISLDNLTTFHYLNAQKMDLGFDSSKEQLKMVTSIEESLLDSKKVYTP | |
| | FTRTAHSVNERISGIAESYLFYQWLKTVINDFTDELNQKSNTDKVADISWI | |
| | IPYVGPALNIGLDLSHGDFTKAFEDLGVSILFAIAPEFATISLVALSIYENIE | |
| | EDSQKEKVINKVENTLARRIEKWHQVYAFMVAQWWGMVHTQIDTRIH | |
| | QMYESLSHQIIAIKANMEYQLSHYKGPDNDKLLLKDYIYEAEIALNTSAN | |
| | RAMKNIERFMIESSISYLKNNLIPSVVENLKKFDADTKKNLDQFIDKNSSV | |
| | LGSDLHILKSQVDLELNPTTKVAFNIQSIPDFDINALIDRLGIQIKDNSILD | |
| | MRYENNKFIDISGYGSNISINGDVYIYSTNRNQFGIYSSKPSEVNIAQNNDI | |
| | IYNGRYQNFSISFWVRIPKYFNKVNLNNEYTIIDCIRNNNSGWKISLNYNK | |
| | IIWTLQDTAGNNQKLVFNYTQMISISDYINKWIFVTITNNRLGNSRIYING | |
| | NLIDEKSISNLGDIHVSDNILFKIVGCNDTRYVGIRYFKVFDTELGKTEIET | |
| | LYSDEPDPSILKDFWGNYLLYNKRYYLLNLLRTDKSITQNSNFLNINQQR | |
| | GVYQKPNIFSNTRLYTGVEVIIRKNGSTDISNTDNFVRKNDLAYINVVDR | |
| | DVEYRLYADISIAKPEKIIKLIRTSNSNNSLGQIIVMDSIGNNCTMNFQNNN | |
| | GGNIGLLGFHSNNLVASSWYYNNIRKNTSSNGCFWSFISKEHGWQEN | |
| ^{ci}BoNT/PMP1G | MLQIRVFNYNDPIDGENIVELRYHNRSPVKAFQIVDGIWIIPERYNFTNDT | 102 |
| w/ linker that | KKVPDDRALTILEDEVFAVRENDYLTTDVNEKNSFLNNITKLFKRINSSNI | |
| contains thrombin | GNQLLNYISTSVPYPVVSTNSIKARDYNTIKFDSIDGRRITKSANVLIYGPS | |
| cleavage sites | MKNLLDKQTRAINGEEAKNGIGCLSDIIFSPNYLSVQTVSSSRFVEDPASS | |
| between LC and | LTHQLIHALHNLYGIQYPGEEKFKFGGFIDKLLGTRECIDYEEVLTYGGK | |
| Hn | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCHKAIDGRSLG | |
| | GSLVPRGSGGSAAAYNKTLDCIDVNKEDLYFISDKEGFENIDFSEPEIRYD | |
| | SNVTTATTSSFTDHFLVNRTFNDSDRFPPVELEYAIEPAEIVDNTIMPDIDQ | |
| | KSEISLDNLTTFHYLNAQKMDLGFDSSKEQLKMVTSIEESLLDSKKVYTP | |
| | FTRTAHSVNERISGIAESYLFYQWLKTVINDFTDELNQKSNTDKVADISWI | |
| | IPYVGPALNIGLDLSHGDFTKAFEDLGVSILFAIAPEFATISLVALSIYENIE | |
| | EDSQKEKVINKVENTLARRIEKWHQVYAFMVAQWWGMVHTQIDTRIH | |
| | QMYESLSHQIIAIKANMEYQLSHYKGPDNDKLLLKDYIYEAEIALNTSAN | |
| | RAMKNIERFMIESSISYLKNNLIPSVVENLKKFDADTKKNLDQFIDKNSSV | |
| | LGSDLHILKSQVDLELNPTTKVAFNIQSIPDFDINALIDRLGIQISSNAILSLS | |
| | YRGGRLIDSSGYGATMNVGSDVIFNDIGNGQFKLNNSENSNITAHQSKFV | |
| | VYDSMFDNFSINFWVRTPKYNNNDIQTYLQNEYTIISCIKNDSGWKVSIK | |
| | GNRIIWTLIDVNAKSKSIFFEYSIKDNISDYINKWFSITITNDRLGNANIYIN | |
| | GSLKKSEKILNLDRINSSNDIDFKLINCTDTTKFVWIKDFNIFGRELNATEV | |
| | SSLYWIQSSTNTLKDFWGNPLRYDTQYYLFNQGMQNIYIKYFSKASMGE | |
| | TAPRTNFNNAAINYQNLYLGLRFIIKKASNSRNINNDNIVREGDYIYLNID | |
| | NISDESYRVYVLVNSKEIQTQLFLAPINDDPTFYDVLQIKKYYEKTTYNC | |
| | QILCEKDTKTFGLFGIGKFVKDYGYVWDTYDNYFCISQWYLRRISENINK | |
| | LRLGCNWQFIPVDEGWTE | |
| ^{ci}BoNT/PMP1H | MLQIRVFNYNDPIDGENIVELRYHNRSPVKAFQIVDGIWIIPERYNFTNDT | 103 |
| w/ linker that | KKVPDDRALTILEDEVFAVRENDYLTTDVNEKNSFLNNITKLFKRINSSNI | |
| contains thrombin | GNQLLNYISTSVPYPVVSTNSIKARDYNTIKFDSIDGRRITKSANVLIYGPS | |
| cleavage sites | MKNLLDKQTRAINGEEAKNGIGCLSDIIFSPNYLSVQTVSSSRFVEDPASS | |
| between LC and | LTHQLIHALHNLYGIQYPGEEKFKFGGFIDKLLGTRECIDYEEVLTYGGK | |
| Hn | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKLCHKAIDGRSLG | |
| | GSLVPRGSGGSAAAYNKTLDCIDVNKEDLYFISDKEGFENIDFSEPEIRYD | |
| | SNVTTATTSSFTDHFLVNRTFNDSDRFPPVELEYAIEPAEIVDNTIMPDIDQ | |
| | KSEISLDNLTTFHYLNAQKMDLGFDSSKEQLKMVTSIEESLLDSKKVYTP | |
| | FTRTAHSVNERISGIAESYLFYQWLKTVINDFTDELNQKSNTDKVADISWI | |
| | IPYVGPALNIGLDLSHGDFTKAFEDLGVSILFAIAPEFATISLVALSIYENIE | |
| | EDSQKEKVINKVENTLARRIEKWHQVYAFMVAQWWGMVHTQIDTRIH | |
| | QMYESLSHQIIAIKANMEYQLSHYKGPDNDKLLLKDYIYEAEIALNTSAN | |
| | RAMKNIERFMIESSISYLKNNLIPSVVENLKKFDADTKKNLDQFIDKNSSV | |
| | LGSDLHILKSQVDLELNPTTKVAFNIQSIPDFDINALIDRLGIQLKYNCILNI | |
| | KYEMDRDKLVDSSGYRSRINIGTGVKFSEIDKNQVQLSNLESSKIEVILNN | |
| | GVIYNSMYENFSTSFWIRIPKYFRNINNEYKIISCMQNNSGWEVSLNFSN | |
| | MNSKIIWTLQDTEGIKKTVVFQYTQNINISDYINRWIFVTITNNRLSNSKIY | |
| | INGRLINEESISDLGNIHASNNIMFKLDGCRDPHRYIWIKYFNLFDKELNK | |
| | KEIKDLYDNQSNSGILKDFWGDYLQYDKPYYMLNLYDPNKYLDVNNVG | |
| | IRGYMYLKGPRGRIVTTNIYLNSTLYMGTKFIIKKYASGNKDNIVRNNDR | |
| | VYINVVVKNKEYRLATNASQAGVEKILSAVEIPDVGNLSQVVVMKSEND | |
| | QGIRNKCKMNLQDNNGNDIGFIGFHQFNNIAKLVASNWYNRQIGKASRT | |
| | FGCSWEFIPVDDGWGESSL | |
| ^{ci}LC/X | MKLEINKFNYNDPIDGINVITMRPPRHSDKINKGKGPFKAFQVIKNIWIVP | 39 |
| | ERYNFTNNTNDLNIPSEPIMEADAIYNPNYLNTPSEKDEFLQGVIKVLERI | |
| | KSKPEGEKLLELISSSIPLPLVSNGALTLSDNETIAYQENNNIVSNLQANLV | |
| | IYGPGPDIANNATYGLYSTPISNGEGTLSEVSFSPFYLKPFDESYGNYRSL | |
| | VNIVNKFVKREFAPDPASTLMHQLVHVTHNLYGISNRNFYYNFDTGKIET | |
| | SRQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKI | |
| Hn/X-Hc/A | IEVENKDLFLISNKDSLNDINLSEEKIKPETTVFFKDKLPPQDITLSNYDFT | 40 |
| | EANSIPSISQQNILERNEELYEPIRNSLFEIKTIYVDKLTTFHFLEAQNIDESI | |
| | DSSKIRVELTDSVDEALSNPNKVYSPFKNMSNTINSIETGITSTYIFYQWLR | |
| | SIVKDFSDETGKIDVIDKSSDTLAIVPYIGPLLNIGNDIRHGDFVGAIELAGI | |
| | TALLEYVPEFTIPILVGLEVIGGELAREQVEAIVNNALDKRDQKWAEVYN | |
| | ITKAQWWGTIHLQINTRLAHTYKALSRQANAIKMNMEFQLANYKGNID | |
| | DKAKIKNAISETEILLNKSVEQAMKNTEKFMIKLSNSYLTKEMIPKVQDN | |
| | LKNFDLETKKTLDKFIKEKEDILGTNLSSSLRRKVSIRLNKNIAFDINDIPFS | |
| | EFDDLINQYKNEIEDYEVLNLGAEDGKIKDLSGTTSDINIGSDIEIINTSILN | |
| | LRYESNHLIDLSRYASKINIGSKVNFDPIDKNQIQLFNLESSKIEVILKNAIV | |
| | YNSMYENFSTSFWIRIPKYFNSISLNNEYTIINCMENNSGWKVSLNYGEII | |
| | WTLQDTQEIKQRVVFKYSQMINISDYINRWIFVTITNNRLNNSKIYINGRL | |
| | IDQKPISNLGNIHASNNIMFKLDGCRDTHRYIWIKYFNLFDKELNEKEIKD | |
| | LYDNQSNSGILKDFWGDYLQYDKPYYMLNLYDPNKYVDVNNVGIRGY | |
| | MYLKGPRGSVMTTNIYLNSSLYRGTKFIIKKYASGNKDNIVRNNDRVYIN | |
| | VVVKNKEYRLATNASQAGVEKILSALEIPDVGNLSQVVVMKSKNDQGIT | |
| | NKCKMNLQDNNGNDIGFIGFHQFNNIAKLVASNWYNRQIERSSRTLGCS | |
| | WEFIPVDDGWGERPLQ | |
| Hn/X-Hc/B | IEVENKDLFLISNKDSLNDINLSEEKIKPETTVFFKDKLPPQDITLSNYDFT | 41 |
| | EANSIPSISQQNILERNEELYEPIRNSLFEIKTIYVDKLTTFHFLEAQNIDESI | |
| | DSSKIRVELTDSVDEALSNPNKVYSPFKNMSNTINSIETGITSTYIFYQWLR | |
| | SIVKDFSDETGKIDVIDKSSDTLAIVPYIGPLLNIGNDIRHGDFVGAIELAGI | |
| | TALLEYVPEFTIPILVGLEVIGGELAREQVEAIVNNALDKRDQKWAEVYN | |
| | ITKAQWWGTIHLQINTRLAHTYKALSRQANAIKMNMEFQLANYKGNID | |
| | DKAKIKNAISETEILLNKSVEQAMKNTEKFMIKLSNSYLTKEMIPKVQDN | |
| | LKNFDLETKKTLDKFIKEKEDILGTNLSSSLRRKVSIRLNKNIAFDINDIPFS | |
| | EFDDLINQYKNEIEDYEVLNLGAEDGKIKDLSGTTSDINIGSDIEILNNIILN | |
| | LRYKDNNLIDLSGYGAKVEVYDGVELNDKNQFKLTSSANSKIRVTQNQN | |
| | IIFNSVFLDFSVSFWIRIPKYKNDGIQNYIHNEYTIINCMKNNSGWKISIRG | |
| | NRIIWTLIDINGKTKSVFFEYNIREDISEYINRWFFVTITNNLNNAKIYINGK | |
| | LESNTDIKDIREVIANGEIIFKLDGDIDRTQFIWMKYFSIFNTELSQSNIEER | |
| | YKIQSYSEYLKDFWGNPLMYNKEYYMFNAGNKNSYIKLKKDSPVGEILT | |
| | RSKYNQNSKYINYRDLYIGEKFIIRRKSNSQSINDDIVRKEDYIYLDFFNLN | |
| | QEWRVYTYKYFKKEEEKLFLAPISDSDEFYNTIQIKEYDEQPTYSCQLLF | |
| | KKDEESTDEIGLIGIHRFYESGIVFEEYKDYFCISKWYLKEVKRKPYNLKL | |
| | GCNWQFIPKDEGWTE | |
| Hn/X-Hc/C | IEVENKDLFLISNKDSLNDINLSEEKIKPETTVFFKDKLPPQDITLSNYDFT | 42 |
| | EANSIPSISQQNILERNEELYEPIRNSLFEIKTIYVDKLTTFHFLEAQNIDESI | |
| | DSSKIRVELTDSVDEALSNPNKVYSPFKNMSNTINSIETGITSTYIFYQWLR | |
| | SIVKDFSDETGKIDVIDKSSDTLAIVPYIGPLLNIGNDIRHGDFVGAIELAGI | |
| | TALLEYVPEFTIPILVGLEVIGGELAREQVEAIVNNALDKRDQKWAEVYN | |
| | ITKAQWWGTIHLQINTRLAHTYKALSRQANAIKMNMEFQLANYKGNID | |
| | DKAKIKNAISETEILLNKSVEQAMKNTEKFMIKLSNSYLTKEMIPKVQDN | |
| | LKNFDLETKKTLDKFIKEKEDILGTNLSSSLRRKVSIRLNKNIAFDINDIPFS | |
| | EFDDLINQYKNEIEDYEVLNLGAEDGKIKDLSGTTSDINIGSDIEINDSKIL | |
| | SLQNRKNTLVDTSGYNAEVSEEGDVQLNPIFPFDFKLGSSGEDRGKVIVT | |
| | QNENIVYNSMYESFSISFWIRINKWVSNLPGYTIIDSVKNNSGWSIGIISNF | |
| | LVFTLKQNEDSEQSINFSYDISNNAPGYNKWFFVTVTNNMMGNMKIYIN | |
| | GKLIDTIKVKELTGINFSKTITFEINKIPDTGLITSDSDNINMWIRDFYIFAK | |
| | ELDGKDINILFNSLQYTNVVKDYWGNDLRYNKEYYMVNIDYLNRYMY | |
| | ANSRQIVFNTRRNNNDFNEGYKIIIKRIRGNTNDTRVRGGDILYFDMTINN | |
| | KAYNLFMKNETMYADNHSTEDIYAIGLREQTKDINDNIIFQIQPMNNTYY | |
| | YASQIFKSNFNGENISGICSIGTYRFRLGGDWYRHNYLVPTVKQGNYASL | |
| | LESTSTHWGFVPVSE | |
| Hn/X-Hc/D | IEVENKDLFLISNKDSLNDINLSEEKIKPETTVFFKDKLPPQDITLSNYDFT | 43 |
| | EANSIPSISQQNILERNEELYEPIRNSLFEIKTIYVDKLTTFHFLEAQNIDESI | |
| | DSSKIRVELTDSVDEALSNPNKVYSPFKNMSNTINSIETGITSTYIFYQWLR | |
| | SIVKDFSDETGKIDVIDKSSDTLAIVPYIGPLLNIGNDIRHGDFVGAIELAGI | |
| | TALLEYVPEFTIPILVGLEVIGGELAREQVEAIVNNALDKRDQKWAEVYN | |
| | ITKAQWWGTIHLQINTRLAHTYKALSRQANAIKMNMEFQLANYKGNID | |
| | DKAKIKNAISETEILLNKSVEQAMKNTEKFMIKLSNSYLTKEMIPKVQDN | |
| | LKNFDLETKKTLDKFIKEKEDILGTNLSSSLRRKVSIRLNKNIAFDINDIPFS | |
| | EFDDLINQYKNEIEDYEVLNLGAEDGKIKDLSGTTSDINIGSDIEINDSKIL | |
| | SLQNKKNALVDTSGYNAEVRVGDNVQLNTIYTNDFKLSSSGDKIIVNLN | |
| | NNILYSAIYENSSVSFWIKISKDLTNSHNEYTIINSIEQNSGWKLCIRNGNIE | |
| | WILQDVNRKYKSLIFDYSESLSHTGYTNKWFFVTITNNIMGYMKLYINGE | |
| | LKQSQKIEDLDEVKLDKTIVFGIDENIDENQMLWIRDFNIFSKELSNEDINI | |
| | VYEGQILRNVIKDYWGNPLKFDTEYYIINDNYIDRYIAPESNVLVLVQYP | |
| | DRSKLYTGNPITIKSVSDKNPYSRILNGDNIILHMLYNSRKYMIIRDTDTIY | |
| | ATQGGECSQNCVYALKLQSNLGNYGIGIFSIKNIVSKNKYCSQIFSSFREN | |
| | TMLLADIYKPWRFSFKNAYTPVAVTNYETKLLSTSSFWKFISRDPGWVE | |
| Hn/X-Hc/E | IEVENKDLFLISNKDSLNDINLSEEKIKPETTVFFKDKLPPQDITLSNYDFT | 44 |
| | EANSIPSISQQNILERNEELYEPIRNSLFEIKTIYVDKLTTFHFLEAQNIDESI | |
| | DSSKIRVELTDSVDEALSNPNKVYSPFKNMSNTINSIETGITSTYIFYQWLR | |
| | SIVKDFSDETGKIDVIDKSSDTLAIVPYIGPLLNIGNDIRHGDFVGAIELAGI | |
| | TALLEYVPEFTIPILVGLEVIGGELAREQVEAIVNNALDKRDQKWAEVYN | |
| | ITKAQWWGTIHLQINTRLAHTYKALSRQANAIKMNMEFQLANYKGNID | |
| | DKAKIKNAISETEILLNKSVEQAMKNTEKFMIKLSNSYLTKEMIPKVQDN | |
| | LKNFDLETKKTLDKFIKEKEDILGTNLSSSLRRKVSIRLNKNIAFDINDIPFS | |
| | EFDDLINQYKNEIEDYEVLNLGAEDGKIKDLSGTTSDINIGSDIEIKSSSVL | |
| | NMRYKNDKYVDTSGYDSNININGDVYKYPTNKNQFGIYNDKLSEVNISQ | |
| | NDYIIYDNKYKNFSISFWVRIPNYDNKIVNVNNEYTIINCMRDNNSGWKV | |
| | SLNHNEIIWTLQDNAGINQKLAFNYGNANGISDYINKWIFVTITNDRLGD | |
| | SKLYINGNLIDQKSILNLGNIHVSDNILFKIVNCSYTRYIGIRYFNIFDKELD | |
| | ETEIQTLYSNEPNTNILKDFWGNYLLYDKEYYLLNVLKPNNFIDRRKDST | |
| | LSINNIRSTILLANRLYSGIKVKIQRVNNSSTNDNLVRKNDQVYINFVASK | |
| | THLFPLYADTATTNKEKTIKISSSGNRFNQVVVMNSVGNNCTMNFKNNN | |
| | GNNIGLLGFKADTVVASTWYYTHMRDHTNSNGCFWNFISEEHGWQEK | |
| Hn/X-Hc/F | IEVENKDLFLISNKDSLNDINLSEEKIKPETTVFFKDKLPPQDITLSNYDFT | 45 |
| | EANSIPSISQQNILERNEELYEPIRNSLFEIKTIYVDKLTTFHFLEAQNIDESI | |
| | DSSKIRVELTDSVDEALSNPNKVYSPFKNMSNTINSIETGITSTYIFYQWLR | |
| | SIVKDFSDETGKIDVIDKSSDTLAIVPYIGPLLNIGNDIRHGDFVGAIELAGI | |
| | TALLEYVPEFTIPILVGLEVIGGELAREQVEAIVNNALDKRDQKWAEVYN | |
| | ITKAQWWGTIHLQINTRLAHTYKALSRQANAIKMNMEFQLANYKGNID | |
| | DKAKIKNAISETEILLNKSVEQAMKNTEKFMIKLSNSYLTKEMIPKVQDN | |
| | LKNFDLETKKTLDKFIKEKEDILGTNLSSSLRRKVSIRLNKNIAFDINDIPFS | |
| | EFDDLINQYKNEIEDYEVLNLGAEDGKIKDLSGTTSDINIGSDIEIKDNSIL | |
| | DMRYENNKFIDISGYGSNISINGDVYIYSTNRNQFGIYSSKPSEVNIAQNN | |
| | DIIYNGRYQNFSISFWVRIPKYFNKVNLNNEYTIIDCIRNNNSGWKISLNY | |
| | NKIIWTLQDTAGNNQKLVFNYTQMISISDYINKWIFVTITNNRLGNSRIYI | |
| | NGNLIDEKSISNLGDIHVSDNILFKIVGCNDTRYVGIRYFKVFDTELGKTEI | |
| | ETLYSDEPDPSILKDFWGNYLLYNKRYYLLNLLRTDKSITQNSNFLNINQ | |
| | QRGVYQKPNIFSNTRLYTGVEVIIRKNGSTDISNTDNFVRKNDLAYINVV | |
| | DRDVEYRLYADISIAKPEKIIKLIRTSNSNNSLGQIIVMDSIGNNCTMNFQN | |
| | NNGGNIGLLGFHSNNLVASSWYYNNIRKNTSSNGCFWSFISKEHGWQEN | |
| Hn/X-Hc/G | IEVENKDLFLISNKDSLNDINLSEEKIKPETTVFFKDKLPPQDITLSNYDFT | 46 |
| | EANSIPSISQQNILERNEELYEPIRNSLFEIKTIYVDKLTTFHFLEAQNIDESI | |
| | DSSKIRVELTDSVDEALSNPNKVYSPFKNMSNTINSIETGITSTYIFYQWLR | |
| | SIVKDFSDETGKIDVIDKSSDTLAIVPYIGPLLNIGNDIRHGDFVGAIELAGI | |
| | TALLEYVPEFTIPILVGLEVIGGELAREQVEAIVNNALDKRDQKWAEVYN | |
| | ITKAQWWGTIHLQINTRLAHTYKALSRQANAIKMNMEFQLANYKGNID | |
| | DKAKIKNAISETEILLNKSVEQAMKNTEKFMIKLSNSYLTKEMIPKVQDN | |
| | LKNFDLETKKTLDKFIKEKEDILGTNLSSSLRRKVSIRLNKNIAFDINDIPFS | |
| | EFDDLINQYKNEIEDYEVLNLGAEDGKIKDLSGTTSDINIGSDIEISSNAILS | |
| | LSYRGGRLIDSSGYGATMNVGSDVIFNDIGNGQFKLNNSENSNITAHQSK | |
| | FVVYDSMFDNFSINFWVRTPKYNNNDIQTYLQNEYTIISCIKNDSGWKVS | |
| | IKGNRIIWTLIDVNAKSKSIFFEYSIKDNISDYINKWFSITITNDRLGNANIYI | |
| | NGSLKKSEKILNLDRINSSNDIDFKLINCTDTTKFVWIKDFNIFGRELNATE | |
| | VSSLYWIQSSTNTLKDFWGNPLRYDTQYYLFNQGMQNIYIKYFSKASMG | |
| | ETAPRTNFNNAAINYQNLYLGLRFIIKKASNSRNINNDNIVREGDYIYLNI | |
| | DNISDESYRVYVLVNSKEIQTQLFLAPINDDPTFYDVLQIKKYYEKTTYN | |
| | CQILCEKDTKTFGLFGIGKFVKDYGYVWDTYDNYFCISQWYLRRISENIN | |
| | KLRLGCNWQFIPVDEGWTE | |
| Hn/X-Hc/H | IEVENKDLFLISNKDSLNDINLSEEKIKPETTVFFKDKLPPQDITLSNYDFT | 47 |
| | EANSIPSISQQNILERNEELYEPIRNSLFEIKTIYVDKLTTFHFLEAQNIDESI | |
| | DSSKIRVELTDSVDEALSNPNKVYSPFKNMSNTINSIETGITSTYIFYQWLR | |
| | SIVKDFSDETGKIDVIDKSSDTLAIVPYIGPLLNIGNDIRHGDFVGAIELAGI | |
| | TALLEYVPEFTIPILVGLEVIGGELAREQVEAIVNNALDKRDQKWAEVYN | |
| | ITKAQWWGTIHLQINTRLAHTYKALSRQANAIKMNMEFQLANYKGNID | |
| | DKAKIKNAISETEILLNKSVEQAMKNTEKFMIKLSNSYLTKEMIPKVQDN | |
| | LKNFDLETKKTLDKFIKEKEDILGTNLSSSLRRKVSIRLNKNIAFDINDIPFS | |
| | EFDDLINQYKNEIEDYEVLNLGAEDGKIKDLSGTTSDINIGSDIELKYNCIL | |
| | NIKYEMDRDKLVDSSGYRSRINIGTGVKFSEIDKNQVQLSNLESSKIEVIL | |
| | NNGVIYNSMYENFSTSFWIRIPKYFRNINNEYKIISCMQNNSGWEVSLNFS | |
| | NMNSKIIWTLQDTEGIKKTVVFQYTQNINISDYINRWIFVTITNNRLSNSKI | |
| | YINGRLINEESISDLGNIHASNNIMFKLDGCRDPHRYIWIKYFNLFDKELN | |
| | KKEIKDLYDNQSNSGILKDFWGDYLQYDKPYYMLNLYDPNKYLDVNNV | |
| | GIRGYMYLKGPRGRIVTTNIYLNSTLYMGTKFIIKKYASGNKDNIVRNND | |
| | RVYINVVVKNKEYRLATNASQAGVEKILSAVEIPDVGNLSQVVVMKSEN | |
| | DQGIRNKCKMNLQDNNGNDIGFIGFHQFNNIAKLVASNWYNRQIGKASR | |
| | TFGCSWEFIPVDDGWGESSL | |
| ^{ci}LC/En | MVTINDLHYSDPIDEDNIINMRIPLYDLEVDDQFINHNVPDLKAFQVFPNV | 48 |
| | WVVPERYTFYSTMKNLDAPANPSRSSYYDPTYLQSDAEKEVFLQQMILL | |
| | FKRINSTQEGQQFLNLLSRSIPVPYESNGDVAMGTTQVIKQMDDKGNVL | |
| | KHRRAHIIIYGPGPDLMAKGSKALTKSRETGRGCMAEIYFSPMYHKTYST | |
| | KLTNKNSLVDKSVQEFVPDPAVTLIHQLCHGLHALYGIDLGNVGSWEFN | |
| | SNPNSLFSSWFSSKEAVNFEEVMTFGGEDVKVIKSEIDKKIPGILNLIKTTV | |
| | EPIINKITDPHDEMLQCLQSKYPSLKGTLGQFFFDDTQLEKDIRDLWMVM | |
| | NETMFAENLKALTAARFLVPKVENIVQVDILSPNVYTIDKGFNHLSKGFK | |
| | GQSVSQSYFRKISALARGAVVRA | |
| Hn/En-Hc/A | IEILEDDLFIMSSKDSFTDTDFSEPSVGPVSYKAKKGADTILDSTLSNYDFS | 49 |
| | KEINFTSTVPIITVEDPLETDEDVPVISEDRTVYVDDYTTFHFLEAQKIGKE | |
| | VVPTQTKVVFTTNMEEALFDSKKVYTVFENTASRINEAGTGIANGMMFY | |
| | QWLKGIVQDFTEEATQKDTFDKISDVTMIVPYLGNILNIGNDIRKGDFMG | |
| | AVELGGVTILLEAIPELTLPVLIGLTIIEDELEKEQVSQTVYNVLDKRDEK | |
| | WEEVYGFVKQQWWWMVHTQFETRILHAYQALNHQVEAIKANMTYQL | |
| | ANYRGNQEDKELLEKAIDDTLQSLYYAVDQAMHNIKRFLIQSSKSYLLN | |
| | QMLPKTKEQLLAFDQQTLRNVNDFINKNQGVLGESLAKDLKKKVEKRL | |
| | TSLPVFNLEDLPISEFEDLIHSHEIDIQDSEVLNIGVNNGKIQDLSGENTPLT | |
| | LGENLHIINTSILNLRYESNHLIDLSRYASKINIGSKVNFDPIDKNQIQLFNL | |
| | ESSKIEVILKNAIVYNSMYENFSTSFWIRIPKYFNSISLNNEYTIINCMENNS | |
| | GWKVSLNYGEIIWTLQDTQEIKQRVVFKYSQMINISDYINRWIFVTITNNR | |
| | LNNSKIYINGRLIDQKPISNLGNIHASNNIMFKLDGCRDTHRYIWIKYFNL | |
| | FDKELNEKEIKDLYDNQSNSGILKDFWGDYLQYDKPYYMLNLYDPNKY | |
| | VDVNNVGIRGYMYLKGPRGSVMTTNIYLNSSLYRGTKFIIKKYASGNKD | |
| | NIVRNNDRVYINVVVKNKEYRLATNASQAGVEKILSALEIPDVGNLSQV | |
| | VVMKSKNDQGITNKCKMNLQDNNGNDIGFIGFHQFNNIAKLVASNWYN | |
| | RQIERSSRTLGCSWEFIPVDDGWGERPLQ | |
| Hn/En-Hc/B | IEILEDDLFIMSSKDSFTDTDFSEPSVGPVSYKAKKGADTILDSTLSNYDFS | 50 |
| | KEINFTSTVPIITVEDPLETDEDVPVISEDRTVYVDDYTTFHFLEAQKIGKE | |
| | VVPTQTKVVFTTNMEEALFDSKKVYTVFENTASRINEAGTGIANGMMFY | |
| | QWLKGIVQDFTEEATQKDTFDKISDVTMIVPYLGNILNIGNDIRKGDFMG | |
| | AVELGGVTILLEAIPELTLPVLIGLTIIEDELEKEQVSQTVYNVLDKRDEK | |
| | WEEVYGFVKQQWWWMVHTQFETRILHAYQALNHQVEAIKANMTYQL | |
| | ANYRGNQEDKELLEKAIDDTLQSLYYAVDQAMHNIKRFLIQSSKSYLLN | |
| | QMLPKTKEQLLAFDQQTLRNVNDFINKNQGVLGESLAKDLKKKVEKRL | |
| | TSLPVFNLEDLPISEFEDLIHSHEIDIQDSEVLNIGVNNGKIQDLSGENTPLT | |
| | LGENLHILNNIILNLRYKDNNLIDLSGYGAKVEVYDGVELNDKNQFKLTS | |
| | SANSKIRVTQNQNIIFNSVFLDFSVSFWIRIPKYKNDGIQNYIHNEYTIINC | |
| | MKNNSGWKISIRGNRIIWTLIDINGKTKSVFFEYNIREDISEYINRWFFVTI | |
| | TNNLNNAKIYINGKLESNTDIKDIREVIANGEIIFKLDGDIDRTQFIWMKYF | |
| | SIFNTELSQSNIEERYKIQSYSEYLKDFWGNPLMYNKEYYMFNAGNKNS | |
| | YIKLKKDSPVGEILTRSKYNQNSKYINYRDLYIGEKFIIRRKSNSQSINDDI | |
| | VRKEDYIYLDFFNLNQEWRVYTYKYFKKEEEKLFLAPISDSDEFYNTIQI | |
| | KEYDEQPTYSCQLLFKKDEESTDEIGLIGIHRFYESGIVFEEYKDYFCISK | |
| | WYLKEVKRKPYNLKLGCNWQFIPKDEGWTE | |
| Hn/En-Hc/C | IEILEDDLFIMSSKDSFTDTDFSEPSVGPVSYKAKKGADTILDSTLSNYDFS | 51 |
| | KEINFTSTVPIITVEDPLETDEDVPVISEDRTVYVDDYTTFHFLEAQKIGKE | |
| | VVPTQTKVVFTTNMEEALFDSKKVYTVFENTASRINEAGTGIANGMMFY | |
| | QWLKGIVQDFTEEATQKDTFDKISDVTMIVPYLGNILNIGNDIRKGDFMG | |
| | AVELGGVTILLEAIPELTLPVLIGLTIIEDELEKEQVSQTVYNVLDKRDEK | |
| | WEEVYGFVKQQWWWMVHTQFETRILHAYQALNHQVEAIKANMTYQL | |
| | ANYRGNQEDKELLEKAIDDTLQSLYYAVDQAMHNIKRFLIQSSKSYLLN | |
| | QMLPKTKEQLLAFDQQTLRNVNDFINKNQGVLGESLAKDLKKKVEKRL | |
| | TSLPVFNLEDLPISEFEDLIHSHEIDIQDSEVLNIGVNNGKIQDLSGENTPLT | |
| | LGENLHINDSKILSLQNRKNTLVDTSGYNAEVSEEGDVQLNPIFPFDFKLG | |
| | SSGEDRGKVIVTQNENIVYNSMYESFSISFWIRINKWVSNLPGYTIIDSVK | |
| | NNSGWSIGIISNFLVFTLKQNEDSEQSINFSYDISNNAPGYNKWFFVTVTN | |
| | NMMGNMKIYINGKLIDTIKVKELTGINFSKTITFEINKIPDTGLITSDSDNIN | |
| | MWIRDFYIFAKELDGKDINILFNSLQYTNVVKDYWGNDLRYNKEYYMV | |
| | NIDYLNRYMYANSRQIVFNTRRNNNDFNEGYKIIIKRIRGNTNDTRVRGG | |
| | DILYFDMTINNKAYNLFMKNETMYADNHSTEDIYAIGLREQTKDINDNII | |
| | FQIQPMNNTYYYASQIFKSNFNGENISGICSIGTYRFRLGGDWYRHNYLV | |
| | PTVKQGNYASLLESTSTHWGFVPVSE | |
| Hn/En-Hc/D | IEILEDDLFIMSSKDSFTDTDFSEPSVGPVSYKAKKGADTILDSTLSNYDFS | 52 |
| | KEINFTSTVPIITVEDPLETDEDVPVISEDRTVYVDDYTTFHFLEAQKIGKE | |
| | VVPTQTKVVFTTNMEEALFDSKKVYTVFENTASRINEAGTGIANGMMFY | |
| | QWLKGIVQDFTEEATQKDTFDKISDVTMIVPYLGNILNIGNDIRKGDFMG | |
| | AVELGGVTILLEAIPELTLPVLIGLTIIEDELEKEQVSQTVYNVLDKRDEK | |
| | WEEVYGFVKQQWWWMVHTQFETRILHAYQALNHQVEAIKANMTYQL | |
| | ANYRGNQEDKELLEKAIDDTLQSLYYAVDQAMHNIKRFLIQSSKSYLLN | |
| | QMLPKTKEQLLAFDQQTLRNVNDFINKNQGVLGESLAKDLKKKVEKRL | |
| | TSLPVFNLEDLPISEFEDLIHSHEIDIQDSEVLNIGVNNGKIQDLSGENTPLT | |
| | LGENLHINDSKILSLQNKKNALVDTSGYNAEVRVGDNVQLNTIYTNDFK | |
| | LSSSGDKIIVNLNNNILYSAIYENSSVSFWIKISKDLTNSHNEYTIINSIEQN | |
| | SGWKLCIRNGNIEWILQDVNRKYKSLIFDYSESLSHTGYTNKWFFVTITN | |
| | NIMGYMKLYINGELKQSQKIEDLDEVKLDKTIVFGIDENIDENQMLWIRD | |
| | FNIFSKELSNEDINIVYEGQILRNVIKDYWGNPLKFDTEYYIINDNYIDRYI | |
| | APESNVLVLVQYPDRSKLYTGNPITIKSVSDKNPYSRILNGDNIILHMLYN | |
| | SRKYMIIRDTDTIYATQGGECSQNCVYALKLQSNLGNYGIGIFSIKNIVSK | |
| | NKYCSQIFSSFRENTMLLADIYKPWRFSFKNAYTPVAVTNYETKLLSTSS | |
| | FWKFISRDPGWVE | |
| Hn/En-Hc/E | IEILEDDLFIMSSKDSFTDTDFSEPSVGPVSYKAKKGADTILDSTLSNYDFS | 53 |
| | KEINFTSTVPIITVEDPLETDEDVPVISEDRTVYVDDYTTFHFLEAQKIGKE | |
| | VVPTQTKVVFTTNMEEALFDSKKVYTVFENTASRINEAGTGIANGMMFY | |
| | QWLKGIVQDFTEEATQKDTFDKISDVTMIVPYLGNILNIGNDIRKGDFMG | |
| | AVELGGVTILLEAIPELTLPVLIGLTIIEDELEKEQVSQTVYNVLDKRDEK | |
| | WEEVYGFVKQQWWWMVHTQFETRILHAYQALNHQVEAIKANMTYQL | |
| | ANYRGNQEDKELLEKAIDDTLQSLYYAVDQAMHNIKRFLIQSSKSYLLN | |
| | QMLPKTKEQLLAFDQQTLRNVNDFINKNQGVLGESLAKDLKKKVEKRL | |
| | TSLPVFNLEDLPISEFEDLIHSHEIDIQDSEVLNIGVNNGKIQDLSGENTPLT | |
| | LGENLHIKSSSVLNMRYKNDKYVDTSGYDSNININGDVYKYPTNKNQFG | |
| | IYNDKLSEVNISQNDYIIYDNKYKNFSISFWVRIPNYDNKIVNVNNEYTIIN | |
| | CMRDNNSGWKVSLNHNEIIWTLQDNAGINQKLAFNYGNANGISDYINK | |
| | WIFVTITNDRLGDSKLYINGNLIDQKSILNLGNIHVSDNILFKIVNCSYTRY | |
| | IGIRYFNIFDKELDETEIQTLYSNEPNTNILKDFWGNYLLYDKEYYLLNVL | |
| | KPNNFIDRRKDSTLSINNIRSTILLANRLYSGIKVKIQRVNNSSTNDNLVRK | |
| | NDQVYINFVASKTHLFPLYADTATTNKEKTIKISSSGNRFNQVVVMNSVG | |
| | NNCTMNFKNNNGNNIGLLGFKADTVVASTWYYTHMRDHTNSNGCFWN | |
| | FISEEHGWQEK | |
| Hn/En-Hc/F | IEILEDDLFIMSSKDSFTDTDFSEPSVGPVSYKAKKGADTILDSTLSNYDFS | 54 |
| | KEINFTSTVPIITVEDPLETDEDVPVISEDRTVYVDDYTTFHFLEAQKIGKE | |
| | VVPTQTKVVFTTNMEEALFDSKKVYTVFENTASRINEAGTGIANGMMFY | |
| | QWLKGIVQDFTEEATQKDTFDKISDVTMIVPYLGNILNIGNDIRKGDFMG | |
| | AVELGGVTILLEAIPELTLPVLIGLTIIEDELEKEQVSQTVYNVLDKRDEK | |
| | WEEVYGFVKQQWWWMVHTQFETRILHAYQALNHQVEAIKANMTYQL | |
| | ANYRGNQEDKELLEKAIDDTLQSLYYAVDQAMHNIKRFLIQSSKSYLLN | |
| | QMLPKTKEQLLAFDQQTLRNVNDFINKNQGVLGESLAKDLKKKVEKRL | |
| | TSLPVFNLEDLPISEFEDLIHSHEIDIQDSEVLNIGVNNGKIQDLSGENTPLT | |
| | LGENLHIKDNSILDMRYENNKFIDISGYGSNISINGDVYIYSTNRNQFGIYS | |
| | SKPSEVNIAQNNDIIYNGRYQNFSISFWVRIPKYFNKVNLNNEYTIIDCIRN | |
| | NNSGWKISLNYNKIIWTLQDTAGNNQKLVFNYTQMISISDYINKWIFVTIT | |
| | NNRLGNSRIYINGNLIDEKSISNLGDIHVSDNILFKIVGCNDTRYVGIRYFK | |
| | VFDTELGKTEIETLYSDEPDPSILKDFWGNYLLYNKRYYLLNLLRTDKSIT | |
| | QNSNFLNINQQRGVYQKPNIFSNTRLYTGVEVIIRKNGSTDISNTDNFVRK | |
| | NDLAYINVVDRDVEYRLYADISIAKPEKIIKLIRTSNSNNSLGQIIVMDSIG | |
| | NNCTMNFQNNNGGNIGLLGFHSNNLVASSWYYNNIRKNTSSNGCFWSFI | |
| | SKEHGWQEN | |
| Hn/En-Hc/G | IEILEDDLFIMSSKDSFTDTDFSEPSVGPVSYKAKKGADTILDSTLSNYDFS | 55 |
| | KEINFTSTVPIITVEDPLETDEDVPVISEDRTVYVDDYTTFHFLEAQKIGKE | |
| | VVPTQTKVVFTTNMEEALFDSKKVYTVFENTASRINEAGTGIANGMMFY | |
| | QWLKGIVQDFTEEATQKDTFDKISDVTMIVPYLGNILNIGNDIRKGDFMG | |
| | AVELGGVTILLEAIPELTLPVLIGLTIIEDELEKEQVSQTVYNVLDKRDEK | |
| | WEEVYGFVKQQWWWMVHTQFETRILHAYQALNHQVEAIKANMTYQL | |
| | ANYRGNQEDKELLEKAIDDTLQSLYYAVDQAMHNIKRFLIQSSKSYLLN | |
| | QMLPKTKEQLLAFDQQTLRNVNDFINKNQGVLGESLAKDLKKKVEKRL | |
| | TSLPVFNLEDLPISEFEDLIHSHEIDIQDSEVLNIGVNNGKIQDLSGENTPLT | |
| | LGENLHISSNAILSLSYRGGRLIDSSGYGATMNVGSDVIFNDIGNGQFKLN | |
| | NSENSNITAHQSKFVVYDSMFDNFSINFWVRTPKYNNNDIQTYLQNEYTI | |
| | ISCIKNDSGWKVSIKGNRIIWTLIDVNAKSKSIFFEYSIKDNISDYINKWFSI | |
| | TITNDRLGNANIYINGSLKKSEKILNLDRINSSNDIDFKLINCTDTTKFVWI | |
| | KDFNIFGRELNATEVSSLYWIQSSTNTLKDFWGNPLRYDTQYYLFNQGM | |
| | QNIYIKYFSKASMGETAPRTNFNNAAINYQNLYLGLRFIIKKASNSRNINN | |
| | DNIVREGDYIYLNIDNISDESYRVYVLVNSKEIQTQLFLAPINDDPTFYDV | |
| | LQIKKYYEKTTYNCQILCEKDTKTFGLFGIGKFVKDYGYVWDTYDNYFC | |
| | ISQWYLRRISENINKLRLGCNWQFIPVDEGWTE | |
| Hn/En-Hc/H | IEILEDDLFIMSSKDSFTDTDFSEPSVGPVSYKAKKGADTILDSTLSNYDFS | 56 |
| | KEINFTSTVPIITVEDPLETDEDVPVISEDRTVYVDDYTTFHFLEAQKIGKE | |
| | VVPTQTKVVFTTNMEEALFDSKKVYTVFENTASRINEAGTGIANGMMFY | |
| | QWLKGIVQDFTEEATQKDTFDKISDVTMIVPYLGNILNIGNDIRKGDFMG | |
| | AVELGGVTILLEAIPELTLPVLIGLTIIEDELEKEQVSQTVYNVLDKRDEK | |
| | WEEVYGFVKQQWWWMVHTQFETRILHAYQALNHQVEAIKANMTYQL | |
| | ANYRGNQEDKELLEKAIDDTLQSLYYAVDQAMHNIKRFLIQSSKSYLLN | |
| | QMLPKTKEQLLAFDQQTLRNVNDFINKNQGVLGESLAKDLKKKVEKRL | |
| | TSLPVFNLEDLPISEFEDLIHSHEIDIQDSEVLNIGVNNGKIQDLSGENTPLT | |
| | LGENLHLKYNCILNIKYEMDRDKLVDSSGYRSRINIGTGVKFSEIDKNQV | |
| | QLSNLESSKIEVILNNGVIYNSMYENFSTSFWIRIPKYFRNINNEYKIISCM | |
| | QNNSGWEVSLNFSNMNSKIIWTLQDTEGIKKTVVFQYTQNINISDYINRW | |
| | IFVTITNNRLSNSKIYINGRLINEESISDLGNIHASNNIMFKLDGCRDPHRYI | |
| | WIKYFNLFDKELNKKEIKDLYDNQSNSGILKDFWGDYLQYDKPYYMLN | |
| | LYDPNKYLDVNNVGIRGYMYLKGPRGRIVTTNIYLNSTLYMGTKFIIKKY | |
| | ASGNKDNIVRNNDRVYINVVVKNKEYRLATNASQAGVEKILSAVEIPDV | |
| | GNLSQVVVMKSENDQGIRNKCKMNLQDNNGNDIGFIGFHQFNNIAKLV | |
| | ASNWYNRQIGKASRTFGCSWEFIPVDDGWGESSL | |
| ^{ci}LC/PMP1 | MLQIRVFNYNDPIDGENIVELRYHNRSPVKAFQIVDGIWIIPERYNFTNDT | 104 |
| | KKVPDDRALTILEDEVFAVRENDYLTTDVNEKNSFLNNITKLFKRINSSNI | |
| | GNQLLNYISTSVPYPVVSTNSIKARDYNTIKFDSIDGRRITKSANVLIYGPS | |
| | MKNLLDKQTRAINGEEAKNGIGCLSDIIFSPNYLSVQTVSSSRFVEDPASS | |
| | LTHQLIHALHNLYGIQYPGEEKFKFGGFIDKLLGTRECIDYEEVLTYGGK | |
| | DSEIIRKKIDKSLYPDDFVNKYGEMYKRIKGSNPYYPDEKKLKQSFLNRM | |
| | NPFDQNGTFDTKEFKNHLMDLWFGLNESEFAKEKKILVAKHFITKQINPK | |
| | YTELTNDVYTEDKGFVNGQSIDNQNFKIIDDLISKKVKL | |
| Hn/PMP1-Hc/A | IDVNKEDLYFISDKEGFENIDFSEPEIRYDSNVTTATTSSFTDHFLVNRTFN | 105 |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQIINTSILNLRYESNHLIDLSRYASKINIGSKV | |
| | NFDPIDKNQIQLFNLESSKIEVILKNAIVYNSMYENFSTSFWIRIPKYFNSIS | |
| | LNNEYTIINCMENNSGWKVSLNYGEIIWTLQDTQEIKQRVVFKYSQMINI | |
| | SDYINRWIFVTITNNRLNNSKIYINGRLIDQKPISNLGNIHASNNIMFKLDG | |
| | CRDTHRYIWIKYFNLFDKELNEKEIKDLYDNQSNSGILKDFWGDYLQYD | |
| | KPYYMLNLYDPNKYVDVNNVGIRGYMYLKGPRGSVMTTNIYLNSSLYR | |
| | GTKFIIKKYASGNKDNIVRNNDRVYINVVVKNKEYRLATNASQAGVEKI | |
| | LSALEIPDVGNLSQVVVMKSKNDQGITNKCKMNLQDNNGNDIGFIGFHQ | |
| | FNNIAKLVASNWYNRQIERSSRTLGCSWEFIPVDDGWGERPLQ | |
| Hn/PMP1-Hc/B | IDVNKEDLYFISDKEGFENIDFSEPEIRYDSNVTTATTSSFTDHFLVNRTFN | 106 |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQILNNIILNLRYKDNNLIDLSGYGAKVEVYD | |
| | GVELNDKNQFKLTSSANSKIRVTQNQNIIFNSVFLDFSVSFWIRIPKYKND | |
| | GIQNYIHNEYTIINCMKNNSGWKISIRGNRIIWTLIDINGKTKSVFFEYNIR | |
| | EDISEYINRWFFVTITNNLNNAKIYINGKLESNTDIKDIREVIANGEIIFKLD | |
| | GDIDRTQFIWMKYFSIFNTELSQSNIEERYKIQSYSEYLKDFWGNPLMYN | |
| | KEYYMFNAGNKNSYIKLKKDSPVGEILTRSKYNQNSKYINYRDLYIGEKF | |
| | IIRRKSNSQSINDDIVRKEDYIYLDFFNLNQEWRVYTYKYFKKEEEKLFLA | |
| | PISDSDEFYNTIQIKEYDEQPTYSCQLLFKKDEESTDEIGLIGIHRFYESGIV | |
| | FEEYKDYFCISKWYLKEVKRKPYNLKLGCNWQFIPKDEGWTE | |
| Hn/PMP1-Hc/C | IDVNKEDLYFISDKEGFENIDFSEPEIRYDSNVTTATTSSFTDHFLVNRTFN | 107 |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQINDSKILSLQNRKNTLVDTSGYNAEVSEEG | |
| | DVQLNPIFPFDFKLGSSGEDRGKVIVTQNENIVYNSMYESFSISFWIRINK | |
| | WVSNLPGYTIIDSVKNNSGWSIGIISNFLVFTLKQNEDSEQSINFSYDISNN | |
| | APGYNKWFFVTVTNNMMGNMKIYINGKLIDTIKVKELTGINFSKTITFEI | |
| | NKIPDTGLITSDSDNINMWIRDFYIFAKELDGKDINILFNSLQYTNVVKDY | |
| | WGNDLRYNKEYYMVNIDYLNRYMYANSRQIVFNTRRNNNDFNEGYKIII | |
| | KRIRGNTNDTRVRGGDILYFDMTINNKAYNLFMKNETMYADNHSTEDIY | |
| | AIGLREQTKDINDNIIFQIQPMNNTYYYASQIFKSNFNGENISGICSIGTYRF | |
| | RLGGDWYRHNYLVPTVKQGNYASLLESTSTHWGFVPVSE | |
| Hn/PMP1-Hc/D | IDVNKEDLYFISDKEGFENIDFSEPEIRYDSNVTTATTSSFTDHFLVNRTFN | 108 |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSIYLKNNLI | |
| | PSVVENLKKFDADTKKNLDQFIDKNSSVLGSDLHILKSQVDLELNPTTKV | |
| | AFNIQSIPDFDINALIDRLGIQINDSKILSLQNKKNALVDTSGYNAEVRVG | |
| | DNVQLNTIYTNDFKLSSSGDKIIVNLNNNILYSAIYENSSVSFWIKISKDLT | |
| | NSHNEYTIINSIEQNSGWKLCIRNGNIEWILQDVNRKYKSLIFDYSESLSHT | |
| | GYTNKWFFVTITNNIMGYMKLYINGELKQSQKIEDLDEVKLDKTIVFGID | |
| | ENIDENQMLWIRDFNIFSKELSNEDINIVYEGQILRNVIKDYWGNPLKFDT | |
| | EYYIINDNYIDRYIAPESNVLVLVQYPDRSKLYTGNPITIKSVSDKNPYSRI | |
| | LNGDNIILHMLYNSRKYMIIRDTDTIYATQGGECSQNCVYALKLQSNLGN | |
| | YGIGIFSIKNIVSKNKYCSQIFSSFRENTMLLADIYKPWRFSFKNAYTPVA | |
| | VTNYETKLLSTSSFWKFISRDPGWVE | |
| Hn/PMP1-Hc/E | IDVNKEDLYFISDKEGFENIDFSEPEIRYDSNVTTATTSSFTDHFLVNRTFN | 109 |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQIKSSSVLNMRYKNDKYVDTSGYDSNINING | |
| | DVYKYPTNKNQFGIYNDKLSEVNISQNDYIIYDNKYKNFSISFWVRIPNY | |
| | DNKIVNVNNEYTIINCMRDNNSGWKVSLNHNEIIWTLQDNAGINQKLAF | |
| | NYGNANGISDYINKWIFVTITNDRLGDSKLYINGNLIDQKSILNLGNIHVS | |
| | DNILFKIVNCSYTRYIGIRYFNIFDKELDETEIQTLYSNEPNTNILKDFWGN | |
| | YLLYDKEYYLLNVLKPNNFIDRRKDSTLSINNIRSTILLANRLYSGIKVKIQ | |
| | RVNNSSTNDNLVRKNDQVYINFVASKTHLFPLYADTATTNKEKTIKISSS | |
| | GNRFNQVVVMNSVGNNCTMNFKNNNGNNIGLLGFKADTVVASTWYYT | |
| | HMRDHTNSNGCFWNFISEEHGWQEK | |
| Hn/PMP1-Hc/F | IDVNKEDLYFISDKEGFENIDFSEPEIRYDSNVTTATTSSFTDHFLVNRTFN | 110 |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQIKDNSILDMRYENNKFIDISGYGSNISINGD | |
| | VYIYSTNRNQFGIYSSKPSEVNIAQNNDIIYNGRYQNFSISFWVRIPKYFNK | |
| | VNLNNEYTIIDCIRNNNSGWKISLNYNKIIWTLQDTAGNNQKLVFNYTQ | |
| | MISISDYINKWIFVTITNNRLGNSRIYINGNLIDEKSISNLGDIHVSDNILFKI | |
| | VGCNDTRYVGIRYFKVFDTELGKTEIETLYSDEPDPSILKDFWGNYLLYN | |
| | KRYYLLNLLRTDKSITQNSNFLNINQQRGVYQKPNIFSNTRLYTGVEVIIR | |
| | KNGSTDISNTDNFVRKNDLAYINVVDRDVEYRLYADISIAKPEKIIKLIRT | |
| | SNSNNSLGQIIVMDSIGNNCTMNFQNNNGGNIGLLGFHSNNLVASSWYY | |
| | NNIRKNTSSNGCFWSFISKEHGWQEN | |
| Hn/PMP1-Hc/G | IDVNKEDLYFISDKEGFENIDFSEPEIRYDSNVTTATTSSFTDHFLVNRTFN | 111 |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQISSNAILSLSYRGGRLIDSSGYGATMNVGSD | |
| | VIFNDIGNGQFKLNNSENSNITAHQSKFVVYDSMFDNFSINFWVRTPKYN | |
| | NNDIQTYLQNEYTIISCIKNDSGWKVSIKGNRIIWTLIDVNAKSKSIFFEYSI | |
| | KDNISDYINKWFSITITNDRLGNANIYINGSLKKSEKILNLDRINSSNDIDF | |
| | KLINCTDTTKFVWIKDFNIFGRELNATEVSSLYWIQSSTNTLKDFWGNPL | |
| | RYDTQYYLFNQGMQNIYIKYFSKASMGETAPRTNFNNAAINYQNLYLGL | |
| | RFIIKKASNSRNINNDNIVREGDYIYLNIDNISDESYRVYVLVNSKEIQTQL | |
| | FLAPINDDPTFYDVLQIKKYYEKTTYNCQILCEKDTKTFGLFGIGKFVKD | |
| | YGYVWDTYDNYFCISQWYLRRISENINKLRLGCNWQFIPVDEGWTE | |
| Hn/PMP1-Hc/H | IDVNKEDLYFISDKEGFENIDFSEPEIRYDSNVTTATTSSFTDHFLVNRTFN | 112 |
| | DSDRFPPVELEYAIEPAEIVDNTIMPDIDQKSEISLDNLTTFHYLNAQKMD | |
| | LGFDSSKEQLKMVTSIEESLLDSKKVYTPFTRTAHSVNERISGIAESYLFY | |
| | QWLKTVINDFTDELNQKSNTDKVADISWIIPYVGPALNIGLDLSHGDFTK | |
| | AFEDLGVSILFAIAPEFATISLVALSIYENIEEDSQKEKVINKVENTLARRIE | |
| | KWHQVYAFMVAQWWGMVHTQIDTRIHQMYESLSHQIIAIKANMEYQLS | |
| | HYKGPDNDKLLLKDYIYEAEIALNTSANRAMKNIERFMIESSISYLKNNLI | |
| | PSVVENLKKFDADTKKNLDQFIDKNSSVLGSDLHILKSQVDLELNPTTKV | |
| | AFNIQSIPDFDINALIDRLGIQLKYNCILNIKYEMDRDKLVDSSGYRSRINI | |
| | GTGVKFSEIDKNQVQLSNLESSKIEVILNNGVIYNSMYENFSTSFWIRIPK | |
| | YFRNINNEYKIISCMQNNSGWEVSLNFSNMNSKIIWTLQDTEGIKKTVVF | |
| | QYTQNINISDYINRWIFVTITNNRLSNSKIYINGRLINEESISDLGNIHASNN | |
| | IMFKLDGCRDPHRYIWIKYFNLFDKELNKKEIKDLYDNQSNSGILKDFWG | |
| | DYLQYDKPYYMLNLYDPNKYLDVNNVGIRGYMYLKGPRGRIVTTNIYL | |
| | NSTLYMGTKFIIKKYASGNKDNIVRNNDRVYINVVVKNKEYRLATNASQ | |
| | AGVEKILSAVEIPDVGNLSQVVVMKSENDQGIRNKCKMNLQDNNGNDI | |
| | GFIGFHQFNNIAKLVASNWYNRQIGKASRTFGCSWEFIPVDDGWGESSL | |
| Anti-BoNT/A | MGTQVQLVESGGGLVQPGGSLRLSCAASGSIFSIYAMGWYRQAPGKQRE | 57 |
| VHH (referred to | LVAAISSYGSTNYADSVKGRFTISRDNAKNTVYLQMNSLKPEDTAVYYC | |
| herein as A8, also | NADIATMTAVGGFDYWGQGTQVTVSSEPKTPKPQ | |
| referred herein as | | |
| B8) | | |
| immunoglobulin | HVQLQQSGGGLVQPGGSLRLSCAASGSIFSIYAMGWYRQAPGKQRELVA | 58 |
| heavy chain | AISSYGSTNYADSVKGRFTISRDNAKNTVYLQMNSLKPEDTAVYYCNAD | |
| variable region, | IATMTAVGGFDYWGQGTQVTVSSEPKTPKPQP | |
| partial [Vicugna | | |
| pacos] | | |
| ACCESSION | | |
| ACS73863 | | |
| TrxA-thrombin | MSDKIIHLTDDSFDTDVLKADGAILVDFWAEWCGPCKMIAPILDEIADEYQG | 59 |
| site-anti-BoNT/A | KLTVAKLNIDQNPGTAPKYGIRGIPTILLFKNGEVAATKVGALSKGQLKEFLD | |
| VHH (A8) | ANLAGGSLVPRGSGGSEFMGTQVQLVESGGGLVQPGGSLRLSCAASGSIF | |
| | SIYAMGWYRQAPGKQRELVAAISSYGSTNYADSVKGRFTISRDNAKNTV | |
| | YLQMNSLKPEDTAVYYCNADIATMTAVGGFDYWGQGTQVTVSSEPKTP | |
| | KPQ | |
| TrxA-thrombin | MSDKIIHLTDDSFDTDVLKADGAILVDFWAEWCGPCKMIAPILDEIADEYQG | 60 |
| site-ACS73863 | KLTVAKLNIDQNPGTAPKYGIRGIPTILLFKNGEVAATKVGALSKGQLKEFLD | |
| | ANLAGGSLVPRGSGGSEFHVQLQQSGGGLVQPGGSLRLSCAASGSIFSIYA | |
| | MGWYRQAPGKQRELVAAISSYGSTNYADSVKGRFTISRDNAKNTVYLQ | |
| | MNSLKPEDTAVYYCNADIATMTAVGGFDYWGQGTQVTVSSEPKTPKPQ | |
| | P | |
| TrxA-thrombin | MSDKIIHLTDDSFDTDVLKADGAILVDFWAEWCGPCKMIAPILDEIADEYQG | 61 |
| site-anti-BoNT/A | KLTVAKLNIDQNPGTAPKYGIRGIPTILLFKNGEVAATKVGALSKGQLKEFLD | |
| VHH (A8)- | ANLAGGSLVPRGSGGSEFMGTQVQLVESGGGLVQPGGSLRLSCAASGSIF | |
| ^{ci}LC/X | SIYAMGWYRQAPGKQRELVAAISSYGSTNYADSVKGRFTISRDNAKNTV | |
| | YLQMNSLKPEDTAVYYCNADIATMTAVGGFDYWGQGTQVTVSSEPKTP | |
| | KPQGGGGSGGGGSKLMKLEINKFNYNDPIDGINVITMRPPRHSDKINKGK | |
| | GPFKAFQVIKNIWIVPERYNFTNNTNDLNIPSEPIMEADAIYNPNYLNTPS | |
| | EKDEFLQGVIKVLERIKSKPEGEKLLELISSSIPLPLVSNGALTLSDNETIAY | |
| | QENNNIVSNLQANLVIYGPGPDIANNATYGLYSTPISNGEGTLSEVSFSPF | |
| | YLKPFDESYGNYRSLVNIVNKFVKREFAPDPASTLMHQLVHVTHNLYGI | |
| | SNRNFYYNFDTGKIETSRQQNSLIFEELLTFGGIDSKAISSLIIKKIIETAKN | |
| | NYTTLISERLNTVTVENDLLKYIKNKIPVQGRLGNFKLDTAEFEKKLNTIL | |
| | FVLNESNLAQRFSILVAKHFLKERPIDPIYVNILDDNSYSTLEGFNISSQGS | |
| | NDFQGQLLESSYFEKIESNALRAFIKI | |
| TrxA-thrombin | MSDKIIHLTDDSFDTDVLKADGAILVDFWAEWCGPCKMIAPILDEIADEYQG | 62 |
| site-ACS73863- | KLTVAKLNIDQNPGTAPKYGIRGIPTILLFKNGEVAATKVGALSKGQLKEFLD | |
| ^{ci}LC/X | ANLAGGSLVPRGSGGSEFHVQLQQSGGGLVQPGGSLRLSCAASGSIFSIYA | |
| | MGWYRQAPGKQRELVAAISSYGSTNYADSVKGRFTISRDNAKNTVYLQ | |
| | MNS LKPEDTAVYYCNADIATMTAVGGFDYWGQGTQVTVSSEPKTPKPQ | |
| | PGGGGSGGGGSKLMKLEINKFNYNDPIDGINVITMRPPRHSDKINKGKGP | |
| | FKAFQVIKNIWIVPERYNFTNNTNDLNIPSEPIMEADAIYNPNYLNTPSEK | |
| | DEFLQGVIKVLERIKSKPEGEKLLELISSSIPLPLVSNGALTLSDNETIAYQE | |
| | NNNIVSNLQANLVIYGPGPDIANNATYGLYSTPISNGEGTLSEVSFSPFYL | |
| | KPFDESYGNYRSLVNIVNKFVKREFAPDPASTLMHQLVHVTHNLYGISN | |
| | RNFYYNFDTGKIETSRQQNSLIFEELLTFGGIDSKAISSLIIKKIIETAKNNY | |
| | TTLISERLNTVTVENDLLKYIKNKIPVQGRLGNFKLDTAEFEKKLNTILFV | |
| | LNESNLAQRFSILVAKHFLKERPIDPIYVNILDDNSYSTLEGFNISSQGSND | |
| | FQGQLLESSYFEKIESNALRAFIKI | |
| TrxA-thrombin | MSDKIIHLTDDSFDTDVLKADGAILVDFWAEWCGPCKMIAPILDEIADEYQG | 63 |
| site-anti-BoNT/A | KLTVAKLNIDQNPGTAPKYGIRGIPTILLFKNGEVAATKVGALSKGQLKEFLD | |
| VHH (A8)- | ANLAGGSLVPRGSGGSEFMGTQVQLVESGGGLVQPGGSLRLSCAASGSIFSIY | |
| ^{ci}LC/En | AMGWYRQAPGKQRELVAAISSYGSTNYADSVKGRFTISRDNAKNTVYLQMNSL | |
| | KPEDTAVYYCNADIATMTAVGGFDYWGQGTQVTVSSEPKTPKPQGGGGSGG | |
| | GGSKLMVTINDLHYSDPIDEDNIINMRIPLYDLEVDDQFINHNVPDLKAF | |
| | QVFPNVWVVPERYTFYSTMKNLDAPANPSRSSYYDPTYLQSDAEKEVFL | |
| | QQMILLFKRINSTQEGQQFLNLLSRSIPVPYESNGDVAMGTTQVIKQMDD | |
| | KGNVLKHRRAHIIIYGPGPDLMAKGSKALTKSRETGRGCMAEIYFSPMY | |
| | HKTYSTKLTNKNSLVDKSVQEFVPDPAVTLIHQLCHGLHALYGIDLGNV | |
| | GSWEFNSNPNSLFSSWFSSKEAVNFEEVMTFGGEDVKVIKSEIDKKIPGIL | |
| | NLIKTTVEPIINKITDPHDEMLQCLQSKYPSLKGTLGQFFFDDTQLEKDIR | |
| | DLWMVMNETMFAENLKALTAARFLVPKVENIVQVDILSPNVYTIDKGF | |
| | NHLSKGFKGQSVSQSYFRKISALARGAVVRA | |
| TrxA-thrombin | MSDKIIHLTDDSFDTDVLKADGAILVDFWAEWCGPCKMIAPILDEIADEYQG | 64 |
| site-ACS73863- | KLTVAKLNIDQNPGTAPKYGIRGIPTILLFKNGEVAATKVGALSKGQLKEFLD | |
| ^{ci}LC/En | ANLAGGSLVPRGSGGSEFHVQLQQSGGGLVQPGGSLRLSCAASGSIFSIYA | |
| | MGWYRQAPGKQRELVAAISSYGSTNYADSVKGRFTISRDNAKNTVYLQ | |
| | MNSLKPEDTAVYYCNADIATMTAVGGFDYWGQGTQVTVSSEPKTPKPQ | |
| | PMVTINDLHYSDPIDEDNIINMRIPLYDLEVDDQFINHNVPDLKAFQVFPN | |
| | VWVVPERYTFYSTMKNLDAPANPSRSSYYDPTYLQSDAEKEVFLQQMIL | |
| | LFKRINSTQEGQQFLNLLSRSIPVPYESNGDVAMGTTQVIKQMDDKGNV | |
| | LKHRRAHIIIYGPGPDLMAKGSKALTKSRETGRGCMAEIYFSPMYHKTYS | |
| | TKLTNKNSLVDKSVQEFVPDPAVTLIHQLCHGLHALYGIDLGNVGSWEF | |
| | NSNPNSLFSSWFSSKEAVNFEEVMTFGGEDVKVIKSEIDKKIPGILNLIKTT | |
| | VEPIINKITDPHDEMLQCLQSKYPSLKGTLGQFFFDDTQLEKDIRDLWMV | |
| | MNETMFAENLKALTAARFLVPKVENIVQVDILSPNVYTIDKGFNHLSKG | |
| | FKGQSVSQSYFRKISALARGAVVRA | |
| TrxA-thrombin | MSDKIIHLTDDSFDTDVLKADGAILVDFWAEWCGPCKMIAPILDEIADEYQG | 65 |
| site-anti-BoNT/A | KLTVAKLNIDQNPGTAPKYGIRGIPTILLFKNGEVAATKVGALSKGQLKEFLD | |
| VHH (A8)- | ANLAGGSLVPRGSGGSEFMGTQVQLVESGGGLVQPGGSLRLSCAASGSIF | |
| ^{ci}LC/X-Hn/X | SIYAMGWYRQAPGKQRELVAAISSYGSTNYADSVKGRFTISRDNAKNTV | |
| Hc/A-His6 | YLQMNSLKPEDTAVYYCNADIATMTAVGGFDYWGQGTQVTVSSEPKTP | |
| | KPQGGGGSGGGGSKLMKLEINKFNYNDPIDGINVITMRPPRHSDKINKGK | |
| | GPFKAFQVIKNIWIVPERYNFTNNTNDLNIPSEPIMEADAIYNPNYLNTPS | |
| | EKDEFLQGVIKVLERIKSKPEGEKLLELISSSIPLPLVSNGALTLSDNETIAY | |
| | QENNNIVSNLQANLVIYGPGPDIANNATYGLYSTPISNGEGTLSEVSFSPF | |
| | YLKPFDESYGNYRSLVNIVNKFVKREFAPDPASTLMHQLVHVTHNLYGI | |
| | SNRNFYYNFDTGKIETSRQQNSLIFEELLTFGGIDSKAISSLIIKKIIETAKN | |
| | NYTTLISERLNTVTVENDLLKYIKNKIPVQGRLGNFKLDTAEFEKKLNTIL | |
| | FVLNESNLAQRFSILVAKHFLKERPIDPIYVNILDDNSYSTLEGFNISSQGS | |
| | NDFQGQLLESSYFEKIESNALRAFIKICHKAIDGRSLGGSLVPRGSGGSAA | |
| | AYNKTLDCIEVENKDLFLISNKDSLNDINLSEEKIKPETTVFFKDKLPPQDI | |
| | TLSNYDFTEANSIPSISQQNILERNEELYEPIRNSLFEIKTIYVDKLTTFHFL | |
| | EAQNIDESIDSSKIRVELTDSVDEALSNPNKVYSPFKNMSNTINSIETGITS | |
| | TYIFYQWLRSIVKDFSDETGKIDVIDKSSDTLAIVPYIGPLLNIGNDIRHGD | |
| | FVGAIELAGITALLEYVPEFTIPILVGLEVIGGELAREQVEAIVNNALDKRD | |
| | QKWAEVYNITKAQWWGTIHLQINTRLAHTYKALSRQANAIKMNMEFQL | |
| | ANYKGNIDDKAKIKNAISETEILLNKSVEQAMKNTEKFMIKLSNSYLTKE | |
| | MIPKVQDNLKNFDLETKKTLDKFIKEKEDILGTNLSSSLRRKVSIRLNKNI | |
| | AFDINDIPFSEFDDLINQYKNEIEDYEVLNLGAEDGKIKDLSGTTSDINIGS | |
| | 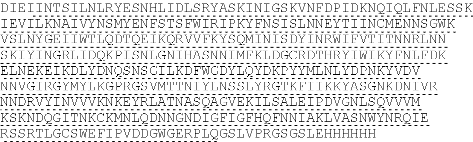 | |
| TrxA-thrombin | MSDKIIHLTDDSFDTDVLKADGAILVDFWAEWCGPCKMIAPILDEIADEYQG | 66 |
| site-anti-BoNT/A | KLTVAKLNIDQNPGTAPKYGIRGIPTILLFKNGEVAATKVGALSKGQLKEFLD | |
| VHH (A8)- | ANLAGGSLVPRGSGGSEFMGTQVQLVESGGGLVQPGGSLRLSCAASGSIF | |
| ^{ci}LC/En-Hn/X- | SIYAMGWYRQAPGKQRELVAAISSYGSTNYADSVKGRFTISRDNAKNTV | |
| Hc/A-His6 | YLQMNSLKPEDTAVYYCNADIATMTAVGGFDYWGQGTQVTVSSEPKTP | |
| | KPQGGGGSGGGGSKMVTINDLHYSDPIDEDNIINMRIPLYDLEVDDQFIN | |
| | HNVPDLKAFQVFPNVWVVPERYTFYSTMKNLDAPANPSRSSYYDPTYL | |
| | QSDAEKEVFLQQMILLFKRINSTQEGQQFLNLLSRSIPVPYESNGDVAMG | |
| | TTQVIKQMDDKGNVLKHRRAHIIIYGPGPDLMAKGSKALTKSRETGRGC | |
| | MAEIYFSPMYHKTYSTKLTNKNSLVDKSVQEFVPDPAVTLIHQLCHGLH | |
| | ALYGIDLGNVGSWEFNSNPNSLFSSWFSSKEAVNFEEVMTFGGEDVKVI | |
| | KSEIDKKIPGILNLIKTTVEPIINKITDPHDEMLQCLQSKYPSLKGTLGQFFF | |
| | DDTQLEKDIRDLWMVMNETMFAENLKALTRARYLVPKVENIVQVDILS | |
| | PNVYTIDKGFNHLSKGFKGQSVSQSYFRKISALARGAVVRACHKAIDGR | |
| | SLGGSLVPRGSGGSAAAYNKTLDCIEILEDDLFIMSSKDSFTDTDFSEPSV | |
| | GPVSYKAKKGADTILDSTLSNYDFSKEINFTSTVPIITVEDPLETDEDVPVI | |
| | SEDRTVYVDDYTTFHFLEAQKIGKEVVPTQTKVVFTTNMEEALFDSKKV | |
| | YTVFENTASRINEAGTGIANGMMFYQWLKGIVQDFTEEATQKDTFDKIS | |
| | DVTMIVPYLGNILNIGNDIRKGDFMGAVELGGVTILLEAIPELTLPVLIGLT | |
| | IIEDELEKEQVSQTVYNVLDKRDEKWEEVYGFVKQQWWWMVHTQFET | |
| | RILHAYQALNHQVEAIKANMTYQLANYRGNQEDKELLEKAIDDTLQSLY | |
| | YAVDQAMHNIKRFLIQSSKSYLLNQMLPKTKEQLLAFDQQTLRNVNDFI | |
| | NKNQGVLGESLAKDLKKKVEKRLTSLPVFNLEDLPISEFEDLIHSHEIDIQ | |
| | 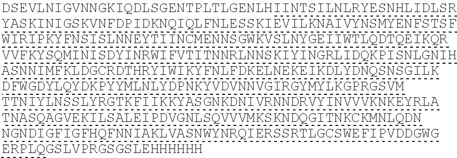 | |
| Anti-BoNT/B VHH | QVQLVESGGGLVQPGGSLRLSCAASGFPFHAYYMSWVRQAPGKGLEWV | 67 |
| (J10VHH-BLc- | SHIGNGGIITRYADSVKGRFTISRDNAKNTLYLQMTNLKPEDTALYYCTL | |
| JNE-B10, referred | GTRDDLGPERGQGTQVTVSSEPKTPKPQ | |
| to herein as B10 | | |
| also referred to | | |
| herein as J10) | | |
| Anti-BoNT/B VHH | QLQLVESGGGMVQPGGSLRLSCAASGFTFSTYDMSWVRQAPGKGPEWV | 113 |
| | SIINAGGGSTYYAASVKGRFAISRDNAKNTLYLQMNNLKPEDTALYYCA | |
| | RVASYYCRGYVCSPPEFDYWGQGTQVTVSSEPKTPKPQ | |
| Anti-BoNT/B VHH | QVQLVESGGGLVQSGGSLRLSCAASGSIDSLYHMGWYRQAPGKERELV | 114 |
| (JLJG3) | ARVQDGGSTAYKDSVKGRFTISRDFSRSTMYLQMNSLKPEDTAIYYCAA | |
| | KSTISTPLSWGQGTQVTVSSEPKTPKPQ | |
| TrxA-thrombin | MSDKIIHLTDDSFDTDVLKADGAILVDFWAEWCGPCKMIAPILDEIADEY | 68 |
| site-anti-BoNT/B | QGKLTVAKLNIDQNPGTAPKYGIRGIPTLLLFKNGEVAATKVGALSKGQ | |
| VHH | LKEFLDANLAGGSLVPRGSGGSEFQVQLVESGGGLVQPGGSLRLSCAAS | |
| | GFPFHAYYMSWVRQAPGKGLEWVSHIGNGGIITRYADSVKGRFTISRDN | |
| | AKNTLYLQMTNLKPEDTALYYCTLGTRDDLGPERGQGTQVTVSSEPKTP | |
| | KPQ | |
| TrxA-thrombin | MSDKIIHLTDDSFDTDVLKADGAILVDFWAEWCGPCKMIAPILDEIADEYQG | 69 |
| site-anti-BoNT/B | KLTVAKLNIDQNPGTAPKYGIRGIPTILLFKNGEVAATKVGALSKGQLKEFLD | |
| VHH-^{ci}LC/X | ANLAGGSLVPRGSGGSEFQVQLVESGGGLVQPGGSLRLSCAASGFPFHAY | |
| | YMSWVRQAPGKGLEWVSHIGNGGIITRYADSVKGRFTISRDNAKNTLYL | |
| | QMTNLKPEDTALYYCTLGTRDDLGPERGQGTQVTVSSEPKTPKPQGGGG | |
| | SGGGGSKLMKLEINKFNYNDPIDGINVITMRPPRHSDKINKGKGPFKAFQ | |
| | VIKNIWIVPERYNFTNNTNDLNIPSEPIMEADAIYNPNYLNTPSEKDEFLQ | |
| | GVIKVLERIKSKPEGEKLLELISSSIPLPLVSNGALTLSDNETIAYQENNNIV | |
| | SNLQANLVIYGPGPDIANNATYGLYSTPISNGEGTLSEVSFSPFYLKPFDE | |
| | SYGNYRSLVNIVNKFVKREFAPDPASTLMHQLVHVTHNLYGISNRNFYY | |
| | NFDTGKIETSRQQNSLIFEELLTFGGIDSKAISSLIIKKIIETAKNNYTTLISE | |
| | RLNTVTVENDLLKYIKNKIPVQGRLGNFKLDTAEFEKKLNTILFVLNESN | |
| | LAQRFSILVAKHFLKERPIDPIYVNILDDNSYSTLEGFNISSQGSNDFQGQL | |
| | LESSYFEKIESNALRAFIKI | |
| TrxA-thrombin | MSDKIIHLTDDSFDTDVLKADGAILVDFWAEWCGPCKMIAPILDEIADEYQG | 70 |
| site-anti-BoNT/B | KLTVAKLNIDQNPGTAPKYGIRGIPTILLFKNGEVAATKVGALSKGQLKEFLD | |
| VHH-^{ci}LC/En | ANLAGGSLVPRGSGGSEFQVQLVESGGGLVQPGGSLRLSCAASGFPFHAY | |
| | YMSWVRQAPGKGLEWVSHIGNGGIITRYADSVKGRFTISRDNAKNTLYL | |
| | QMTNLKPEDTALYYCTLGTRDDLGPERGQGTQVTVSSEPKTPKPQGGGG | |
| | SGGGGSKLMVTINDLHYSDPIDEDNIINMRIPLYDLEVDDQFINHNVPDL | |
| | KAFQVFPNVWVVPERYTFYSTMKNLDAPANPSRSSYYDPTYLQSDAEKE | |
| | VFLQQMILLFKRINSTQEGQQFLNLLSRSIPVPYESNGDVAMGTTQVIKQ | |
| | MDDKGNVLKHRRAHIIIYGPGPDLMAKGSKALTKSRETGRGCMAEIYFS | |
| | PMYHKTYSTKLTNKNSLVDKSVQEFVPDPAVTLIHQLCHGLHALYGIDL | |
| | GNVGSWEFNSNPNSLFSSWFSSKEAVNFEEVMTFGGEDVKVIKSEIDKKI | |
| | PGILNLIKTTVEPIINKITDPHDEMLQCLQSKYPSLKGTLGQFFFDDTQLEK | |
| | DIRDLWMVMNETMFAENLKALTAARFLVPKVENIVQVDILSPNVYTIDK | |
| | GFNHLSKGFKGQSVSQSYFRKISALARGAVVRA | |
| TrxA-anti- | MSDKIIHLTDDSFDTDVLKADGAILVDFWAEWCGPCKMIAPILDEIADEYQG | 71 |
| BoNT/B VHH- | KLTVAKLNIDQNPGTAPKYGIRGIPTILLFKNGEVAATKVGALSKGQLKEFLD | |
| ^{ci}LC/X-Hn/X- | ANLAGGSLVPRGSGGSEFQVQLVESGGGLVQPGGSLRLSCAASGFPFHAY | |
| Hc/A-His6 | YMSWVRQAPGKGLEWVSHIGNGGIITRYADSVKGRFTISRDNAKNTLYL | |
| | QMTNLKPEDTALYYCTLGTRDDLGPERGQGTQVTVSSEPKTPKPQGGGG | |
| | SGGGGSKLMKLEINKFNYNDPIDGINVITMRPPRHSDKINKGKGPFKAFQ | |
| | VIKNIWIVPERYNFTNNTNDLNIPSEPIMEADAIYNPNYLNTPSEKDEFLQ | |
| | GVIKVLERIKSKPEGEKLLELISSSIPLPLVSNGALTLSDNETIAYQENNNIV | |
| | SNLQANLVIYGPGPDIANNATYGLYSTPISNGEGTLSEVSFSPFYLKPFDE | |
| | SYGNYRSLVNIVNKFVKREFAPDPASTLMHQLVHVTHNLYGISNRNFYY | |
| | NFDTGKIETSRQQNSLIFEELLTFGGIDSKAISSLIIKKIIETAKNNYTTLISE | |
| | RLNTVTVENDLLKYIKNKIPVQGRLGNFKLDTAEFEKKLNTILFVLNESN | |
| | LAQRFSILVAKHFLKERPIDPIYVNILDDNSYSTLEGFNISSQGSNDFQGQL | |
| | LESSYFEKIESNALRAFIKICHKAIDGRSLGGSLVPRGSGGSAAAYNKTLD | |
| | CIEVENKDLFLISNKDSLNDINLSEEKIKPETTVFFKDKLPPQDITLSNYDF | |
| | TEANSIPSISQQNILERNEELYEPIRNSLFEIKTIYVDKLTTFHFLEAQNIDES | |
| | IDSSKIRVELTDSVDEALSNPNKVYSPFKNMSNTINSIETGITSTYIFYQWL | |
| | RSIVKDFSDETGKIDVIDKSSDTLAIVPYIGPLLNIGNDIRHGDFVGAIELA | |
| | GITALLEYVPEFTIPILVGLEVIGGELAREQVEAIVNNALDKRDQKWAEV | |
| | YNITKAQWWGTIHLQINTRLAHTYKALSRQANAIKMNMEFQLANYKGN | |
| | IDDKAKIKNAISETEILLNKSVEQAMKNTEKFMIKLSNSYLTKEMIPKVQD | |
| | NLKNFDLETKKTLDKFIKEKEDILGTNLSSSLRRKVSIRLNKNIAFDINDIP | |
| | 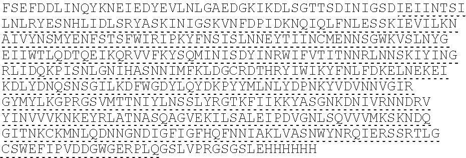 | |
| TrxA-anti- | MSDKIIHLTDDSFDTDVLKADGAILVDFWAEWCGPCKMIAPILDEIADEYQG | 72 |
| BoNT/B VHH- | KLTVAKLNIDQNPGTAPKYGIRGIPTILLFKNGEVAATKVGALSKGQLKEFLD | |
| ^{ci}LC/En-Hn/En- | ANLAGGSLVPRGSGGSEFQVQLVESGGGLVQPGGSLRLSCAASGFPFHAY | |
| Hc/A-His6 | YMSWVRQAPGKGLEWVSHIGNGGIITRYADSVKGRFTISRDNAKNTLYL | |
| | QMTNLKPEDTALYYCTLGTRDDLGPERGQGTQVTVSSEPKTPKPQGGGG | |
| | SGGGGSASMVTINDLHYSDPIDEDNIINMRIPLYDLEVDDQFINHNVPDLK | |
| | AFQVFPNVWVVPERYTFYSTMKNLDAPANPSRSSYYDPTYLQSDAEKEV | |
| | FLQQMILLFKRINSTQEGQQFLNLLSRSIPVPYESNGDVAMGTTQVIKQM | |
| | DDKGNVLKHRRAHIIIYGPGPDLMAKGSKALTKSRETGRGCMAEIYFSP | |
| | MYHKTYSTKLTNKNSLVDKSVQEFVPDPAVTLIHQLCHGLHALYGIDLG | |
| | NVGSWEFNSNPNSLFSSWFSSKEAVNFEEVMTFGGEDVKVIKSEIDKKIP | |
| | GILNLIKTTVEPIINKITDPHDEMLQCLQSKYPSLKGTLGQFFFDDTQLEK | |
| | DIRDLWMVMNETMFAENLKALTAARFLVPKVENIVQVDILSPNVYTIDK | |
| | GFNHLSKGFKGQSVSQSYFRKISALARGAVVRACHKAIDGRSLGGSLVP | |
| | RGSGGSAAAYNKTLDCIEILEDDLFIMSSKDSFTDTDFSEPSVGPVSYKAK | |
| | KGADTILDSTLSNYDFSKEINFTSTVPIITVEDPLETDEDVPVISEDRTVYV | |
| | DDYTTFHFLEAQKIGKEVVPTQTKVVFTTNMEEALFDSKKVYTVFENTA | |
| | SRINEAGTGIANGMMFYQWLKGIVQDFTEEATQKDTFDKISDVTMIVPY | |
| | LGNILNIGNDIRKGDFMGAVELGGVTILLEAIPELTLPVLIGLTIIEDELEKE | |
| | QVSQTVYNVLDKRDEKWEEVYGFVKQQWWWMVHTQFETRILHAYQA | |
| | LNHQVEAIKANMTYQLANYRGNQEDKELLEKAIDDTLQSLYYAVDQAM | |
| | HNIKRFLIQSSKSYLLNQMLPKTKEQLLAFDQQTLRNVNDFINKNQGVLG | |
| | ESLAKDLKKKVEKRLTSLPVFNLEDLPISEFEDLIHSHEIDIQDSEVLNIGV | |
| | 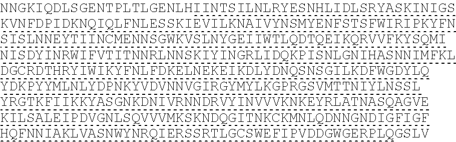 | |
| | PRGSGSLEHHHHHH | |
| TrxA-anti- | MSDKIIHLTDDSFDTDVLKADGAILVDFWAEWCGPCKMIAPILDEIADEYQG | 73 |
| BoNT/A VHH- | KLTVAKLNIDQNPGTAPKYGIRGIPTILLFKNGEVAATKVGALSKGQLKEFLD | |
| anti-BoNT/B | ANLAGGSLVPRGSGGSEFMGTQVQLVESGGGLVQPGGSLRLSCAASGSIFSIY | |
| VHH-^{ci}LC/X | AMGWYRQAPGKQRELVAAISSYGSTNYADSVKGRFTISRDNAKNTVYLQMNSL | |
| | KPEDTAVYYCNADIATMTAVGGFDYWGQGTQVTVSSEPKTPKPQGGGGSGG | |
| | GGSASQVQLVESGGGLVQPGGSLRLSCAASGFPFHAYYMSWVRQAPGK | |
| | GLEWVSHIGNGGIITRYADSVKGRFTISRDNAKNTLYLQMTNLKPEDTAL | |
| | YYCTLGTRDDLGPERGQGTQVTVSSEPKTPKPQGGGGSGGGGSKLMKL | |
| | EINKFNYNDPIDGINVITMRPPRHSDKINKGKGPFKAFQVIKNIWIVPERY | |
| | NFTNNTNDLNIPSEPIMEADAIYNPNYLNTPSEKDEFLQGVIKVLERIKSK | |
| | PEGEKLLELISSSIPLPLVSNGALTLSDNETIAYQENNNIVSNLQANLVIYG | |
| | PGPDIANNATYGLYSTPISNGEGTLSEVSFSPFYLKPFDESYGNYRSLVNI | |
| | VNKFVKREFAPDPASTLMHQLVHVTHNLYGISNRNFYYNFDTGKIETSR | |
| | QQNSLIFEELLTFGGIDSKAISSLIIKKIIETAKNNYTTLISERLNTVTVENDL | |
| | LKYIKNKIPVQGRLGNFKLDTAEFEKKLNTILFVLNESNLAQRFSILVAKH | |
| | FLKERPIDPIYVNILDDNSYSTLEGFNISSQGSNDFQGQLLESSYFEKIESN | |
| | ALRAFIKI | |
| TrxA-anti- | MSDKIIHLTDDSFDTDVLKADGAILVDFWAEWCGPCKMIAPILDEIADEYQG | 74 |
| BoNT/A VHH | KLTVAKLNIDQNPGTAPKYGIRGIPTILLFKNGEVAATKVGALSKGQLKEFLD | |
| (A8)-anti-BoNT/B | ANLAGGSLVPRGSGGSEFMGTQVQLVESGGGLVQPGGSLRLSCAASGSIF | |
| VHH-^{ci}LC/En | SIYAMGWYRQAPGKQRELVAAISSYGSTNYADSVKGRFTISRDNAKNTV | |
| | YLQMNSLKPEDTAVYYCNADIATMTAVGGFDYWGQGTQVTVSSEPKTP | |
| | KPQGGGGSGGGGSASQVQLVESGGGLVQPGGSLRLSCAASGFPFHAYY | |
| | MSWVRQAPGKGLEWVSHIGNGGIITRYADSVKGRFTISRDNAKNTLYLQ | |
| | MTNLKPEDTALYYCTLGTRDDLGPERGQGTQVTVSSEPKTPKPQGGGGS | |
| | GGGGSKLMVTINDLHYSDPIDEDNIINMRIPLYDLEVDDQFINHNVPDLK | |
| | AFQVFPNVWVVPERYTFYSTMKNLDAPANPSRSSYYDPTYLQSDAEKEV | |
| | FLQQMILLFKRINSTQEGQQFLNLLSRSIPVPYESNGDVAMGTTQVIKQM | |
| | DDKGNVLKHRRAHIIIYGPGPDLMAKGSKALTKSRETGRGCMAEIYFSP | |
| | MYHKTYSTKLTNKNSLVDKSVQEFVPDPAVTLIHQLCHGLHALYGIDLG | |
| | NVGSWEFNSNPNSLFSSWFSSKEAVNFEEVMTFGGEDVKVIKSEIDKKIP | |
| | GILNLIKTTVEPIINKITDPHDEMLQCLQSKYPSLKGTLGQFFFDDTQLEK | |
| | DIRDLWMVMNETMFAENLKALTAARFLVPKVENIVQVDILSPNVYTIDK | |
| | GFNHLSKGFKGQSVSQSYFRKISALARGAVVRA | |
| TrxA-anti- | MSDKIIHLTDDSFDTDVLKADGAILVDFWAEWCGPCKMIAPILDEIADEYQG | 75 |
| BoNT/A VHH | KLTVAKLNIDQNPGTAPKYGIRGIPTILLFKNGEVAATKVGALSKGQLKEFLD | |
| (A8)-anti-BoNT/B | ANLAGGSLVPRGSGGSEFMGTQVQLVESGGGLVQPGGSLRLSCAASGSIFSIY | |
| VHH-^{ci}LC/X- | AMGWYRQAPGKQRELVAAISSYGSTNYADSVKGRFTISRDNAKNTVYLQMNSL | |
| Hn/X-Hc/A-His6 | KPEDTAVYYCNADIATMTAVGGFDYWGQGTQVTVSSEPKTPKPQGGGGSGG | |
| | GGSASQVQLVESGGGLVQPGGSLRLSCAASGFPFHAYYMSWVRQAPGK | |
| | GLEWVSHIGNGGIITRYADSVKGRFTISRDNAKNTLYLQMTNLKPEDTAL | |
| | YYCTLGTRDDLGPERGQGTQVTVSSEPKTPKPQGGGGSGGGGSKLMKL | |
| | EINKFNYNDPIDGINVITMRPPRHSDKINKGKGPFKAFQVIKNIWIVPERY | |
| | NFTNNTNDLNIPSEPIMEADAIYNPNYLNTPSEKDEFLQGVIKVLERIKSK | |
| | PEGEKLLELISSSIPLPLVSNGALTLSDNETIAYQENNNIVSNLQANLVIYG | |
| | PGPDIANNATYGLYSTPISNGEGTLSEVSFSPFYLKPFDESYGNYRSLVNI | |
| | VNKFVKREFAPDPASTLMHQLVHVTHNLYGISNRNFYYNFDTGKIETSR | |
| | QQNSLIFEELLTFGGIDSKAISSLIIKKIIETAKNNYTTLISERLNTVTVENDL | |
| | LKYIKNKIPVQGRLGNFKLDTAEFEKKLNTILFVLNESNLAQRFSILVAKH | |
| | FLKERPIDPIYVNILDDNSYSTLEGFNISSQGSNDFQGQLLESSYFEKIESN | |
| | ALRAFIKICHKAIDGRSLGGSLVPRGSGGSAAAYNKTLDCIEVENKDLFLI | |
| | SNKDSLNDINLSEEKIKPETTVFFKDKLPPQDITLSNYDFTEANSIPSISQQN | |
| | ILERNEELYEPIRNSLFEIKTIYVDKLTTFHFLEAQNIDESIDSSKIRVELTDS | |
| | VDEALSNPNKVYSPFKNMSNTINSIETGITSTYIFYQWLRSIVKDFSDETG | |
| | KIDVIDKSSDTLAIVPYIGPLLNIGNDIRHGDFVGAIELAGITALLEYVPEFT | |
| | IPILVGLEVIGGELAREQVEAIVNNALDKRDQKWAEVYNITKAQWWGTI | |
| | HLQINTRLAHTYKALSRQANAIKMNMEFQLANYKGNIDDKAKIKNAISE | |
| | TEILLNKSVEQAMKNTEKFMIKLSNSYLTKEMIPKVQDNLKNFDLETKKT | |
| | LDKFIKEKEDILGTNLSSSLRRKVSIRLNKNIAFDINDIPFSEFDDLINQYKN | |
| | 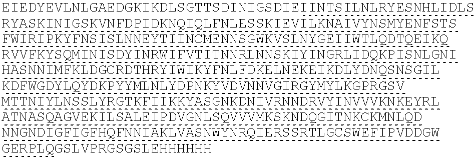 | |
| TrxA-anti- | MSDKIIHLTDDSFDTDVLKADGAILVDFWAEWCGPCKMIAPILDEIADEYQG | 76 |
| BoNT/A VHH | KLTVAKLNIDQNPGTAPKYGIRGIPTILLFKNGEVAATKVGALSKGQLKEFLD | |
| (A8)-anti-BoNT/B | ANLAGGSLVPRGSGGSEFMGTQVQLVESGGGLVQPGGSLRLSCAASGSIFSIY | |
| VHH-^{ci}LC/En- | AMGWYRQAPGKQRELVAAISSYGSTNYADSVKGRFTISRDNAKNTVYLQMNSL | |
| Hn/En-Hc/A-His6 | KPEDTAVYYCNADIATMTAVGGFDYWGQGTQVTVSSEPKTPKPQGGGGSGG | |
| | GGSASQVQLVESGGGLVQPGGSLRLSCAASGFPFHAYYMSWVRQAPGK | |
| | GLEWVSHIGNGGIITRYADSVKGRFTISRDNAKNTLYLQMTNLKPEDTAL | |
| | YYCTLGTRDDLGPERGQGTQVTVSSEPKTPKPQGGGGSGGGGSKLMVTI | |
| | NDLHYSDPIDEDNIINMRIPLYDLEVDDQFINHNVPDLKAFQVFPNVWVV | |
| | PERYTFYSTMKNLDAPANPSRSSYYDPTYLQSDAEKEVFLQQMILLFKRI | |
| | NSTQEGQQFLNLLSRSIPVPYESNGDVAMGTTQVIKQMDDKGNVLKHRR | |
| | AHIIIYGPGPDLMAKGSKALTKSRETGRGCMAEIYFSPMYHKTYSTKLTN | |
| | KNSLVDKSVQEFVPDPAVTLIHQLCHGLHALYGIDLGNVGSWEFNSNPN | |
| | SLFSSWFSSKEAVNFEEVMTFGGEDVKVIKSEIDKKIPGILNLIKTTVEPIIN | |
| | KITDPHDEMLQCLQSKYPSLKGTLGQFFFDDTQLEKDIRDLWMVMNET | |
| | MFAENLKALTAARFLVPKVENIVQVDILSPNVYTIDKGFNHLSKGFKGQS | |
| | VSQSYFRKISALARGAVVRACHKAIDGRSLGGSLVPRGSGGSAAAYNKT | |
| | LDCIEILEDDLFIMSSKDSFTDTDFSEPSVGPVSYKAKKGADTILDSTLSNY | |
| | DFSKEINFTSTVPIITVEDPLETDEDVPVISEDRTVYVDDYTTFHFLEAQKI | |
| | GKEVVPTQTKVVFTTNMEEALFDSKKVYTVFENTASRINEAGTGIANGM | |
| | MFYQWLKGIVQDFTEEATQKDTFDKISDVTMIVPYLGNILNIGNDIRKGD | |
| | FMGAVELGGVTILLEAIPELTLPVLIGLTIIEDELEKEQVSQTVYNVLDKR | |
| | DEKWEEVYGFVKQQWWWMVHTQFETRILHAYQALNHQVEAIKANMT | |
| | YQLANYRGNQEDKELLEKAIDDTLQSLYYAVDQAMHNIKRFLIQSSKSY | |
| | LLNQMLPKTKEQLLAFDQQTLRNVNDFINKNQGVLGESLAKDLKKKVE | |
| | KRLTSLPVFNLEDLPISEFEDLIHSHEIDIQDSEVLNIGVNNGKIQDLSGEN | |
| | 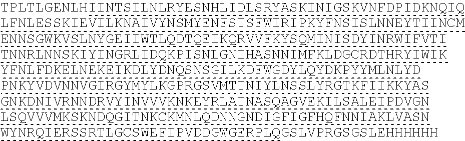 | |
| TrxA-thrombin | MSDKIIHLTDDSFDTDVLKADGAILVDFWAEWCGPCKMIAPILDEIADEY | 116 |
| site-anti-BoNT/B | QGKLTVAKLNIDQNPGTAPKYGIRGIPTLLLFKNGEVAATKVGALSKGQ | |
| VHH (B10)-anti- | LKEFLDANLAGGSLVPRGSGGSQLQLVESGGGMVQPGGSLRLSCAASGF | |
| BoNT/A VHH | TFSTYDMSWVRQAPGKGPEWVSIINAGGGSTYYAASVKGRFAISRDNAK | |
| (A8) | NTLYLQMNNLKPEDTALYYCARVASYYCRGYVCSPPEFDYWGQGTQVT | |
| | VSSEPKTPKPQGGGGSGGGGEFMGTQVQLVESGGGLVQPGGSLRLSCAA | |
| | SGSIFSIYAMGWYRQAPGKQRELVAAISSYGSTNYADSVKGRFTISRDNA | |
| | KNTVYLQMNSLKPEDTAVYYCNADIATMTAVGGFDYWGQGTQVTVSS | |
| | EPKTPKPQ | |
| TrxA-thrombin | MSDKIIHLTDDSFDTDVLKADGAILVDFWAEWCGPCKMIAPILDEIADEY | 117 |
| site-anti-BoNT/B | QGKLTVAKLNIDQNPGTAPKYGIRGIPTLLLFKNGEVAATKVGALSKGQ | |
| VHH (JLJG3)- | LKEFLDANLAGGSLVPRGSGGSQVQLVESGGGLVQSGGSLRLSCAASGSI | |
| anti-BoNT/A VHH | DSLYHMGWYRQAPGKERELVARVQDGGSTAYKDSVKGRFTISRDFSRS | |
| (A8) | TMYLQMNSLKPEDTAIYYCAAKSTISTPLSWGQGTQVTVSSEPKTPKPQG | |
| | GGGSGGGGEFMGTQVQLVESGGGLVQPGGSLRLSCAASGSIFSIYAMGW | |
| | YRQAPGKQRELVAAISSYGSTNYADSVKGRFTISRDNAKNTVYLQMNSL | |
| | KPEDTAVYYCNADIATMTAVGGFDYWGQGTQVTVSSEPKTPKPQ | |
| TrxA-thrombin | MSDKIIHLTDDSFDTDVLKADGAILVDFWAEWCGPCKMIAPILDEIADEY | 118 |
| site-anti-BoNT/B | QGKLTVAKLNIDQNPGTAPKYGIRGIPTLLLFKNGEVAATKVGALSKGQ | |
| VHH (J10)-anti- | LKEFLDANLAGGSLVPRGSGGSQVQLVESGGGLVQPGGSLRLSCAASGF | |
| BoNT/A VHH | PFHAYYMSWVRQAPGKGLEWVSHIGNGGIITRYADSVKGRFTISRDNAK | |
| (A8) | NTLYLQMTNLKPEDTALYYCTLGTRDDLGPERGQGTQVTVSSEPKTPKP | |
| | QGGGGSGGGGEFMGTQVQLVESGGGLVQPGGSLRLSCAASGSIFSIYAM | |
| | GWYRQAPGKQRELVAAISSYGSTNYADSVKGRFTISRDNAKNTVYLQM | |
| | NSLKPEDTAVYYCNADIATMTAVGGFDYWGQGTQVTVSSEPKTPKPQ | |
| TrxA-thrombin | MSDKIIHLTDDSFDTDVLKADGAILVDFWAEWCGPCKMIAPILDEIADEY | 119 |
| site-anti-BoNT/B | QGKLTVAKLNIDQNPGTAPKYGIRGIPTLLLFKNGEVAATKVGALSKGQ | |
| VHH (J10)-anti- | LKEFLDANLAGGSLVPRGSGGSQVQLVESGGGLVQPGGSLRLSCAASGF | |
| BoNT/A VHH | PFHAYYMSWVRQAPGKGLEWVSHIGNGGIITRYADSVKGRFTISRDNAK | |
| (A8)-^{ci}BoNT/X-A | NTLYLQMTNLKPEDTALYYCTLGTRDDLGPERGQGTQVTVSSEPKTPKP | |
| | QGGGGSGGGGEFMGTQVQLVESGGGLVQPGGSLRLSCAASGSIFSIYAM | |
| | GWYRQAPGKQRELVAAISSYGSTNYADSVKGRFTISRDNAKNTVYLQM | |
| | NSLKPEDTAVYYCNADIATMTAVGGFDYWGQGTQVTVSSEPKTPKPQG | |
| | GGGSGGGGSKLMKLEINKFNYNDPIDGINVITMRPPRHSDKINKGKGPFK | |
| | AFQVIKNIWIVPERYNFTNNTNDLNIPSEPIMEADAIYNPNYLNTPSEKDE | |
| | FLQGVIKVLERIKSKPEGEKLLELISSSIPLPLVSNGALTLSDNETIAYQEN | |
| | NNIVSNLQANLVIYGPGPDIANNATYGLYSTPISNGEGTLSEVSFSPFYLK | |
| | PFDESYGNYRSLVNIVNKFVKREFAPDPASTLMHQLVHVTHNLYGISNR | |
| | NFYYNFDTGKIETSRQQNSLIFEELLTFGGIDSKAISSLIIKKIIETAKNNYT | |
| | TLISERLNTVTVENDLLKYIKNKIPVQGRLGNFKLDTAEFEKKLNTILFVL | |
| | NESNLAQRFSILVAKHFLKERPIDPIYVNILDDNSYSTLEGFNISSQGSNDF | |
| | QGQLLESSYFEKIESNALRAFIKICHKAIDGRSLGGSLVPRGSGGSAAAYN | |
| | KTLDCIEVENKDLFLISNKDSLNDINLSEEKIKPETTVFFKDKLPPQDITLS | |
| | NYDFTEANSIPSISQQNILERNEELYEPIRNSLFEIKTIYVDKLTTFHFLEAQ | |
| | NIDESIDSSKIRVELTDSVDEALSNPNKVYSPFKNMSNTINSIETGITSTYIF | |
| | YQWLRSIVKDFSDETGKIDVIDKSSDTLAIVPYIGPLLNIGNDIRHGDFVG | |
| | AIELAGITALLEYVPEFTIPILVGLEVIGGELAREQVEAIVNNALDKRDQK | |
| | WAEVYNITKAQWWGTIHLQINTRLAHTYKALSRQANAIKMNMEFQLAN | |
| | YKGNIDDKAKIKNAISETEILLNKSVEQAMKNTEKFMIKLSNSYLTKEMIP | |
| | KVQDNLKNFDLETKKTLDKFIKEKEDILGTNLSSSLRRKVSIRLNKNIAFD | |
| | INDIPFSEFDDLINQYKNEIEDYEVLNLGAEDGKIKDLSGTTSDINIGSDIEII | |
| | NTSILNLRYESNHLIDLSRYASKINIGSKVNFDPIDKNQIQLFNLESSKIEVI | |
| | LKNAIVYNSMYENFSTSFWIRIPKYFNSISLNNEYTIINCMENNSGWKVSL | |
| | NYGEIIWTLQDTQEIKQRVVFKYSQMINISDYINRWIFVTITNNRLNNSKI | |
| | YINGRLIDQKPISNLGNIHASNNIMFKLDGCRDTHRYIWIKYFNLFDKELN | |
| | EKEIKDLYDNQSNSGILKDFWGDYLQYDKPYYMLNLYDPNKYVDVNNV | |
| | GIRGYMYLKGPRGSVMTTNIYLNSSLYRGTKFIIKKYASGNKDNIVRNND | |
| | RVYINVVVKNKEYRLATNASQAGVEKILSALEIPDVGNLSQVVVMKSKN | |
| | DQGITNKCKMNLQDNNGNDIGFIGFHQFNNIAKLVASNWYNRQIERSSR | |
| | TLGCSWEFIPVDDGWGERPLQ | |
| TrxA-thrombin | MSDKIIHLTDDSFDTDVLKADGAILVDFWAEWCGPCKMIAPILDEIADEY | 120 |
| site-anti-BoNT/B | QGKLTVAKLNIDQNPGTAPKYGIRGIPTLLLFKNGEVAATKVGALSKGQ | |
| VHH (J10)-anti- | LKEFLDANLAGGSLVPRGSGGSQVQLVESGGGLVQPGGSLRLSCAASGF | |
| BoNT/A VHH | PFHAYYMSWVRQAPGKGLEWVSHIGNGGIITRYADSVKGRFTISRDNAK | |
| (A8)-^{ci}BoNT/En- | NTLYLQMTNLKPEDTALYYCTLGTRDDLGPERGQGTQVTVSSEPKTPKP | |
| A | QGGGGSGGGGEFMGTQVQLVESGGGLVQPGGSLRLSCAASGSIFSIYAM | |
| | GWYRQAPGKQRELVAAISSYGSTNYADSVKGRFTISRDNAKNTVYLQM | |
| | NSLKPEDTAVYYCNADIATMTAVGGFDYWGQGTQVTVSSEPKTPKPQG | |
| | GGGSGGGGSKLMVTINDLHYSDPIDEDNIINMRIPLYDLEVDDQFINHNV | |
| | PDLKAFQVFPNVWVVPERYTFYSTMKNLDAPANPSRSSYYDPTYLQSDA | |
| | EKEVFLQQMILLFKRINSTQEGQQFLNLLSRSIPVPYESNGDVAMGTTQVI | |
| | KQMDDKGNVLKHRRAHIIIYGPGPDLMAKGSKALTKSRETGRGCMAEIY | |
| | FSPMYHKTYSTKLTNKNSLVDKSVQEFVPDPAVTLIHQLCHGLHALYGI | |
| | DLGNVGSWEFNSNPNSLFSSWFSSKEAVNFEEVMTFGGEDVKVIKSEIDK | |
| | KIPGILNLIKTTVEPIINKITDPHDEMLQCLQSKYPSLKGTLGQFFFDDTQL | |
| | EKDIRDLWMVMNETMFAENLKALTAARFLVPKVENIVQVDILSPNVYTI | |
| | DKGFNHLSKGFKGQSVSQSYFRKISALARGAVVRACHKAIDGRSLGGSL | |
| | VPRGSGGSAAAYNKTLDCIEILEDDLFIMSSKDSFTDTDFSEPSVGPVSYK | |
| | AKKGADTILDSTLSNYDFSKEINFTSTVPIITVEDPLETDEDVPVISEDRTV | |
| | YVDDYTTFHFLEAQKIGKEVVPTQTKVVFTTNMEEALFDSKKVYTVFEN | |
| | TASRINEAGTGIANGMMFYQWLKGIVQDFTEEATQKDTFDKISDVTMIV | |
| | PYLGNILNIGNDIRKGDFMGAVELGGVTILLEAIPELTLPVLIGLTIIEDELE | |
| | KEQVSQTVYNVLDKRDEKWEEVYGFVKQQWWWMVHTQFETRILHAY | |
| | QALNHQVEAIKANMTYQLANYRGNQEDKELLEKAIDDTLQSLYYAVDQ | |
| | AMHNIKRFLIQSSKSYLLNQMLPKTKEQLLAFDQQTLRNVNDFINKNQG | |
| | VLGESLAKDLKKKVEKRLTSLPVFNLEDLPISEFEDLIHSHEIDIQDSEVLN | |
| | IGVNNGKIQDLSGENTPLTLGENLHIINTSILNLRYESNHLIDLSRYASKINI | |
| | GSKVNFDPIDKNQIQLFNLESSKIEVILKNAIVYNSMYENFSTSFWIRIPKY | |
| | FNSISLNNEYTIINCMENNSGWKVSLNYGEIIWTLQDTQEIKQRVVFKYS | |
| | QMINISDYINRWIFVTITNNRLNNSKIYINGRLIDQKPISNLGNIHASNNIMF | |
| | KLDGCRDTHRYIWIKYFNLFDKELNEKEIKDLYDNQSNSGILKDFWGDY | |
| | LQYDKPYYMLNLYDPNKYVDVNNVGIRGYMYLKGPRGSVMTTNIYLNS | |
| | SLYRGTKFIIKKYASGNKDNIVRNNDRVYINVVVKNKEYRLATNASQAG | |
| | VEKILSALEIPDVGNLSQVVVMKSKNDQGITNKCKMNLQDNNGNDIGFI | |
| | GFHQFNNIAKLVASNWYNRQIERSSRTLGCSWEFIPVDDGWGERPLQ | |
| TrxA-thrombin | MSDKIIHLTDDSFDTDVLKADGAILVDFWAEWCGPCKMIAPILDEIADEY | 121 |
| site-anti-BoNT/B | QGKLTVAKLNIDQNPGTAPKYGIRGIPTLLLFKNGEVAATKVGALSKGQ | |
| VHH (J10)-anti- | LKEFLDANLAGGSLVPRGSGGSQVQLVESGGGLVQPGGSLRLSCAASGF | |
| BoNT/A VHH | PFHAYYMSWVRQAPGKGLEWVSHIGNGGIITRYADSVKGRFTISRDNAK | |
| (A8)- | NTLYLQMTNLKPEDTALYYCTLGTRDDLGPERGQGTQVTVSSEPKTPKP | |
| ^{ci}BoNT/PMP1-A | QGGGGSGGGGEFMGTQVQLVESGGGLVQPGGSLRLSCAASGSIFSIYAM | |
| | GWYRQAPGKQRELVAAISSYGSTNYADSVKGRFTISRDNAKNTVYLQM | |
| | NSLKPEDTAVYYCNADIATMTAVGGFDYWGQGTQVTVSSEPKTPKPQG | |
| | GGGSGGGGSKLLQIRVFNYNDPIDGENIVELRYHNRSPVKAFQIVDGIWII | |
| | PERYNFTNDTKKVPDDRALTILEDEVFAVRENDYLTTDVNEKNSFLNNIT | |
| | KLFKRINSSNIGNQLLNYISTSVPYPVVSTNSIKARDYNTIKFDSIDGRRIT | |
| | KSANVLIYGPSMKNLLDKQTRAINGEEAKNGIGCLSDIIFSPNYLSVQTVS | |
| | SSRFVEDPASSLTHQLIHALHNLYGIQYPGEEKFKFGGFIDKLLGTRECID | |
| | YEEVLTYGGKDSEIIRKKIDKSLYPDDFVNKYGEMYKRIKGSNPYYPDEK | |
| | KLKQSFLNRMNPFDQNGTFDTKEFKNHLMDLWFGLNESEFAKEKKILVA | |
| | KHFITKQINPKYTELTNDVYTEDKGFVNGQSIDNQNFKIIDDLISKKVKLC | |
| | HKAIDGRSLGGSLVPRGSGGSAAAYNKTLDCIDVNKEDLYFISDKEGFEN | |
| | IDFSEPEIRYDSNVTTATTSSFTDHFLVNRTFNDSDRFPPVELEYAIEPAEIV | |
| | DNTIMPDIDQKSEISLDNLTTFHYLNAQKMDLGFDSSKEQLKMVTSIEES | |
| | LLDSKKVYTPFTRTAHSVNERISGIAESYLFYQWLKTVINDFTDELNQKS | |
| | NTDKVADISWIIPYVGPALNIGLDLSHGDFTKAFEDLGVSILFAIAPEFATI | |
| | SLVALSIYENIEEDSQKEKVINKVENTLARRIEKWHQVYAFMVAQWWG | |
| | MVHTQIDTRIHQMYESLSHQIIAIKANMEYQLSHYKGPDNDKLLLKDYIY | |
| | EAEIALNTSANRAMKNIERFMIESSISYLKNNLIPSVVENLKKFDADTKKN | |
| | LDQFIDKNSSVLGSDLHILKSQVDLELNPTTKVAFNIQSIPDFDINALIDRL | |
| | GIQIINTSILNLRYESNHLIDLSRYASKINIGSKVNFDPIDKNQIQLFNLESS | |
| | KIEVILKNAIVYNSMYENFSTSFWIRIPKYFNSISLNNEYTIINCMENNSGW | |
| | KVSLNYGEIIWTLQDTQEIKQRVVFKYSQMINISDYINRWIFVTITNNRLN | |
| | NSKIYINGRLIDQKPISNLGNIHASNNIMFKLDGCRDTHRYIWIKYFNLFD | |
| | KELNEKEIKDLYDNQSNSGILKDFWGDYLQYDKPYYMLNLYDPNKYVD | |
| | VNNVGIRGYMYLKGPRGSVMTTNIYLNSSLYRGTKFIIKKYASGNKDNIV | |
| | RNNDRVYINVVVKNKEYRLATNASQAGVEKILSALEIPDVGNLSQVVVM | |
| | KSKNDQGITNKCKMNLQDNNGNDIGFIGFHQFNNIAKLVASNWYNRQIE | |
| | RSSRTLGCSWEFIPVDDGWGERPLQ | |
| Anti-BoNT/A | MGTQVQLVESGGGLVQPGGSLRLSCAASGSIFSIYAMGWYRQAPGKQRE | 124 |
| VHH (A8)- | LVAAISSYGSTNYADSVKGRFTISRDNAKNTVYLQMNSLKPEDTAVYYC | |
| ^{ci}LC/X | NADIATMTAVGGFDYWGQGTQVTVSSEPKTPKPQGGGGSGGGGSKLM | |
| | KLEINKFNYNDPIDGINVITMRPPRHSDKINKGKGPFKAFQVIKNIWIVPE | |
| | RYNFTNNTNDLNIPSEPIMEADAIYNPNYLNTPSEKDEFLQGVIKVLERIK | |
| | SKPEGEKLLELISSSIPLPLVSNGALTLSDNETIAYQENNNIVSNLQANLVI | |
| | YGPGPDIANNATYGLYSTPISNGEGTLSEVSFSPFYLKPFDESYGNYRSLV | |
| | NIVNKFVKREFAPDPASTLMHQLVHVTHNLYGISNRNFYYNFDTGKIETS | |
| | RQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKI | |
| ACS73863-^{ci}LC/X | HVQLQQSGGGLVQPGGSLRLSCAASGSIFSIYAMGWYRQAPGKQRELVA | 125 |
| | AISSYGSTNYADSVKGRFTISRDNAKNTVYLQMNSLKPEDTAVYYCNAD | |
| | IATMTAVGGFDYWGQGTQVTVSSEPKTPKPQPGGGGSGGGGSKLMKLEI | |
| | NKFNYNDPIDGINVITMRPPRHSDKINKGKGPFKAFQVIKNIWIVPERYNF | |
| | TNNTNDLNIPSEPIMEADAIYNPNYLNTPSEKDEFLQGVIKVLERIKSKPE | |
| | GEKLLELISSSIPLPLVSNGALTLSDNETIAYQENNNIVSNLQANLVIYGPG | |
| | PDIANNATYGLYSTPISNGEGTLSEVSFSPFYLKPFDESYGNYRSLVNIVN | |
| | KFVKREFAPDPASTLMHQLVHVTHNLYGISNRNFYYNFDTGKIETSRQQ | |
| | NSLIFEELLTFGGIDSKAISSLIIKKIIETAKNNYTTLISERLNTVTVENDLLK | |
| | YIKNKIPVQGRLGNFKLDTAEFEKKLNTILFVLNESNLAQRFSILVAKHFL | |
| | KERPIDPIYVNILDDNSYSTLEGFNISSQGSNDFQGQLLESSYFEKIESNAL | |
| | RAFIKI | |
| Anti-BoNT/A | MGTQVQLVESGGGLVQPGGSLRLSCAASGSIFSIYAMGWYRQAPGKQRELVA | 126 |
| VHH (A8)- | AISSYGSTNYADSVKGRFTISRDNAKNTVYLQMNSLKPEDTAVYYCNADIATMT | |
| ^{ci}LC/En | AVGGFDYWGQGTQVTVSSEPKTPKPQGGGGSGGGGSKLMVTINDLHYSD | |
| | PIDEDNIINMRIPLYDLEVDDQFINHNVPDLKAFQVFPNVWVVPERYTFY | |
| | STMKNLDAPANPSRSSYYDPTYLQSDAEKEVFLQQMILLFKRINSTQEGQ | |
| | QFLNLLSRSIPVPYESNGDVAMGTTQVIKQMDDKGNVLKHRRAHIIIYGP | |
| | GPDLMAKGSKALTKSRETGRGCMAEIYFSPMYHKTYSTKLTNKNSLVD | |
| | KSVQEFVPDPAVTLIHQLCHGLHALYGIDLGNVGSWEFNSNPNSLFSSWF | |
| | SSKEAVNFEEVMTFGGEDVKVIKSEIDKKIPGILNLIKTTVEPIINKITDPHD | |
| | EMLQCLQSKYPSLKGTLGQFFFDDTQLEKDIRDLWMVMNETMFAENLK | |
| | ALTAARFLVPKVENIVQVDILSPNVYTIDKGFNHLSKGFKGQSVSQSYFR | |
| | KISALARGAVVRA | |
| ACS73863- | HVQLQQSGGGLVQPGGSLRLSCAASGSIFSIYAMGWYRQAPGKQRELVA | 127 |
| ciLC/En | AISSYGSTNYADSVKGRFTISRDNAKNTVYLQMNSLKPEDTAVYYCNAD | |
| | IATMTAVGGFDYWGQGTQVTVSSEPKTPKPQPMVTINDLHYSDPIDEDNI | |
| | INMRIPLYDLEVDDQFINHNVPDLKAFQVFPNVWVVPERYTFYSTMKNL | |
| | DAPANPSRSSYYDPTYLQSDAEKEVFLQQMILLFKRINSTQEGQQFLNLL | |
| | SRSIPVPYESNGDVAMGTTQVIKQMDDKGNVLKHRRAHIIIYGPGPDLM | |
| | AKGSKALTKSRETGRGCMAEIYFSPMYHKTYSTKLTNKNSLVDKSVQEF | |
| | VPDPAVTLIHQLCHGLHALYGIDLGNVGSWEFNSNPNSLFSSWFSSKEAV | |
| | NFEEVMTFGGEDVKVIKSEIDKKIPGILNLIKTTVEPIINKITDPHDEMLQC | |
| | LQSKYPSLKGTLGQFFFDDTQLEKDIRDLWMVMNETMFAENLKALTAA | |
| | RFLVPKVENIVQVDILSPNVYTIDKGFNHLSKGFKGQSVSQSYFRKISALA | |
| | RGAVVRA | |
| Anti-BoNT/A | MGTQVQLVESGGGLVQPGGSLRLSCAASGSIFSIYAMGWYRQAPGKQRE | 128 |
| VHH (A8)- | LVAAISSYGSTNYADSVKGRFTISRDNAKNTVYLQMNSLKPEDTAVYYC | |
| ciLC/X-Hn/X- | NADIATMTAVGGFDYWGQGTQVTVSSEPKTPKPQGGGGSGGGGSKLM | |
| Hc/A-His6 | KLEINKFNYNDPIDGINVITMRPPRHSDKINKGKGPFKAFQVIKNIWIVPE | |
| | RYNFTNNTNDLNIPSEPIMEADAIYNPNYLNTPSEKDEFLQGVIKVLERIK | |
| | SKPEGEKLLELISSSIPLPLVSNGALTLSDNETIAYQENNNIVSNLQANLVI | |
| | YGPGPDIANNATYGLYSTPISNGEGTLSEVSFSPFYLKPFDESYGNYRSLV | |
| | NIVNKFVKREFAPDPASTLMHQLVHVTHNLYGISNRNFYYNFDTGKIETS | |
| | RQQNSLIFEELLTFGGIDSKAISSLIIKKIIETAKNNYTTLISERLNTVTVEN | |
| | DLLKYIKNKIPVQGRLGNFKLDTAEFEKKLNTILFVLNESNLAQRFSILVA | |
| | KHFLKERPIDPIYVNILDDNSYSTLEGFNISSQGSNDFQGQLLESSYFEKIE | |
| | SNALRAFIKICHKAIDGRSLGGSLVPRGSGGSAAAYNKTLDCIEVENKDL | |
| | FLISNKDSLNDINLSEEKIKPETTVFFKDKLPPQDITLSNYDFTEANSIPSIS | |
| | QQNILERNEELYEPIRNSLFEIKTIYVDKLTTFHFLEAQNIDESIDSSKIRVE | |
| | LTDSVDEALSNPNKVYSPFKNMSNTINSIETGITSTYIFYQWLRSIVKDFSD | |
| | ETGKIDVIDKSSDTLAIVPYIGPLLNIGNDIRHGDFVGAIELAGITALLEYV | |
| | PEFTIPILVGLEVIGGELAREQVEAIVNNALDKRDQKWAEVYNITKAQW | |
| | WGTIHLQINTRLAHTYKALSRQANAIKMNMEFQLANYKGNIDDKAKIKN | |
| | AISETEILLNKSVEQAMKNTEKFMIKLSNSYLTKEMIPKVQDNLKNFDLE | |
| | TKKTLDKFIKEKEDILGTNLSSSLRRKVSIRLNKNIAFDINDIPFSEFDDLIN | |
| | QYKNEIEDYEVLNLGAEDGKIKDLSGTTSDINIGSDIEIINTSILNLRYESN | |
| | HLIDLSRYASKINIGSKVNFDPIDKNQIQLFNLESSKIEVILKNAIVYNSMY | |
| | ENFSTSFWIRIPKYFNSISLNNEYTIINCMENNSGWKVSLNYGEIIWTLQDT | |
| | QEIKQRVVFKYSQMINISDYINRWIFVTITNNRLNNSKIYINGRLIDQKPIS | |
| | NLGNIHASNNIMFKLDGCRDTHRYIWIKYFNLFDKELNEKEIKDLYDNQS | |
| | NSGILKDFWGDYLQYDKPYYMLNLYDPNKYVDVNNVGIRGYMYLKGP | |
| | RGSVMTTNIYLNSSLYRGTKFIIKKYASGNKDNIVRNNDRVYINVVVKN | |
| | KEYRLATNASQAGVEKILSALEIPDVGNLSQVVVMKSKNDQGITNKCKM | |
| | NLQDNNGNDIGFIGFHQFNNIAKLVASNWYNRQIERSSRTLGCSWEFIPV | |
| | DDGWGERPLQGSLVPRGSGSLEHHHHHH | |
| Anti-BoNT/A | MGTQVQLVESGGGLVQPGGSLRLSCAASGSIFSIYAMGWYRQAPGKQRE | 129 |
| VHH (A8)- | LVAAISSYGSTNYADSVKGRFTISRDNAKNTVYLQMNSLKPEDTAVYYC | |
| ciLC/En-Hn/X- | NADIATMTAVGGFDYWGQGTQVTVSSEPKTPKPQGGGGSGGGGSKMV | |
| Hc/A-His6 | TINDLHYSDPIDEDNIINMRIPLYDLEVDDQFINHNVPDLKAFQVFPNVW | |
| | VVPERYTFYSTMKNLDAPANPSRSSYYDPTYLQSDAEKEVFLQQMILLF | |
| | KRINSTQEGQQFLNLLSRSIPVPYESNGDVAMGTTQVIKQMDDKGNVLK | |
| | HRRAHIIIYGPGPDLMAKGSKALTKSRETGRGCMAEIYFSPMYHKTYSTK | |
| | LTNKNSLVDKSVQEFVPDPAVTLIHQLCHGLHALYGIDLGNVGSWEFNS | |
| | NPNSLFSSWFSSKEAVNFEEVMTFGGEDVKVIKSEIDKKIPGILNLIKTTVE | |
| | PIINKITDPHDEMLQCLQSKYPSLKGTLGQFFFDDTQLEKDIRDLWMVMN | |
| | ETMFAENLKALTRARYLVPKVENIVQVDILSPNVYTIDKGFNHLSKGFKG | |
| | QSVSQSYFRKISALARGAVVRACHKAIDGRSLGGSLVPRGSGGSAAAYN | |
| | KTLDCIEILEDDLFIMSSKDSFTDTDFSEPSVGPVSYKAKKGADTILDSTLS | |
| | NYDFSKEINFTSTVPIITVEDPLETDEDVPVISEDRTVYVDDYTTFHFLEAQ | |
| | KIGKEVVPTQTKVVFTTNMEEALFDSKKVYTVFENTASRINEAGTGIANG | |
| | MMFYQWLKGIVQDFTEEATQKDTFDKISDVTMIVPYLGNILNIGNDIRKG | |
| | DFMGAVELGGVTILLEAIPELTLPVLIGLTIIEDELEKEQVSQTVYNVLDK | |
| | RDEKWEEVYGFVKQQWWWMVHTQFETRILHAYQALNHQVEAIKANM | |
| | TYQLANYRGNQEDKELLEKAIDDTLQSLYYAVDQAMHNIKRFLIQSSKS | |
| | YLLNQMLPKTKEQLLAFDQQTLRNVNDFINKNQGVLGESLAKDLKKKV | |
| | EKRLTSLPVFNLEDLPISEFEDLIHSHEIDIQDSEVLNIGVNNGKIQDLSGE | |
| | NTPLTLGENLHIINTSILNLRYESNHLIDLSRYASKINIGSKVNFDPIDKNQI | |
| | QLFNLESSKIEVILKNAIVYNSMYENFSTSFWIRIPKYFNSISLNNEYTIINC | |
| | MENNSGWKVSLNYGEIIWTLQDTQEIKQRVVFKYSQMINISDYINRWIFV | |
| | TITNNRLNNSKIYINGRLIDQKPISNLGNIHASNNIMFKLDGCRDTHRYIWI | |
| | KYFNLFDKELNEKEIKDLYDNQSNSGILKDFWGDYLQYDKPYYMLNLY | |
| | DPNKYVDVNNVGIRGYMYLKGPRGSVMTTNIYLNSSLYRGTKFIIKKYA | |
| | SGNKDNIVRNNDRVYINVVVKNKEYRLATNASQAGVEKILSALEIPDVG | |
| | NLSQVVVMKSKNDQGITNKCKMNLQDNNGNDIGFIGFHQFNNIAKLVAS | |
| | NWYNRQIERSSRTLGCSWEFIPVDDGWGERPLQGSLVPRGSGSLEHHHH | |
| | HH | |
| Anti-BoNT/B VHH | QVQLVESGGGLVQPGGSLRLSCAASGFPFHAYYMSWVRQAPGKGLEWV | 130 |
| | SHIGNGGIITRYADSVKGRFTISRDNAKNTLYLQMTNLKPEDTALYYCTL | |
| | GTRDDLGPERGQGTQVTVSSEPKTPKPQ | |
| Anti-BoNT/B VHH- | QVQLVESGGGLVQPGGSLRLSCAASGFPFHAYYMSWVRQAPGKGLEWV | 131 |
| ciLC/X | SHIGNGGIITRYADSVKGRFTISRDNAKNTLYLQMTNLKPEDTALYYCTL | |
| | GTRDDLGPERGQGTQVTVSSEPKTPKPQGGGGSGGGGSKLMKLEINKFN | |
| | YNDPIDGINVITMRPPRHSDKINKGKGPFKAFQVIKNIWIVPERYNFTNNT | |
| | NDLNIPSEPIMEADAIYNPNYLNTPSEKDEFLQGVIKVLERIKSKPEGEKLL | |
| | ELISSSIPLPLVSNGALTLSDNETIAYQENNNIVSNLQANLVIYGPGPDIAN | |
| | NATYGLYSTPISNGEGTLSEVSFSPFYLKPFDESYGNYRSLVNIVNKFVKR | |
| | EFAPDPASTLMHQLVHVTHNLYGISNRNFYYNFDTGKIETSRQQNSLIFE | |
| | ELLTFGGIDSKAISSLIIKKIIETAKNNYTTLISERLNTVTVENDLLKYIKNK | |
| | IPVQGRLGNFKLDTAEFEKKLNTILFVLNESNLAQRFSILVAKHFLKERPI | |
| | DPIYVNILDDNSYSTLEGFNISSQGSNDFQGQLLESSYFEKIESNALRAFIKI | |
| Anti-BoNT/B VHH- | QVQLVESGGGLVQPGGSLRLSCAASGFPFHAYYMSWVRQAPGKGLEWV | 132 |
| ciLC/En | SHIGNGGIITRYADSVKGRFTISRDNAKNTLYLQMTNLKPEDTALYYCTL | |
| | GTRDDLGPERGQGTQVTVSSEPKTPKPQGGGGSGGGGSKLMVTINDLHY | |
| | SDPIDEDNIINMRIPLYDLEVDDQFINHNVPDLKAFQVFPNVWVVPERYT | |
| | FYSTMKNLDAPANPSRSSYYDPTYLQSDAEKEVFLQQMILLFKRINSTQE | |
| | GQQFLNLLSRSIPVPYESNGDVAMGTTQVIKQMDDKGNVLKHRRAHIIIY | |
| | GPGPDLMAKGSKALTKSRETGRGCMAEIYFSPMYHKTYSTKLTNKNSLV | |
| | DKSVQEFVPDPAVTLIHQLCHGLHALYGIDLGNVGSWEFNSNPNSLFSS | |
| | WFSSKEAVNFEEVMTFGGEDVKVIKSEIDKKIPGILNLIKTTVEPIINKITD | |
| | PHDEMLQCLQSKYPSLKGTLGQFFFDDTQLEKDIRDLWMVMNETMFAE | |
| | NLKALTAARFLVPKVENIVQVDILSPNVYTIDKGFNHLSKGFKGQSVSQS | |
| | YFRKISALARGAVVRA | |
| Anti-BoNT/B | QVQLVESGGGLVQPGGSLRLSCAASGFPFHAYYMSWVRQAPGKGLEWV | 133 |
| VHH-ciLC/X- | SHIGNGGIITRYADSVKGRFTISRDNAKNTLYLQMTNLKPEDTALYYCTL | |
| Hn/X-Hc/A-His 6 | GTRDDLGPERGQGTQVTVSSEPKTPKPQGGGGSGGGGSKLMKLEINKFN | |
| | YNDPIDGINVITMRPPRHSDKINKGKGPFKAFQVIKNIWIVPERYNFTNNT | |
| | NDLNIPSEPIMEADAIYNPNYLNTPSEKDEFLQGVIKVLERIKSKPEGEKLL | |
| | ELISSSIPLPLVSNGALTLSDNETIAYQENNNIVSNLQANLVIYGPGPDIAN | |
| | NATYGLYSTPISNGEGTLSEVSFSPFYLKPFDESYGNYRSLVNIVNKFVKR | |
| | EFAPDPASTLMHQLVHVTHNLYGISNRNFYYNFDTGKIETSRQQNSLIFE | |
| | ELLTFGGIDSKAISSLIIKKIIETAKNNYTTLISERLNTVTVENDLLKYIKNK | |
| | IPVQGRLGNFKLDTAEFEKKLNTILFVLNESNLAQRFSILVAKHFLKERPI | |
| | DPIYVNILDDNSYSTLEGFNISSQGSNDFQGQLLESSYFEKIESNALRAFIKI | |
| | CHKAIDGRSLGGSLVPRGSGGSAAAYNKTLDCIEVENKDLFLISNKDSLN | |
| | DINLSEEKIKPETTVFFKDKLPPQDITLSNYDFTEANSIPSISQQNILERNEE | |
| | LYEPIRNSLFEIKTIYVDKLTTFHFLEAQNIDESIDSSKIRVELTDSVDEALS | |
| | NPNKVYSPFKNMSNTINSIETGITSTYIFYQWLRSIVKDFSDETGKIDVIDK | |
| | SSDTLAIVPYIGPLLNIGNDIRHGDFVGAIELAGITALLEYVPEFTIPILVGL | |
| | EVIGGELAREQVEAIVNNALDKRDQKWAEVYNITKAQWWGTIHLQINT | |
| | RLAHTYKALSRQANAIKMNMEFQLANYKGNIDDKAKIKNAISETEILLN | |
| | KSVEQAMKNTEKFMIKLSNSYLTKEMIPKVQDNLKNFDLETKKTLDKFI | |
| | KEKEDILGTNLSSSLRRKVSIRLNKNIAFDINDIPFSEFDDLINQYKNEIEDY | |
| | EVLNLGAEDGKIKDLSGTTSDINIGSDIEIINTSILNLRYESNHLIDLSRYAS | |
| | KINIGSKVNFDPIDKNQIQLFNLESSKIEVILKNAIVYNSMYENFSTSFWIRI | |
| | PKYFNSISLNNEYTIINCMENNSGWKVSLNYGEIIWTLQDTQEIKQRVVF | |
| | KYSQMINISDYINRWIFVTITNNRLNNSKIYINGRLIDQKPISNLGNIHASN | |
| | NIMFKLDGCRDTHRYIWIKYFNLFDKELNEKEIKDLYDNQSNSGILKDFW | |
| | GDYLQYDKPYYMLNLYDPNKYVDVNNVGIRGYMYLKGPRGSVMTTNI | |
| | YLNSSLYRGTKFIIKKYASGNKDNIVRNNDRVYINVVVKNKEYRLATNA | |
| | SQAGVEKILSALEIPDVGNLSQVVVMKSKNDQGITNKCKMNLQDNNGN | |
| | DIGFIGFHQFNNIAKLVASNWYNRQIERSSRTLGCSWEFIPVDDGWGERP | |
| | LQGSLVPRGSGSLEHHHHHH | |
| Anti-BoNT/B | QVQLVESGGGLVQPGGSLRLSCAASGFPFHAYYMSWVRQAPGKGLEWV | 134 |
| VHH-ciLC/En- | SHIGNGGIITRYADSVKGRFTISRDNAKNTLYLQMTNLKPEDTALYYCTL | |
| Hn/En-Hc/A-His6 | GTRDDLGPERGQGTQVTVSSEPKTPKPQGGGGSGGGGSASMVTINDLHY | |
| | SDPIDEDNIINMRIPLYDLEVDDQFINHNVPDLKAFQVFPNVWVVPERYT | |
| | FYSTMKNLDAPANPSRSSYYDPTYLQSDAEKEVFLQQMILLFKRINSTQE | |
| | GQQFLNLLSRSIPVPYESNGDVAMGTTQVIKQMDDKGNVLKHRRAHIIIY | |
| | GPGPDLMAKGSKALTKSRETGRGCMAEIYFSPMYHKTYSTKLTNKNSLV | |
| | DKSVQEFVPDPAVTLIHQLCHGLHALYGIDLGNVGSWEFNSNPNSLFSS | |
| | WFSSKEAVNFEEVMTFGGEDVKVIKSEIDKKIPGILNLIKTTVEPIINKITD | |
| | PHDEMLQCLQSKYPSLKGTLGQFFFDDTQLEKDIRDLWMVMNETMFAE | |
| | NLKALTAARFLVPKVENIVQVDILSPNVYTIDKGFNHLSKGFKGQSVSQS | |
| | YFRKISALARGAVVRACHKAIDGRSLGGSLVPRGSGGSAAAYNKTLDCI | |
| | EILEDDLFIMSSKDSFTDTDFSEPSVGPVSYKAKKGADTILDSTLSNYDFS | |
| | KEINFTSTVPIITVEDPLETDEDVPVISEDRTVYVDDYTTFHFLEAQKIGKE | |
| | VVPTQTKVVFTTNMEEALFDSKKVYTVFENTASRINEAGTGIANGMMFY | |
| | QWLKGIVQDFTEEATQKDTFDKISDVTMIVPYLGNILNIGNDIRKGDFMG | |
| | AVELGGVTILLEAIPELTLPVLIGLTIIEDELEKEQVSQTVYNVLDKRDEK | |
| | WEEVYGFVKQQWWWMVHTQFETRILHAYQALNHQVEAIKANMTYQL | |
| | ANYRGNQEDKELLEKAIDDTLQSLYYAVDQAMHNIKRFLIQSSKSYLLN | |
| | QMLPKTKEQLLAFDQQTLRNVNDFINKNQGVLGESLAKDLKKKVEKRL | |
| | TSLPVFNLEDLPISEFEDLIHSHEIDIQDSEVLNIGVNNGKIQDLSGENTPLT | |
| | LGENLHIINTSILNLRYESNHLIDLSRYASKINIGSKVNFDPIDKNQIQLFNL | |
| | ESSKIEVILKNAIVYNSMYENFSTSFWIRIPKYFNSISLNNEYTIINCMENNS | |
| | GWKVSLNYGEIIWTLQDTQEIKQRVVFKYSQMINISDYINRWIFVTITNNR | |
| | LNNSKIYINGRLIDQKPISNLGNIHASNNIMFKLDGCRDTHRYIWIKYFNL | |
| | FDKELNEKEIKDLYDNQSNSGILKDFWGDYLQYDKPYYMLNLYDPNKY | |
| | VDVNNVGIRGYMYLKGPRGSVMTTNIYLNSSLYRGTKFIIKKYASGNKD | |
| | NIVRNNDRVYINVVVKNKEYRLATNASQAGVEKILSALEIPDVGNLSQV | |
| | VVMKSKNDQGITNKCKMNLQDNNGNDIGFIGFHQFNNIAKLVASNWYN | |
| | RQIERSSRTLGCSWEFIPVDDGWGERPLQGSLVPRGSGSLEHHHHHH | |
| Anti-BoNT/A | MGTQVQLVESGGGLVQPGGSLRLSCAASGSIFSIYAMGWYRQAPGKQRE | 135 |
| VHH-anti-BoNT/B | LVAAISSYGSTNYADSVKGRFTISRDNAKNTVYLQMNSLKPEDTAVYYC | |
| VHH-ciLC/X | NADIATMTAVGGFDYWGQGTQVTVSSEPKTPKPQGGGGSGGGGSASQV | |
| | QLVESGGGLVQPGGSLRLSCAASGFPFHAYYMSWVRQAPGKGLEWVSH | |
| | IGNGGIITRYADSVKGRFTISRDNAKNTLYLQMTNLKPEDTALYYCTLGT | |
| | RDDLGPERGQGTQVTVSSEPKTPKPQGGGGSGGGGSKLMKLEINKFNYN | |
| | DPIDGINVITMRPPRHSDKINKGKGPFKAFQVIKNIWIVPERYNFTNNTND | |
| | LNIPSEPIMEADAIYNPNYLNTPSEKDEFLQGVIKVLERIKSKPEGEKLLEL | |
| | ISSSIPLPLVSNGALTLSDNETIAYQENNNIVSNLQANLVIYGPGPDIANNA | |
| | TYGLYSTPISNGEGTLSEVSFSPFYLKPFDESYGNYRSLVNIVNKFVKREF | |
| | APDPASTLMHQLVHVTHNLYGISNRNFYYNFDTGKIETSRQQNSLIFEEL | |
| | LTFGGIDSKAISSLIIKKIIETAKNNYTTLISERLNTVTVENDLLKYIKNKIP | |
| | VQGRLGNFKLDTAEFEKKLNTILFVLNESNLAQRFSILVAKHFLKERPIDP | |
| | IYVNILDDNSYSTLEGFNISSQGSNDFQGQLLESSYFEKIESNALRAFIKI | |
| Anti-BoNT/A | MGTQVQLVESGGGLVQPGGSLRLSCAASGSIFSIYAMGWYRQAPGKQRE | 136 |
| VHH (A8)-anti- | LVAAISSYGSTNYADSVKGRFTISRDNAKNTVYLQMNSLKPEDTAVYYC | |
| BoNT/B VHH- | NADIATMTAVGGFDYWGQGTQVTVSSEPKTPKPQGGGGSGGGGSASQV | |
| ciLC/En | QLVESGGGLVQPGGSLRLSCAASGFPFHAYYMSWVRQAPGKGLEWVSH | |
| | IGNGGIITRYADSVKGRFTISRDNAKNTLYLQMTNLKPEDTALYYCTLGT | |
| | RDDLGPERGQGTQVTVSSEPKTPKPQGGGGSGGGGSKLMVTINDLHYSD | |
| | PIDEDNIINMRIPLYDLEVDDQFINHNVPDLKAFQVFPNVWVVPERYTFY | |
| | STMKNLDAPANPSRSSYYDPTYLQSDAEKEVFLQQMILLFKRINSTQEGQ | |
| | QFLNLLSRSIPVPYESNGDVAMGTTQVIKQMDDKGNVLKHRRAHIIIYGP | |
| | GPDLMAKGSKALTKSRETGRGCMAEIYFSPMYHKTYSTKLTNKNSLVD | |
| | KSVQEFVPDPAVTLIHQLCHGLHALYGIDLGNVGSWEFNSNPNSLFSSWF | |
| | SSKEAVNFEEVMTFGGEDVKVIKSEIDKKIPGILNLIKTTVEPIINKITDPHD | |
| | EMLQCLQSKYPSLKGTLGQFFFDDTQLEKDIRDLWMVMNETMFAENLK | |
| | ALTAARFLVPKVENIVQVDILSPNVYTIDKGFNHLSKGFKGQSVSQSYFR | |
| | KISALARGAVVRA | |
| TrxA-anti- | MGTQVQLVESGGGLVQPGGSLRLSCAASGSIFSIYAMGWYRQAPGKQRE | 137 |
| BoNT/A VHH | LVAAISSYGSTNYADSVKGRFTISRDNAKNTVYLQMNSLKPEDTAVYYC | |
| (A8)-anti-BoNT/B | NADIATMTAVGGFDYWGQGTQVTVSSEPKTPKPQGGGGSGGGGSASQV | |
| VHH-ciLC/X- | QLVESGGGLVQPGGSLRLSCAASGFPFHAYYMSWVRQAPGKGLEWVSH | |
| Hn/X-Hc/A-His6 | IGNGGIITRYADSVKGRFTISRDNAKNTLYLQMTNLKPEDTALYYCTLGT | |
| | RDDLGPERGQGTQVTVSSEPKTPKPQGGGGSGGGGSKLMKLEINKFNYN | |
| | DPIDGINVITMRPPRHSDKINKGKGPFKAFQVIKNIWIVPERYNFTNNTND | |
| | LNIPSEPIMEADAIYNPNYLNTPSEKDEFLQGVIKVLERIKSKPEGEKLLEL | |
| | ISSSIPLPLVSNGALTLSDNETIAYQENNNIVSNLQANLVIYGPGPDIANNA | |
| | TYGLYSTPISNGEGTLSEVSFSPFYLKPFDESYGNYRSLVNIVNKFVKREF | |
| | APDPASTLMHQLVHVTHNLYGISNRNFYYNFDTGKIETSRQQNSLIFEEL | |
| | LTFGGIDSKAISSLIIKKIIETAKNNYTTLISERLNTVTVENDLLKYIKNKIP | |
| | VQGRLGNFKLDTAEFEKKLNTILFVLNESNLAQRFSILVAKHFLKERPIDP | |
| | IYVNILDDNSYSTLEGFNISSQGSNDFQGQLLESSYFEKIESNALRAFIKIC | |
| | HKAIDGRSLGGSLVPRGSGGSAAAYNKTLDCIEVENKDLFLISNKDSLND | |
| | INLSEEKIKPETTVFFKDKLPPQDITLSNYDFTEANSIPSISQQNILERNEEL | |
| | YEPIRNSLFEIKTIYVDKLTTFHFLEAQNIDESIDSSKIRVELTDSVDEALSN | |
| | PNKVYSPFKNMSNTINSIETGITSTYIFYQWLRSIVKDFSDETGKIDVIDKS | |
| | SDTLAIVPYIGPLLNIGNDIRHGDFVGAIELAGITALLEYVPEFTIPILVGLE | |
| | VIGGELAREQVEAIVNNALDKRDQKWAEVYNITKAQWWGTIHLQINTR | |
| | LAHTYKALSRQANAIKMNMEFQLANYKGNIDDKAKIKNAISETEILLNKS | |
| | VEQAMKNTEKFMIKLSNSYLTKEMIPKVQDNLKNFDLETKKTLDKFIKE | |
| | KEDILGTNLSSSLRRKVSIRLNKNIAFDINDIPFSEFDDLINQYKNEIEDYEV | |
| | LNLGAEDGKIKDLSGTTSDINIGSDIEIINTSILNLRYESNHLIDLSRYASKI | |
| | NIGSKVNFDPIDKNQIQLFNLESSKIEVILKNAIVYNSMYENFSTSFWIRIP | |
| | KYFNSISLNNEYTIINCMENNSGWKVSLNYGEIIWTLQDTQEIKQRVVFK | |
| | YSQMINISDYINRWIFVTITNNRLNNSKIYINGRLIDQKPISNLGNIHASNNI | |
| | MFKLDGCRDTHRYIWIKYFNLFDKELNEKEIKDLYDNQSNSGILKDFWG | |
| | DYLQYDKPYYMLNLYDPNKYVDVNNVGIRGYMYLKGPRGSVMTTNIY | |
| | LNSSLYRGTKFIIKKYASGNKDNIVRNNDRVYINVVVKNKEYRLATNAS | |
| | QAGVEKILSALEIPDVGNLSQVVVMKSKNDQGITNKCKMNLQDNNGNDI | |
| | GFIGFHQFNNIAKLVASNWYNRQIERSSRTLGCSWEFIPVDDGWGERPLQ | |
| | GSLVPRGSGSLEHHHHHH | |
| Anti-BoNT/A | MGTQVQLVESGGGLVQPGGSLRLSCAASGSIFSIYAMGWYRQAPGKQRE | 138 |
| VHH (A8)-anti- | LVAAISSYGSTNYADSVKGRFTISRDNAKNTVYLQMNSLKPEDTAVYYC | |
| BoNT/B VHH- | NADIATMTAVGGFDYWGQGTQVTVSSEPKTPKPQGGGGSGGGGSASQV | |
| ciLC/En-Hn/En- | QLVESGGGLVQPGGSLRLSCAASGFPFHAYYMSWVRQAPGKGLEWVSH | |
| Hc/A-His6 | IGNGGIITRYADSVKGRFTISRDNAKNTLYLQMTNLKPEDTALYYCTLGT | |
| | RDDLGPERGQGTQVTVSSEPKTPKPQGGGGSGGGGSKLMVTINDLHYSD | |
| | PIDEDNIINMRIPLYDLEVDDQFINHNVPDLKAFQVFPNVWVVPERYTFY | |
| | STMKNLDAPANPSRSSYYDPTYLQSDAEKEVFLQQMILLFKRINSTQEGQ | |
| | QFLNLLSRSIPVPYESNGDVAMGTTQVIKQMDDKGNVLKHRRAHIIIYGP | |
| | GPDLMAKGSKALTKSRETGRGCMAEIYFSPMYHKTYSTKLTNKNSLVD | |
| | KSVQEFVPDPAVTLIHQLCHGLHALYGIDLGNVGSWEFNSNPNSLFSSWF | |
| | SSKEAVNFEEVMTFGGEDVKVIKSEIDKKIPGILNLIKTTVEPIINKITDPHD | |
| | EMLQCLQSKYPSLKGTLGQFFFDDTQLEKDIRDLWMVMNETMFAENLK | |
| | ALTAARFLVPKVENIVQVDILSPNVYTIDKGFNHLSKGFKGQSVSQSYFR | |
| | KISALARGAVVRACHKAIDGRSLGGSLVPRGSGGSAAAYNKTLDCIEILE | |
| | DDLFIMSSKDSFTDTDFSEPSVGPVSYKAKKGADTILDSTLSNYDFSKEIN | |
| | FTSTVPIITVEDPLETDEDVPVISEDRTVYVDDYTTFHFLEAQKIGKEVVPT | |
| | QTKVVFTTNMEEALFDSKKVYTVFENTASRINEAGTGIANGMMFYQWL | |
| | KGIVQDFTEEATQKDTFDKISDVTMIVPYLGNILNIGNDIRKGDFMGAVE | |
| | LGGVTILLEAIPELTLPVLIGLTIIEDELEKEQVSQTVYNVLDKRDEKWEE | |
| | VYGFVKQQWWWMVHTQFETRILHAYQALNHQVEAIKANMTYQLANY | |
| | RGNQEDKELLEKAIDDTLQSLYYAVDQAMHNIKRFLIQSSKSYLLNQML | |
| | PKTKEQLLAFDQQTLRNVNDFINKNQGVLGESLAKDLKKKVEKRLTSLP | |
| | VFNLEDLPISEFEDLIHSHEIDIQDSEVLNIGVNNGKIQDLSGENTPLTLGE | |
| | NLHIINTSILNLRYESNHLIDLSRYASKINIGSKVNFDPIDKNQIQLFNLESS | |
| | KIEVILKNAIVYNSMYENFSTSFWIRIPKYFNSISLNNEYTIINCMENNSGW | |
| | KVSLNYGEIIWTLQDTQEIKQRVVFKYSQMINISDYINRWIFVTITNNRLN | |
| | NSKIYINGRLIDQKPISNLGNIHASNNIMFKLDGCRDTHRYIWIKYFNLFD | |
| | KELNEKEIKDLYDNQSNSGILKDFWGDYLQYDKPYYMLNLYDPNKYVD | |
| | VNNVGIRGYMYLKGPRGSVMTTNIYLNSSLYRGTKFIIKKYASGNKDNIV | |
| | RNNDRVYINVVVKNKEYRLATNASQAGVEKILSALEIPDVGNLSQVVVM | |
| | KSKNDQGITNKCKMNLQDNNGNDIGFIGFHQFNNIAKLVASNWYNRQIE | |
| | RSSRTLGCSWEFIPVDDGWGERPLQGSLVPRGSGSLEHHHHHH | |
| Anti-BoNT/B VHH | QLQLVESGGGMVQPGGSLRLSCAASGFTFSTYDMSWVRQAPGKGPEWV | 139 |
| (B10)-anti- | SIINAGGGSTYYAASVKGRFAISRDNAKNTLYLQMNNLKPEDTALYYCA | |
| BoNT/A VHH | RVASYYCRGYVCSPPEFDYWGQGTQVTVSSEPKTPKPQGGGGSGGGGE | |
| (A8) | FMGTQVQLVESGGGLVQPGGSLRLSCAASGSIFSIYAMGWYRQAPGKQR | |
| | ELVAAISSYGSTNYADSVKGRFTISRDNAKNTVYLQMNSLKPEDTAVYY | |
| | CNADIATMTAVGGFDYWGQGTQVTVSSEPKTPKPQ | |
| Anti-BoNT/B VHH | QVQLVESGGGLVQSGGSLRLSCAASGSIDSLYHMGWYRQAPGKERELV | 140 |
| (JLJG3)-anti- | ARVQDGGSTAYKDSVKGRFTISRDFSRSTMYLQMNSLKPEDTAIYYCAA | |
| BoNT/A VHH | KSTISTPLSWGQGTQVTVSSEPKTPKPQGGGGSGGGGEFMGTQVQLVES | |
| (A8) | GGGLVQPGGSLRLSCAASGSIFSIYAMGWYRQAPGKQRELVAAISSYGST | |
| | NYADSVKGRFTISRDNAKNTVYLQMNSLKPEDTAVYYCNADIATMTAV | |
| | GGFDYWGQGTQVTVSSEPKTPKPQ | |
| Anti-BoNT/B VHH | QVQLVESGGGLVQPGGSLRLSCAASGFPFHAYYMSWVRQAPGKGLEWV | 141 |
| (J10)-anti-BoNT/A | SHIGNGGIITRYADSVKGRFTISRDNAKNTLYLQMTNLKPEDTALYYCTL | |
| VHH (A8) | GTRDDLGPERGQGTQVTVSSEPKTPKPQGGGGSGGGGEFMGTQVQLVE | |
| | SGGGLVQPGGSLRLSCAASGSIFSIYAMGWYRQAPGKQRELVAAISSYGS | |
| | TNYADSVKGRFTISRDNAKNTVYLQMNSLKPEDTAVYYCNADIATMTA | |
| | VGGFDYWGQGTQVTVSSEPKTPKPQ | |
| Anti-BoNT/B VHH | QVQLVESGGGLVQPGGSLRLSCAASGFPFHAYYMSWVRQAPGKGLEWV | 142 |
| (J10)-anti-BoNT/A | SHIGNGGIITRYADSVKGRFTISRDNAKNTLYLQMTNLKPEDTALYYCTL | |
| VHH (A8)- | GTRDDLGPERGQGTQVTVSSEPKTPKPQGGGGSGGGGEFMGTQVQLVE | |
| ciBoNT/X-A | SGGGLVQPGGSLRLSCAASGSIFSIYAMGWYRQAPGKQRELVAAISSYGS | |
| | TNYADSVKGRFTISRDNAKNTVYLQMNSLKPEDTAVYYCNADIATMTA | |
| | VGGFDYWGQGTQVTVSSEPKTPKPQGGGGSGGGGSKLMKLEINKFNYN | |
| | DPIDGINVITMRPPRHSDKINKGKGPFKAFQVIKNIWIVPERYNFTNNTND | |
| | LNIPSEPIMEADAIYNPNYLNTPSEKDEFLQGVIKVLERIKSKPEGEKLLEL | |
| | ISSSIPLPLVSNGALTLSDNETIAYQENNNIVSNLQANLVIYGPGPDIANNA | |
| | TYGLYSTPISNGEGTLSEVSFSPFYLKPFDESYGNYRSLVNIVNKFVKREF | |
| | APDPASTLMHQLVHVTHNLYGISNRNFYYNFDTGKIETSRQQNSLIFEEL | |
| | LTFGGIDSKAISSLIIKKIIETAKNNYTTLISERLNTVTVENDLLKYIKNKIP | |
| | VQGRLGNFKLDTAEFEKKLNTILFVLNESNLAQRFSILVAKHFLKERPIDP | |
| | IYVNILDDNSYSTLEGFNISSQGSNDFQGQLLESSYFEKIESNALRAFIKIC | |
| | HKAIDGRSLGGSLVPRGSGGSAAAYNKTLDCIEVENKDLFLISNKDSLND | |
| | INLSEEKIKPETTVFFKDKLPPQDITLSNYDFTEANSIPSISQQNILERNEEL | |
| | YEPIRNSLFEIKTIYVDKLTTFHFLEAQNIDESIDSSKIRVELTDSVDEALSN | |
| | PNKVYSPFKNMSNTINSIETGITSTYIFYQWLRSIVKDFSDETGKIDVIDKS | |
| | SDTLAIVPYIGPLLNIGNDIRHGDFVGAIELAGITALLEYVPEFTIPILVGLE | |
| | VIGGELAREQVEAIVNNALDKRDQKWAEVYNITKAQWWGTIHLQINTR | |
| | LAHTYKALSRQANAIKMNMEFQLANYKGNIDDKAKIKNAISETEILLNKS | |
| | VEQAMKNTEKFMIKLSNSYLTKEMIPKVQDNLKNFDLETKKTLDKFIKE | |
| | KEDILGTNLSSSLRRKVSIRLNKNIAFDINDIPFSEFDDLINQYKNEIEDYEV | |
| | LNLGAEDGKIKDLSGTTSDINIGSDIEIINTSILNLRYESNHLIDLSRYASKI | |
| | NIGSKVNFDPIDKNQIQLFNLESSKIEVILKNAIVYNSMYENFSTSFWIRIP | |
| | KYFNSISLNNEYTIINCMENNSGWKVSLNYGEIIWTLQDTQEIKQRVVFK | |
| | YSQMINISDYINRWIFVTITNNRLNNSKIYINGRLIDQKPISNLGNIHASNNI | |
| | MFKLDGCRDTHRYIWIKYFNLFDKELNEKEIKDLYDNQSNSGILKDFWG | |
| | DYLQYDKPYYMLNLYDPNKYVDVNNVGIRGYMYLKGPRGSVMTTNIY | |
| | LNSSLYRGTKFIIKKYASGNKDNIVRNNDRVYINVVVKNKEYRLATNAS | |
| | QAGVEKILSALEIPDVGNLSQVVVMKSKNDQGITNKCKMNLQDNNGNDI | |
| | GFIGFHQFNNIAKLVASNWYNRQIERSSRTLGCSWEFIPVDDGWGERPLQ | |
| Anti-BoNT/B VHH | QVQLVESGGGLVQPGGSLRLSCAASGFPFHAYYMSWVRQAPGKGLEWV | 143 |
| J(10)-anti-BoNT/A | SHIGNGGIITRYADSVKGRFTISRDNAKNTLYLQMTNLKPEDTALYYCTL | |
| VHH (A8)- | GTRDDLGPERGQGTQVTVSSEPKTPKPQGGGGSGGGGEFMGTQVQLVE | |
| ciBoNT/En-A | SGGGLVQPGGSLRLSCAASGSIFSIYAMGWYRQAPGKQRELVAAISSYGS | |
| | TNYADSVKGRFTISRDNAKNTVYLQMNSLKPEDTAVYYCNADIATMTA | |
| | VGGFDYWGQGTQVTVSSEPKTPKPQGGGGSGGGGSKLMVTINDLHYSD | |
| | PIDEDNIINMRIPLYDLEVDDQFINHNVPDLKAFQVFPNVWVVPERYTFY | |
| | STMKNLDAPANPSRSSYYDPTYLQSDAEKEVFLQQMILLFKRINSTQEGQ | |
| | QFLNLLSRSIPVPYESNGDVAMGTTQVIKQMDDKGNVLKHRRAHIIIYGP | |
| | GPDLMAKGSKALTKSRETGRGCMAEIYFSPMYHKTYSTKLTNKNSLVD | |
| | KSVQEFVPDPAVTLIHQLCHGLHALYGIDLGNVGSWEFNSNPNSLFSSWF | |
| | SSKEAVNFEEVMTFGGEDVKVIKSEIDKKIPGILNLIKTTVEPIINKITDPHD | |
| | EMLQCLQSKYPSLKGTLGQFFFDDTQLEKDIRDLWMVMNETMFAENLK | |
| | ALTAARFLVPKVENIVQVDILSPNVYTIDKGFNHLSKGFKGQSVSQSYFR | |
| | KISALARGAVVRACHKAIDGRSLGGSLVPRGSGGSAAAYNKTLDCIEILE | |
| | DDLFIMSSKDSFTDTDFSEPSVGPVSYKAKKGADTILDSTLSNYDFSKEIN | |
| | FTSTVPIITVEDPLETDEDVPVISEDRTVYVDDYTTFHFLEAQKIGKEVVPT | |
| | QTKVVFTTNMEEALFDSKKVYTVFENTASRINEAGTGIANGMMFYQWL | |
| | KGIVQDFTEEATQKDTFDKISDVTMIVPYLGNILNIGNDIRKGDFMGAVE | |
| | LGGVTILLEAIPELTLPVLIGLTIIEDELEKEQVSQTVYNVLDKRDEKWEE | |
| | VYGFVKQQWWWMVHTQFETRILHAYQALNHQVEAIKANMTYQLANY | |
| | RGNQEDKELLEKAIDDTLQSLYYAVDQAMHNIKRFLIQSSKSYLLNQML | |
| | PKTKEQLLAFDQQTLRNVNDFINKNQGVLGESLAKDLKKKVEKRLTSLP | |
| | VFNLEDLPISEFEDLIHSHEIDIQDSEVLNIGVNNGKIQDLSGENTPLTLGE | |
| | NLHIINTSILNLRYESNHLIDLSRYASKINIGSKVNFDPIDKNQIQLFNLESS | |
| | KIEVILKNAIVYNSMYENFSTSFWIRIPKYFNSISLNNEYTIINCMENNSGW | |
| | KVSLNYGEIIWTLQDTQEIKQRVVFKYSQMINISDYINRWIFVTITNNRLN | |
| | NSKIYINGRLIDQKPISNLGNIHASNNIMFKLDGCRDTHRYIWIKYFNLFD | |
| | KELNEKEIKDLYDNQSNSGILKDFWGDYLQYDKPYYMLNLYDPNKYVD | |
| | VNNVGIRGYMYLKGPRGSVMTTNIYLNSSLYRGTKFIIKKYASGNKDNIV | |
| | RNNDRVYINVVVKNKEYRLATNASQAGVEKILSALEIPDVGNLSQVVVM | |
| | KSKNDQGITNKCKMNLQDNNGNDIGFIGFHQFNNIAKLVASNWYNRQIE | |
| | RSSRTLGCSWEFIPVDDGWGERPLQ | |
| Anti-BoNT/B VHH | QVQLVESGGGLVQPGGSLRLSCAASGFPFHAYYMSWVRQAPGKGLEWV | 144 |
| (J10)-anti-BoNT/A | SHIGNGGIITRYADSVKGRFTISRDNAKNTLYLQMTNLKPEDTALYYCTL | |
| VHH (A8)- | GTRDDLGPERGQGTQVTVSSEPKTPKPQGGGGSGGGGEFMGTQVQLVE | |
| ciBoNT/PMP1-A | SGGGLVQPGGSLRLSCAASGSIFSIYAMGWYRQAPGKQRELVAAISSYGS | |
| | TNYADSVKGRFTISRDNAKNTVYLQMNSLKPEDTAVYYCNADIATMTA | |
| | VGGFDYWGQGTQVTVSSEPKTPKPQGGGGSGGGGSKLLQIRVFNYNDPI | |
| | DGENIVELRYHNRSPVKAFQIVDGIWIIPERYNFTNDTKKVPDDRALTILE | |
| | DEVFAVRENDYLTTDVNEKNSFLNNITKLFKRINSSNIGNQLLNYISTSVP | |
| | YPVVSTNSIKARDYNTIKFDSIDGRRITKSANVLIYGPSMKNLLDKQTRAI | |
| | NGEEAKNGIGCLSDIIFSPNYLSVQTVSSSRFVEDPASSLTHQLIHALHNLY | |
| | GIQYPGEEKFKFGGFIDKLLGTRECIDYEEVLTYGGKDSEIIRKKIDKSLYP | |
| | DDFVNKYGEMYKRIKGSNPYYPDEKKLKQSFLNRMNPFDQNGTFDTKE | |
| | FKNHLMDLWFGLNESEFAKEKKILVAKHFITKQINPKYTELTNDVYTED | |
| | KGFVNGQSIDNQNFKIIDDLISKKVKLCHKAIDGRSLGGSLVPRGSGGSA | |
| | AAYNKTLDCIDVNKEDLYFISDKEGFENIDFSEPEIRYDSNVTTATTSSFT | |
| | DHFLVNRTFNDSDRFPPVELEYAIEPAEIVDNTIMPDIDQKSEISLDNLTTF | |
| | HYLNAQKMDLGFDSSKEQLKMVTSIEESLLDSKKVYTPFTRTAHSVNERI | |
| | SGIAESYLFYQWLKTVINDFTDELNQKSNTDKVADISWIIPYVGPALNIGL | |
| | DLSHGDFTKAFEDLGVSILFAIAPEFATISLVALSIYENIEEDSQKEKVINK | |
| | VENTLARRIEKWHQVYAFMVAQWWGMVHTQIDTRIHQMYESLSHQIIAI | |
| | KANMEYQLSHYKGPDNDKLLLKDYIYEAEIALNTSANRAMKNIERFMIE | |
| | SSISYLKNNLIPSVVENLKKFDADTKKNLDQFIDKNSSVLGSDLHILKSQV | |
| | DLELNPTTKVAFNIQSIPDFDINALIDRLGIQIINTSILNLRYESNHLIDLSRY | |
| | ASKINIGSKVNFDPIDKNQIQLFNLESSKIEVILKNAIVYNSMYENFSTSFW | |
| | IRIPKYFNSISLNNEYTIINCMENNSGWKVSLNYGEIIWTLQDTQEIKQRV | |
| | VFKYSQMINISDYINRWIFVTITNNRLNNSKIYINGRLIDQKPISNLGNIHA | |
| | SNNIMFKLDGCRDTHRYIWIKYFNLFDKELNEKEIKDLYDNQSNSGILKD | |
| | FWGDYLQYDKPYYMLNLYDPNKYVDVNNVGIRGYMYLKGPRGSVMTT | |
| | NIYLNSSLYRGTKFIIKKYASGNKDNIVRNNDRVYINVVVKNKEYRLATN | |
| | ASQAGVEKILSALEIPDVGNLSQVVVMKSKNDQGITNKCKMNLQDNNG | |
| | NDIGFIGFHQFNNIAKLVASNWYNRQIERSSRTLGCSWEFIPVDDGWGER | |
| | PLQ | |
| TrxA-thrombin | MSDKIIHLTDDSFDTDVLKADGAILVDFWAEWCGPCKMIAPILDEIADEYQG | 145 |
| site-anti-BoNT/A | KLTVAKLNIDQNPGTAPKYGIRGIPTILLFKNGEVAATKVGALSKGQLKEFLD | |
| VHH (A8)- | ANLAGGSLVPRGSGGSEFMGTQVQLVESGGGLVQPGGSLRLSCAASGSIF | |
| ciBoNT/PMP1-A | SIYAMGWYRQAPGKQRELVAAISSYGSTNYADSVKGRFTISRDNAKNTV | |
| | YLQMNSLKPEDTAVYYCNADIATMTAVGGFDYWGQGTQVTVSSEPKTP | |
| | KPQGGGGSGGGGSKLLQIRVFNYNDPIDGENIVELRYHNRSPVKAFQIVD | |
| | GIWIIPERYNFTNDTKKVPDDRALTILEDEVFAVRENDYLTTDVNEKNSF | |
| | LNNITKLFKRINSSNIGNQLLNYISTSVPYPVVSTNSIKARDYNTIKFDSID | |
| | GRRITKSANVLIYGPSMKNLLDKQTRAINGEEAKNGIGCLSDIIFSPNYLS | |
| | VQTVSSSRFVEDPASSLTHQLIHALHNLYGIQYPGEEKFKFGGFIDKLLGT | |
| | RECIDYEEVLTYGGKDSEIIRKKIDKSLYPDDFVNKYGEMYKRIKGSNPY | |
| | YPDEKKLKQSFLNRMNPFDQNGTFDTKEFKNHLMDLWFGLNESEFAKE | |
| | KKILVAKHFITKQINPKYTELTNDVYTEDKGFVNGQSIDNQNFKIIDDLIS | |
| | KKVKLCHKAIDGRSLGGSLVPRGSGGSAAAYNKTLDCIDVNKEDLYFIS | |
| | DKEGFENIDFSEPEIRYDSNVTTATTSSFTDHFLVNRTFNDSDRFPPVELE | |
| | YAIEPAEIVDNTIMPDIDQKSEISLDNLTTFHYLNAQKMDLGFDSSKEQLK | |
| | MVTSTEESLLDSKKVYTPFTRTAHSVNERISGIAESYLFYQWLKTVINDFT | |
| | DELNQKSNTDKVADISWIIPYVGPALNIGLDLSHGDFTKAFEDLGVSILFA | |
| | IAPEFATISLVALSIYENIEEDSQKEKVINKVENTLARRIEKWHQVYAFMV | |
| | AQWWGMVHTQIDTRIHQMYESLSHQIIAIKANMEYQLSHYKGPDNDKL | |
| | LLKDYIYEAEIALNTSANRAMKNIERFMIESSISYLKNNLIPSVVENLKKF | |
| | DADTKKNLDQFIDKNSSVLGSDLHILKSQVDLELNPTTKVAFNIQSIPDFD | |
| | INALIDRLGIQIINTSILNLRYESNHLIDLSRYASKINIGSKVNFDPIDKNQIQ | |
| | LFNLESSKIEVILKNAIVYNSMYENFSTSFWIRIPKYFNSISLNNEYTIINCM | |
| | ENNSGWKVSLNYGEIIWTLQDTQEIKQRVVFKYSQMINISDYINRWIFVTI | |
| | TNNRLNNSKIYINGRLIDQKPISNLGNIHASNNIMFKLDGCRDTHRYIWIK | |
| | YFNLFDKELNEKEIKDLYDNQSNSGILKDFWGDYLQYDKPYYMLNLYD | |
| | PNKYVDVNNVGIRGYMYLKGPRGSVMTTNIYLNSSLYRGTKFIIKKYAS | |
| | GNKDNIVRNNDRVYINVVVKNKEYRLATNASQAGVEKILSALEIPDVGN | |
| | LSQVVVMKSKNDQGITNKCKMNLQDNNGNDIGFIGFHQFNNIAKLVASN | |
| | WYNRQIERSSRTLGCSWEFIPVDDGWGERPLQ | |
| TrxA-thrombin | MSDKIIHLTDDSFDTDVLKADGAILVDFWAEWCGPCKMIAPILDEIADEYQG | 146 |
| site-anti-BoNT/B | KLTVAKLNIDQNPGTAPKYGIRGIPTILLFKNGEVAATKVGALSKGQLKEFLD | |
| VHH (J10)- | ANLAGGSLVPRGSGGSEFQVQLVESGGGLVQPGGSLRLSCAASGFPFHAY | |
| ciBoNT/PMP1-A | YMSWVRQAPGKGLEWVSHIGNGGIITRYADSVKGRFTISRDNAKNTLYL | |
| | QMTNLKPEDTALYYCTLGTRDDLGPERGQGTQVTVSSEPKTPKPQGGGG | |
| | SGGGGSKLLQIRVFNYNDPIDGENIVELRYHNRSPVKAFQIVDGIWIIPER | |
| | YNFTNDTKKVPDDRALTILEDEVFAVRENDYLTTDVNEKNSFLNNITKLF | |
| | KRINSSNIGNQLLNYISTSVPYPVVSTNSIKARDYNTIKFDSIDGRRITKSA | |
| | NVLIYGPSMKNLLDKQTRAINGEEAKNGIGCLSDIIFSPNYLSVQTVSSSR | |
| | FVEDPASSLTHQLIHALHNLYGIQYPGEEKFKFGGFIDKLLGTRECIDYEE | |
| | VLTYGGKDSEIIRKKIDKSLYPDDFVNKYGEMYKRIKGSNPYYPDEKKLK | |
| | QSFLNRMNPFDQNGTFDTKEFKNHLMDLWFGLNESEFAKEKKILVAKHF | |
| | ITKQINPKYTELTNDVYTEDKGFVNGQSIDNQNFKIIDDLISKKVKLCHKA | |
| | IDGRSLGGSLVPRGSGGSAAAYNKTLDCIDVNKEDLYFISDKEGFENIDFS | |
| | EPEIRYDSNVTTATTSSFTDHFLVNRTFNDSDRFPPVELEYAIEPAEIVDNT | |
| | IMPDIDQKSEISLDNLTTFHYLNAQKMDLGFDSSKEQLKMVTSIEESLLDS | |
| | KKVYTPFTRTAHSVNERISGIAESYLFYQWLKTVINDFTDELNQKSNTDK | |
| | VADISWIIPYVGPALNIGLDLSHGDFTKAFEDLGVSILFAIAPEFATISLVA | |
| | LSIYENIEEDSQKEKVINKVENTLARRIEKWHQVYAFMVAQWWGMVHT | |
| | QIDTRIHQMYESLSHQIIAIKANMEYQLSHYKGPDNDKLLLKDYIYEAEIA | |
| | LNTSANRAMKNIERFMIESSISYLKNNLIPSVVENLKKFDADTKKNLDQFI | |
| | DKNSSVLGSDLHILKSQVDLELNPTTKVAFNIQSIPDFDINALIDRLGIQIIN | |
| | TSILNLRYESNHLIDLSRYASKINIGSKVNFDPIDKNQIQLFNLESSKIEVIL | |
| | KNAIVYNSMYENFSTSFWIRIPKYFNSISLNNEYTIINCMENNSGWKVSLN | |
| | YGEIIWTLQDTQEIKQRVVFKYSQMINISDYINRWIFVTITNNRLNNSKIYI | |
| | NGRLIDQKPISNLGNIHASNNIMFKLDGCRDTHRYIWIKYFNLFDKELNE | |
| | KEIKDLYDNQSNSGILKDFWGDYLQYDKPYYMLNLYDPNKYVDVNNV | |
| | GIRGYMYLKGPRGSVMTTNIYLNSSLYRGTKFIIKKYASGNKDNIVRNND | |
| | RVYINVVVKNKEYRLATNASQAGVEKILSALEIPDVGNLSQVVVMKSKN | |
| | DQGITNKCKMNLQDNNGNDIGFIGFHQFNNIAKLVASNWYNRQIERSSR | |
| | TLGCSWEFIPVDDGWGERPLQ | |
| TrxA-thrombin | MSDKIIHLTDDSFDTDVLKADGAILVDFWAEWCGPCKMIAPILDEIADEYQG | 147 |
| site-anti-BoNT/B | KLTVAKLNIDQNPGTAPKYGIRGIPTILLFKNGEVAATKVGALSKGQLKEFLD | |
| VHH (A8)-anti- | ANLAGGSLVPRGSGGSEFMGTQVQLVESGGGLVQPGGSLRLSCAASGSIFSIY | |
| BoNT/B VHH | AMGWYRQAPGKQRELVAAISSYGSTNYADSVKGRFTISRDNAKNTVYLQMNSL | |
| (J10)- | KPEDTAVYYCNADIATMTAVGGFDYWGQGTQVTVSSEPKTPKPQGGGGSGG | |
| ciBoNT/PMP1-A | GGSASQVQLVESGGGLVQPGGSLRLSCAASGFPFHAYYMSWVRQAPGK | |
| | GLEWVSHIGNGGIITRYADSVKGRFTISRDNAKNTLYLQMTNLKPEDTAL | |
| | YYCTLGTRDDLGPERGQGTQVTVSSEPKTPKPQGGGGSGGGGSKLLQIR | |
| | VFNYNDPIDGENIVELRYHNRSPVKAFQIVDGIWIIPERYNFTNDTKKVPD | |
| | DRALTILEDEVFAVRENDYLTTDVNEKNSFLNNITKLFKRINSSNIGNQLL | |
| | NYISTSVPYPVVSTNSIKARDYNTIKFDSIDGRRITKSANVLIYGPSMKNLL | |
| | DKQTRAINGEEAKNGIGCLSDIIFSPNYLSVQTVSSSRFVEDPASSLTHQLI | |
| | HALHNLYGIQYPGEEKFKFGGFIDKLLGTRECIDYEEVLTYGGKDSEIIRK | |
| | KIDKSLYPDDFVNKYGEMYKRIKGSNPYYPDEKKLKQSFLNRMNPFDQN | |
| | GTFDTKEFKNHLMDLWFGLNESEFAKEKKILVAKHFITKQINPKYTELTN | |
| | DVYTEDKGFVNGQSIDNQNFKIIDDLISKKVKLCHKAIDGRSLGGSLVPR | |
| | GSGGSAAAYNKTLDCIDVNKEDLYFISDKEGFENIDFSEPEIRYDSNVTTA | |
| | TTSSFTDHFLVNRTFNDSDRFPPVELEYAIEPAEIVDNTIMPDIDQKSEISL | |
| | DNLTTFHYLNAQKMDLGFDSSKEQLKMVTSIEESLLDSKKVYTPFTRTA | |
| | HSVNERISGIAESYLFYQWLKTVINDFTDELNQKSNTDKVADISWIIPYVG | |
| | PALNIGLDLSHGDFTKAFEDLGVSILFAIAPEFATISLVALSIYENIEEDSQK | |
| | EKVINKVENTLARRIEKWHQVYAFMVAQWWGMVHTQIDTRIHQMYES | |
| | LSHQIIAIKANMEYQLSHYKGPDNDKLLLKDYIYEAEIALNTSANRAMKN | |
| | IERFMIESSISYLKNNLIPSVVENLKKFDADTKKNLDQFIDKNSSVLGSDL | |
| | HILKSQVDLELNPTTKVAFNIQSIPDFDINALIDRLGIQIINTSILNLRYESN | |
| | HLIDLSRYASKINIGSKVNFDPIDKNQIQLFNLESSKIEVILKNAIVYNSMY | |
| | ENFSTSFWIRIPKYFNSISLNNEYTIINCMENNSGWKVSLNYGEIIWTLQDT | |
| | QEIKQRVVFKYSQMINISDYINRWIFVTITNNRLNNSKIYINGRLIDQKPIS | |
| | NLGNIHASNNIMFKLDGCRDTHRYIWIKYFNLFDKELNEKEIKDLYDNQS | |
| | NSGILKDFWGDYLQYDKPYYMLNLYDPNKYVDVNNVGIRGYMYLKGP | |
| | RGSVMTTNIYLNSSLYRGTKFIIKKYASGNKDNIVRNNDRVYINVVVKN | |
| | KEYRLATNASQAGVEKILSALEIPDVGNLSQVVVMKSKNDQGITNKCKM | |
| | NLQDNNGNDIGFIGFHQFNNIAKLVASNWYNRQIERSSRTLGCSWEFIPV | |
| | DDGWGERPLQ | |
| Anti-BoNT/A | MGTQVQLVESGGGLVQPGGSLRLSCAASGSIFSIYAMGWYRQAPGKQRE | 148 |
| VHH (A8)- | LVAAISSYGSTNYADSVKGRFTISRDNAKNTVYLQMNSLKPEDTAVYYC | |
| ciBoNT/PMP1-A | NADIATMTAVGGFDYWGQGTQVTVSSEPKTPKPQGGGGSGGGGSKLLQ | |
| | IRVFNYNDPIDGENIVELRYHNRSPVKAFQIVDGIWIIPERYNFTNDTKKV | |
| | PDDRALTILEDEVFAVRENDYLTTDVNEKNSFLNNITKLFKRINSSNIGNQ | |
| | LLNYISTSVPYPVVSTNSIKARDYNTIKFDSIDGRRITKSANVLIYGPSMKN | |
| | LLDKQTRAINGEEAKNGIGCLSDIIFSPNYLSVQTVSSSRFVEDPASSLTHQ | |
| | LIHALHNLYGIQYPGEEKFKFGGFIDKLLGTRECIDYEEVLTYGGKDSEIIR | |
| | KKIDKSLYPDDFVNKYGEMYKRIKGSNPYYPDEKKLKQSFLNRMNPFDQ | |
| | NGTFDTKEFKNHLMDLWFGLNESEFAKEKKILVAKHFITKQINPKYTELT | |
| | NDVYTEDKGFVNGQSIDNQNFKIIDDLISKKVKLCHKAIDGRSLGGSLVP | |
| | RGSGGSAAAYNKTLDCIDVNKEDLYFISDKEGFENIDFSEPEIRYDSNVTT | |
| | ATTSSFTDHFLVNRTFNDSDRFPPVELEYAIEPAEIVDNTIMPDIDQKSEIS | |
| | LDNLTTFHYLNAQKMDLGFDSSKEQLKMVTSIEESLLDSKKVYTPFTRT | |
| | AHSVNERISGIAESYLFYQWLKTVINDFTDELNQKSNTDKVADISWIIPYV | |
| | GPALNIGLDLSHGDFTKAFEDLGVSILFAIAPEFATISLVALSIYENIEEDSQ | |
| | KEKVINKVENTLARRIEKWHQVYAFMVAQWWGMVHTQIDTRIHQMYE | |
| | SLSHQIIAIKANMEYQLSHYKGPDNDKLLLKDYIYEAEIALNTSANRAMK | |
| | NIERFMIESSISYLKNNLIPSVVENLKKFDADTKKNLDQFIDKNSSVLGSD | |
| | LHILKSQVDLELNPTTKVAFNIQSIPDFDINALIDRLGIQIINTSILNLRYESN | |
| | HLIDLSRYASKINIGSKVNFDPIDKNQIQLFNLESSKIEVILKNAIVYNSMY | |
| | ENFSTSFWIRIPKYFNSISLNNEYTIINCMENNSGWKVSLNYGEIIWTLQDT | |
| | QEIKQRVVFKYSQMINISDYINRWIFVTITNNRLNNSKIYINGRLIDQKPIS | |
| | NLGNIHASNNIMFKLDGCRDTHRYIWIKYFNLFDKELNEKEIKDLYDNQS | |
| | NSGILKDFWGDYLQYDKPYYMLNLYDPNKYVDVNNVGIRGYMYLKGP | |
| | RGSVMTTNIYLNSSLYRGTKFIIKKYASGNKDNIVRNNDRVYINVVVKN | |
| | KEYRLATNASQAGVEKILSALEIPDVGNLSQVVVMKSKNDQGITNKCKM | |
| | NLQDNNGNDIGFIGFHQFNNIAKLVASNWYNRQIERSSRTLGCSWEFIPV | |
| | DDGWGERPLQ | |
| Anti-BoNT/B VHH | QVQLVESGGGLVQPGGSLRLSCAASGFPFHAYYMSWVRQAPGKGLEWV | 149 |
| (J10)- | SHIGNGGIITRYADSVKGRFTISRDNAKNTLYLQMTNLKPEDTALYYCTL | |
| ciBoNT/PMP1-A | GTRDDLGPERGQGTQVTVSSEPKTPKPQGGGGSGGGGSKLLQIRVFNYN | |
| | DPIDGENIVELRYHNRSPVKAFQIVDGIWIIPERYNFTNDTKKVPDDRALT | |
| | ILEDEVFAVRENDYLTTDVNEKNSFLNNITKLFKRINSSNIGNQLLNYISTS | |
| | VPYPVVSTNSIKARDYNTIKFDSIDGRRITKSANVLIYGPSMKNLLDKQTR | |
| | AINGEEAKNGIGCLSDIIFSPNYLSVQTVSSSRFVEDPASSLTHQLIHALHN | |
| | LYGIQYPGEEKFKFGGFIDKLLGTRECIDYEEVLTYGGKDSEIIRKKIDKSL | |
| | YPDDFVNKYGEMYKRIKGSNPYYPDEKKLKQSFLNRMNPFDQNGTFDT | |
| | KEFKNHLMDLWFGLNESEFAKEKKILVAKHFITKQINPKYTELTNDVYTE | |
| | DKGFVNGQSIDNQNFKIIDDLISKKVKLCHKAIDGRSLGGSLVPRGSGGS | |
| | AAAYNKTLDCIDVNKEDLYFISDKEGFENIDFSEPEIRYDSNVTTATTSSF | |
| | TDHFLVNRTFNDSDRFPPVELEYAIEPAEIVDNTIMPDIDQKSEISLDNLTT | |
| | FHYLNAQKMDLGFDSSKEQLKMVTSIEESLLDSKKVYTPFTRTAHSVNE | |
| | RISGIAESYLFYQWLKTVINDFTDELNQKSNTDKVADISWIIPYVGPALNI | |
| | GLDLSHGDFTKAFEDLGVSILFAIAPEFATISLVALSIYENIEEDSQKEKVI | |
| | NKVENTLARRIEKWHQVYAFMVAQWWGMVHTQIDTRIHQMYESLSHQ | |
| | IIAIKANMEYQLSHYKGPDNDKLLLKDYIYEAEIALNTSANRAMKNIERF | |
| | MIESSISYLKNNLIPSVVENLKKFDADTKKNLDQFIDKNSSVLGSDLHILK | |
| | SQVDLELNPTTKVAFNIQSIPDFDINALIDRLGIQIINTSILNLRYESNHLIDL | |
| | SRYASKINIGSKVNFDPIDKNQIQLFNLESSKIEVILKNAIVYNSMYENFST | |
| | SFWIRIPKYFNSISLNNEYTIINCMENNSGWKVSLNYGEIIWTLQDTQEIK | |
| | QRVVFKYSQMINISDYINRWIFVTITNNRLNNSKIYINGRLIDQKPISNLGN | |
| | IHASNNIMFKLDGCRDTHRYIWIKYFNLFDKELNEKEIKDLYDNQSNSGI | |
| | LKDFWGDYLQYDKPYYMLNLYDPNKYVDVNNVGIRGYMYLKGPRGSV | |
| | MTTNIYLNSSLYRGTKFIIKKYASGNKDNIVRNNDRVYINVVVKNKEYRL | |
| | ATNASQAGVEKILSALEIPDVGNLSQVVVMKSKNDQGITNKCKMNLQD | |
| | NNGNDIGFIGFHQFNNIAKLVASNWYNRQIERSSRTLGCSWEFIPVDDGW | |
| | GERPLQ | |
| Anti-BoNT/B VHH | QVQLVESGGGLVQPGGSLRLSCAASGFPFHAYYMSWVRQAPGKGLEWV | 150 |
| (A8)-anti-BoNT/B | SHIGNGGIITRYADSVKGRFTISRDNAKNTLYLQMTNLKPEDTALYYCTL | |
| VHH (J10)- | GTRDDLGPERGQGTQVTVSSEPKTPKPQGGGGSGGGGSKLLQIRVFNYN | |
| ciBoNT/PMP1-A | DPIDGENIVELRYHNRSPVKAFQIVDGIWIIPERYNFTNDTKKVPDDRALT | |
| | ILEDEVFAVRENDYLTTDVNEKNSFLNNITKLFKRINSSNIGNQLLNYISTS | |
| | VPYPVVSTNSIKARDYNTIKFDSIDGRRITKSANVLIYGPSMKNLLDKQTR | |
| | AINGEEAKNGIGCLSDIIFSPNYLSVQTVSSSRFVEDPASSLTHQLIHALHN | |
| | LYGIQYPGEEKFKFGGFIDKLLGTRECIDYEEVLTYGGKDSEIIRKKIDKSL | |
| | YPDDFVNKYGEMYKRIKGSNPYYPDEKKLKQSFLNRMNPFDQNGTFDT | |
| | KEFKNHLMDLWFGLNESEFAKEKKILVAKHFITKQINPKYTELTNDVYTE | |
| | DKGFVNGQSIDNQNFKIIDDLISKKVKLCHKAIDGRSLGGSLVPRGSGGS | |
| | AAAYNKTLDCIDVNKEDLYFISDKEGFENIDFSEPEIRYDSNVTTATTSSF | |
| | TDHFLVNRTFNDSDRFPPVELEYAIEPAEIVDNTIMPDIDQKSEISLDNLTT | |
| | FHYLNAQKMDLGFDSSKEQLKMVTSIEESLLDSKKVYTPFTRTAHSVNE | |
| | RISGIAESYLFYQWLKTVINDFTDELNQKSNTDKVADISWIIPYVGPALNI | |
| | GLDLSHGDFTKAFEDLGVSILFAIAPEFATISLVALSIYENIEEDSQKEKVI | |
| | NKVENTLARRIEKWHQVYAFMVAQWWGMVHTQIDTRIHQMYESLSHQ | |
| | IIAIKANMEYQLSHYKGPDNDKLLLKDYIYEAEIALNTSANRAMKNIERF | |
| | MIESSISYLKNNLIPSVVENLKKFDADTKKNLDQFIDKNSSVLGSDLHILK | |
| | SQVDLELNPTTKVAFNIQSIPDFDINALIDRLGIQIINTSILNLRYESNHLIDL | |
| | SRYASKINIGSKVNFDPIDKNQIQLFNLESSKIEVILKNAIVYNSMYENFST | |
| | SFWIRIPKYFNSISLNNEYTIINCMENNSGWKVSLNYGEIIWTLQDTQEIK | |
| | QRVVFKYSQMINISDYINRWIFVTITNNRLNNSKIYINGRLIDQKPISNLGN | |
| | IHASNNIMFKLDGCRDTHRYIWIKYFNLFDKELNEKEIKDLYDNQSNSGI | |
| | LKDFWGDYLQYDKPYYMLNLYDPNKYVDVNNVGIRGYMYLKGPRGSV | |
| | MTTNIYLNSSLYRGTKFIIKKYASGNKDNIVRNNDRVYINVVVKNKEYRL | |
| | ATNASQAGVEKILSALEIPDVGNLSQVVVMKSKNDQGITNKCKMNLQD | |
| | NNGNDIGFIGFHQFNNIAKLVASNWYNRQIERSSRTLGCSWEFIPVDDGW | |
| | GERPLQ | |

### REFERENCES

1. Schiavo, G., Matteoli, M. & Montecucco, C. Neurotoxins affecting neuroexocytosis. Physiol Rev 80, 717-766 (2000).
2. Bade, S., Rummel, A., Reisinger, C., Karnath, T., Ahnert-Hilger, G., Bigalke, H. & Binz, T. Botulinum neurotoxin type D enables cytosolic delivery of enzymatically active cargo proteins to neurones via unfolded translocation intermediates. J Neurochem 91, 1461-1472 (2004).
3. Vazquez-Cintron, E. J., Beske, P. H., Tenezaca, L., Tran, B. Q., Oyler, J. M., Glotfelty, E. J., Angeles, C. A., Syngkon, A., Mukherjee, J., Kalb, S. R., Band, P. A., McNutt, P. M., Shoemaker, C. B. & Ichtchenko, K. Engineering Botulinum Neurotoxin Cl as a Molecular Vehicle for Intra-Neuronal Drug Delivery. Sci Rep 7, 42923 (2017).
4. Montal, M. Botulinum neurotoxin: a marvel of protein design. Annu Rev Biochem 79, 591-617 (2010).
5. Webb, R. P., Smith, T. J., Smith, L. A., Wright, P. M., Guernieri, R. L., Brown, J. L. & Skerry, J. C. Recombinant Botulinum Neurotoxin Hc Subunit (BoNT Hc) and Catalytically Inactive Clostridium botulinum Holoproteins (^{ci}BoNT HPs) as Vaccine Candidates for the Prevention of Botulism. Toxins (Basel) 9(2017).
6. Zhang, S., Lebreton, F., Mansfield, M. J., Miyashita, S. I., Zhang, J., Schwartzman, J. A., Tao, L., Masuyer, G., Martinez-Carranza, M., Stenmark, P., Gilmore, M. S., Doxey, A. C. & Dong, M. Identification of a Botulinum Neurotoxin-like Toxin in a Commensal Strain of Enterococcus faecium. Cell Host Microbe 23, 169-176 e166 (2018).
7. Zhang, S., Masuyer, G., Zhang, J., Shen, Y., Lundin, D., Henriksson, L., Miyashita, S. I., Martinez-Carranza, M., Dong, M. & Stenmark, P. Identification and characterization of a novel botulinum neurotoxin. Nat Commun 8, 14130 (2017).
8. Tremblay, J. M., Kuo, C. L., Abeijon, C., Sepulveda, J., Oyler, G., Hu, X., Jin, M. M. & Shoemaker, C. B. Camelid single domain antibodies (VHHs) as neuronal cell intrabody binding agents and inhibitors of Clostridium botulinum neurotoxin (BoNT) proteases. Toxicon 56, 990-998 (2010).
9. Peng, L., Tepp, W. H., Johnson, E. A. & Dong, M. Botulinum neurotoxin D uses synaptic vesicle protein SV2 and gangliosides as receptors. PLoS Pathog 7, e1002008 (2011).
10. M. Dong, G. Masuyer, P. Stenmark, Botulinum and Tetanus Neurotoxins. Annual review of biochemistry 88, 811-837 (2019).
11. M. Pirazzini, O. Rossetto, R. Eleopra, C. Montecucco, Botulinum Neurotoxins: Biology, Pharmacology, and Toxicology. Pharmacol Rev 69, 200-235 (2017).
12. O. Rossetto, M. Pirazzini, C. Montecucco, Botulinum neurotoxins: genetic, structural and mechanistic insights. Nat Rev Microbiol 12, 535-549 (2014).
13. M. Montal, Botulinum neurotoxin: a marvel of protein design. Annual review of biochemistry 79, 591-617 (2010).
14. A. Rummel, The long journey of botulinum neurotoxins into the synapse. Toxicon 107, 9-24 (2015).
15. G. Schiavo, M. Matteoli, C. Montecucco, Neurotoxins affecting neuroexocytosis. Physiological reviews 80, 717-766 (2000).
16. S. S. Arnon, R. Schechter, T. V. Inglesby, D. A. Henderson, J. G. Bartlett, M. S. Ascher, E. Eitzen, A. D. Fine, J. Hauer, M. Layton, S. Lillibridge, M. T. Osterholm, T. O'Toole, G. Parker, T. M. Perl, P. K. Russell, D. L. Swerdlow, K. Tonat, Botulinum toxin as a biological weapon: medical and public health management. Jama 285, 1059-1070 (2001).
17. C. L. Hatheway, in Clostridial neurotoxins, C. Montecucco, Ed. (Springer-Verlag, Berlin, 1995), vol. 195, pp. 55-75.
18. J. Sobel, Botulism. Clin Infect Dis 41, 1167-1173 (2005).
19. J. E. Keller, E. A. Neale, G. Oyler, M. Adler, Persistence of botulinum neurotoxin action in cultured spinal cord cells. FEBS Lett 456, 137-142 (1999).
20. R. C. Whitemarsh, W. H. Tepp, E. A. Johnson, S. Pellett, Persistence of botulinum neurotoxin a subtypes 1-5 in primary rat spinal cord cells. PLoS ONE 9, e90252 (2014).
21. Y. C. Tsai, A. Kotiya, E. Kiris, M. Yang, S. Bavari, L. Tessarollo, G. A. Oyler, A. M. Weissman, Deubiquitinating enzyme VCIP135 dictates the duration of botulinum neurotoxin type A intoxication. Proceedings of the National Academy of Sciences of the United States of America 114, E5158-E5166 (2017).
22. S. Pellett, W. H. Tepp, R. C. Whitemarsh, M. Bradshaw, E. A. Johnson, In vivo onset and duration of action varies for botulinum neurotoxin A subtypes 1-5. Toxicon 107, 37-42 (2015).
23. P. G. Foran, N. Mohammed, G. O. Lisk, S. Nagwaney, G. W. Lawrence, E. Johnson, L. Smith, K. R. Aoki, J. O. Dolly, Evaluation of the therapeutic usefulness of botulinum neurotoxin B, Cl, E, and F compared with the long lasting type A. Basis for distinct durations of inhibition of exocytosis in central neurons. The Journal of biological chemistry 278, 1363-1371 (2003).
24. A. Nowakowski, C. Wang, D. B. Powers, P. Amersdorfer, T. J. Smith, V. A. Montgomery, R. Sheridan, R. Blake, L. A. Smith, J. D. Marks, Potent neutralization of botulinum neurotoxin by recombinant oligoclonal antibody. Proceedings of the National Academy of Sciences of the United States of America 99, 11346-11350 (2002).
25. S. S. Arnon, R. Schechter, S. E. Maslanka, N. P. Jewell, C. L. Hatheway, Human botulism immune globulin for the treatment of infant botulism. The New England journal of medicine 354, 462-471 (2006).
26. S. Kodihalli, A. Emanuel, T. Takla, Y. Hua, C. Hobbs, R. LeClaire, D. C. O'Donnell, Therapeutic efficacy of equine botulism antitoxin in Rhesus macaques. PLoS ONE 12, e0186892 (2017).
27. P. A. Yu, N. H. Lin, B. E. Mahon, J. Sobel, Y. Yu, R. K. Mody, W. Gu, J. Clements, H. J. Kim, A. K. Rao, Safety and Improved Clinical Outcomes in Patients Treated With New Equine-Derived Heptavalent Botulinum Antitoxin. Clin Infect Dis 66, S57-S64 (2017).
28. E. A. Johnson, Clostridial toxins as therapeutic agents: benefits of nature's most toxic proteins. Annu Rev Microbiol 53, 551-575 (1999).
29. T. C. Sudhof, J. E. Rothman, Membrane fusion: grappling with SNARE and SM proteins. Science (New York, N.Y. 323, 474-477 (2009).
30. R. Jahn, R. H. Scheller, SNAREs—engines for membrane fusion. Nature reviews 7, 631-643 (2006).
31. S. Bade, A. Rummel, C. Reisinger, T. Karnath, G. Ahnert-Hilger, H. Bigalke, T. Binz, Botulinum neurotoxin type D enables cytosolic delivery of enzymatically active cargo proteins to neurones via unfolded translocation intermediates. Journal of neurochemistry 91, 1461-1472 (2004).
32. M. C. Goodnough, G. Oyler, P. S. Fishman, E. A. Johnson, E. A. Neale, J. E. Keller, W. H. Tepp, M. Clark, S. Hartz, M. Adler, Development of a delivery vehicle for intracellular transport of botulinum neurotoxin antagonists. FEBS Lett 513, 163-168 (2002).
33. M. Ho, L. H. Chang, M. Pires-Alves, B. Thyagarajan, J. E. Bloom, Z. Gu, K. K. Aberle, S. A. Teymorian, Y. Bannai, S. C. Johnson, J. J. McArdle, B. A. Wilson, Recombinant botulinum neurotoxin A heavy chain-based delivery vehicles for neuronal cell targeting. Protein Eng Des Sel 24, 247-253 (2011).
34. P. A. Band, S. Blais, T. A. Neubert, T. J. Cardozo, K. Ichtchenko, Recombinant derivatives of botulinum neurotoxin A engineered for trafficking studies and neuronal delivery. Protein expression and purification 71, 62-73 (2010).
35. G. Schiavo, B. Poulain, O. Rossetto, F. Benfenati, L. Tauc, C. Montecucco, Tetanus toxin is a zinc protein and its inhibition of neurotransmitter release and protease activity depend on zinc. The EMBO journal 11, 3577-3583 (1992).
36. T. Binz, S. Bade, A. Rummel, A. Kollewe, J. Alves, Arg(362) and Tyr(365) of the botulinum neurotoxin type a light chain are involved in transition state stabilization. Biochemistry 41, 1717-1723 (2002).
37. E. J. Vazquez-Cintron, P. H. Beske, L. Tenezaca, B. Q. Tran, J. M. Oyler, E. J. Glotfelty, C. A. Angeles, A. Syngkon, J. Mukherjee, S. R. Kalb, P. A. Band, P. M. McNutt, C. B. Shoemaker, K. Ichtchenko, Engineering Botulinum Neurotoxin Cl as a Molecular Vehicle for Intra-Neuronal Drug Delivery. Sci Rep 7, 42923 (2017).
38. S. Pellett, W. H. Tepp, L. H. Stanker, P. A. Band, E. A. Johnson, K. Ichtchenko, Neuronal targeting, internalization, and biological activity of a recombinant atoxic derivative of botulinum neurotoxin A. Biochemical and biophysical research communications 405, 673-677 (2011).
39. R. P. Webb, T. J. Smith, L. A. Smith, P. M. Wright, R. L. Guernieri, J. L. Brown, J. C. Skerry, Recombinant Botulinum Neurotoxin Hc Subunit (BoNT Hc) and Catalytically Inactive Clostridium botulinum Holoproteins (^{ci}BoNT HPs) as Vaccine Candidates for the Prevention of Botulism. Toxins (Basel) 9, (2017).
40. S. Zhang, G. Masuyer, J. Zhang, Y. Shen, D. Lundin, L. Henriksson, S. I. Miyashita, M. Martinez-Carranza, M. Dong, P. Stenmark, Identification and characterization of a novel botulinum neurotoxin. Nat Commun 8, 14130 (2017).
41. C. L. Kuo, G. A. Oyler, C. B. Shoemaker, Accelerated neuronal cell recovery from Botulinum neurotoxin intoxication by targeted ubiquitination. PLoS ONE 6, e20352 (2011).
42. J. M. Tremblay, C. L. Kuo, C. Abeijon, J. Sepulveda, G. Oyler, X. Hu, M. M. Jin, C. B. Shoemaker, Camelid single domain antibodies (VHHs) as neuronal cell intrabody binding agents and inhibitors of Clostridium botulinum neurotoxin (BoNT) proteases. Toxicon 56, 990-998 (2010).
43. M. Pirazzini, D. Azarnia Tehran, G. Zanetti, A. Megighian, M. Scorzeto, S. Fillo, C. C. Shone, T. Binz, O. Rossetto, F. Lista, C. Montecucco, Thioredoxin and its reductase are present on synaptic vesicles, and their inhibition prevents the paralysis induced by botulinum neurotoxins. Cell Rep 8, 1870-1878 (2014).
44. M. W. Popp, J. M. Antos, G. M. Grotenbreg, E. Spooner, H. L. Ploegh, Sortagging: a versatile method for protein labeling. Nat Chem Biol 3, 707-708 (2007).
45. J. Blasi, E. R. Chapman, E. Link, T. Binz, S. Yamasaki, P. De Camilli, T. C. Sudhof, H. Niemann, R. Jahn, Botulinum neurotoxin A selectively cleaves the synaptic protein SNAP-25. Nature 365, 160-163 (1993).
46. G. Schiavo, A. Santucci, B. R. Dasgupta, P. P. Mehta, J. Jontes, F. Benfenati, M. C. Wilson, C. Montecucco, Botulinum neurotoxins serotypes A and E cleave SNAP-25 at distinct COOH-terminal peptide bonds. FEBS Lett 335, 99-103 (1993).
47. M. Dong, F. Yeh, W. H. Tepp, C. Dean, E. A. Johnson, R. Janz, E. R. Chapman, SV2 is the protein receptor for botulinum neurotoxin A. Science (New York, N.Y. 312, 592-596 (2006).
48. S. Mahrhold, A. Rummel, H. Bigalke, B. Davletov, T. Binz, The synaptic vesicle protein 2C mediates the uptake of botulinum neurotoxin A into phrenic nerves. FEBS Lett 580, 2011-2014 (2006).
49. L. Peng, H. Liu, H. Ruan, W. H. Tepp, W. H. Stoothoff, R. H. Brown, E. A. Johnson, W. D. Yao, S. C. Zhang, M. Dong, Cytotoxicity of botulinum neurotoxins reveals a direct role of syntaxin 1 and SNAP-25 in neuron survival. Nat Commun 4, 1472 (2013).
50. S. Pellett, W. H. Tepp, J. M. Scherf, E. A. Johnson, Botulinum Neurotoxins Can Enter Cultured Neurons Independent of Synaptic Vesicle Recycling. PLoS ONE 10, e0133737 (2015).
51. K. R. Aoki, A comparison of the safety margins of botulinum neurotoxin serotypes A, B, and F in mice. Toxicon 39, 1815-1820 (2001).
52. S. Zhang, F. Lebreton, M. J. Mansfield, S. I. Miyashita, J. Zhang, J. A. Schwartzman, L. Tao, G. Masuyer, M. Martinez-Carranza, P. Stenmark, M. S. Gilmore, A. C. Doxey, M. Dong, Identification of a Botulinum Neurotoxin-like Toxin in a Commensal Strain of Enterococcus faecium. Cell Host Microbe 23, 169-176 e166 (2018).
53. J. Brunt, A. T. Carter, S. C. Stringer, M. W. Peck, Identification of a novel botulinum neurotoxin gene cluster in Enterococcus. FEBS Lett 592, 310-317 (2018).
54. E. Contreras, G. Masuyer, N. Qureshi, S. Chawla, H. S. Dhillon, H. L. Lee, J. Chen, P. Stenmark, S. S. Gill, A neurotoxin that specifically targets Anopheles mosquitoes. Nat Commun 10, 2869 (2019).
55. Z. Yang, D. Schmidt, W. Liu, S. Li, L. Shi, J. Sheng, K. Chen, H. Yu, J. M. Tremblay, X. Chen, K. H. Piepenbrink, E. J. Sundberg, C. P. Kelly, G. Bai, C. B. Shoemaker, H. Feng, A novel multivalent, single-domain antibody targeting TcdA and TcdB prevents fulminant Clostridium difficile infection in mice. J Infect Dis 210, 964-972 (2014).
56. A. R. Schneekloth, M. Pucheault, H. S. Tae, C. M. Crews, Targeted intracellular protein degradation induced by a small molecule: En route to chemical proteomics. Bioorg Med Chem Lett 18, 5904-5908 (2008).
57. S. L. Paiva, C. M. Crews, Targeted protein degradation: elements of PROTAC design. Curr Opin Chem Biol 50, 111-119 (2019).
58. E. Caussinus, O. Kanca, M. Affolter, Fluorescent fusion protein knockout mediated by anti-GFP nanobody. Nature structural & molecular biology 19, 117-121 (2011).
59. N. Yamaguchi, T. Colak-Champollion, H. Knaut, zGrad is a nanobody-based degron system that inactivates proteins in zebrafish. Elife 8, (2019).
60. S. Nagarkar-Jaiswal, P. T. Lee, M. E. Campbell, K. Chen, S. Anguiano-Zarate, M. C. Gutierrez, T. Busby, W. W. Lin, Y. He, K. L. Schulze, B. W. Booth, M. Evans-Holm, K. J. Venken, R. W. Levis, A. C. Spradling, R. A. Hoskins, H. J. Bellen, A library of MiMICs allows tagging of genes and reversible, spatial and temporal knockdown of proteins in Drosophila. Elife 4, (2015).
61. S. Wang, N. H. Tang, P. Lara-Gonzalez, Z. Zhao, D. K. Cheerambathur, B. Prevo, A. D. Chisholm, A. Desai, K. Oegema, A toolkit for GFP-mediated tissue-specific protein degradation in C. elegans. Development 144, 2694-2701 (2017).
62. Y. J. Shin, S. K. Park, Y. J. Jung, Y. N. Kim, K. S. Kim, 0. K. Park, S. H. Kwon, S. H. Jeon, A. Trinh le, S. E. Fraser, Y. Kee, B. J. Hwang, Nanobody-targeted E3-ubiquitin ligase complex degrades nuclear proteins. Sci Rep 5, 14269 (2015).
63. E. Contreras, G. Masuyer, N. Qureshi, S. Chawla, H. Dhillon, H. Lee, J. Chen, P. Stenmark, S. Gill, A neurotoxin that specifically targets Anopheles mosquitoes. Nat Commun 10 (1), 2869 (2019).

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A catalytically inactive neurotoxin (BoNT) from Clostridium botulinum, serotype X (BoNT/X) comprising an inactive protease domain and a translocation domain.

2. The catalytically inactive BoNT/X of claim 1, wherein the inactive protease domain comprises one or more substitution mutation(s) in a position corresponding to R360, Y363, H227, E228, or H231 in SEQ ID NO: 1.

3. The catalytically inactive BoNT/X of claim 2, wherein the inactive protease domain comprises amino acid substitutions corresponding to E228Q, R360A, and Y363F in SEQ ID NO: 1.

4. The catalytically inactive BoNT/X of any one of claims 1-3, comprising the amino acid sequence of any one of SEQ ID NO: 3 or SEQ ID NO: 21.

5. A catalytically inactive neurotoxin from Enterococcus faecium (BoNT/En) comprising an inactive protease domain and a translocation domain.

6. The catalytically inactive BoNT/En of claim 5, wherein the inactive protease domain comprises one or more substitution mutation(s) in a position corresponding to H225, E226, H229, R364, or Y367 in SEQ ID NO: 2.

7. The catalytically inactive BoNT/En of claim 6, wherein the inactive protease domain comprises amino acid substitutions corresponding to E226Q, R364A, and Y367F in SEQ ID NO: 2.

8. The catalytically inactive BoNT/En of any one of claims 5-7, comprising the amino acid sequence of any one of SEQ ID NO: 4 or SEQ ID NO: 22.

9. A catalytically inactive neurotoxin from Paraclostridium bifermentans (BoNT/PMP1) comprising an inactive protease domain and a translocation domain.

10. The catalytically inactive BoNT/PMP1 of claim 9, wherein the inactive protease domain comprises one or more substitution mutation(s) in a position corresponding to H208, E209, H212, R344, or Y347 in SEQ ID NO: 85.

11. The catalytically inactive BoNT/PMP1 of claim 10, wherein the inactive protease domain comprises amino acid substitutions corresponding to E209Q, R344A, and Y347F in SEQ ID NO: 85.

12. The catalytically inactive BoNT of any one of claims 9-11, comprising the amino acid sequence of any one of SEQ ID NO: 86 or SEQ ID NO: 95.

13. A chimeric Clostridium botulinum neurotoxin (BoNT) comprising:
(a) a light chain comprising an inactive protease domain,
(b) a heavy chain comprising:
(i) a translocation domain, and
(ii) a receptor binding domain,
wherein (a) and (b)(i) are from a neurotoxin in Clostridium botulinum, serotype X, Enterococcus faecium or Paraclostridium bifermentans, and wherein (b)(ii) is from a BoNT in Clostridium botulinum, serotype A, B, C, D, E, F, G or H.

14. The chimeric BoNT of claim 13, comprising a modified linker between (a) and (b)(i).

15. The chimeric BoNT of claim 14, wherein the modified linker comprises a protease cleavage site.

16. The chimeric BoNT of any one of claims 13-15, wherein (a) and (b)(i) are from a neurotoxin in Clostridium botulinum, serotype X.

17. The chimeric BoNT of claim 16, wherein the inactive protease domain comprises one or more substitution mutation(s) in a position corresponding to R360, Y363, H227, E228, or H231 in SEQ ID NO: 1.

18. The chimeric BoNT of claim 17, wherein the inactive protease domain comprises amino acid substitutions corresponding to E228Q, R360A, and Y363F in SEQ ID NO: 1.

19. The chimeric BoNT of any one of claims 16-18, wherein the b(ii) is from BoNT in Clostridium botulinum, serotype A (BoNT/A).

20. The chimeric BoNT of any one of claims 16-18, wherein b(ii) is from BoNT in Clostridium botulinum, serotype B (BoNT/B).

21. The chimeric BoNT of any one of claims 16-18, wherein b(ii) is from BoNT in Clostridium botulinum, serotype C (BoNT/C).

22. The chimeric BoNT of any one of claims 16-18, wherein b(ii) is from BoNT in Clostridium botulinum, serotype D (BoNT/D).

23. The chimeric BoNT of any one of claims 16-18, wherein b(ii) is from BoNT in Clostridium botulinum, serotype E (BoNT/E).

24. The chimeric BoNT of any one of claims 16-18, wherein b(ii) is from BoNT in Clostridium botulinum, serotype F (BoNT/F).

25. The chimeric BoNT of any one of claims 16-18, wherein b(ii) is from BoNT in Clostridium botulinum, serotype G (BoNT/G).

26. The chimeric BoNT of any one of claims 16-18, wherein b(ii) is from BoNT in Clostridium botulinum, serotype H (BoNT/H).

27. The chimeric BoNT of any one of claims 16-26, wherein the chimeric BoNT comprises an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 5-12 and 23-30, and comprises amino acid substitutions corresponding to E228Q, R360A, and Y363F in SEQ ID NO: 1.

28. The chimeric BoNT of claim 27, wherein the chimeric BoNT comprises the amino acid sequence of any one of SEQ ID NOs: 5-12 and 23-30.

29. The chimeric BoNT of claim 28, wherein the chimeric BoNT consists of the amino acid sequence of any one of SEQ ID NOs: 5-12 and 23-30.

30. The chimeric BoNT of any one of claims 13-15, wherein (a) and (b)(i) are from a neurotoxin in Enterococcus faecium.

31. The chimeric BoNT of claim 30, wherein the inactive protease domain comprises one or more substitution mutation(s) in a position corresponding to H225, E226, H229, R364, or Y367 in SEQ ID NO: 2.

32. The chimeric BoNT of claim 31, wherein the inactive protease domain comprises amino acid substitutions corresponding to E226Q, R364A, or Y367F in SEQ ID NO: 2.

33. The chimeric BoNT of any one of claims 30-32, wherein b(ii) is from BoNT in Clostridium botulinum, serotype A (BoNT/A).

34. The chimeric BoNT of any one of claims 30-32, wherein b(ii) is from BoNT in Clostridium botulinum, serotype B (BoNT/B).

35. The chimeric BoNT of any one of claims 30-32, wherein b(ii) is from BoNT in Clostridium botulinum, serotype C (BoNT/C).

36. The chimeric BoNT of any one of claims 30-32, wherein b(ii) is from BoNT in Clostridium botulinum, serotype D (BoNT/D).

37. The chimeric BoNT of any one of claims 30-32, wherein b(ii) is from BoNT in Clostridium botulinum, serotype E (BoNT/E).

38. The chimeric BoNT of any one of claims 30-32, wherein b(ii) is from BoNT in Clostridium botulinum, serotype F (BoNT/F).

39. The chimeric BoNT of any one of claims 30-32, wherein b(ii) is from BoNT in Clostridium botulinum, serotype G (BoNT/G).

40. The chimeric BoNT of any one of claims 30-32, wherein b(ii) is from BoNT in Clostridium botulinum, serotype H (BoNT/H).

41. The chimeric BoNT of any one of claims 30-40, wherein the chimeric BoNT comprises an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 13-20 and 31-38, and comprises amino acid substitutions corresponding to E226Q, R364A, and Y367F in SEQ ID NO: 2.

42. The chimeric BoNT of claim 41, wherein the chimeric BoNT comprises the amino acid sequence of any one of SEQ ID NOs: 13-20 and 31-38.

43. The chimeric BoNT of claim 42, wherein the chimeric BoNT consists of the amino acid sequence of any one of SEQ ID NOs: 13-20 and 31-38.

44. The chimeric BoNT of any one of claims 13-15, wherein (a) and (b)(i) are from a neurotoxin in Paraclostridium bifermentans.

45. The chimeric BoNT of claim 44, wherein the inactive protease domain comprises one or more substitution mutation(s) in a position corresponding to H208, E209, H212, R344, or Y347 in SEQ ID NO: 85.

46. The chimeric BoNT of claim 45, wherein the inactive protease domain comprises amino acid substitutions corresponding to E209Q, R344A, and Y347F in SEQ ID NO: 85.

47. The chimeric BoNT of any one of claims 44-46, wherein b(ii) is from BoNT in Clostridium botulinum, serotype A (BoNT/A).

48. The chimeric BoNT of any one of claims 44-46, wherein b(ii) is from BoNT in Clostridium botulinum, serotype B (BoNT/B).

49. The chimeric BoNT of any one of claims 44-46, wherein b(ii) is from BoNT in Clostridium botulinum, serotype C (BoNT/C).

50. The chimeric BoNT of any one of claims 44-46, wherein b(ii) is from BoNT in Clostridium botulinum, serotype D (BoNT/D).

51. The chimeric BoNT of any one of claims 44-46, wherein b(ii) is from BoNT in Clostridium botulinum, serotype E (BoNT/E).

52. The chimeric BoNT of any one of claims 44-46, wherein b(ii) is from BoNT in Clostridium botulinum, serotype F (BoNT/F).

53. The chimeric BoNT of any one of claims 44-46, wherein b(ii) is from BoNT in Clostridium botulinum, serotype G (BoNT/G).

54. The chimeric BoNT of any one of claims 44-46, wherein b(ii) is from BoNT in Clostridium botulinum, serotype H (BoNT/H).

55. The chimeric BoNT of any one of claims 44-54, wherein the chimeric BoNT comprises an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 87-94 and 96-103, and comprises amino acid substitutions corresponding to E209Q, R344A, and Y347F in SEQ ID NO: 85.

56. The chimeric BoNT of claim 41, wherein the chimeric BoNT comprises the amino acid sequence of any one of SEQ ID NOs: 87-94 and 96-103.

57. The chimeric BoNT of claim 42, wherein the chimeric BoNT consists of the amino acid sequence of any one of SEQ ID NOs: 87-94 and 96-103.

58. The chimeric BoNT of any one of claims 13-26, 30-40, and 44-54, wherein the light chain and the heavy chain are linked by a di-sulfide bond.

59. A nucleic acid encoding the catalytically inactive BoNT/X of any one of claims 1-4, the catalytically inactive BoNT/EN of any one of claims 5-7, the catalytically inactive BoNT/PMP1 of claims 9-12, or chimeric BoNT of any one of claims 13-57.

60. A vector comprising the nucleic acid of claim 59.

61. A cell comprising the catalytically inactive BoNT/X of any one of claims 1-4, the catalytically inactive BoNT/EN of any one of claims 5-7, the catalytically inactive BoNT/PMP1 of claims 9-12, chimeric BoNT of any one of claims 13-57, the nucleic acid of claim 59, or the vector of claim 60.

62. A composition comprising the catalytically inactive BoNT/X of any one of claims 1-4, the catalytically inactive BoNT/EN of any one of claims 5-7, the catalytically inactive BoNT/PMP1 of claims 9-12, or the chimeric BoNT of any one of claims 13-57.

63. The composition of claim 62, wherein the composition is a pharmaceutical composition.

64. The composition of claim 63, further comprising a pharmaceutically acceptable carrier.

65. Use of the catalytically inactive BoNT/X of any one of claims 1-4, the catalytically inactive BoNT/EN of any one of claims 5-7, the catalytically inactive BoNT/PMP1 of claims 9-12, or the chimeric BoNT of any one of claims 13-57 as a delivery vehicle.

66. A complex comprising the catalytically inactive BoNT/X of any one of claims 1-4, the catalytically inactive BoNT/EN of any one of claims 5-7, the catalytically inactive BoNT/PMP1 of claims 9-12, or the chimeric BoNT of any one of claims 13-57 associated with an agent.

67. The complex of claim 66, wherein the agent is associate with the catalytically inactive BoNT/X, the catalytically inactive BoNT/En, the catalytically inactive BoNT/PMP1, or the chimeric BoNT non-covalently.

68. The complex of claim 66, wherein the agent is fused to the catalytically inactive BoNT/X, the catalytically inactive BoNT/EN, the catalytically inactive BoNT/PMP1, or the chimeric BoNT via a covalent bond.

69. The complex of claim 68, wherein the agent is associated with the light chain or the heavy chain of the catalytically inactive BoNT/X, the catalytically inactive BoNT/En, the catalytically inactive BoNT/PMP1, or the chimeric BoNT.

70. A complex comprising a chimeric BoNT associated with an agent, wherein the BoNT comprises:
(a) a light chain comprising an inactive protease domain,
(b) a heavy chain comprising:
(i) a translocation domain, and
(ii) a receptor binding domain,
wherein (a) and (b)(i) are from a neurotoxin in Clostridium botulinum, serotype X, and wherein (b)(ii) is from a BoNT in Clostridium botulinum, serotype A, B, C, D, E, F, G, or H,
and wherein the light chain and the heavy chain are linked via a disulfide bond.

71. A complex comprising a chimeric BoNT associated with an agent, wherein the BoNT comprises:
(a) a light chain comprising an inactive protease domain,
(b) a heavy chain comprising:
(i) a translocation domain, and
(ii) a receptor binding domain,
wherein (a) and (b)(i) are from a neurotoxin in Enterococcus faecium, and wherein (b)(ii) is from a BoNT in Clostridium botulinum, serotype A, B, C, D, E, F, G, or H,
and wherein the light chain and the heavy chain are linked via a disulfide bond.

72. A complex comprising a chimeric BoNT associated with an agent, wherein the BoNT comprises:
(a) a light chain comprising an inactive protease domain,
(b) a heavy chain comprising:
(i) a translocation domain, and
(ii) a receptor binding domain,
wherein (a) and (b)(i) are from a neurotoxin in Enterococcus faecium, and wherein (b)(ii) is from a BoNT in Clostridium botulinum, serotype A, B, C, D, E, F, G, or H,
and wherein the light chain and the heavy chain are linked via a disulfide bond.

73. The complex of any one of claims 66-72, wherein (b)(ii) is from a BoNT in Clostridium botulinum, serotype A.

74. The complex of any one of claims 70-72, wherein the agent is fused to the N-terminus of the light chain.

75. The complex of any one of claims 66-74, wherein the agent is a nucleic acid, a peptide or protein, or a small molecule.

76. The complex of any one of claims 66-75, wherein the agent is a diagnostic agent.

77. The complex of any one of claims 66-75, wherein the agent is a therapeutic agent.

78. The complex of claim 77, wherein the therapeutic agent is an antibody.

79. The complex of claim 78, wherein the antibody is a VHH.

80. The complex of claim 78 or claim 79, wherein the antibody is an antibody against a BoNT light chain.

81. The complex of claim 80, wherein the antibody comprises the amino acid sequence of any one of SEQ ID NO: 57, SEQ ID NO: 58, SEQ ID NO: 67, SEQ ID NO: 113, and SEQ ID NO: 114.

82. The complex of claim 77, wherein the therapeutic agent is a fusion protein comprising two VHHs.

83. The complex of claim 82, wherein the fusion protein comprises a VHH against BoNT/A light chain fused to a VHH against BoNT/B light chain.

84. The complex of any one of claims 66-83, wherein the complex comprises an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the amino acid sequence of any one of SEQ ID NOs: 65, 66, 71, 75, 76, 119, 128, 129, 133, 134, 137, 138, and 142-150.

85. The complex of claim 84, wherein the complex comprises an amino acid sequence of any one of SEQ ID NOs: 65, 66, 71, 75, 76, 119, 128, 129, 133, 134, 137, 138, and 142-150.

86. A composition comprising the complex of any one of claims 66-85.

87. The composition of claim 86, wherein the composition is a pharmaceutical composition.

88. The composition of claim 87, further comprising a pharmaceutically acceptable carrier.

89. Use of the complex of any one of claims 66-85 or the composition of any one of claims 86-88 for delivering the agent to a cell.

90. Use of the complex of any one of claims 66-85 or the composition of any one of claims 86-88 in treating or diagnosing a disease.

91. A method of delivering an agent to a cell, comprising contacting the cell with the complex of any one of claims 66-85 or the composition of any one of claims 86-88.

92. The method of claim 91, wherein the cell is in vitro.

93. The method of claim 91, wherein the cell is in vivo.

94. The method of claim 91, wherein the cell is ex vivo.

95. The method of any one of claims 91-94, wherein the cell is a neuron.

96. A method of diagnosing a disease, comprising administering to a subject in need thereof an effective amount of the complex of any one of 66-85 or the composition of any one of claims 86-88, wherein the agent is a diagnostic agent.

97. A method of treating a disease, comprising administering to a subject in need thereof an effective amount of the complex of any one of 66-85 or the composition of any one of claims 86-88, wherein the agent is a therapeutic agent.

98. The method of claim 97, wherein the disease is botulism.

99. The method of claim 98, wherein the subject has previously been administered a BoNT or been in contact with a BoNT.

100. The method of claim 99, wherein the therapeutic agent neutralizes the BoNT.

101. The method of any one of claims 96-100, wherein the complex is administered by injection.

102. The method of any one of claims 96-100, wherein the subject is human.

103. The method of any one of claims 96-100, wherein the subject is a rodent.

104. The method of claim 103, wherein the rodent is a mouse or a rat.
